(19) United States
(12) Patent Application Publication
(10) Pub. No.: US 2024/0023434 A1
(43) Pub. Date: Jan. 18, 2024

(54) ORGANIC OPTOELECTRONIC DEVICE AND DISPLAY DEVICE

(71) Applicant: SAMSUNG SDI CO., LTD., Yongin-si (KR)

(72) Inventors: Jongwoo WON, Suwon-si (KR); Youngmook LIM, Suwon-si (KR); Youngkyoung JO, Suwon-si (KR); Sunwoong SHIN, Suwon-si (KR); Sunghyun JUNG, Suwon-si (KR); Junmo PARK, Suwon-si (KR); Jaehoon KIM, Suwon-si (KR); Hokuk JUNG, Suwon-si (KR); Hayun SONG, Suwon-si (KR); ChanWoo KIM, Suwon-si (KR)

(21) Appl. No.: 18/222,598

(22) Filed: Jul. 17, 2023

(30) Foreign Application Priority Data
Jul. 15, 2022 (KR) 10-2022-0087763
Jul. 13, 2023 (KR) 10-2023-0091247

(51) Int. Cl. H10K 85/60; C07D 403/14; C09K 11/06; C07D 209/86; C07C 211/61

(52) U.S. Cl. CPC H10K 85/6572; C07D 403/14; C09K 11/06; H10K 85/654; C07D 209/86; C07C 211/61; H10K 85/633; H10K 50/156

(57) ABSTRACT

An organic optoelectronic device and a display device, the organic optoelectronic device includes an anode and a cathode facing each other, a light emitting layer between the anode and the cathode, a hole transport layer between the anode and the light emitting layer, and a hole transport auxiliary layer between the light emitting layer and the hole transport layer, wherein the light emitting layer includes a first compound represented by Chemical Formula 1, and a second compound represented by Chemical Formula 2 or a combination of Chemical Formula 3 and Chemical Formula 4, the hole transport auxiliary layer includes a third compound represented by Chemical Formula 5.

### CROSS-REFERENCE TO RELATED APPLICATION

This application claims priority to and the benefit of Korean Patent Application No. 10-2022-0087763 filed in the Korean Intellectual Property Office on Jul. 15, 2022 and Korean Patent Application No. 10-2023-0091247 filed in the Korean Intellectual Property Office on Jul. 13, 2023, the entire contents of which are incorporated herein by reference.

### BACKGROUND

### 1. Field

Embodiments relate to an organic optoelectronic device and a display device.

### 2. Description of the Related Art

An organic optoelectronic device (e.g., organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device may include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### SUMMARY

The embodiments may be realized by providing an organic optoelectronic device including an anode and a cathode facing each other, a light emitting layer between the anode and the cathode, a hole transport layer between the anode and the light emitting layer, and a hole transport auxiliary layer between the light emitting layer and the hole transport layer, wherein the light emitting layer includes a first compound represented by Chemical Formula 1, and a second compound represented by Chemical Formula 2 or a combination of Chemical Formula 3 and Chemical Formula 4, the hole transport auxiliary layer includes a third compound represented by Chemical Formula 5:

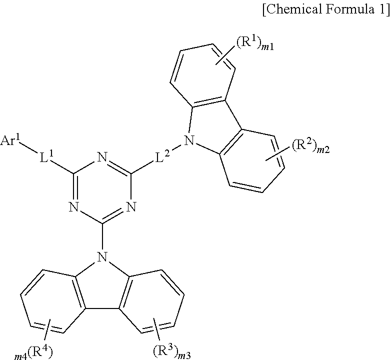

in Chemical Formula 1, Ar¹ is a substituted or unsubstituted C6 to C30 aryl group, a substituted or unsubstituted dibenzofuranyl group, or a substituted or unsubstituted dibenzothiophenyl group, L¹ is a single bond or a substituted or unsubstituted C6 to C20 arylene group, L² is a substituted or unsubstituted C6 to C20 arylene group, R¹ to R⁴ are each independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and m1 to m4 are each independently an integer of 1 to 4;

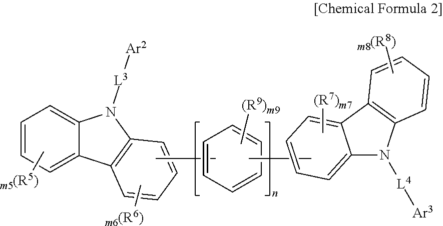

in Chemical Formula 2, Ar² and Ar³ are each independently a substituted or unsubstituted C6 to C20 aryl group or a substituted or unsubstituted C2 to C30 heterocyclic group, L³ and L⁴ are each independently a single bond or a substituted or unsubstituted C6 to C20 arylene group, R⁵ to R⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, m5, m8, and m9 are each independently an integer of 1 to 4, m6 and m7 are each independently an integer of 1 to 3, and n is an integer of 0 to 2;

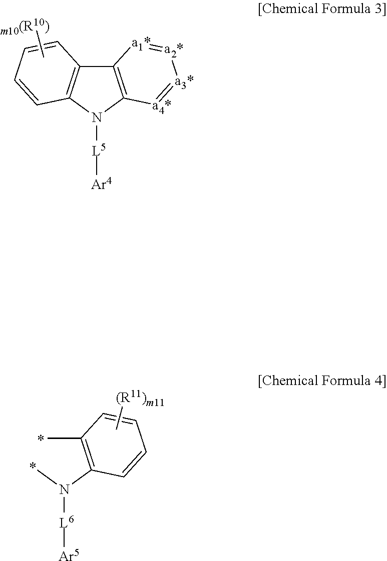

in Chemical Formula 3 and Chemical Formula 4, Ar⁴ and Ar⁵ are each independently a substituted or unsubstituted C6 to C20 aryl group or a substituted or unsubstituted C2 to C30 heterocyclic group, two adjacent ones of a₁* to a₄* of Chemical Formula 3 are linking carbons linked at * of Chemical Formula 4, the remaining two of a1* to a4* of Chemical Formula 3, not linked at * of Chemical Formula 4, are C-L^{a}-R^{a}, L^{a}, L⁵, and L⁶ are each independently a single bond or a substituted or unsubstituted C6 to C20 arylene group, R^{a}, R¹⁰, and R¹¹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and m10 and m11 are each independently an integer of 1 to 4;

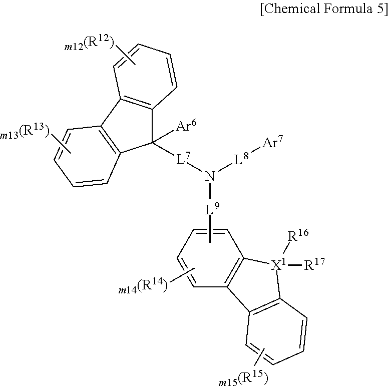

in Chemical Formula 5, X¹ is C or Si, R¹² to R¹⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, R¹⁶ and R¹⁷ are each independently a substituted or unsubstituted C1 to C30 alkyl group or a substituted or unsubstituted C6 to C30 aryl group, Ar⁶ is a substituted or unsubstituted C6 to C30 aryl group, Ar⁷ is a substituted or unsubstituted C6 to C30 aryl group or a substituted or unsubstituted dibenzosilolyl group, L⁷ to L⁹ are each independently a single bond or a substituted or unsubstituted C6 to C20 arylene group, m12, m13, and m15 are each independently an integer of 1 to 4, and m14 is an integer of 1 to 3.

The embodiments may be realized by providing a display device including the organic optoelectronic device according to an embodiment.

### BRIEF DESCRIPTION OF THE DRAWING

Features will be apparent to those of skill in the art by describing in detail exemplary embodiments with reference to the attached drawing in which:

the FIGURE is a cross-sectional view illustrating an organic light emitting diode according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present invention, the “substituted” refers to replacement of at least one hydrogen of a substituent or a compound by deuterium, a C1 to C20 alkyl group, a C6 to C30 aryl group, or a cyano group. In specific example of the present invention, the “substituted” refers to replacement of at least one hydrogen of a substituent or a compound by deuterium, a C1 to C5 alkyl group, a C6 to C18 aryl group, or a cyano group. In specific example of the present invention, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

As used herein, “unsubstituted” refers to non-replacement of a hydrogen atom by another substituent and remaining of the hydrogen atom.

As used herein, “hydrogen” may include “deuterium substitution (-D)” or “tritium substitution (-T).” In addition, any hydrogen in any compound described herein may be protium, deuterium, or tritium (e.g., based on natural or artificial substitution).

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “a heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “a heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzofuranpyrimidinyl group, a substituted or unsubstituted benzothiophenepyrimidinyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUNO) level.

Hereinafter, an organic optoelectronic device according to an embodiment is described.

The organic optoelectronic device may be any device to convert electrical energy into photoenergy and vice versa, and may be, e.g., an organic photoelectric device, an organic light emitting diode, an organic solar cell, or an organic photoconductor drum.

Herein, an organic light emitting diode as one example of an organic optoelectronic device is described, but the embodiments may be applied to other organic optoelectronic device in the same way.

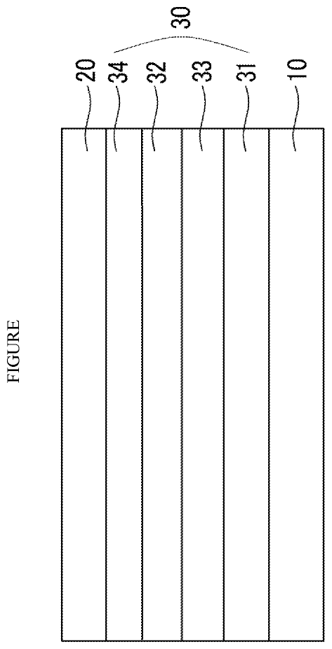

The FIG. 1s a cross-sectional view showing organic light emitting diodes according to embodiments.

Referring to the FIGURE, an organic light emitting diode according to an embodiment may include an anode 10 and a cathode 20 facing each other and an organic layer 30 between the anode 10 and cathode 20.

The anode 10 may be made of a conductor having a large work function to help hole injection, and may be for example a metal, a metal oxide and/or a conductive polymer. The anode 10 may be, for example a metal such as nickel, platinum, vanadium, chromium, copper, zinc, gold, or the like or an alloy thereof; a metal oxide such as zinc oxide, indium oxide, indium tin oxide (ITO), indium zinc oxide (IZO), or the like; a combination of a metal and an oxide such as ZnO and Al or SnO₂ and Sb; or a conductive polymer such as poly (3-methylthiophene), poly (3,4-(ethylene-1,2-dioxy)thiophene) (PEDOT), polypyrrole, or polyaniline.

The cathode 20 may be made of a conductor having a small work function to help electron injection, and may be for example a metal, a metal oxide, and/or a conductive polymer. The cathode 20 may be for example a metal such as magnesium, calcium, sodium, potassium, titanium, indium, yttrium, lithium, gadolinium, aluminum, silver, tin, lead, cesium, barium, or the like, or an alloy thereof; or a multi-layer structure material such as LiF/Al, LiO₂/Al, LiF/Ca, or BaF₂/Ca.

The organic layer 30 may include a hole transport layer 31, a light emitting layer 32, and a hole transport auxiliary layer 33 between the hole transport layer 31 and the light emitting layer 32.

The hole transport layer 31 may be a layer for facilitating hole transfer from the anode 10 to the light emitting layer 32, and may include, for example, an amine compound.

The amine compound may include, for example, an aryl group or a heteroaryl group. The amine compound may be represented by, for example, Chemical Formula a or Chemical Formula b.

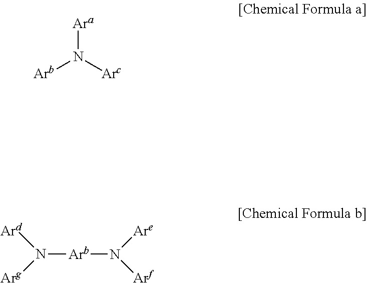

In Chemical Formula a and b,

Ar^{a} to Ar^{g} may each independently be or include, e.g., hydrogen, deuterium, a substituted or unsubstituted C1 to C20 alkyl group, a substituted or unsubstituted C6 to C30 aryl group, a substituted or unsubstituted C2 to C30 heteroaryl group, or a combination thereof.

At least one of Ar^{a} to Ar^{c} and at least one of Ar^{d} to Ar^{g} may be a substituted or unsubstituted C6 to C30 aryl group, a substituted or unsubstituted C2 to C30 heteroaryl group, or a combination thereof.

Ar^{h} may be or include, e.g., a single bond, a substituted or unsubstituted C1 to C20 alkylene group, a substituted or unsubstituted C6 to C30 arylene group, a substituted or unsubstituted C2 to C30 heteroarylene group or a combination thereof.

The light emitting layer 32 may include at least two types of hosts and dopants. The host may include a first compound having bipolar characteristics with relatively strong electronic characteristics and a second compound having bipolar characteristics with relatively strong hole characteristics.

The first compound may be represented by Chemical Formula 1.

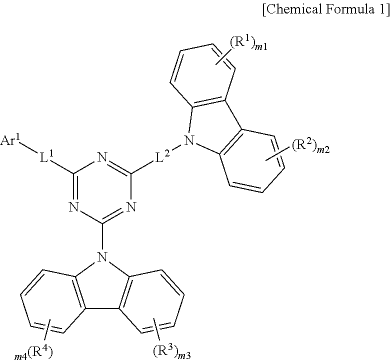

In Chemical Formula 1, Ar¹ may be or include, e.g., a substituted or unsubstituted C6 to C30 aryl group, a substituted or unsubstituted dibenzofuranyl group, or a substituted or unsubstituted dibenzothiophenyl group.

L¹ may be or include, e.g., a single bond or a substituted or unsubstituted C6 to C20 arylene group.

L² may be or include, e.g., a substituted or unsubstituted C6 to C20 arylene group.

R¹ to R⁴ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof.

m1 to m4 may each independently be, e.g., an integer of 1 to 4.

The first compound may have a structure in which one carbazole group is directly linked to the triazine in the N-direction without a linking group, and another carbazole group is linked to the triazine through a linking group in the N-direction with the triazine as the center.

One carbazole group may be directly linked to the triazine in the N-direction, e.g., at the 9-position, without a linking group, so that it has a relatively deep LUMO energy level, which may be advantageous for electron injection and movement.

In addition, another carbazole group may be linked to the triazine in the N-direction, e.g., at the 9-position, through a linking group, the it-bond through the C—N bond is broken, an electron cloud between HOMO-LUMO may be clearly localized into a hole transport moiety and an electron transport moiety, this localization may be more effectively separated by linking the carbazole group to the triazine through a linking group, and thus life-span improvement effect may be maximized.

In an implementation, in a device including the compound represented by Chemical Formula 1, hole/electron injection and movement are advantageous, and electron clouds may be effectively localized to implement a structure stable for both electrons and holes, so that it may have more favorable characteristics for life-span.

In an implementation, by including triazine as a central core, fast electron injection and mobility may be ensured, resulting in charge balance with the carbazole moiety having strong hole mobility, which also greatly contributes to long life-span characteristics.

In an implementation, L¹ in Chemical Formula 1 may be, e.g., a single bond, a substituted or unsubstituted phenylene group, or a substituted or unsubstituted biphenylene group.

In an implementation, L² in Chemical Formula 1 may be, e.g., a substituted or unsubstituted phenylene group, a substituted or unsubstituted biphenylene group, or a substituted or unsubstituted terphenylene group.

In an implementation, L² may be a substituted or unsubstituted phenylene group, e.g., a substituted or unsubstituted meta-phenylene group or a substituted or unsubstituted ortho-phenylene group.

In an implementation, L² in Chemical Formula 1 may be a linking group of Group I.

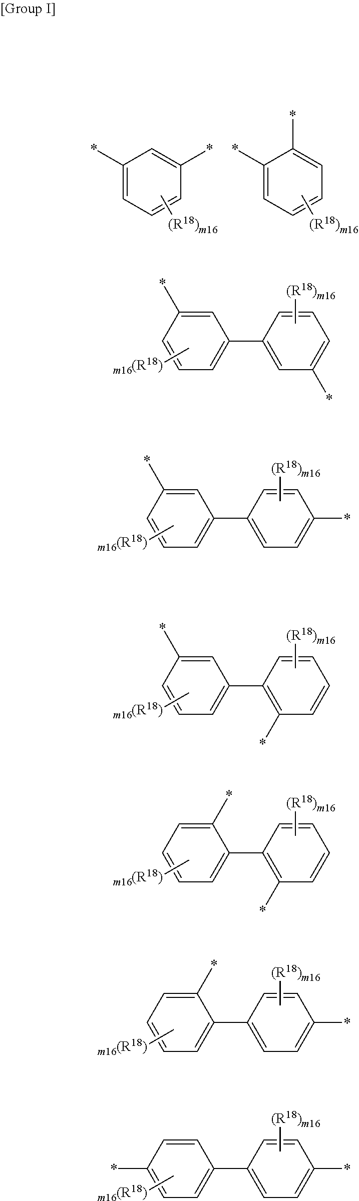

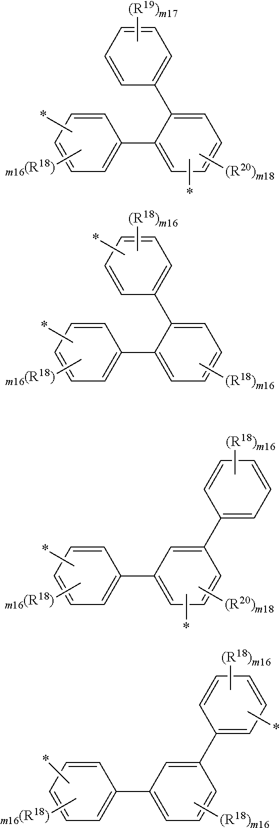

In Group I, R¹⁸ to R²⁰ may each independently be, e.g., hydrogen, deuterium, a halogen, a substituted or unsubstituted C1 to C20 alkyl group, a substituted or unsubstituted C3 to C20 cycloalkyl group, a substituted or unsubstituted C1 to C20 alkoxy group, a substituted or unsubstituted C3 to C20 cycloalkoxy group, a substituted or unsubstituted C1 to C20 alkylthio group, a substituted or unsubstituted C6 to C30 aralkyl group, a substituted or unsubstituted C6 to C30 aryl group, a substituted or unsubstituted C6 to C30 aryloxy group, a substituted or unsubstituted C6 to C30 arylthio group, a substituted or unsubstituted C2 to C30 heteroaryl group, a substituted or unsubstituted C2 to C30 amino group, a substituted or unsubstituted C3 to C30 silyl group, a cyano group, a nitro group, a hydroxyl group, a carboxyl group, or a combination thereof.

m16 may be, e.g., an integer of 1 to 4.

m17 may be, e.g., an integer of 1 to 5.

m18 may be, e.g., an integer of 1 to 3.

* is a linking point.

In an implementation, Ar¹ in Chemical Formula 1 may be, e.g., a substituted or unsubstituted phenyl group, a substituted or unsubstituted biphenyl group, a substituted or unsubstituted terphenyl group, a substituted or unsubstituted quaterphenyl group, a substituted or unsubstituted fluorenyl group, a substituted or unsubstituted dibenzofuranyl group, or a substituted or unsubstituted dibenzothiophenyl group.

In an implementation, Ar¹ in Chemical Formula 1 may be, e.g., a substituted or unsubstituted phenyl group, a substituted or unsubstituted biphenyl group, a substituted or unsubstituted fluorenyl group, a substituted or unsubstituted dibenzofuranyl group, or a substituted or unsubstituted dibenzothiophenyl group.

R¹ in Chemical Formula 1 may be two or more, in which case each R¹ may be the same or different.

R² in Chemical Formula 1 may be two or more, in which case each R² may be the same or different.

R³ in Chemical Formula 1 may be two or more, in which case each R³ may be the same or different.

R⁴ in Chemical Formula 1 may be two or more, in which case each R⁴ may be the same or different.

In an implementation, R¹ to R⁴ in Chemical Formula 1 may independently be, e.g., hydrogen, deuterium, a cyano group, or a substituted or unsubstituted phenyl group.

In an implementation, the first compound may be a compound of Group 1.

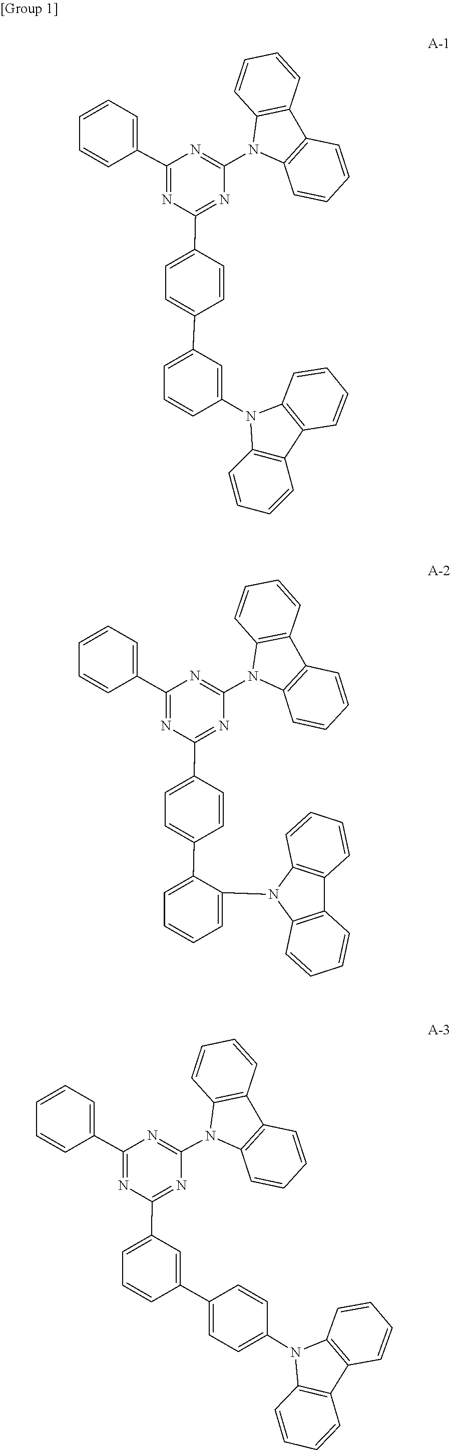 [SMILES: c1ccc(-c2nc(-c3ccc(-c4cccc(-n5c6ccccc6c6ccccc65)c4)cc3)nc(-n3c4ccccc4c4ccccc43)n2)cc1 ; c1ccc(-c2nc(-c3ccc(-c4ccccc4-n4c5ccccc5c5ccccc54)cc3)nc(-n3c4ccccc4c4ccccc43)n2)cc1 ; c1ccc(-c2nc(-c3cccc(-c4ccc(-n5c6ccccc6c6ccccc65)cc4)c3)nc(-n3c4ccccc4c4ccccc43)n2)cc1]

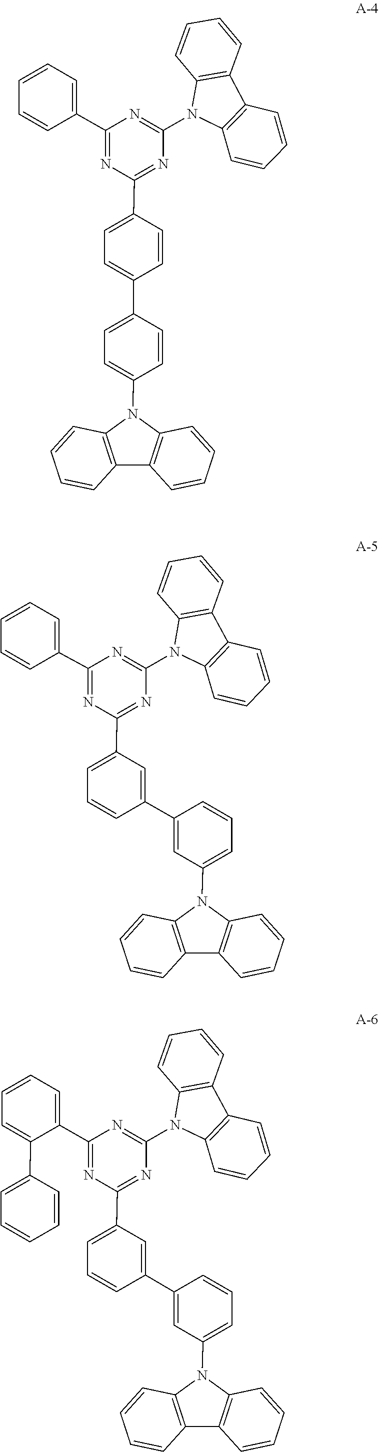 [SMILES: c1ccc(-c2nc(-c3ccc(-c4ccc(-n5c6ccccc6c6ccccc65)cc4)cc3)nc(-n3c4ccccc4c4ccccc43)n2)cc1 ; c1ccc(-c2nc(-c3cccc(-c4cccc(-n5c6ccccc6c6ccccc65)c4)c3)nc(-n3c4ccccc4c4ccccc43)n2)cc1 ; c1ccc(-c2ccccc2-c2nc(-c3cccc(-c4cccc(-n5c6ccccc6c6ccccc65)c4)c3)nc(-n3c4ccccc4c4ccccc43)n2)cc1]

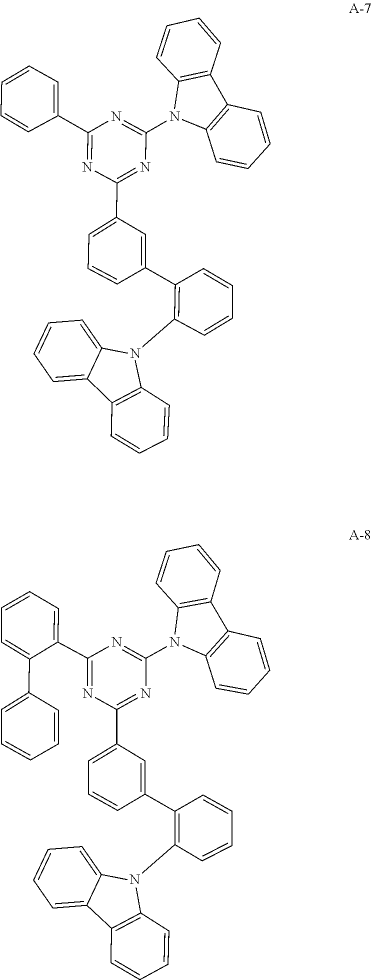 [SMILES: c1ccc(-c2nc(-c3cccc(-c4ccccc4-n4c5ccccc5c5ccccc54)c3)nc(-n3c4ccccc4c4ccccc43)n2)cc1 ; c1ccc(-c2ccccc2-c2nc(-c3cccc(-c4ccccc4-n4c5ccccc5c5ccccc54)c3)nc(-n3c4ccccc4c4ccccc43)n2)cc1]

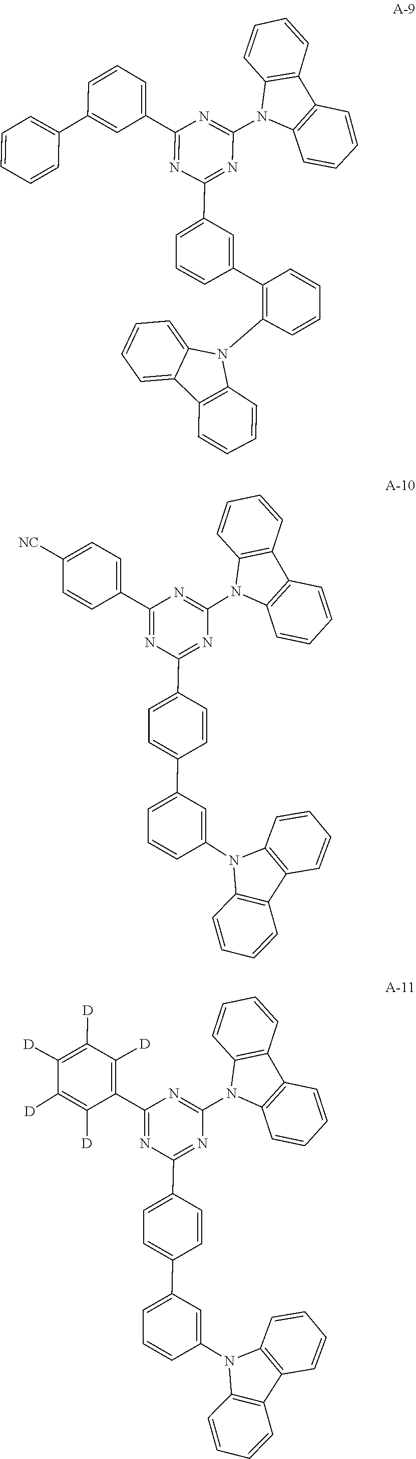

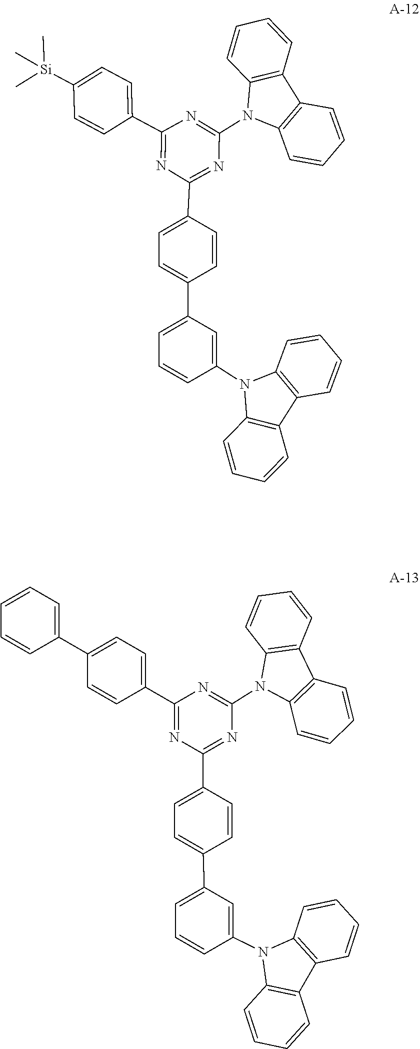 [SMILES: C[Si](C)(C)c1ccc(-c2nc(-c3ccc(-c4cccc(-n5c6ccccc6c6ccccc65)c4)cc3)nc(-n3c4ccccc4c4ccccc43)n2)cc1 ; c1ccc(-c2ccc(-c3nc(-c4ccc(-c5cccc(-n6c7ccccc7c7ccccc76)c5)cc4)nc(-n4c5ccccc5c5ccccc54)n3)cc2)cc1]

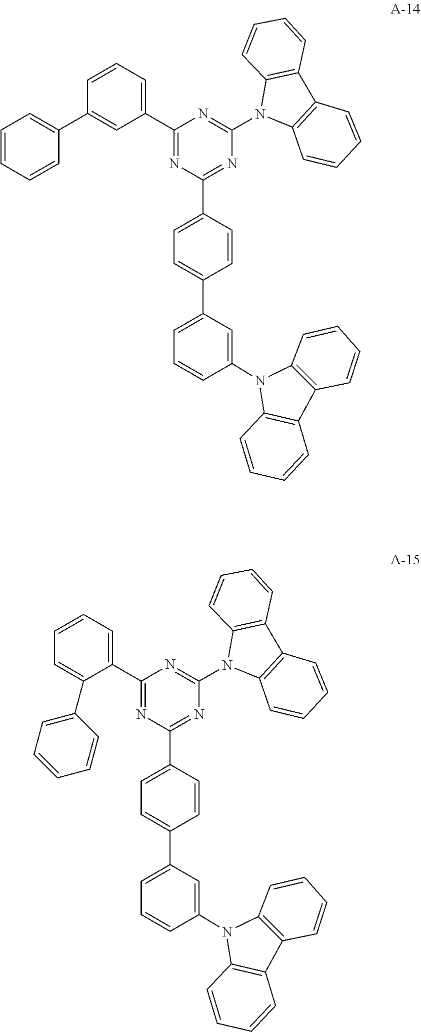 [SMILES: c1ccc(-c2cccc(-c3nc(-c4ccc(-c5cccc(-n6c7ccccc7c7ccccc76)c5)cc4)nc(-n4c5ccccc5c5ccccc54)n3)c2)cc1 ; c1ccc(-c2ccccc2-c2nc(-c3ccc(-c4cccc(-n5c6ccccc6c6ccccc65)c4)cc3)nc(-n3c4ccccc4c4ccccc43)n2)cc1]

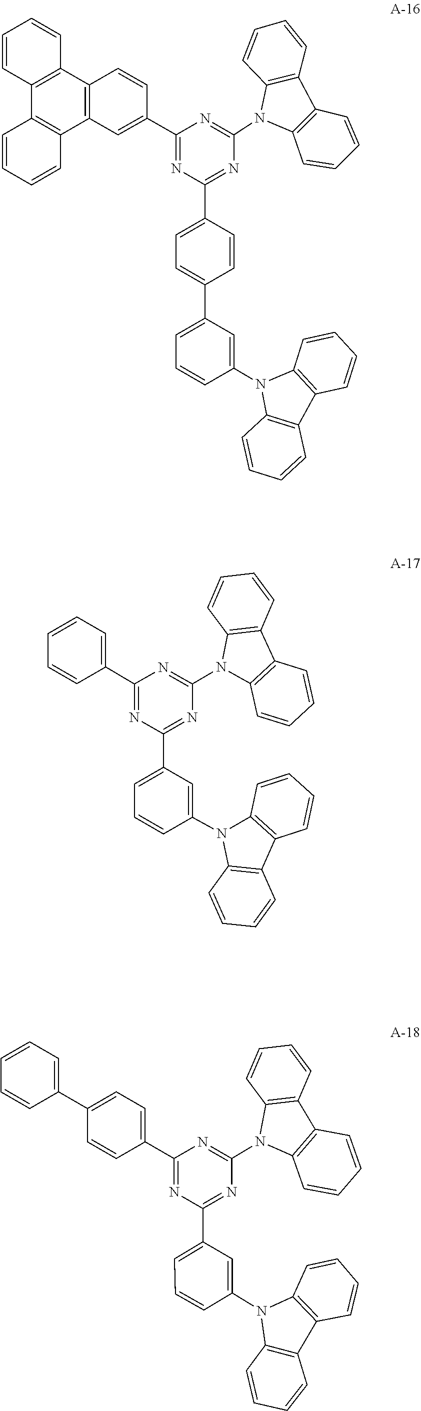

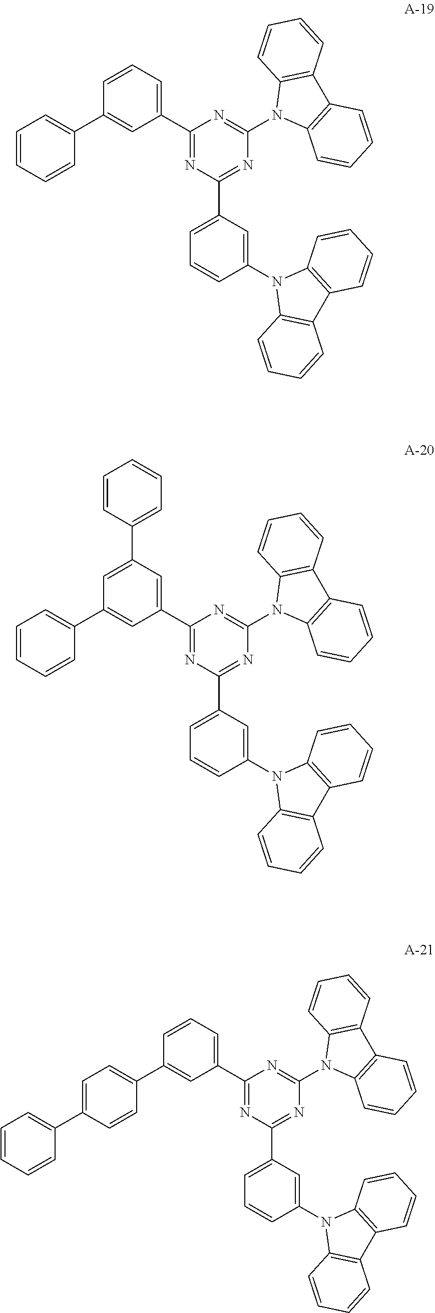 [SMILES: c1ccc(-c2cccc(-c3nc(-c4cccc(-n5c6ccccc6c6ccccc65)c4)nc(-n4c5ccccc5c5ccccc54)n3)c2)cc1 ; c1ccc(-c2cc(-c3ccccc3)cc(-c3nc(-c4cccc(-n5c6ccccc6c6ccccc65)c4)nc(-n4c5ccccc5c5ccccc54)n3)c2)cc1 ; c1ccc(-c2ccc(-c3cccc(-c4nc(-c5cccc(-n6c7ccccc7c7ccccc76)c5)nc(-n5c6ccccc6c6ccccc65)n4)c3)cc2)cc1]

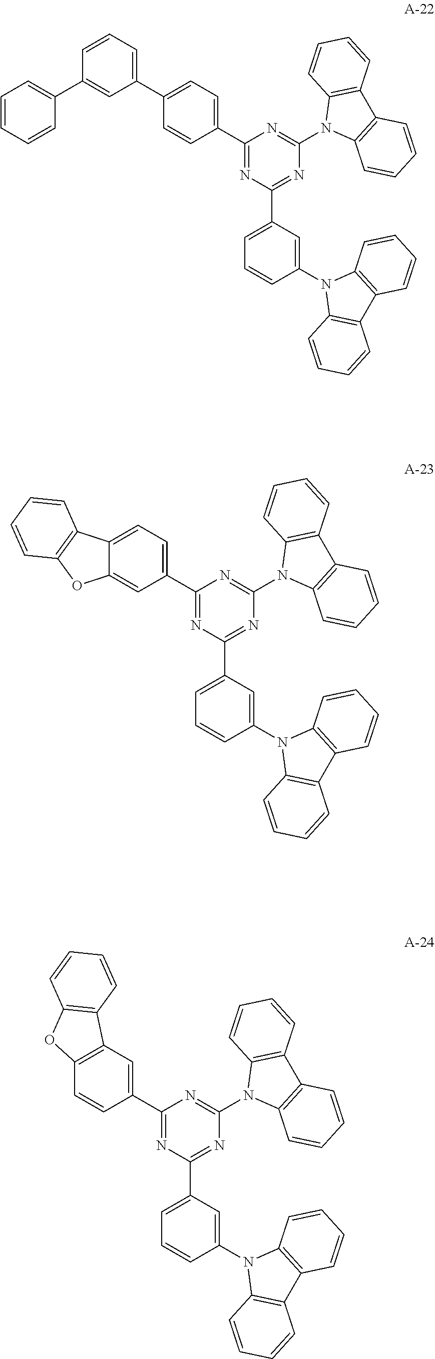 [SMILES: c1ccc(-c2cccc(-c3ccc(-c4nc(-c5cccc(-n6c7ccccc7c7ccccc76)c5)nc(-n5c6ccccc6c6ccccc65)n4)cc3)c2)cc1 ; c1cc(-c2nc(-c3ccc4c(c3)oc3ccccc34)nc(-n3c4ccccc4c4ccccc43)n2)cc(-n2c3ccccc3c3ccccc32)c1 ; c1cc(-c2nc(-c3ccc4oc5ccccc5c4c3)nc(-n3c4ccccc4c4ccccc43)n2)cc(-n2c3ccccc3c3ccccc32)c1]

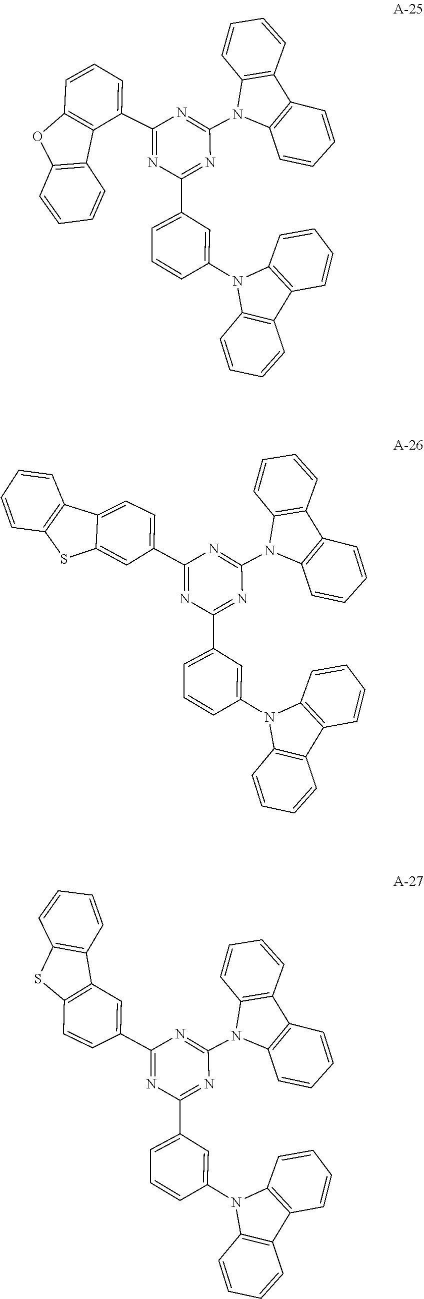 [SMILES: c1cc(-c2nc(-c3cccc4oc5ccccc5c34)nc(-n3c4ccccc4c4ccccc43)n2)cc(-n2c3ccccc3c3ccccc32)c1 ; c1cc(-c2nc(-c3ccc4c(c3)sc3ccccc34)nc(-n3c4ccccc4c4ccccc43)n2)cc(-n2c3ccccc3c3ccccc32)c1 ; c1cc(-c2nc(-c3ccc4sc5ccccc5c4c3)nc(-n3c4ccccc4c4ccccc43)n2)cc(-n2c3ccccc3c3ccccc32)c1]

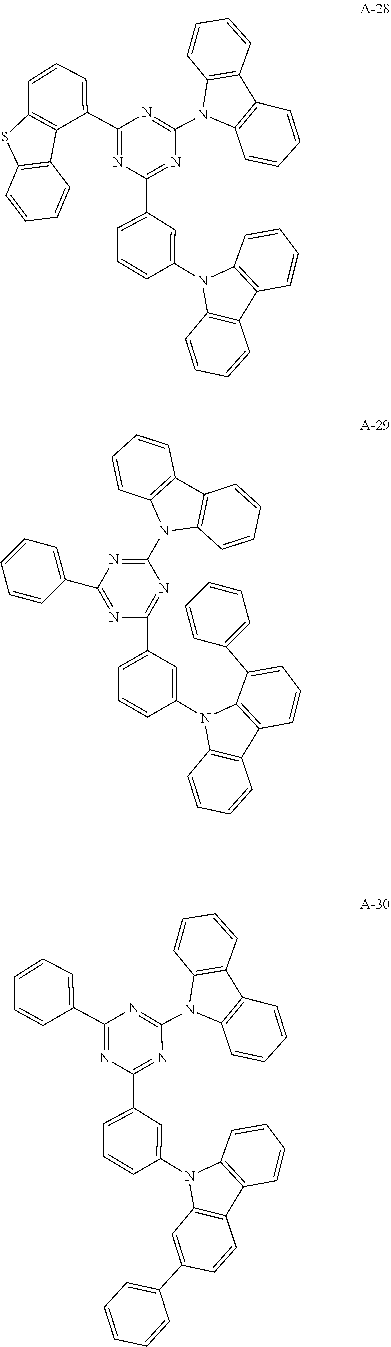 [SMILES: c1cc(-c2nc(-c3cccc4sc5ccccc5c34)nc(-n3c4ccccc4c4ccccc43)n2)cc(-n2c3ccccc3c3ccccc32)c1 ; c1ccc(-c2nc(-c3cccc(-n4c5ccccc5c5cccc(-c6ccccc6)c54)c3)nc(-n3c4ccccc4c4ccccc43)n2)cc1 ; c1ccc(-c2ccc3c4ccccc4n(-c4cccc(-c5nc(-c6ccccc6)nc(-n6c7ccccc7c7ccccc76)n5)c4)c3c2)cc1]

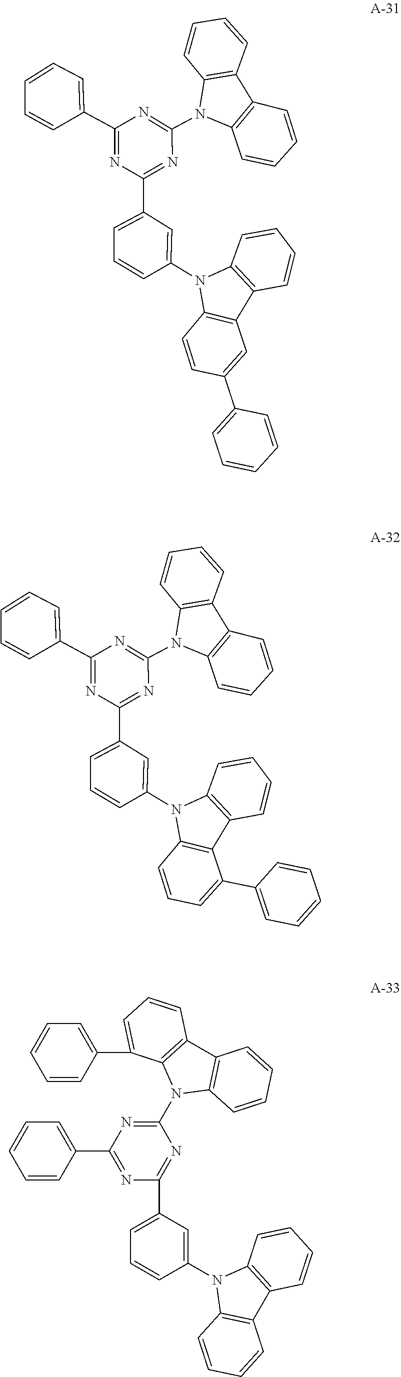 [SMILES: c1ccc(-c2ccc3c(c2)c2ccccc2n3-c2cccc(-c3nc(-c4ccccc4)nc(-n4c5ccccc5c5ccccc54)n3)c2)cc1 ; c1ccc(-c2nc(-c3cccc(-n4c5ccccc5c5c(-c6ccccc6)cccc54)c3)nc(-n3c4ccccc4c4ccccc43)n2)cc1 ; c1ccc(-c2nc(-c3cccc(-n4c5ccccc5c5ccccc54)c3)nc(-n3c4ccccc4c4cccc(-c5ccccc5)c43)n2)cc1]

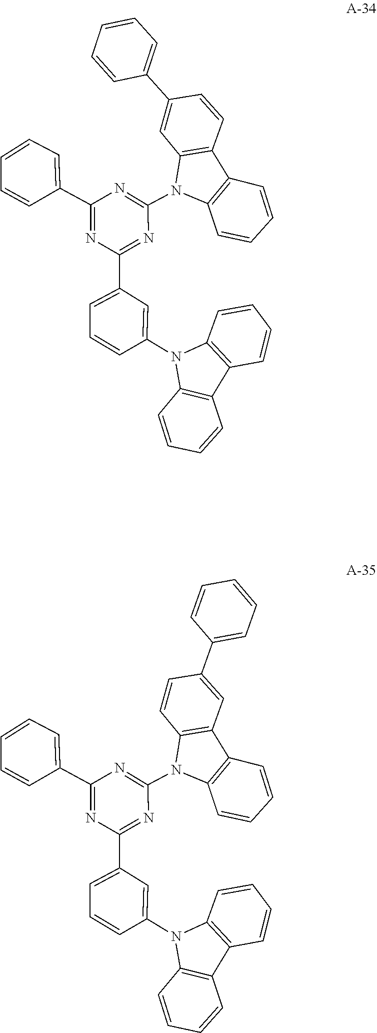 [SMILES: c1ccc(-c2ccc3c4ccccc4n(-c4nc(-c5ccccc5)nc(-c5cccc(-n6c7ccccc7c7ccccc76)c5)n4)c3c2)cc1 ; c1ccc(-c2ccc3c(c2)c2ccccc2n3-c2nc(-c3ccccc3)nc(-c3cccc(-n4c5ccccc5c5ccccc54)c3)n2)cc1]

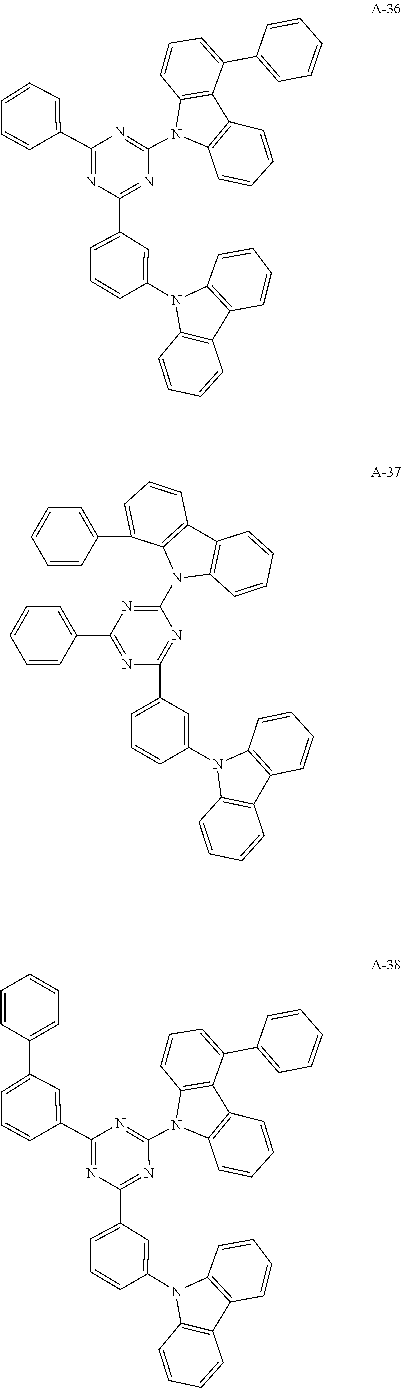 [SMILES: c1ccc(-c2nc(-c3cccc(-n4c5ccccc5c5ccccc54)c3)nc(-n3c4ccccc4c4c(-c5ccccc5)cccc43)n2)cc1 ; c1ccc(-c2nc(-c3cccc(-n4c5ccccc5c5ccccc54)c3)nc(-n3c4ccccc4c4cccc(-c5ccccc5)c43)n2)cc1 ; c1ccc(-c2cccc(-c3nc(-c4cccc(-n5c6ccccc6c6ccccc65)c4)nc(-n4c5ccccc5c5c(-c6ccccc6)cccc54)n3)c2)cc1]

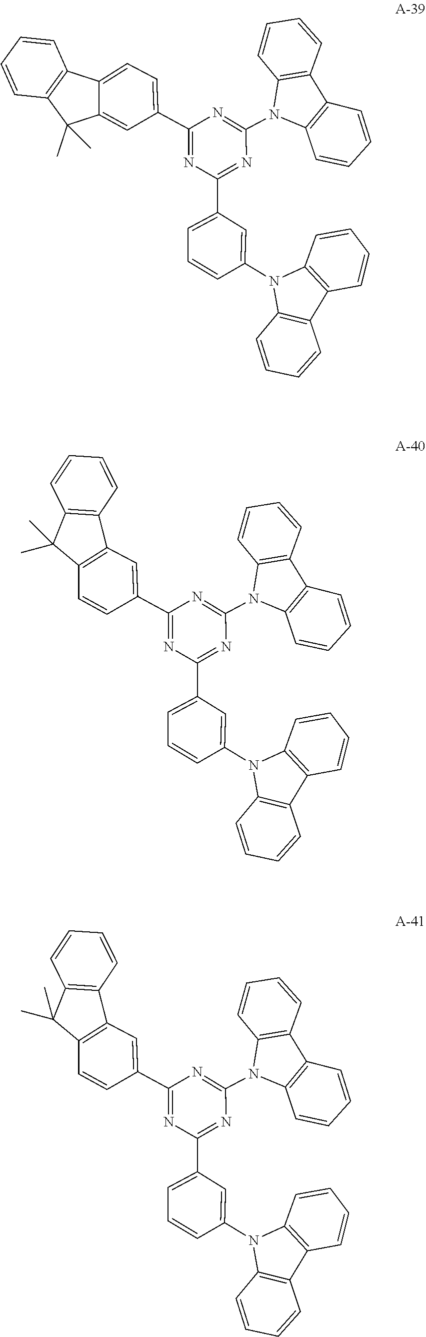 [SMILES: CC1(C)c2ccccc2-c2ccc(-c3nc(-c4cccc(-n5c6ccccc6c6ccccc65)c4)nc(-n4c5ccccc5c5ccccc54)n3)cc21 ; CC1(C)c2ccccc2-c2cc(-c3nc(-c4cccc(-n5c6ccccc6c6ccccc65)c4)nc(-n4c5ccccc5c5ccccc54)n3)ccc21 ; CC1(C)c2ccccc2-c2cc(-c3nc(-c4cccc(-n5c6ccccc6c6ccccc65)c4)nc(-n4c5ccccc5c5ccccc54)n3)ccc21]

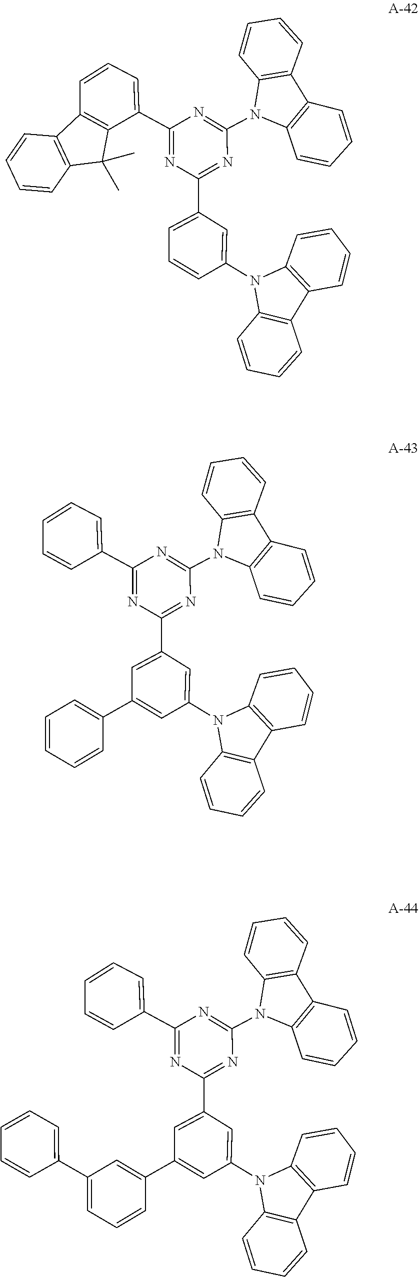 [SMILES: CC1(C)c2ccccc2-c2cccc(-c3nc(-c4cccc(-n5c6ccccc6c6ccccc65)c4)nc(-n4c5ccccc5c5ccccc54)n3)c21 ; c1ccc(-c2cc(-c3nc(-c4ccccc4)nc(-n4c5ccccc5c5ccccc54)n3)cc(-n3c4ccccc4c4ccccc43)c2)cc1 ; c1ccc(-c2cccc(-c3cc(-c4nc(-c5ccccc5)nc(-n5c6ccccc6c6ccccc65)n4)cc(-n4c5ccccc5c5ccccc54)c3)c2)cc1]

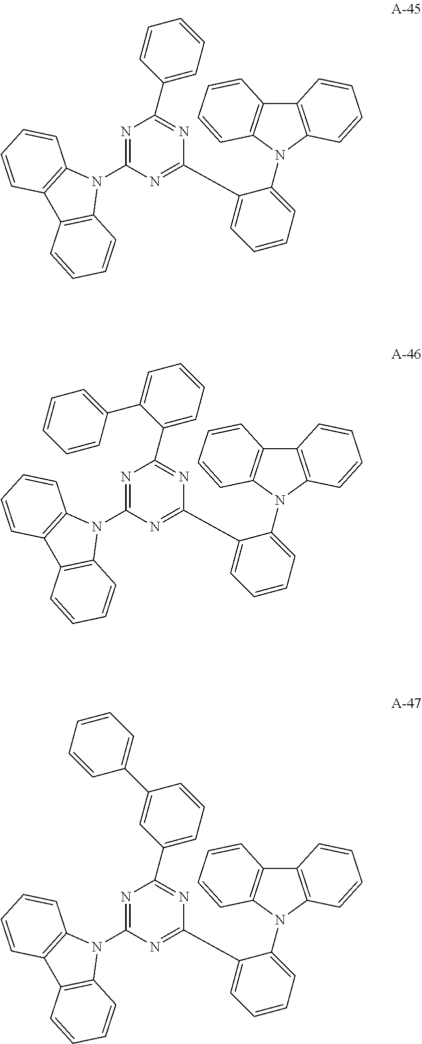 [SMILES: c1ccc(-c2nc(-c3ccccc3-n3c4ccccc4c4ccccc43)nc(-n3c4ccccc4c4ccccc43)n2)cc1 ; c1ccc(-c2ccccc2-c2nc(-c3ccccc3-n3c4ccccc4c4ccccc43)nc(-n3c4ccccc4c4ccccc43)n2)cc1 ; c1ccc(-c2cccc(-c3nc(-c4ccccc4-n4c5ccccc5c5ccccc54)nc(-n4c5ccccc5c5ccccc54)n3)c2)cc1]

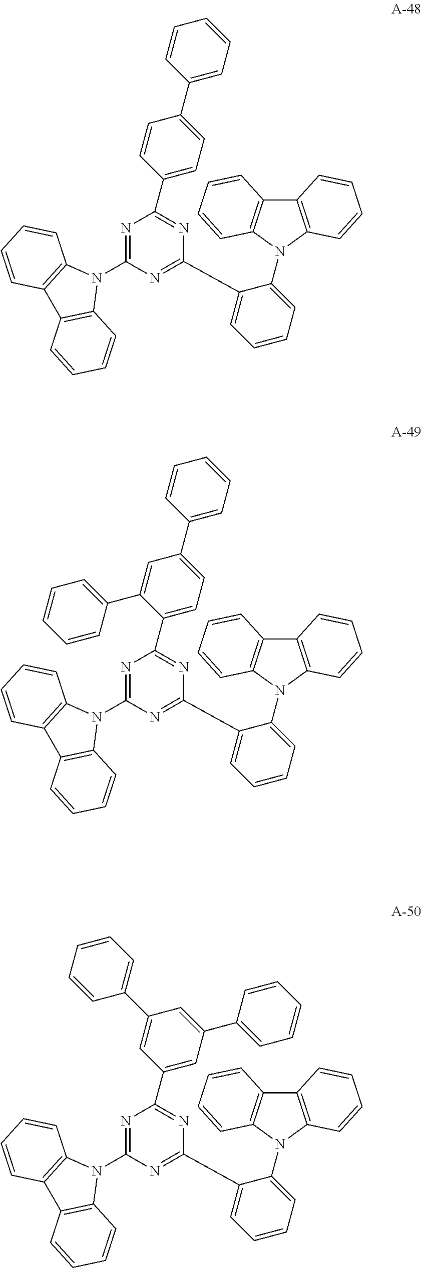 [SMILES: c1ccc(-c2ccc(-c3nc(-c4ccccc4-n4c5ccccc5c5ccccc54)nc(-n4c5ccccc5c5ccccc54)n3)cc2)cc1 ; c1ccc(-c2ccc(-c3nc(-c4ccccc4-n4c5ccccc5c5ccccc54)nc(-n4c5ccccc5c5ccccc54)n3)c(-c3ccccc3)c2)cc1 ; c1ccc(-c2cc(-c3ccccc3)cc(-c3nc(-c4ccccc4-n4c5ccccc5c5ccccc54)nc(-n4c5ccccc5c5ccccc54)n3)c2)cc1]

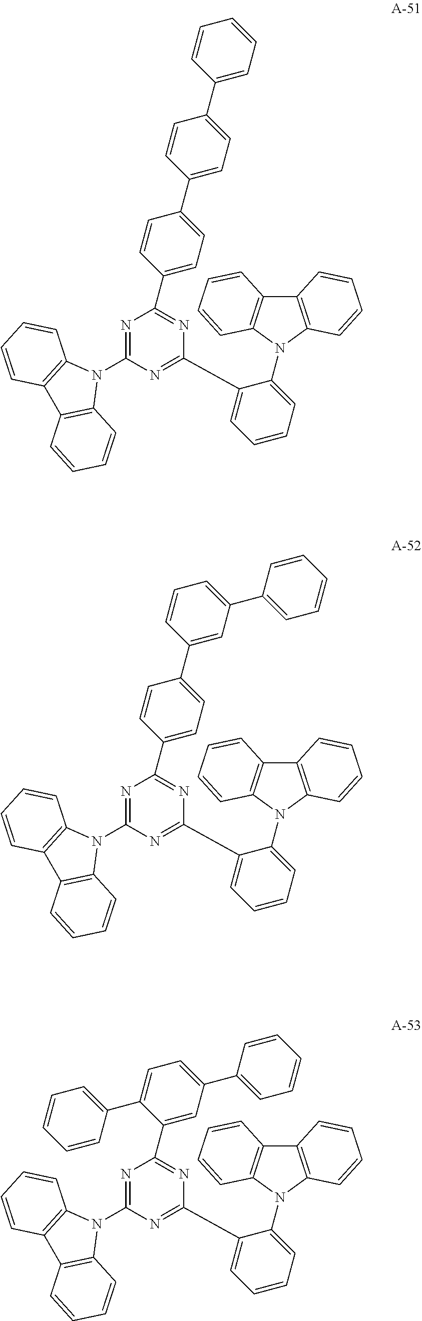 [SMILES: c1ccc(-c2ccc(-c3ccc(-c4nc(-c5ccccc5-n5c6ccccc6c6ccccc65)nc(-n5c6ccccc6c6ccccc65)n4)cc3)cc2)cc1 ; c1ccc(-c2cccc(-c3ccc(-c4nc(-c5ccccc5-n5c6ccccc6c6ccccc65)nc(-n5c6ccccc6c6ccccc65)n4)cc3)c2)cc1 ; c1ccc(-c2ccc(-c3ccccc3)c(-c3nc(-c4ccccc4-n4c5ccccc5c5ccccc54)nc(-n4c5ccccc5c5ccccc54)n3)c2)cc1]

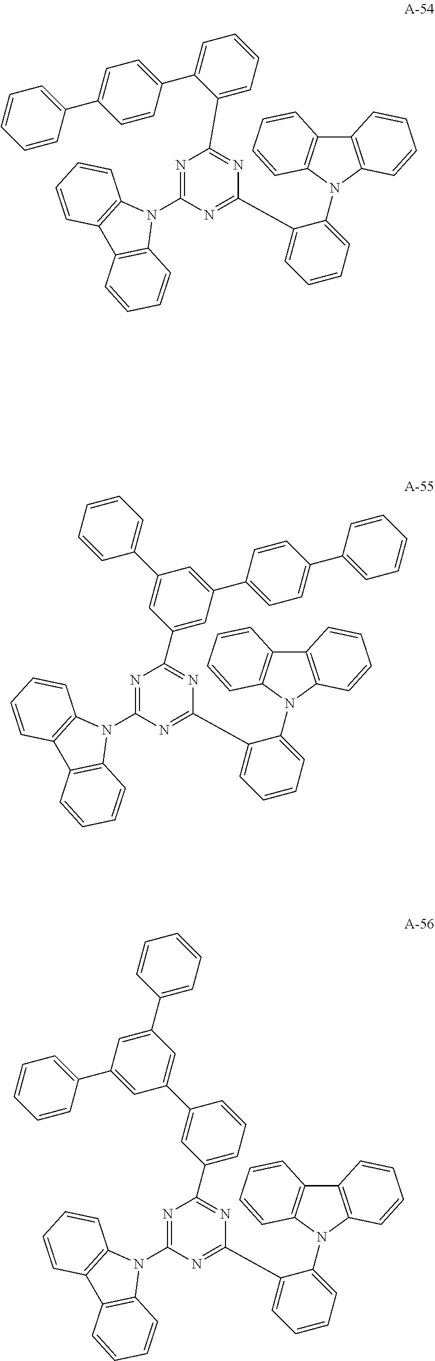 [SMILES: c1ccc(-c2ccc(-c3ccccc3-c3nc(-c4ccccc4-n4c5ccccc5c5ccccc54)nc(-n4c5ccccc5c5ccccc54)n3)cc2)cc1 ; c1ccc(-c2ccc(-c3cc(-c4ccccc4)cc(-c4nc(-c5ccccc5-n5c6ccccc6c6ccccc65)nc(-n5c6ccccc6c6ccccc65)n4)c3)cc2)cc1 ; c1ccc(-c2cc(-c3ccccc3)cc(-c3cccc(-c4nc(-c5ccccc5-n5c6ccccc6c6ccccc65)nc(-n5c6ccccc6c6ccccc65)n4)c3)c2)cc1]

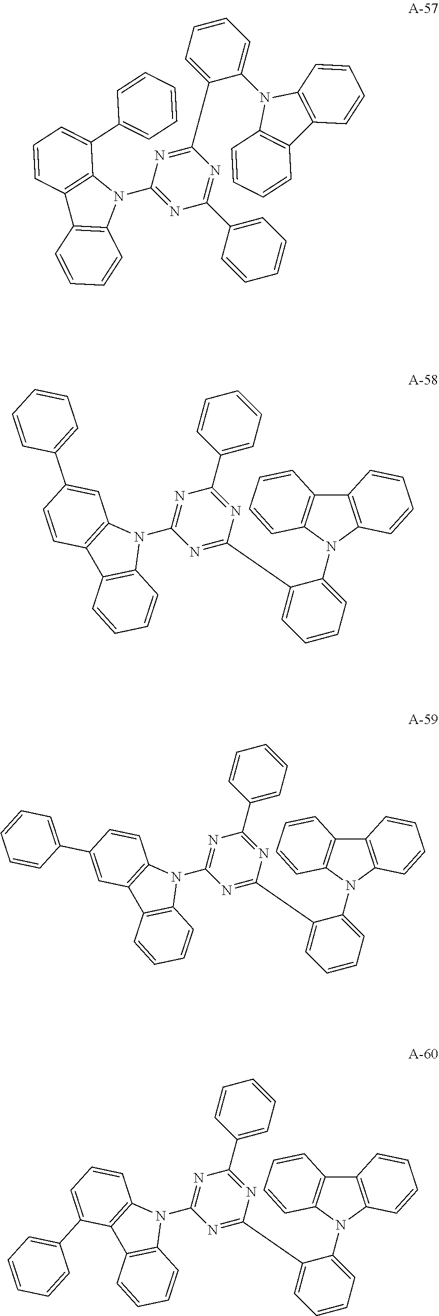 [SMILES: c1ccc(-c2nc(-c3ccccc3-n3c4ccccc4c4ccccc43)nc(-n3c4ccccc4c4cccc(-c5ccccc5)c43)n2)cc1 ; c1ccc(-c2ccc3c4ccccc4n(-c4nc(-c5ccccc5)nc(-c5ccccc5-n5c6ccccc6c6ccccc65)n4)c3c2)cc1 ; c1ccc(-c2ccc3c(c2)c2ccccc2n3-c2nc(-c3ccccc3)nc(-c3ccccc3-n3c4ccccc4c4ccccc43)n2)cc1 ; c1ccc(-c2nc(-c3ccccc3-n3c4ccccc4c4ccccc43)nc(-n3c4ccccc4c4c(-c5ccccc5)cccc43)n2)cc1]

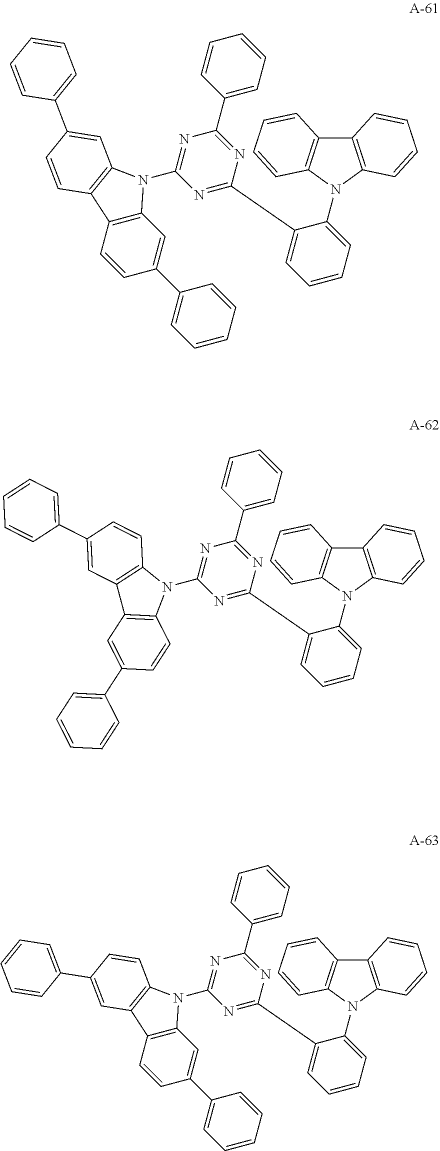 [SMILES: c1ccc(-c2ccc3c4ccc(-c5ccccc5)cc4n(-c4nc(-c5ccccc5)nc(-c5ccccc5-n5c6ccccc6c6ccccc65)n4)c3c2)cc1 ; c1ccc(-c2ccc3c(c2)c2cc(-c4ccccc4)ccc2n3-c2nc(-c3ccccc3)nc(-c3ccccc3-n3c4ccccc4c4ccccc43)n2)cc1 ; c1ccc(-c2ccc3c(c2)c2ccc(-c4ccccc4)cc2n3-c2nc(-c3ccccc3)nc(-c3ccccc3-n3c4ccccc4c4ccccc43)n2)cc1]

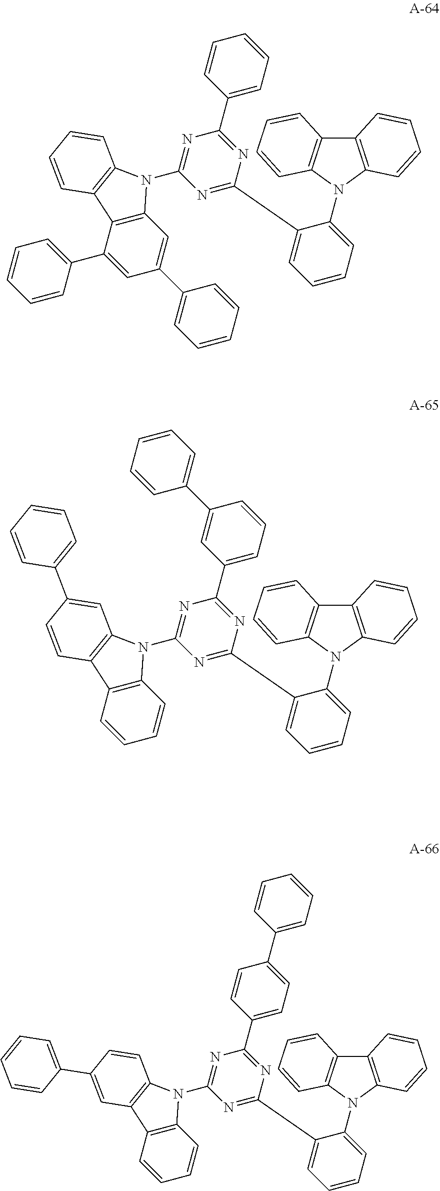 [SMILES: c1ccc(-c2cc(-c3ccccc3)c3c4ccccc4n(-c4nc(-c5ccccc5)nc(-c5ccccc5-n5c6ccccc6c6ccccc65)n4)c3c2)cc1 ; c1ccc(-c2cccc(-c3nc(-c4ccccc4-n4c5ccccc5c5ccccc54)nc(-n4c5ccccc5c5ccc(-c6ccccc6)cc54)n3)c2)cc1 ; c1ccc(-c2ccc(-c3nc(-c4ccccc4-n4c5ccccc5c5ccccc54)nc(-n4c5ccccc5c5cc(-c6ccccc6)ccc54)n3)cc2)cc1]

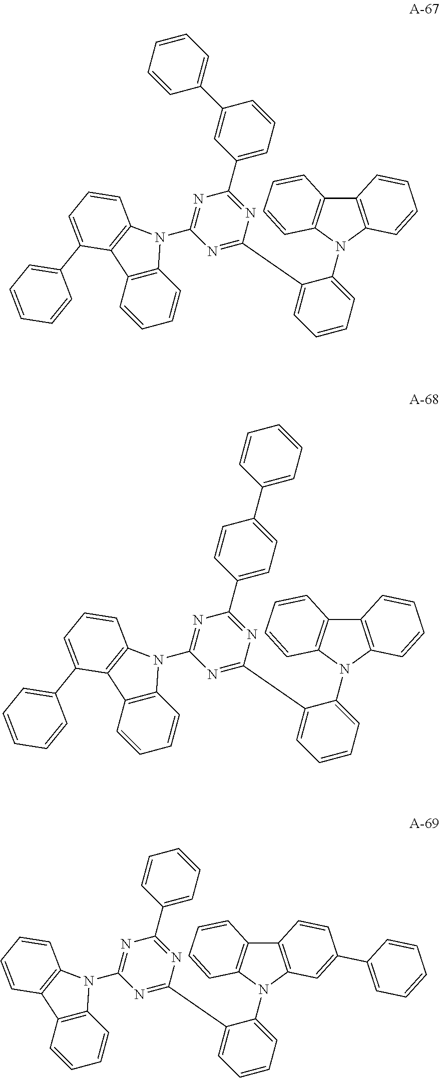 [SMILES: c1ccc(-c2cccc(-c3nc(-c4ccccc4-n4c5ccccc5c5ccccc54)nc(-n4c5ccccc5c5c(-c6ccccc6)cccc54)n3)c2)cc1 ; c1ccc(-c2ccc(-c3nc(-c4ccccc4-n4c5ccccc5c5ccccc54)nc(-n4c5ccccc5c5c(-c6ccccc6)cccc54)n3)cc2)cc1 ; c1ccc(-c2ccc3c4ccccc4n(-c4ccccc4-c4nc(-c5ccccc5)nc(-n5c6ccccc6c6ccccc65)n4)c3c2)cc1]

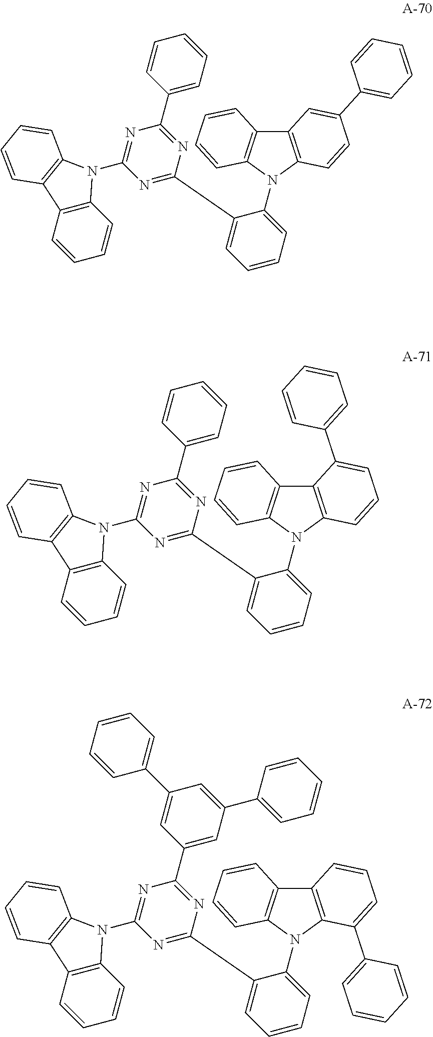 [SMILES: c1ccc(-c2ccc3c(c2)c2ccccc2n3-c2ccccc2-c2nc(-c3ccccc3)nc(-n3c4ccccc4c4ccccc43)n2)cc1 ; c1ccc(-c2nc(-c3ccccc3-n3c4ccccc4c4c(-c5ccccc5)cccc43)nc(-n3c4ccccc4c4ccccc43)n2)cc1 ; c1ccc(-c2cc(-c3ccccc3)cc(-c3nc(-c4ccccc4-n4c5ccccc5c5cccc(-c6ccccc6)c54)nc(-n4c5ccccc5c5ccccc54)n3)c2)cc1]

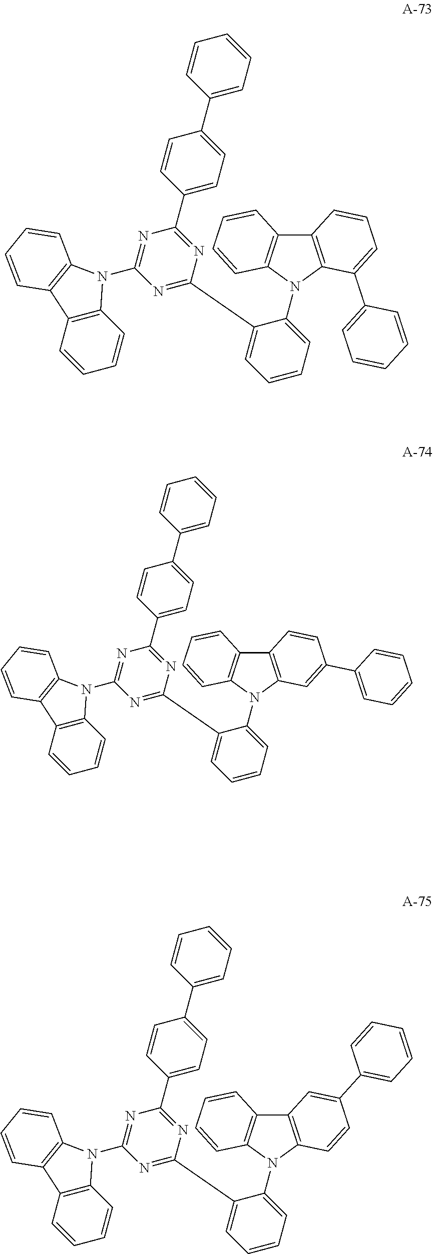 [SMILES: c1ccc(-c2ccc(-c3nc(-c4ccccc4-n4c5ccccc5c5cccc(-c6ccccc6)c54)nc(-n4c5ccccc5c5ccccc54)n3)cc2)cc1 ; c1ccc(-c2ccc(-c3nc(-c4ccccc4-n4c5ccccc5c5ccc(-c6ccccc6)cc54)nc(-n4c5ccccc5c5ccccc54)n3)cc2)cc1 ; c1ccc(-c2ccc(-c3nc(-c4ccccc4-n4c5ccccc5c5cc(-c6ccccc6)ccc54)nc(-n4c5ccccc5c5ccccc54)n3)cc2)cc1]

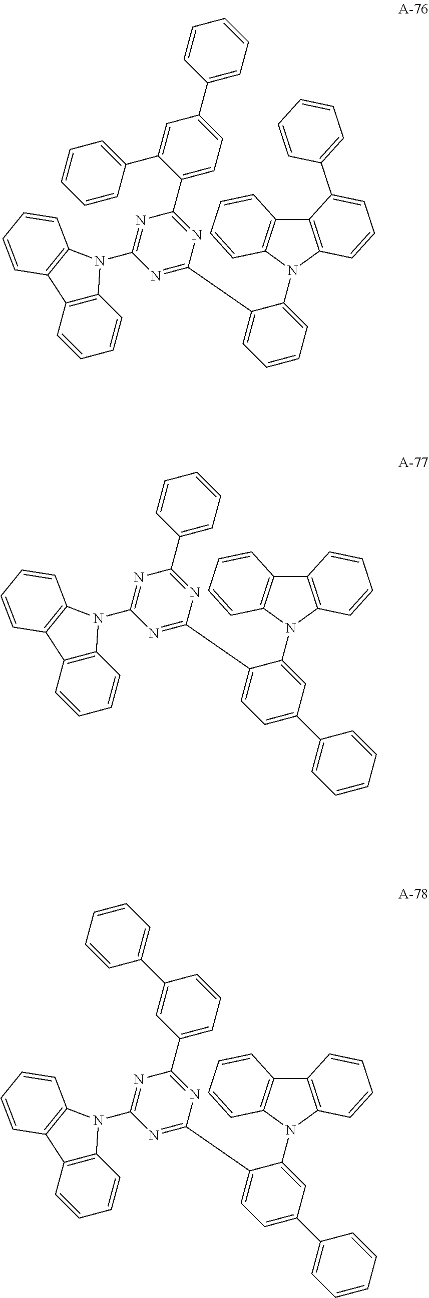 [SMILES: c1ccc(-c2ccc(-c3nc(-c4ccccc4-n4c5ccccc5c5c(-c6ccccc6)cccc54)nc(-n4c5ccccc5c5ccccc54)n3)c(-c3ccccc3)c2)cc1 ; c1ccc(-c2ccc(-c3nc(-c4ccccc4)nc(-n4c5ccccc5c5ccccc54)n3)c(-n3c4ccccc4c4ccccc43)c2)cc1 ; c1ccc(-c2cccc(-c3nc(-c4ccc(-c5ccccc5)cc4-n4c5ccccc5c5ccccc54)nc(-n4c5ccccc5c5ccccc54)n3)c2)cc1]

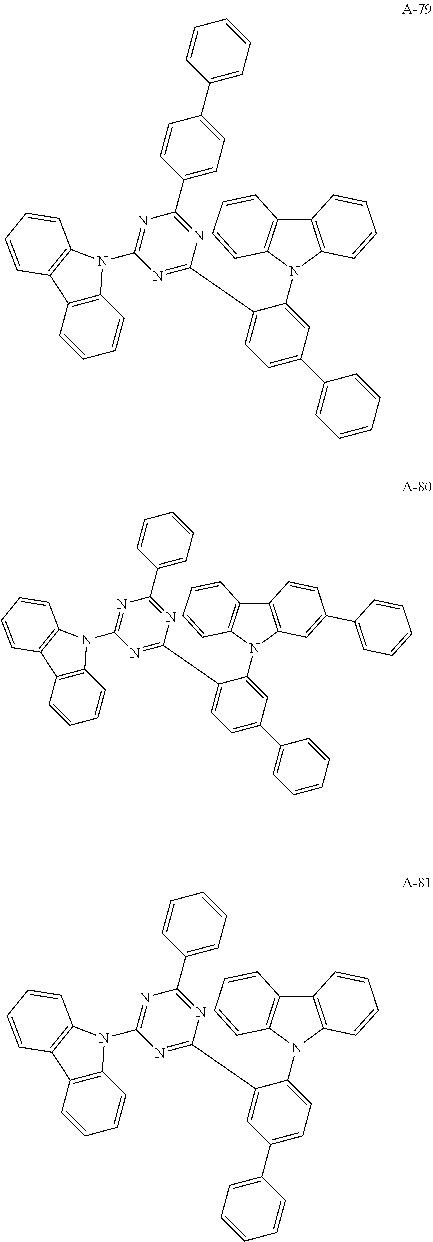 [SMILES: c1ccc(-c2ccc(-c3nc(-c4ccc(-c5ccccc5)cc4-n4c5ccccc5c5ccccc54)nc(-n4c5ccccc5c5ccccc54)n3)cc2)cc1 ; c1ccc(-c2ccc(-c3nc(-c4ccccc4)nc(-n4c5ccccc5c5ccccc54)n3)c(-n3c4ccccc4c4ccc(-c5ccccc5)cc43)c2)cc1 ; c1ccc(-c2ccc(-n3c4ccccc4c4ccccc43)c(-c3nc(-c4ccccc4)nc(-n4c5ccccc5c5ccccc54)n3)c2)cc1]

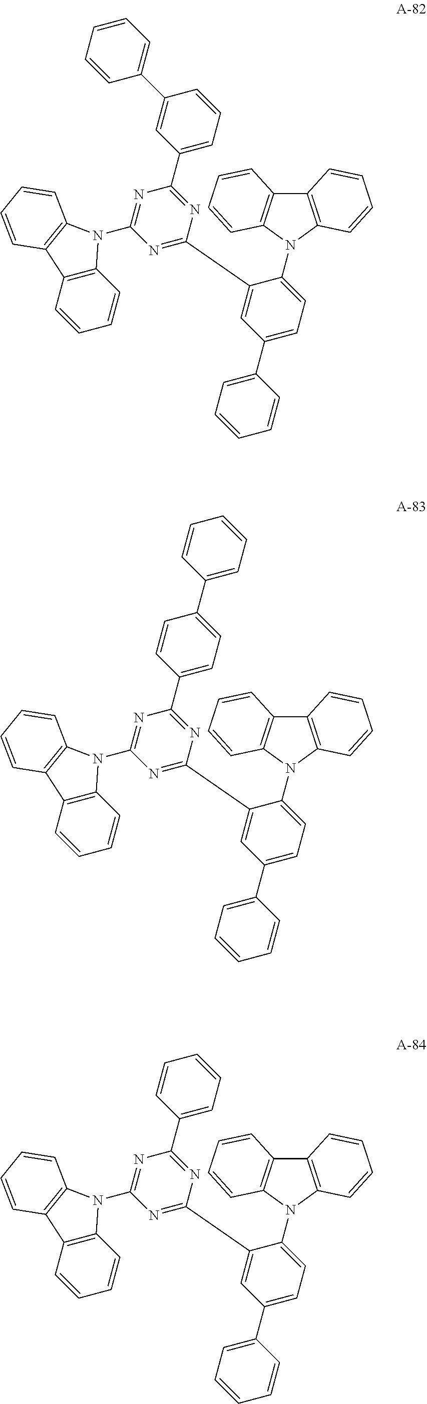 [SMILES: c1ccc(-c2cccc(-c3nc(-c4cc(-c5ccccc5)ccc4-n4c5ccccc5c5ccccc54)nc(-n4c5ccccc5c5ccccc54)n3)c2)cc1 ; c1ccc(-c2ccc(-c3nc(-c4cc(-c5ccccc5)ccc4-n4c5ccccc5c5ccccc54)nc(-n4c5ccccc5c5ccccc54)n3)cc2)cc1 ; c1ccc(-c2ccc(-n3c4ccccc4c4ccccc43)c(-c3nc(-c4ccccc4)nc(-n4c5ccccc5c5ccccc54)n3)c2)cc1]

The first compound may be used as each one type alone or a mixture of two types or more.

The second compound may be used (e.g., mixed) with the first compound in the light emitting layer to increase charge mobility and stability, resultantly improving luminous efficiency and life-span characteristics.

The second compound may be, e.g., represented by Chemical Formula 2.

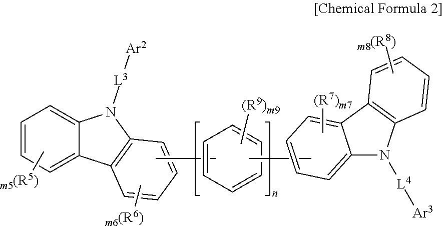

In Chemical Formula 2, Ar² and Ar³ may each independently be or include, e.g., a substituted or unsubstituted C6 to C20 aryl group or a substituted or unsubstituted C2 to C30 heterocyclic group.

L³ and L⁴ may each independently be or include, e.g., a single bond or a substituted or unsubstituted C6 to C20 arylene group.

R⁵ to R⁹ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

m5, m8, and m9 may each independently be, e.g., an integer of 1 to 4.

m6 and m7 may each independently be, e.g., an integer of 1 to 3.

n may be, e.g., an integer of 0 to 2.

In an implementation, Ar² and Ar³ in Chemical Formula 2 may independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group,

In an implementation, L³ and L⁴ in Chemical Formula 2 may independently be, e.g., a single bond, a substituted or unsubstituted phenylene group, or a substituted or unsubstituted biphenylene group.

In an implementation, R⁵ to R⁹ in Chemical Formula 2 may independently be, e.g., hydrogen, deuterium, or a substituted or unsubstituted C6 to C12 aryl group.

In an implementation, n may be 0 or 1.

In an implementation, “substituted” in Chemical Formula 2 means that at least one hydrogen is substituted with deuterium, a C1 to C4 alkyl group, a C6 to C18 aryl group, or a C2 to C30 heteroaryl group.

In an implementation, Chemical Formula 2 may be represented by one of Chemical Formula 2-1 to Chemical Formula 2-15.

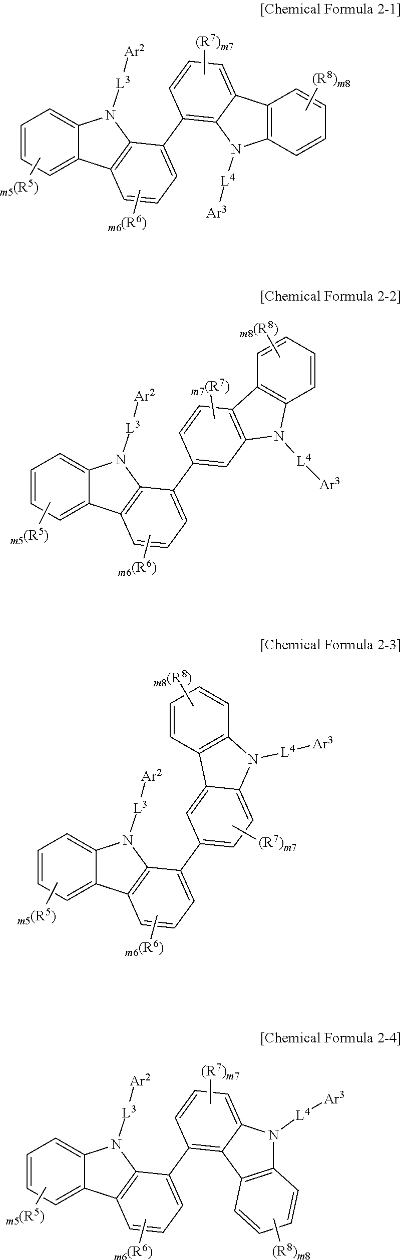

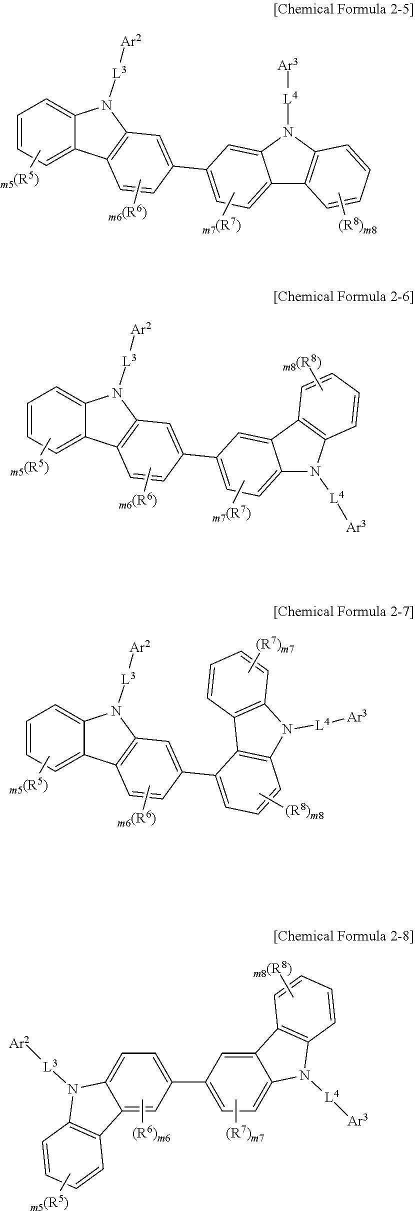

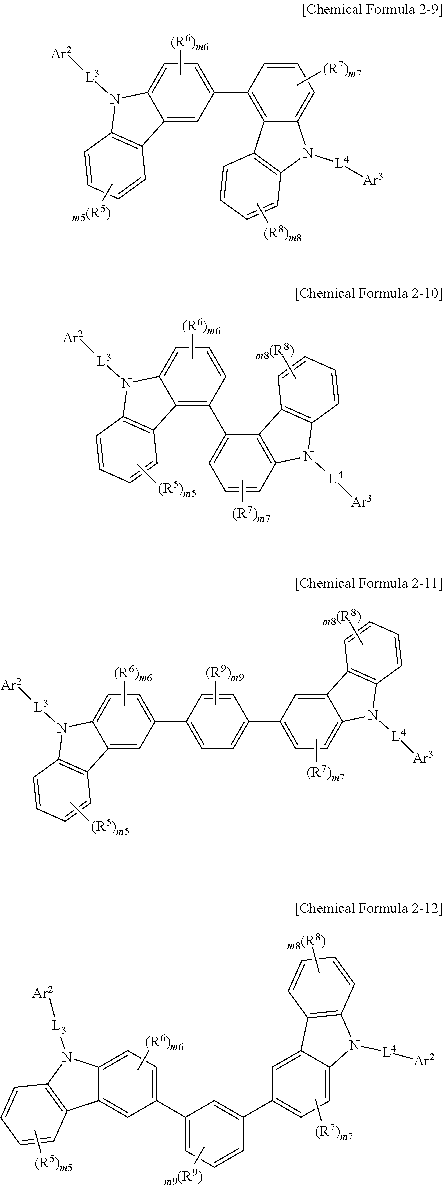

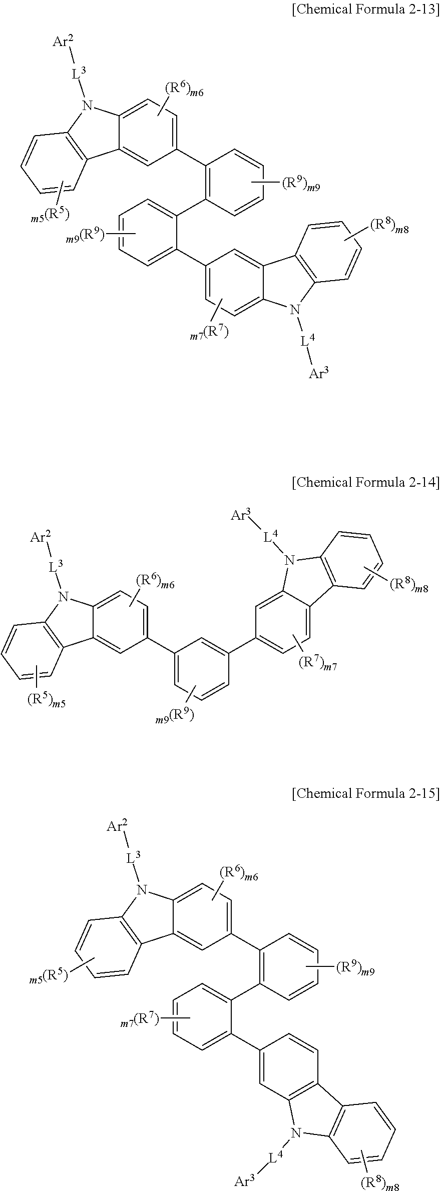

In Chemical Formula 2-1 to Chemical Formula 2-15, R⁵ to R⁹ may independently be, e.g., hydrogen, deuterium, or a substituted or unsubstituted C6 to C12 aryl group, and moieties *-L³-Ar² and *-L⁴-Ar³ may independently be a moiety of Group II.

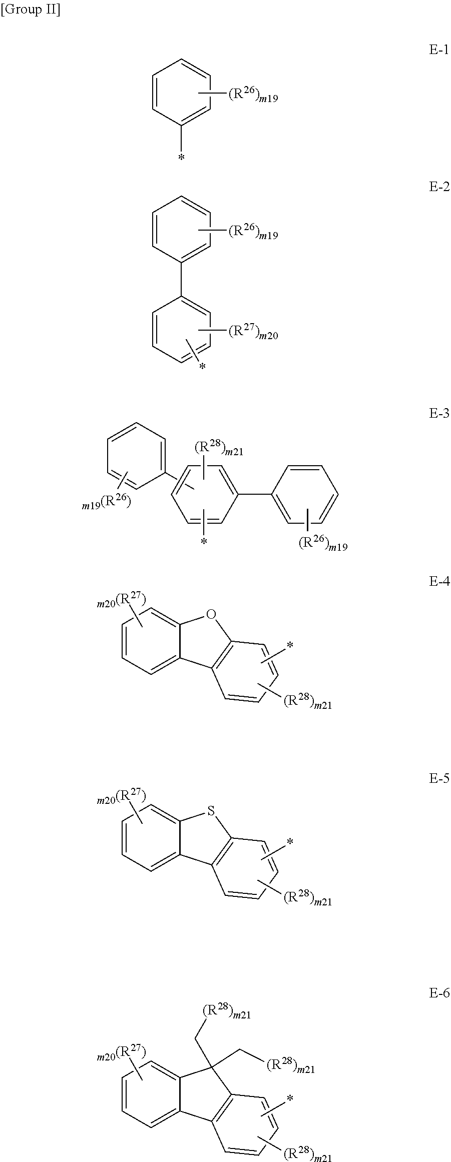

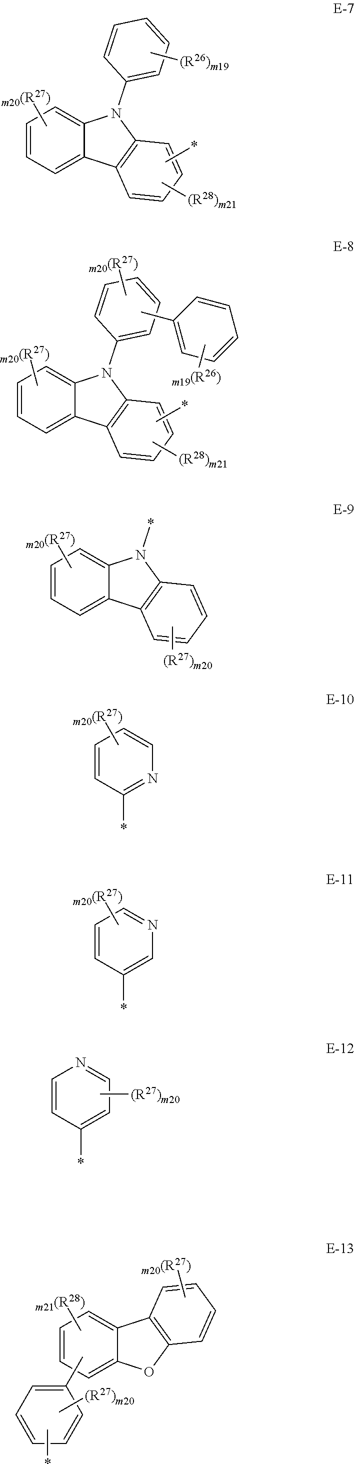

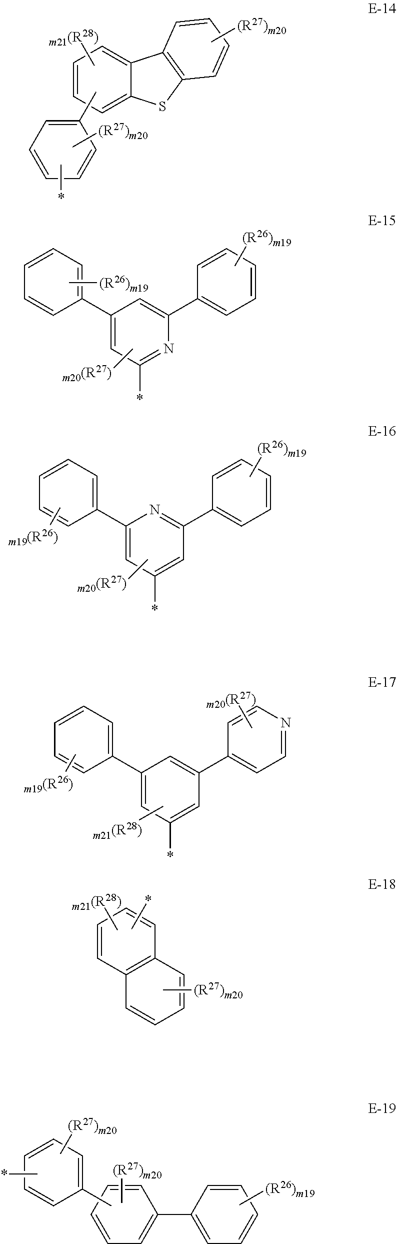

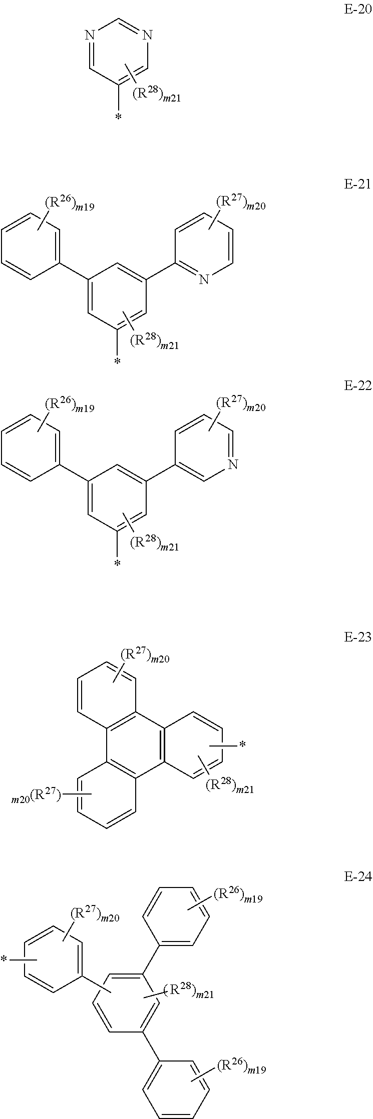

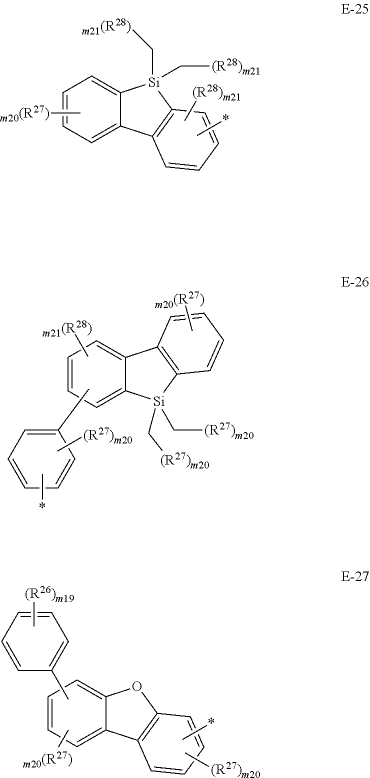

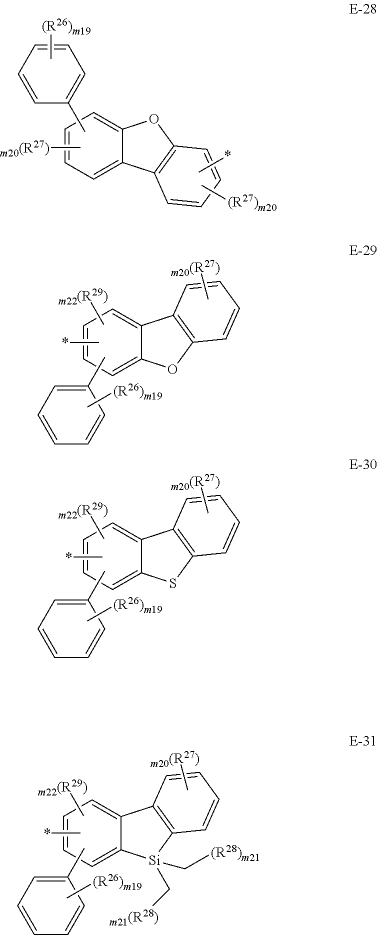

In Group II, R²⁶ to R²⁹ may each independently be, e.g., hydrogen, deuterium, a cyano group, a substituted or unsubstituted C1 to C10 alkyl group, or a substituted or unsubstituted C6 to C12 aryl group.

m19 may be, e.g., an integer of 1 to 5.

m20 may be, e.g., an integer of 1 to 4.

m21 may be, e.g., an integer of 1 to 3.

m22 may be, e.g., 1 or 2.

* is a linking point.

In an implementation, Chemical Formula 2 may be represented by Chemical Formula 2-8.

In an implementation, moieties *-L³-Ar² and *-L⁴-Ar³ in Chemical Formula 2-8 may independently be a moiety of Group II, e.g., E-1, E-2, E-3, E-4, E-7, E-8, or E-9.

The second compound may be represented by, e.g., a combination of Chemical Formula 3 and Chemical Formula 4.

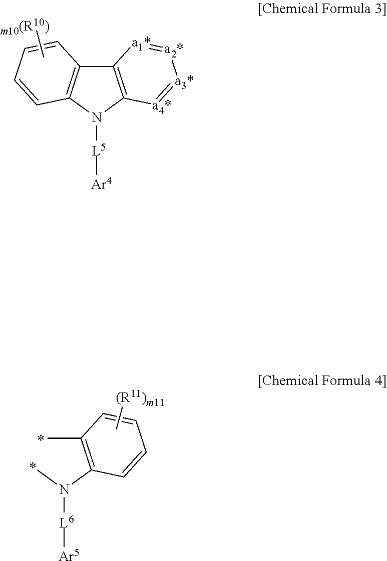

In Chemical Formula 3 and Chemical Formula 4, Ar⁴ and Ar⁵ may each independently be or include, e.g., a substituted or unsubstituted C6 to C20 aryl group or a substituted or unsubstituted C2 to C30 heterocyclic group.

Two adjacent ones of a₁* to a₄* in Chemical Formula 3 are linking carbons linked at * of Chemical Formula 4, the remaining two of a1* to a4* of Chemical Formula 3, not linked at * of Chemical Formula 4, are C-L^{a}-R^{a}. As used herein, the term “linking carbon” refers to a shared carbon at which fused rings are linked.

L^{a}, L⁵, and L⁶ may each independently be or include, e.g., a single bond or a substituted or unsubstituted C6 to C20 arylene group.

R^{a}, R¹⁰, and R¹¹ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

m10 and m11 may each independently be, e.g., an integer of 1 to 4.

In an implementation, the second compound represented by the combination of Chemical Formula 3 and Chemical Formula 4 may be represented by any one of Chemical Formula 3A, Chemical Formula 3B, Chemical Formula 3C, Chemical Formula 3D, and Chemical Formula 3E.

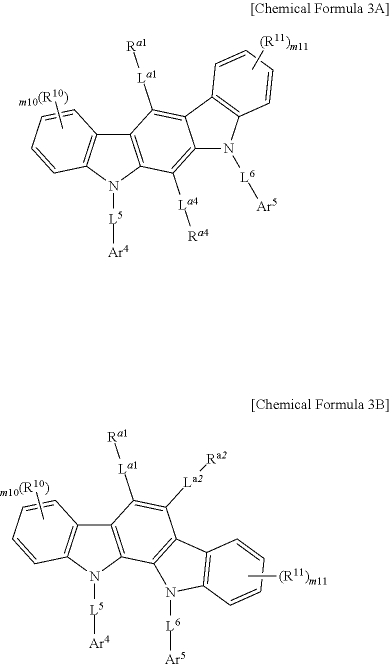

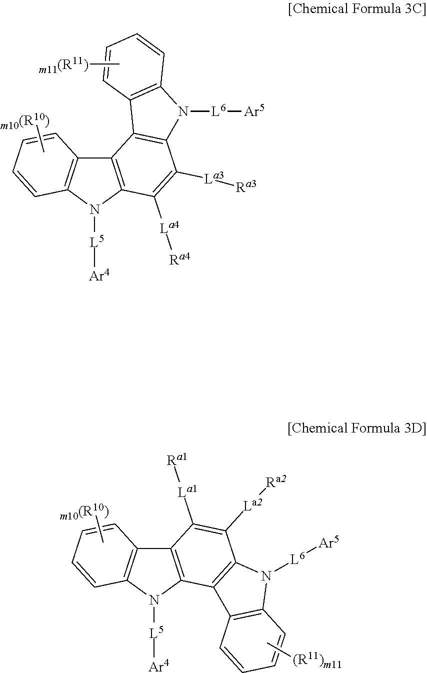

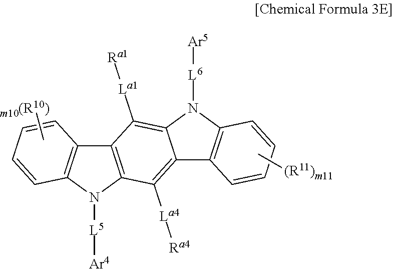

In Chemical Formula 3A to Chemical Formula 3E, Ar⁴, Ar⁵, L⁵, L⁶, R¹⁰ and R¹¹ may be the same as described above.

L^{a1} to L^{a4} may be defined the same as L⁵ and L⁶ described above.

R^{a1} to R^{a4} may be defined the same as R¹⁰ and R¹¹.

In an implementation, Ar⁴ and Ar⁵ in Chemical Formulae 3 and 4 may each independently be, e.g., be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

R^{a1} to R^{a4}, R¹⁰, and R¹¹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, Ar⁴ and Ar⁵ in Chemical Formula 3 and 4 may independently be, e.g., a. group of Group II.

In an implementation, R^{a1} to R^{a4}, R¹⁰, and R¹¹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{a1} to R^{a4}, R¹⁰, and R¹¹ may each independently be, e.g., hydrogen, deuterium, a cyano group, or a substituted or unsubstituted phenyl group.

In an implementation, R^{a1} to R^{a4}, R¹⁰, and R¹¹ may each independently be, e.g., hydrogen, deuterium, or a substituted or unsubstituted phenyl group.

In an implementation, the second compound may be represented by Chemical Formula 2-8. In an implementation, in Chemical Formula 2-8, Ar² and Ar³ may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L³ and L⁴ may each independently be, e.g., a single bond, or a substituted or unsubstituted C6 to C20 arylene group, and R⁵ to R⁸ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, the second compound may be represented by Chemical Formula 3C. In an implementation, in Chemical Formula 3C, L^{a3} and L^{a4} may be a single bond, L⁵ and L⁶ may each independently be, e.g., a single bond or a substituted or unsubstituted C6 to C12 arylene group, R¹⁰, R¹¹, R^{a3} and R^{a4} may each independently be, e.g., hydrogen, deuterium, or a substituted or unsubstituted phenyl group, and Ar⁴ and Ar⁵ may each independently be, e.g., a substituted or unsubstituted phenyl group, a substituted or unsubstituted biphenyl group, or a substituted or unsubstituted terphenyl group.

In an implementation, the second compound may be a compound Group 2.

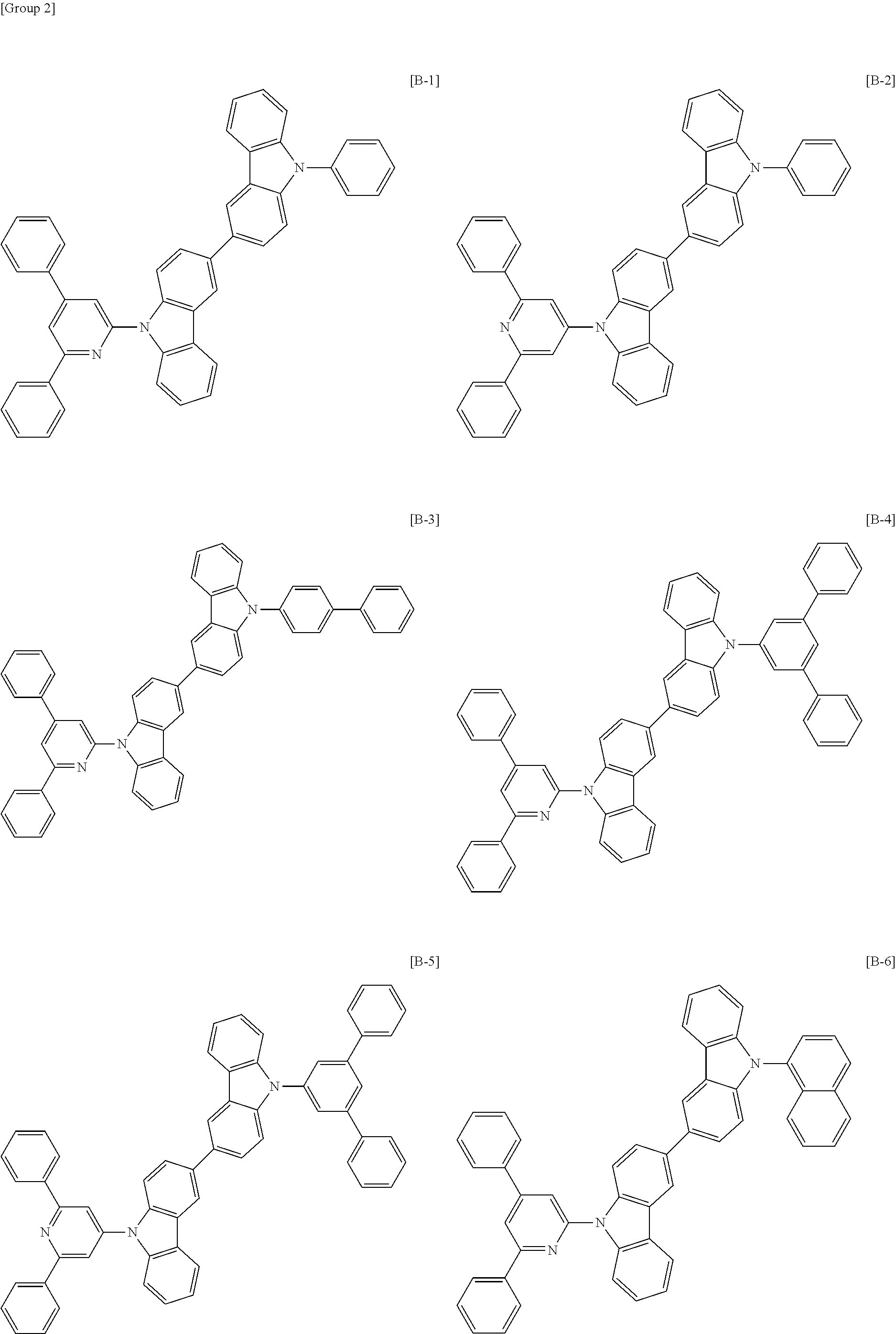

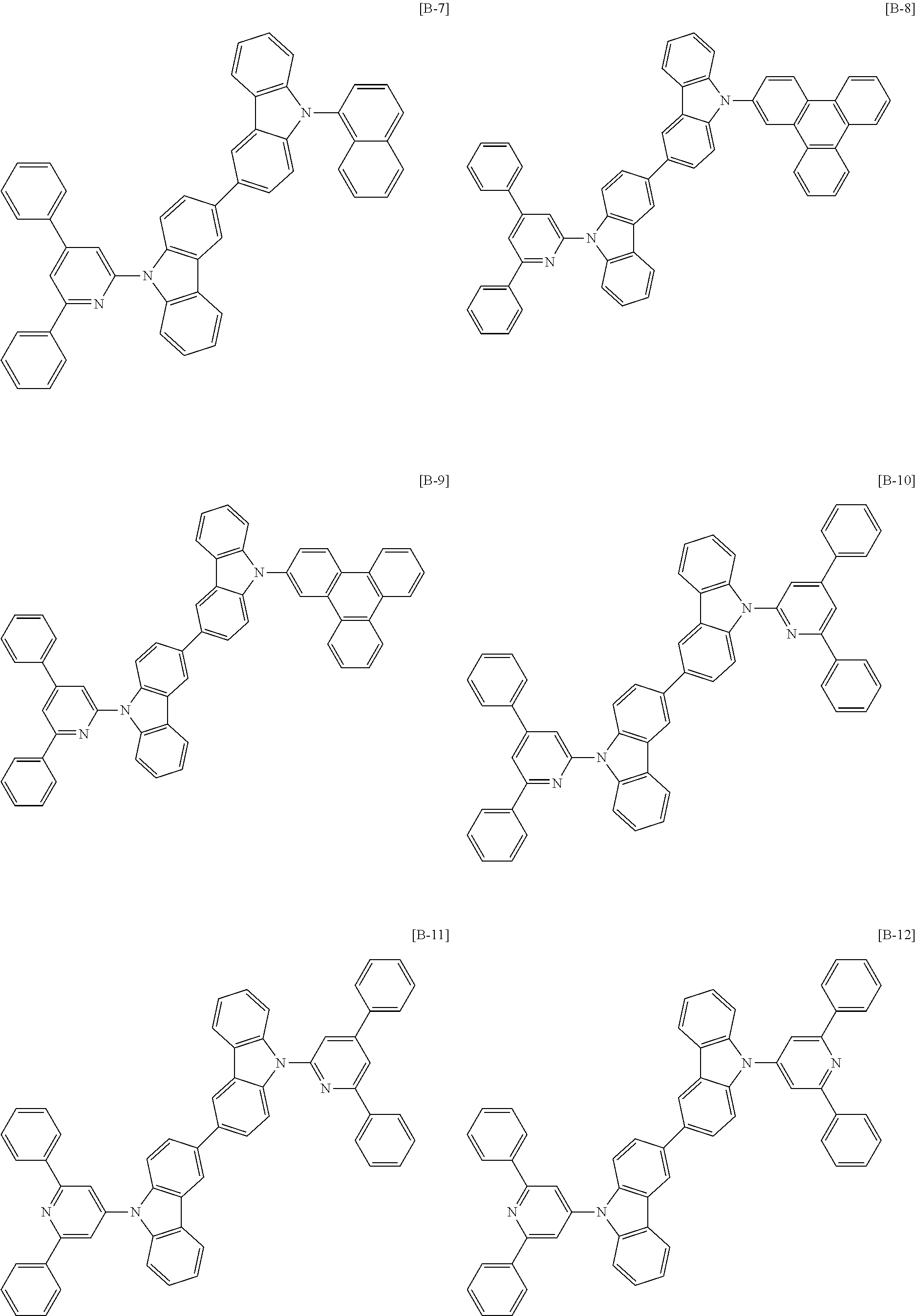

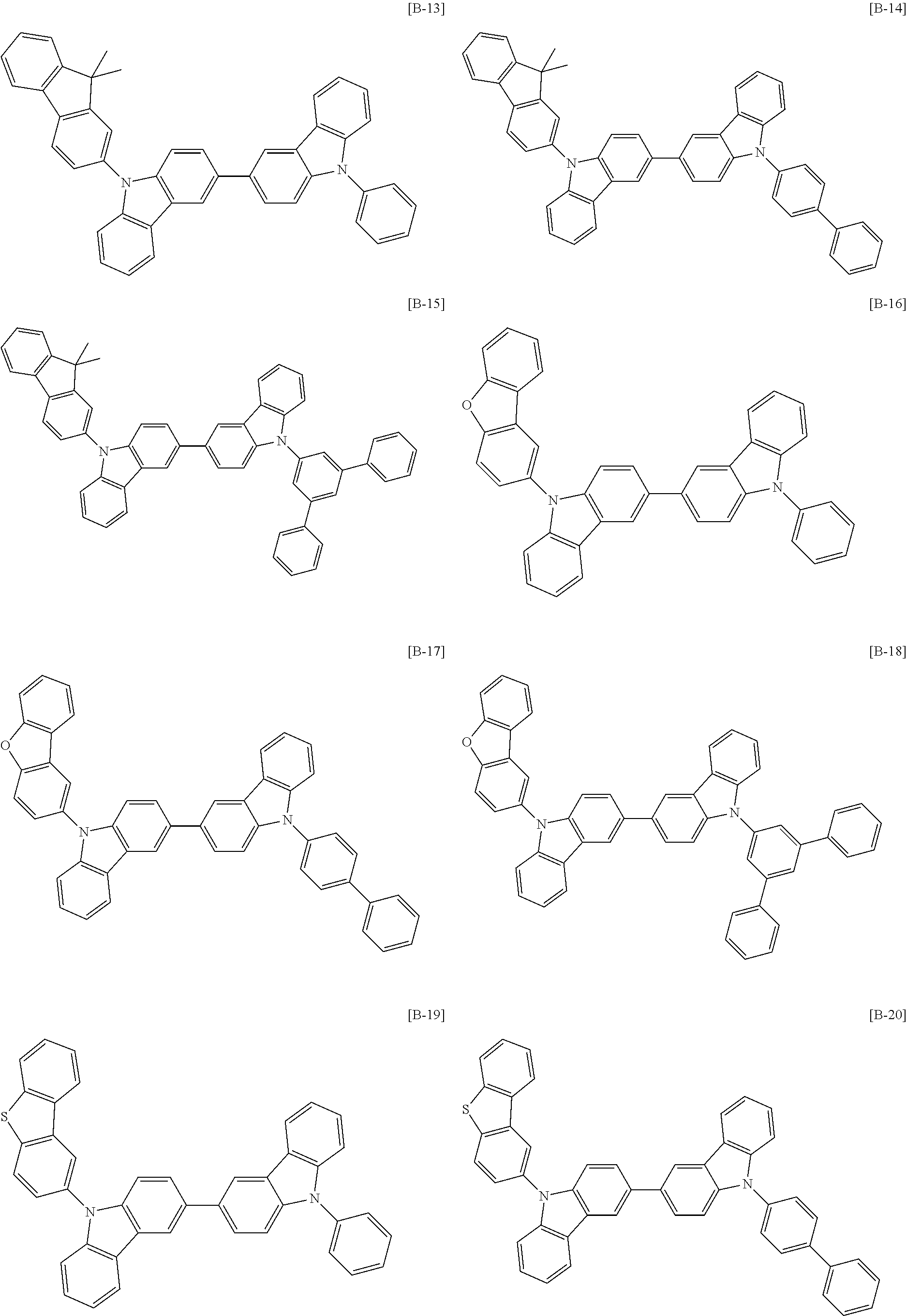

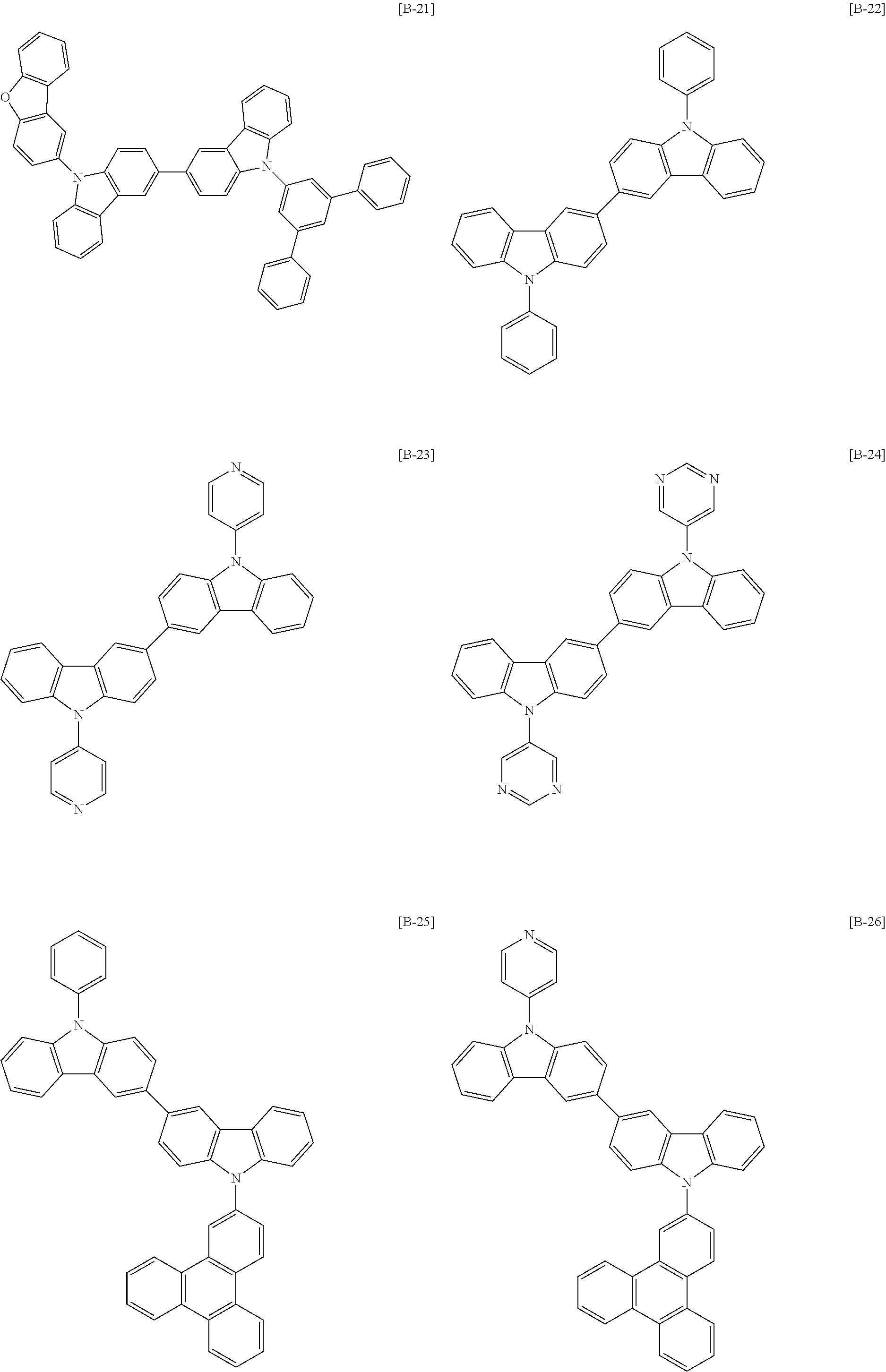 [SMILES: c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21]

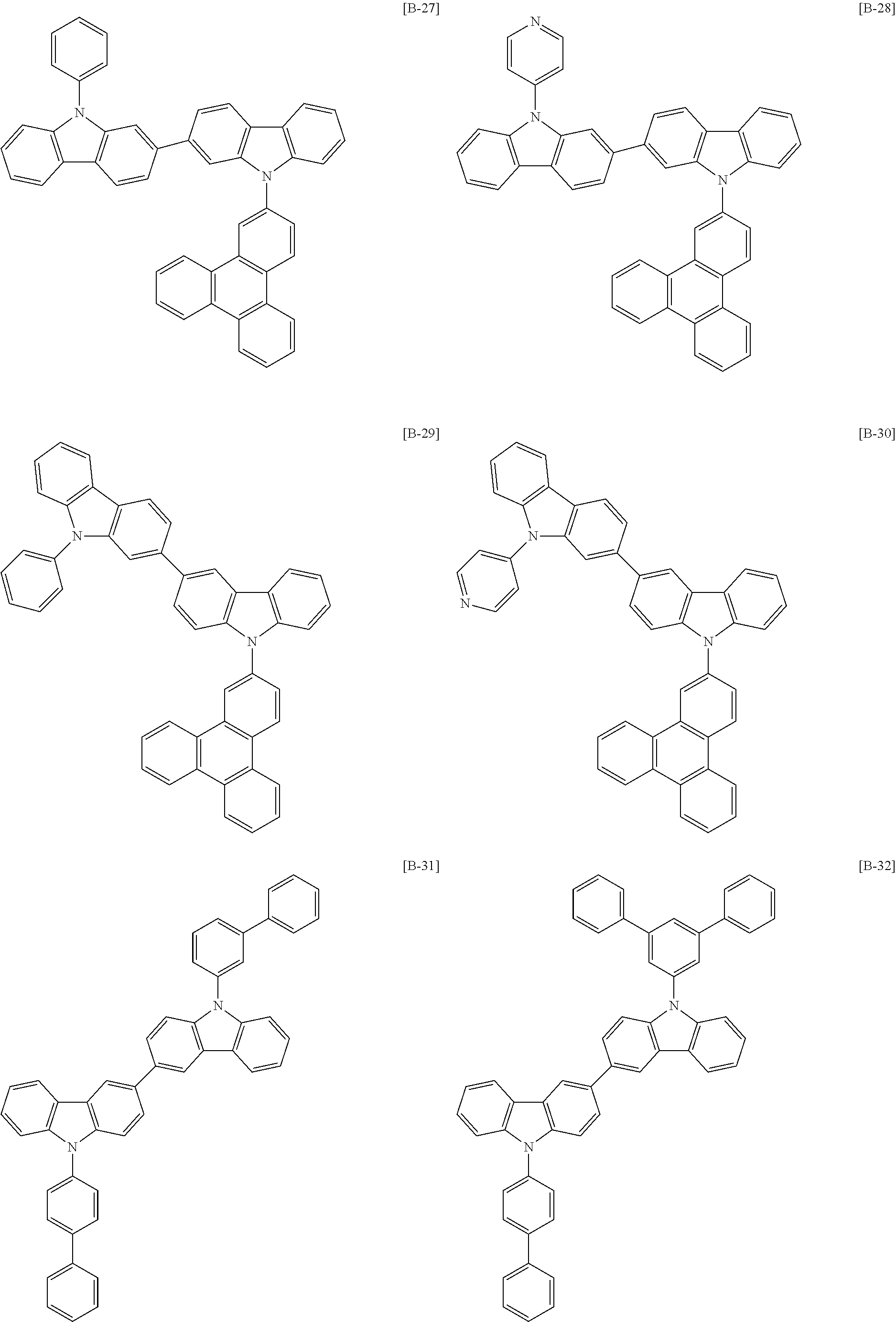

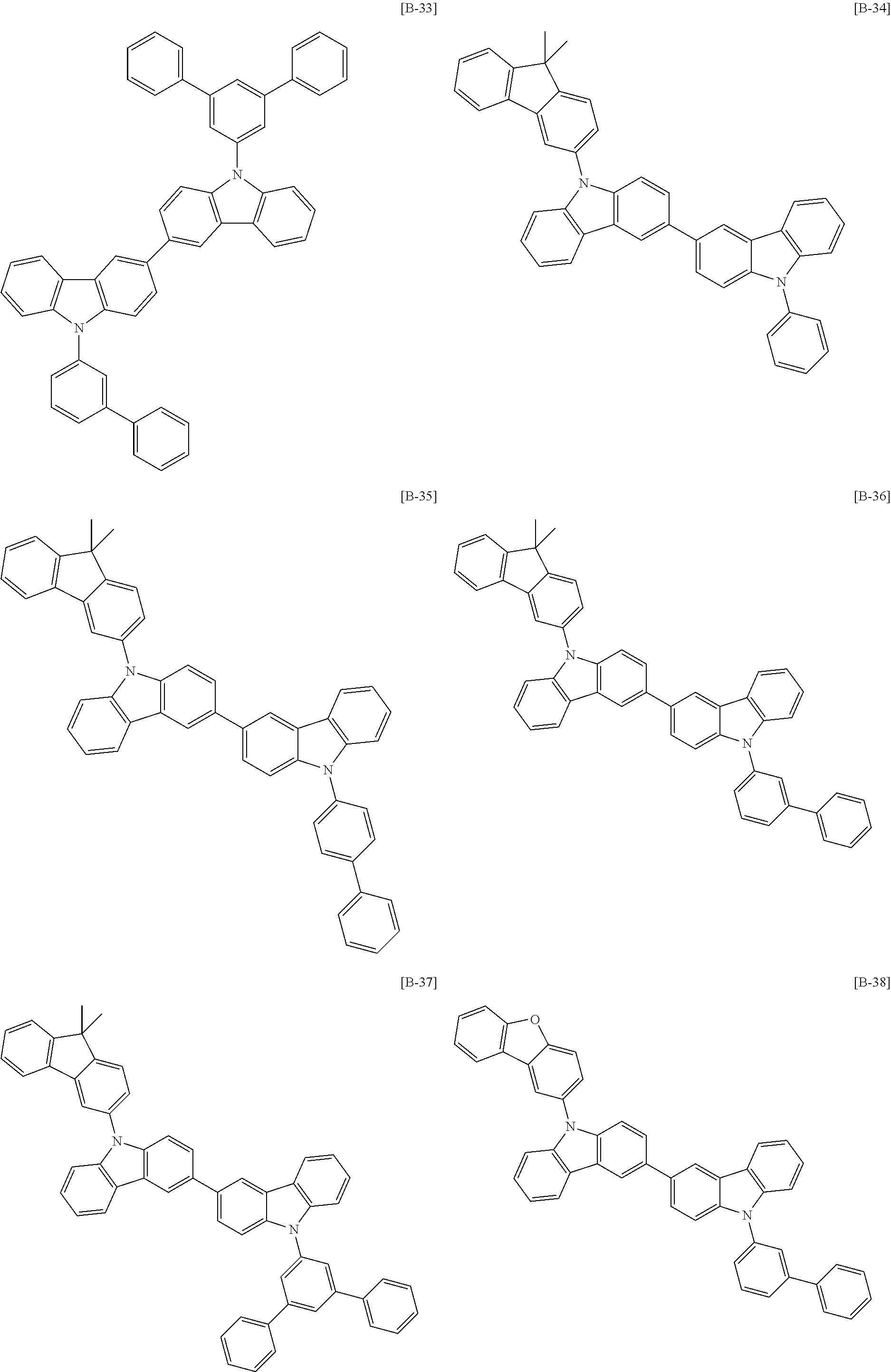

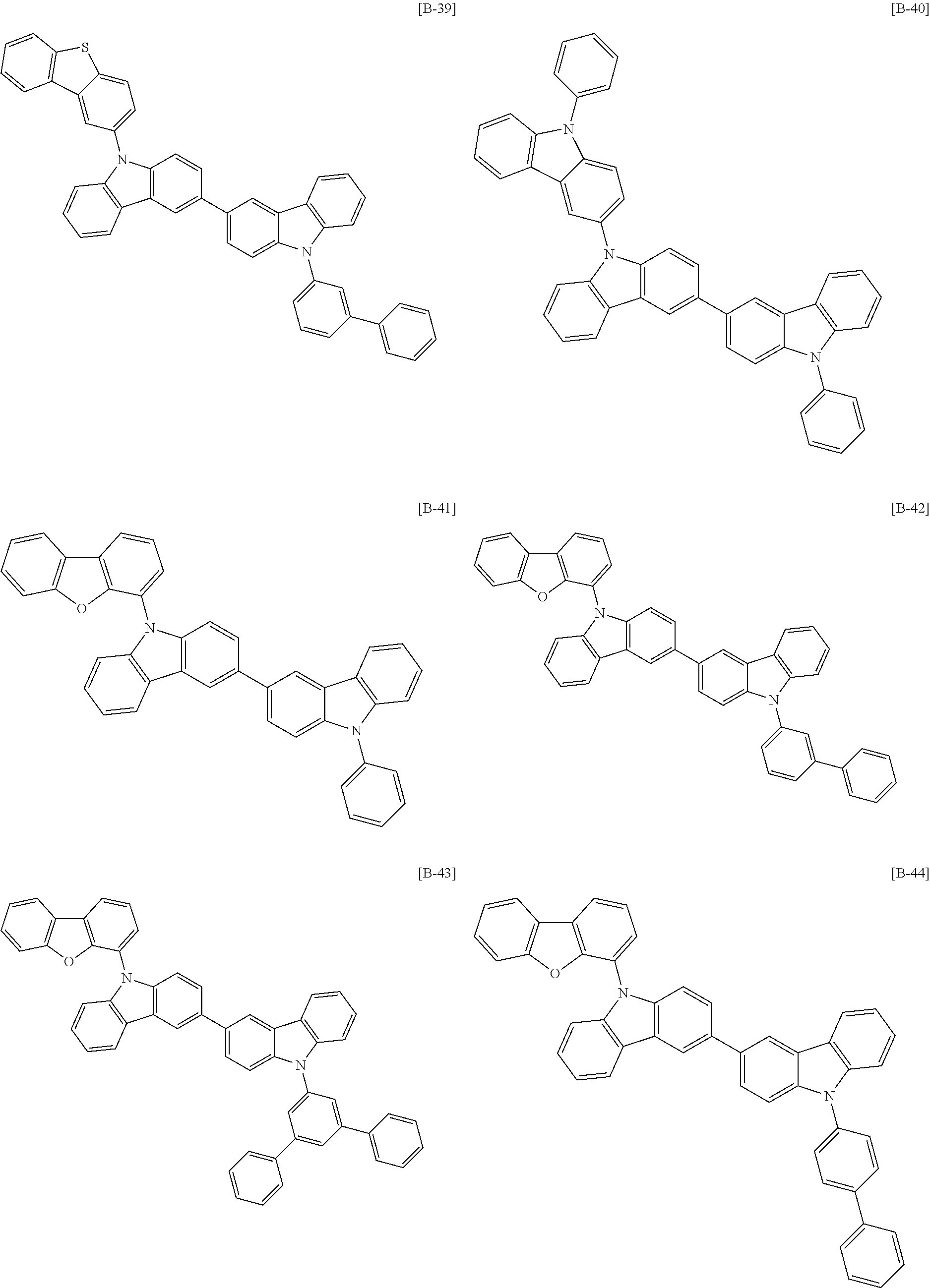

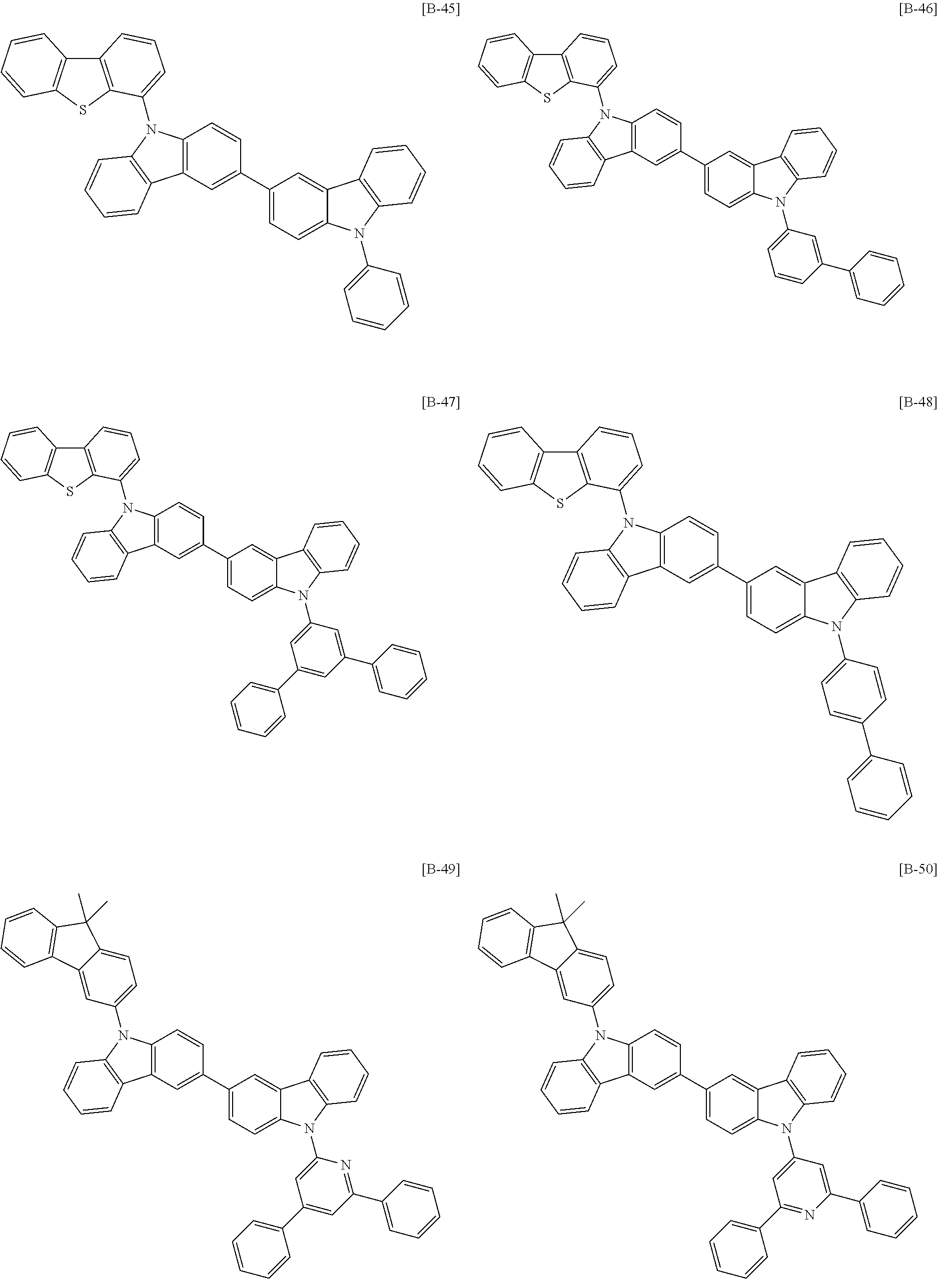

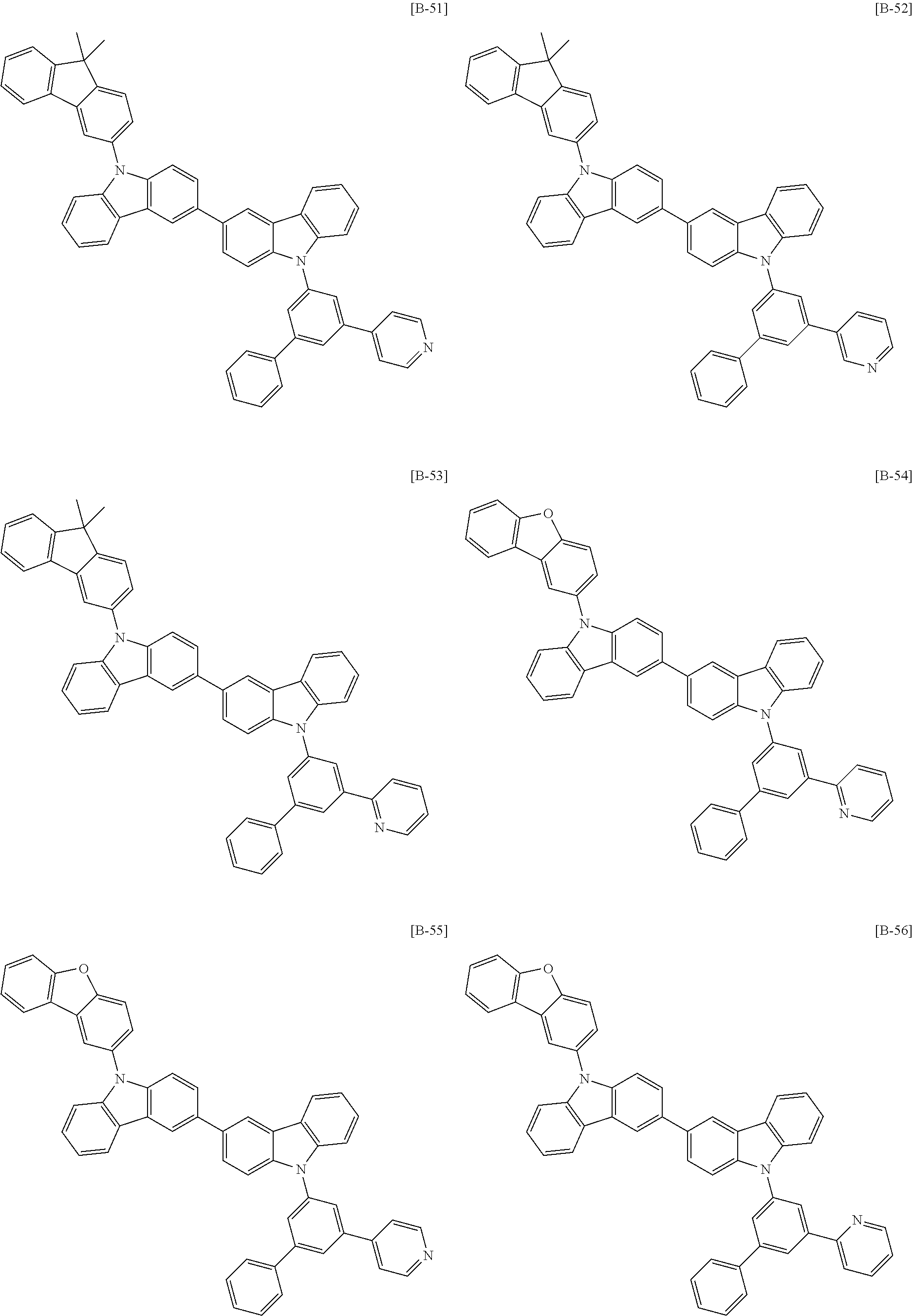

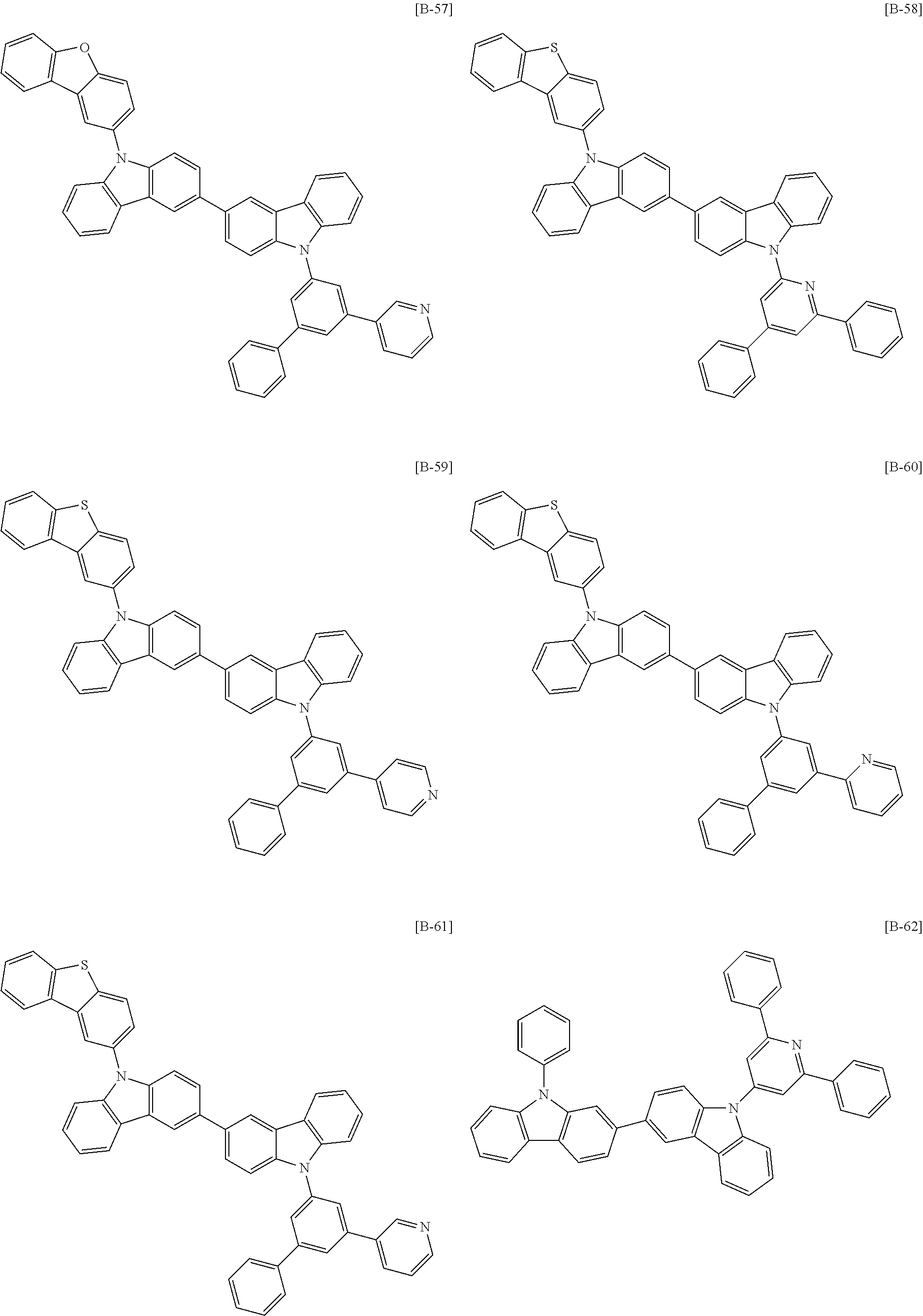

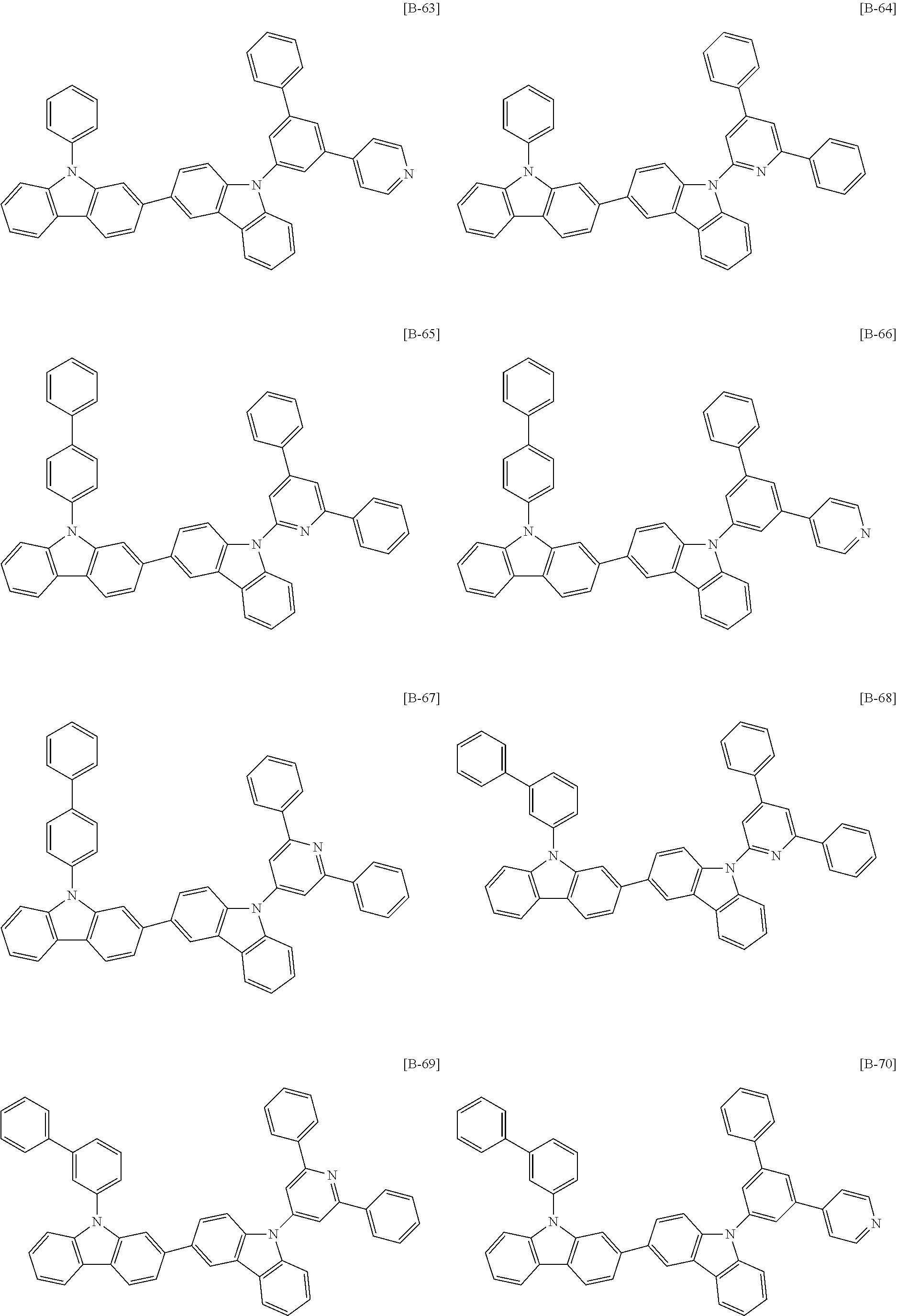

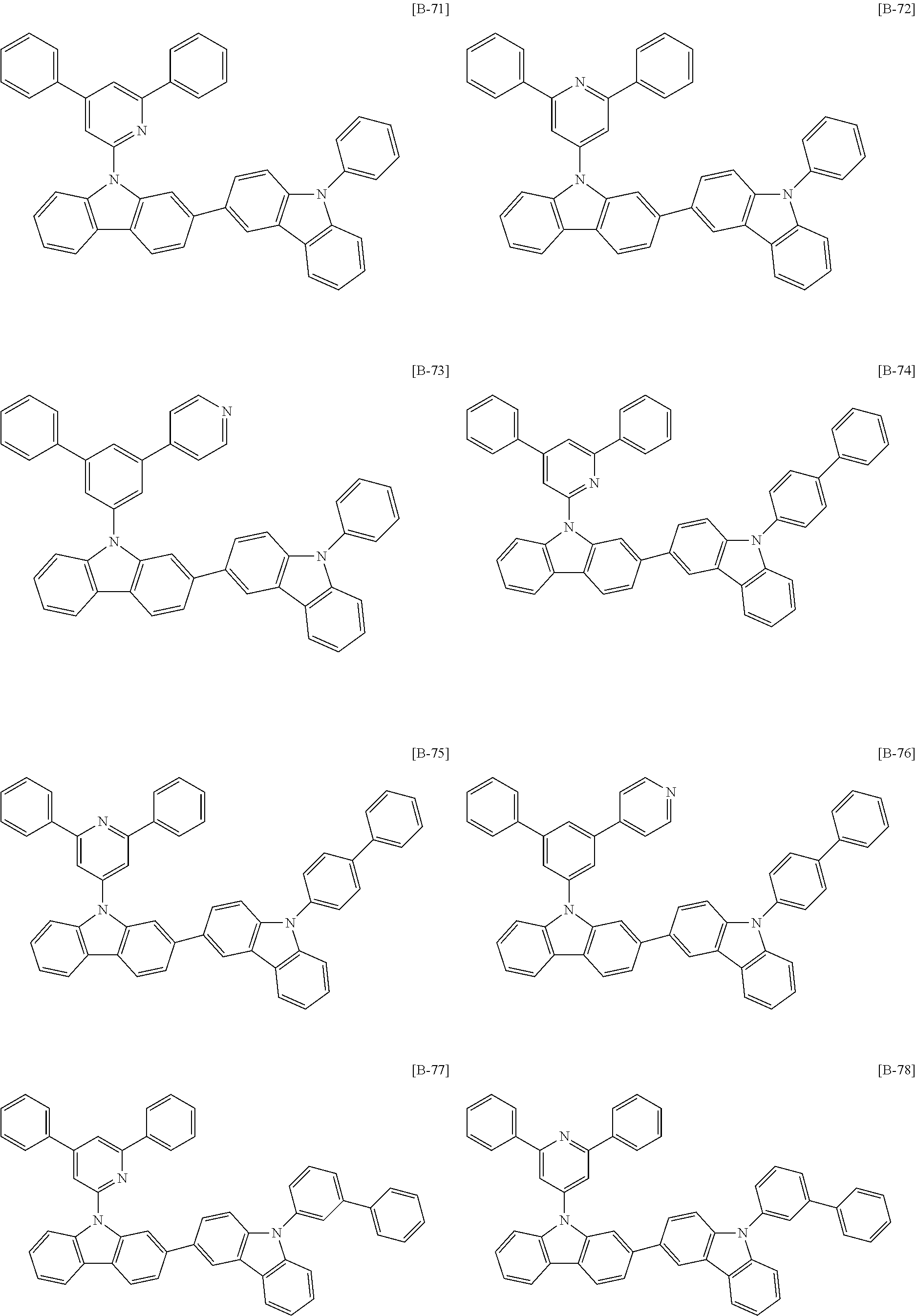

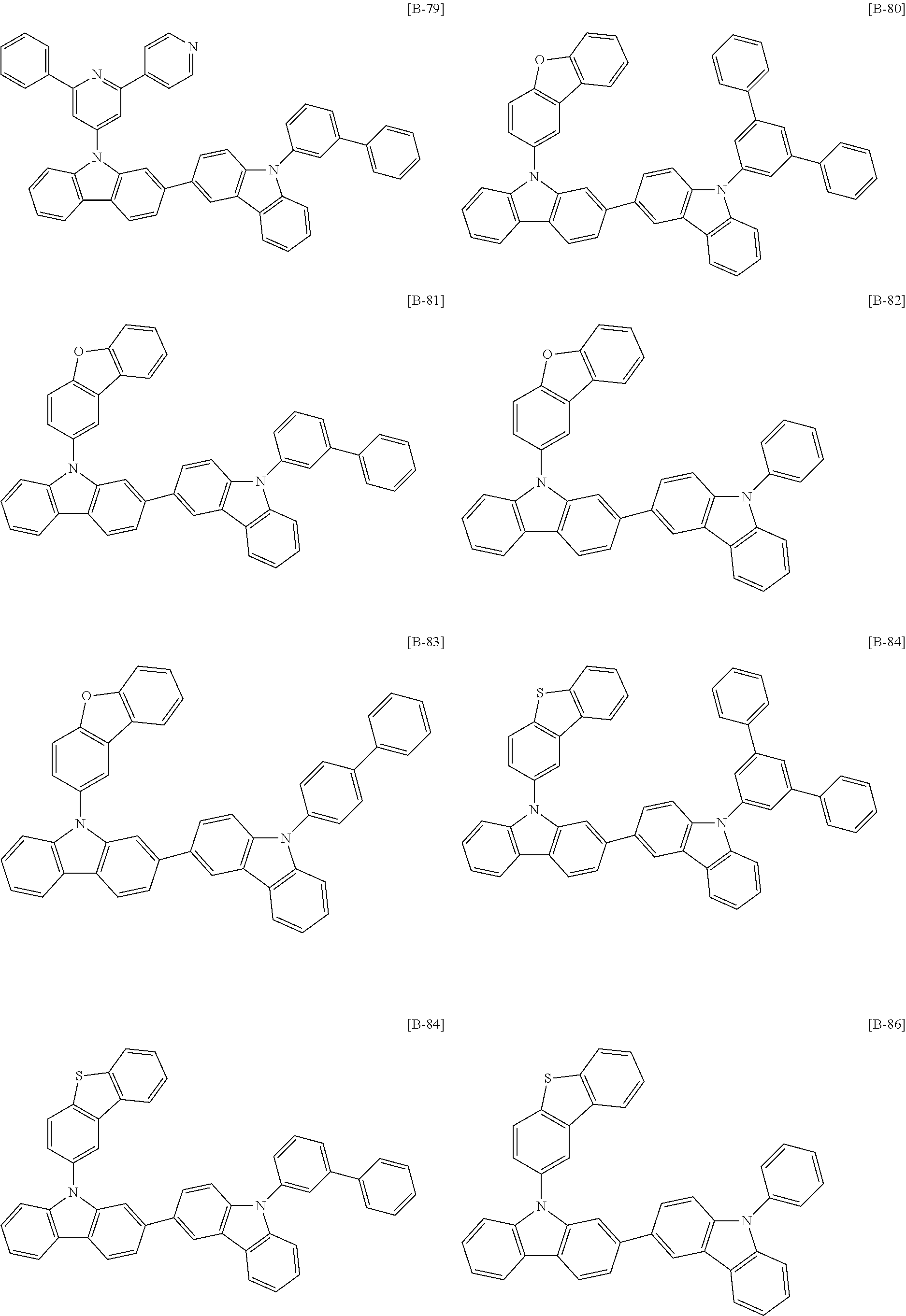

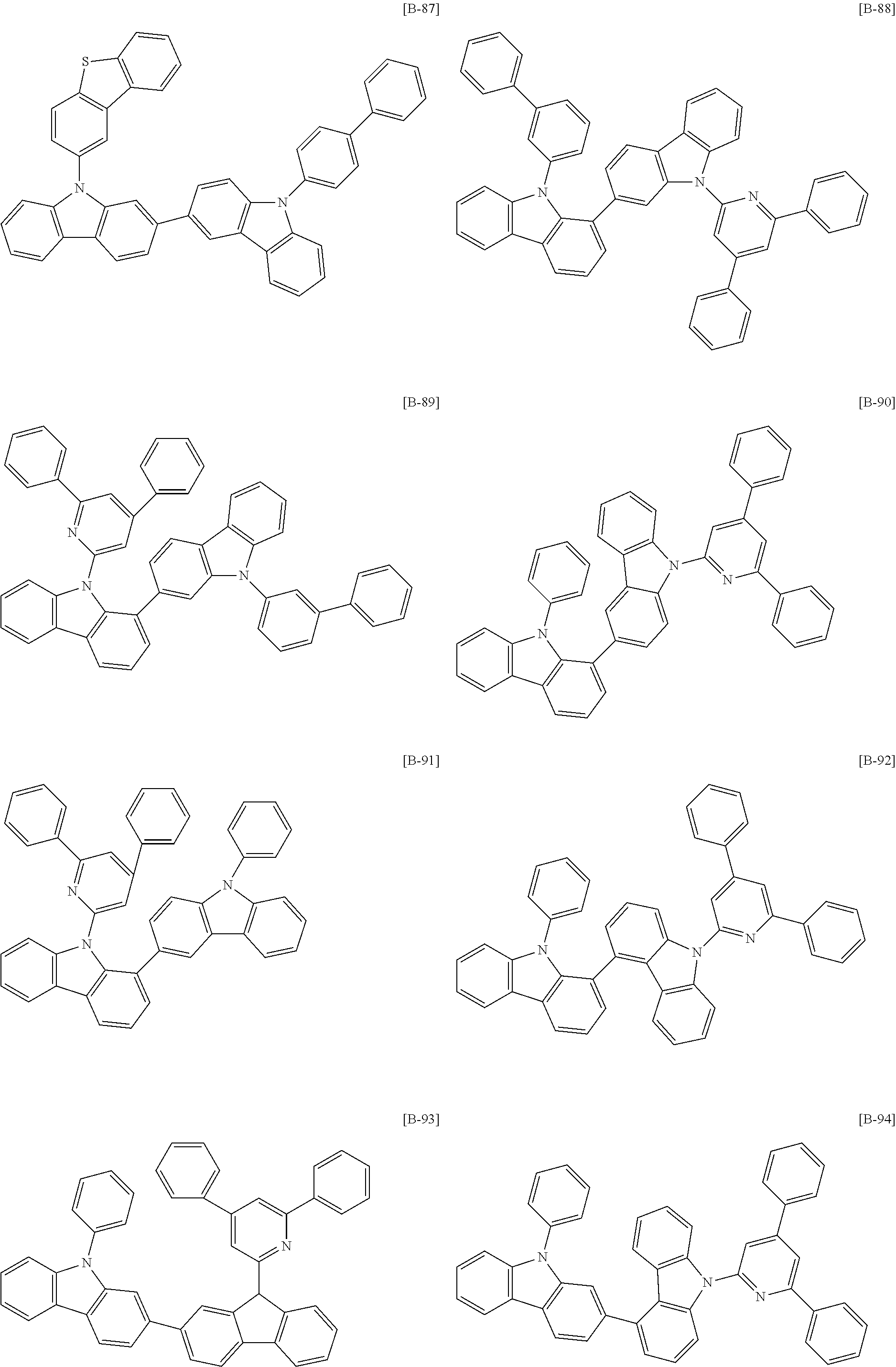

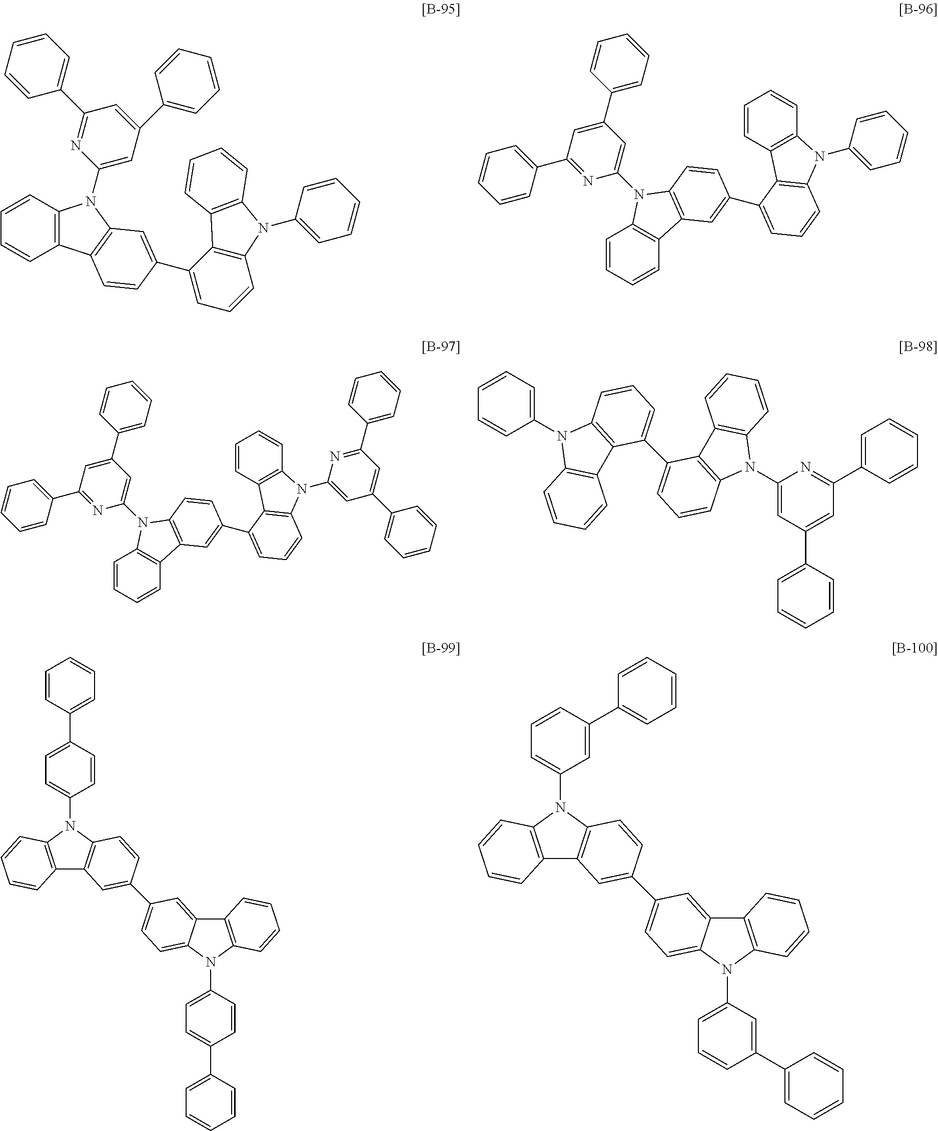

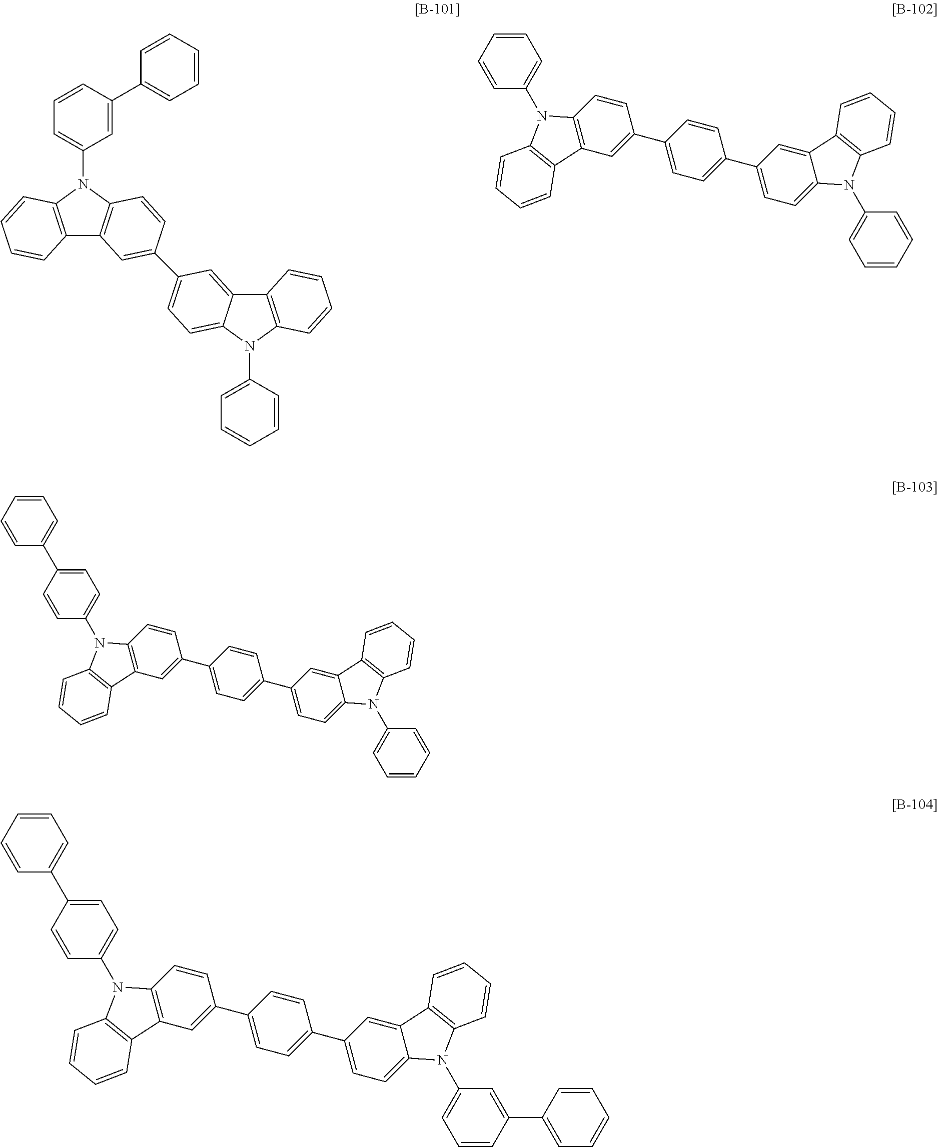 [SMILES: c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1 ; c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1]

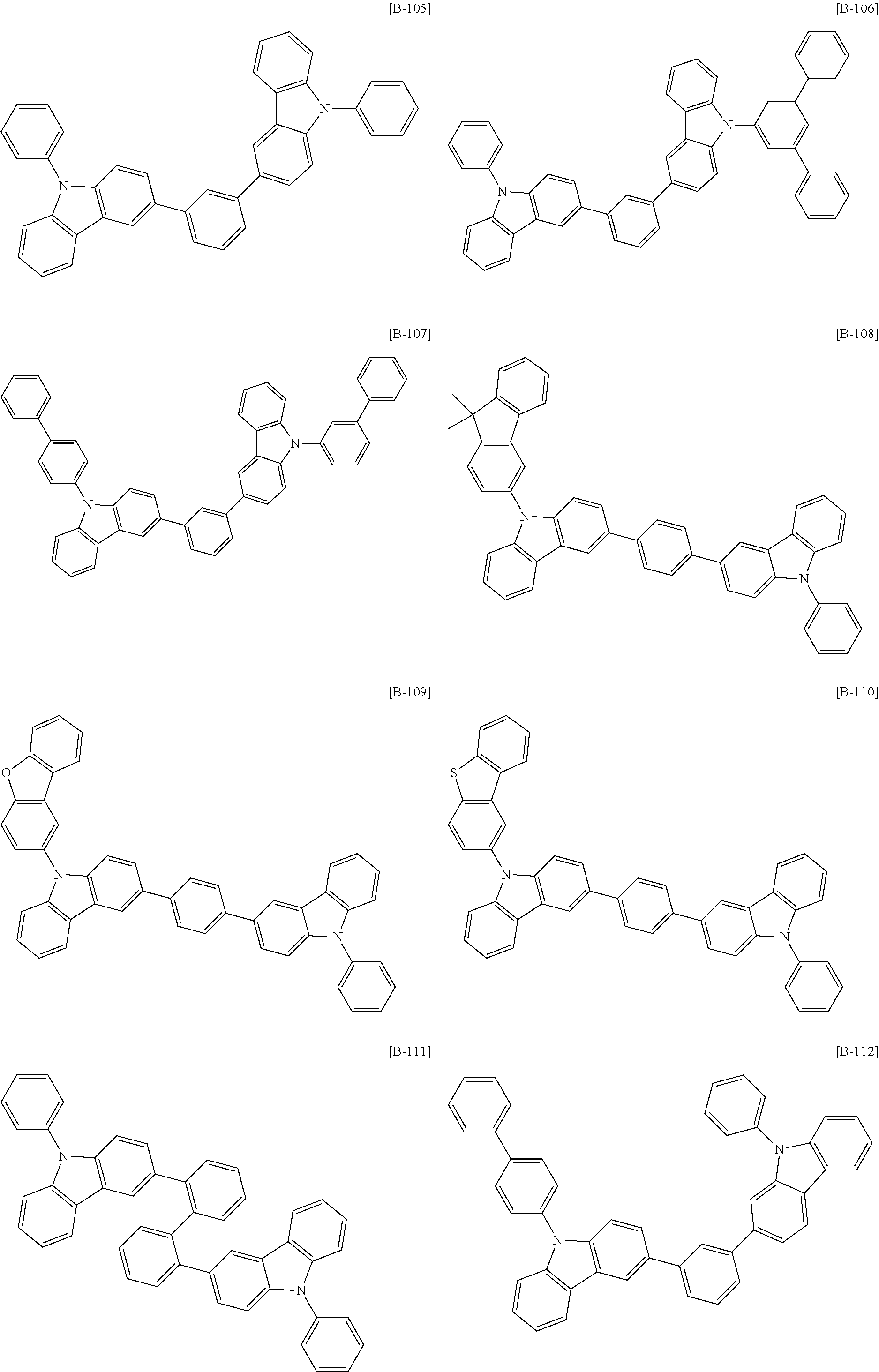

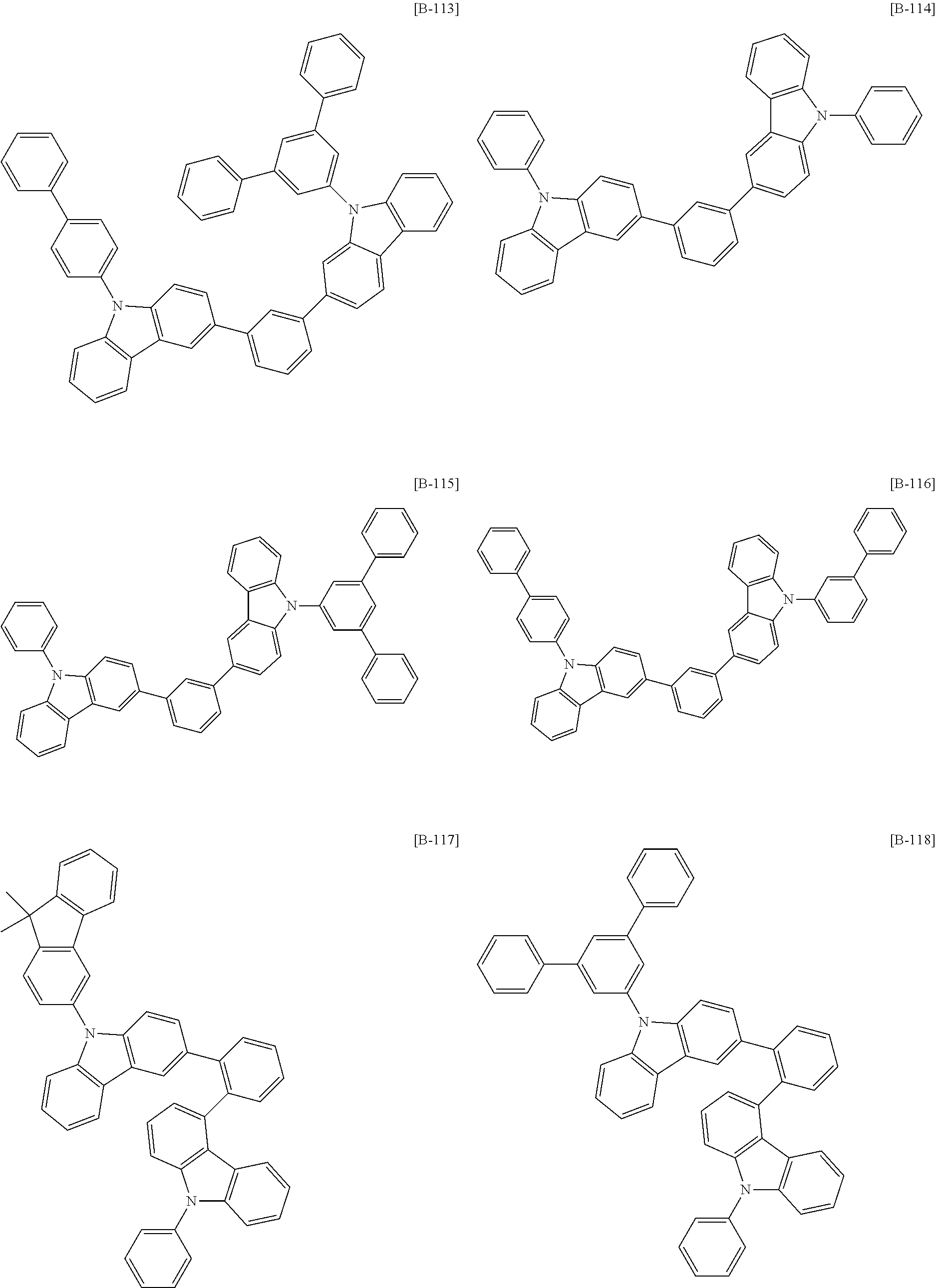

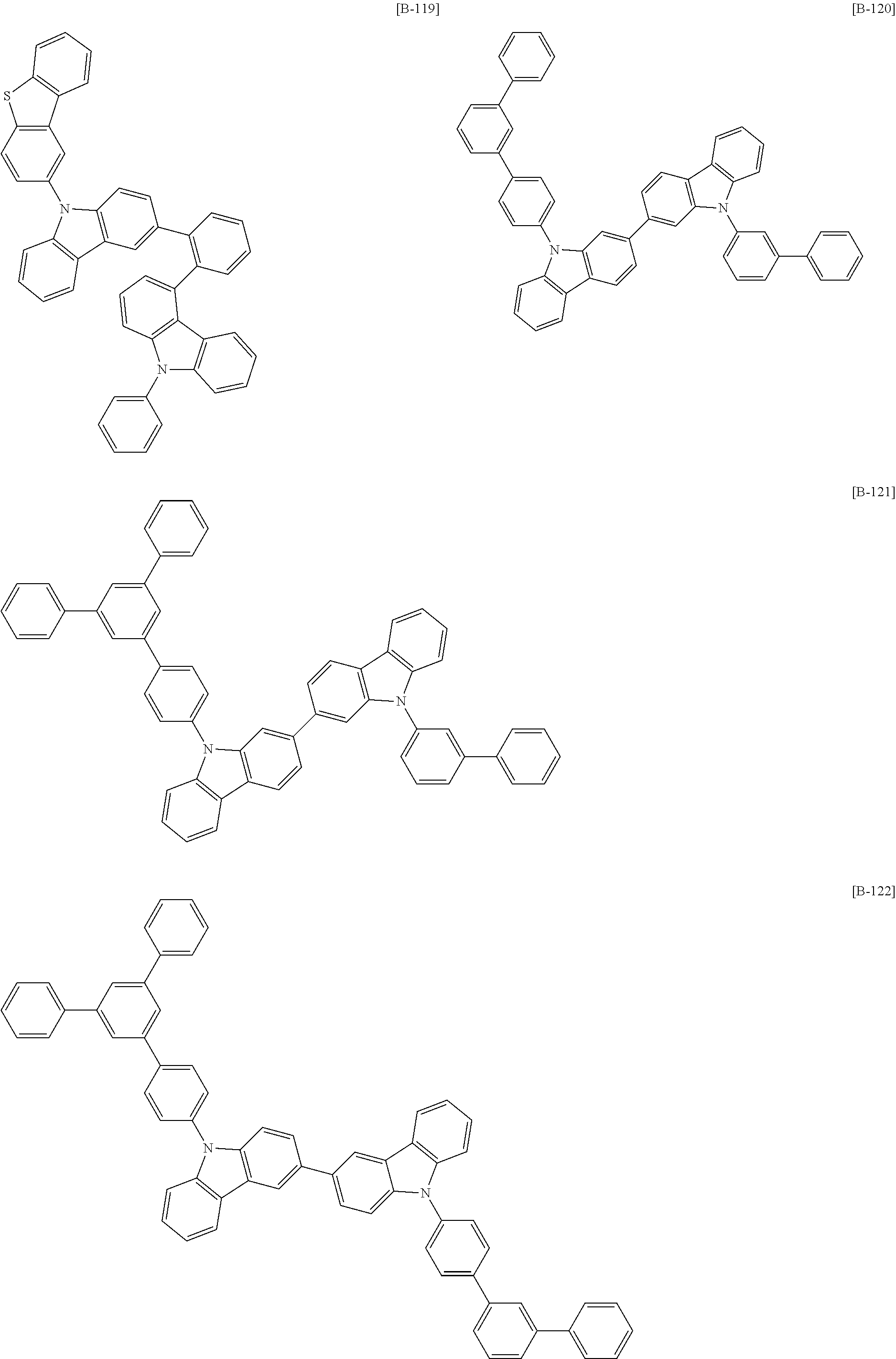 [SMILES: c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

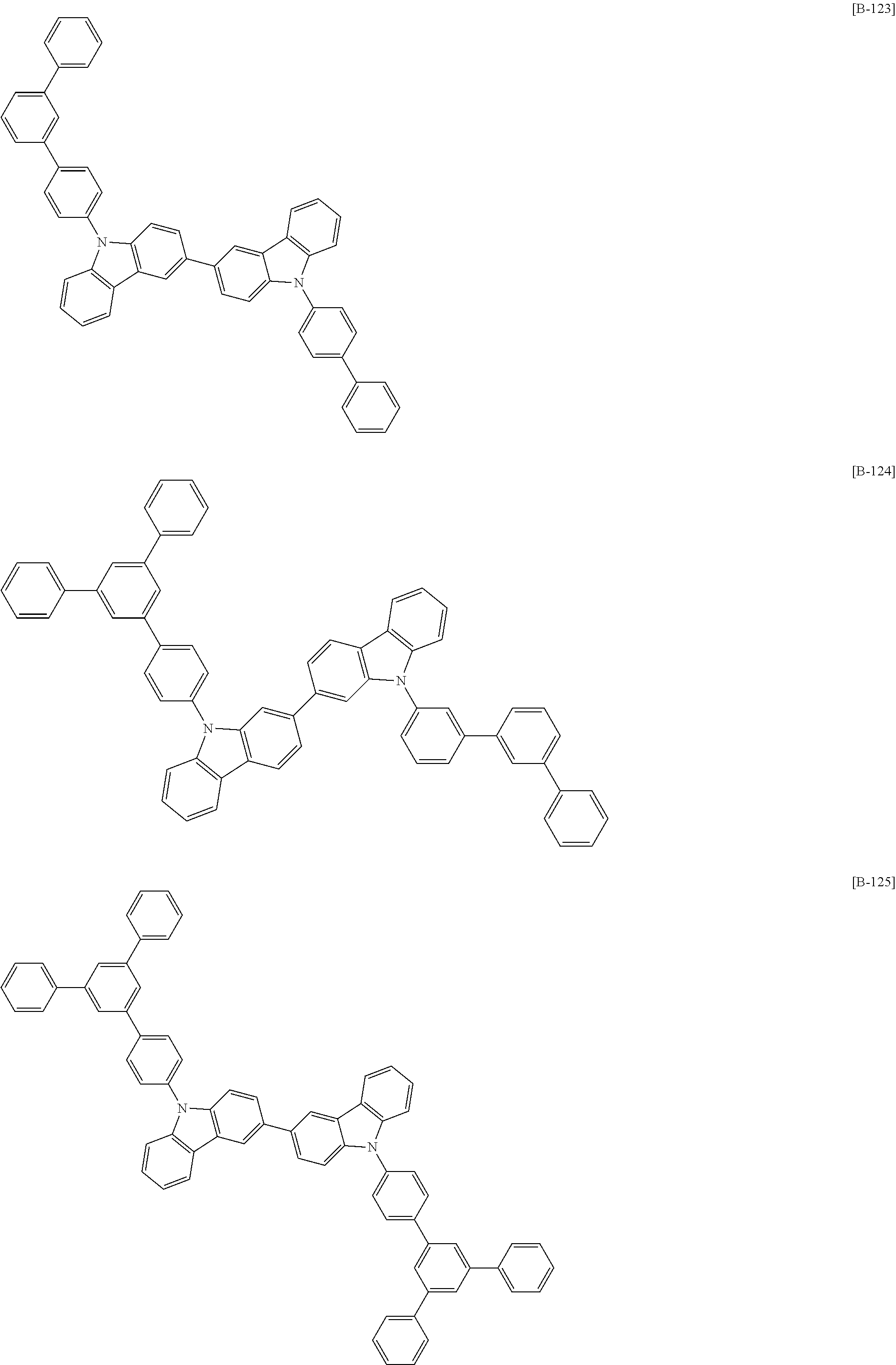 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1 ; c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

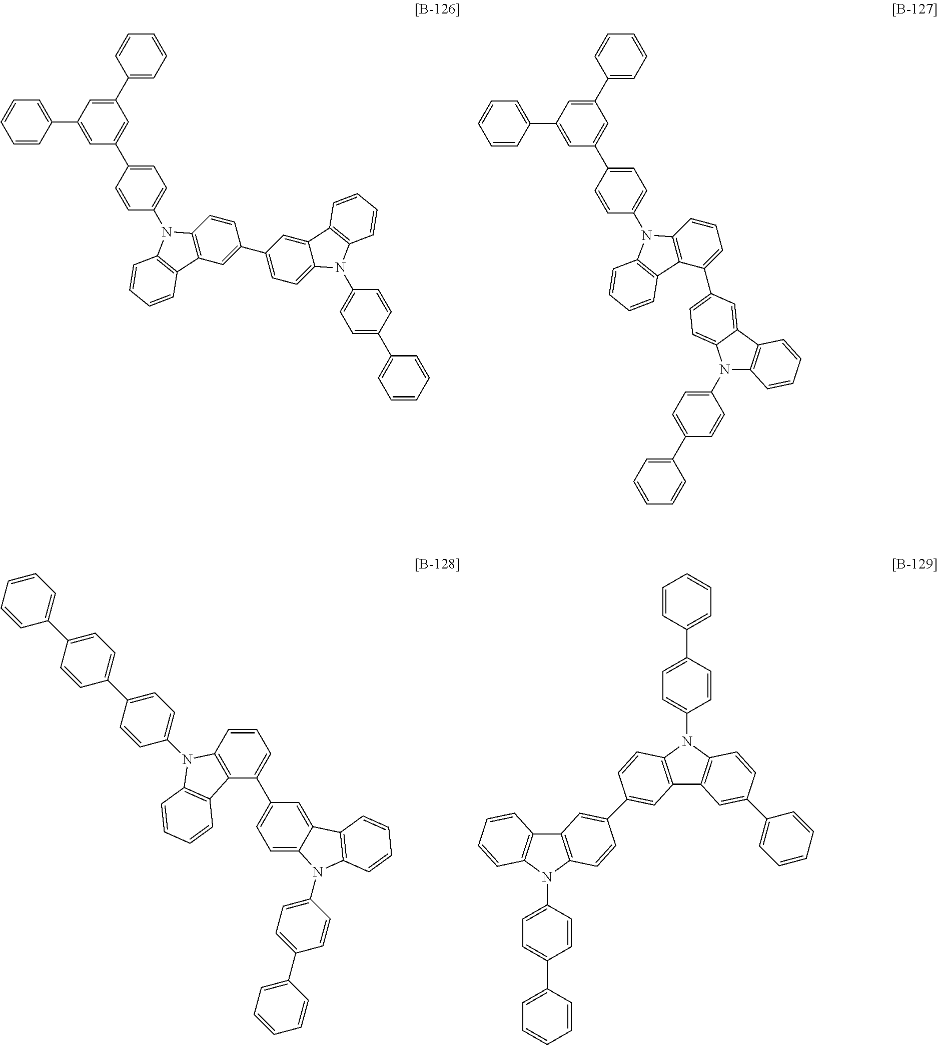 [SMILES: c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1 ; c1ccc(-c2ccc(-n3c4ccccc4c4cc(-c5cccc6c5c5ccccc5n6-c5ccc(-c6cc(-c7ccccc7)cc(-c7ccccc7)c6)cc5)ccc43)cc2)cc1 ; c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1]

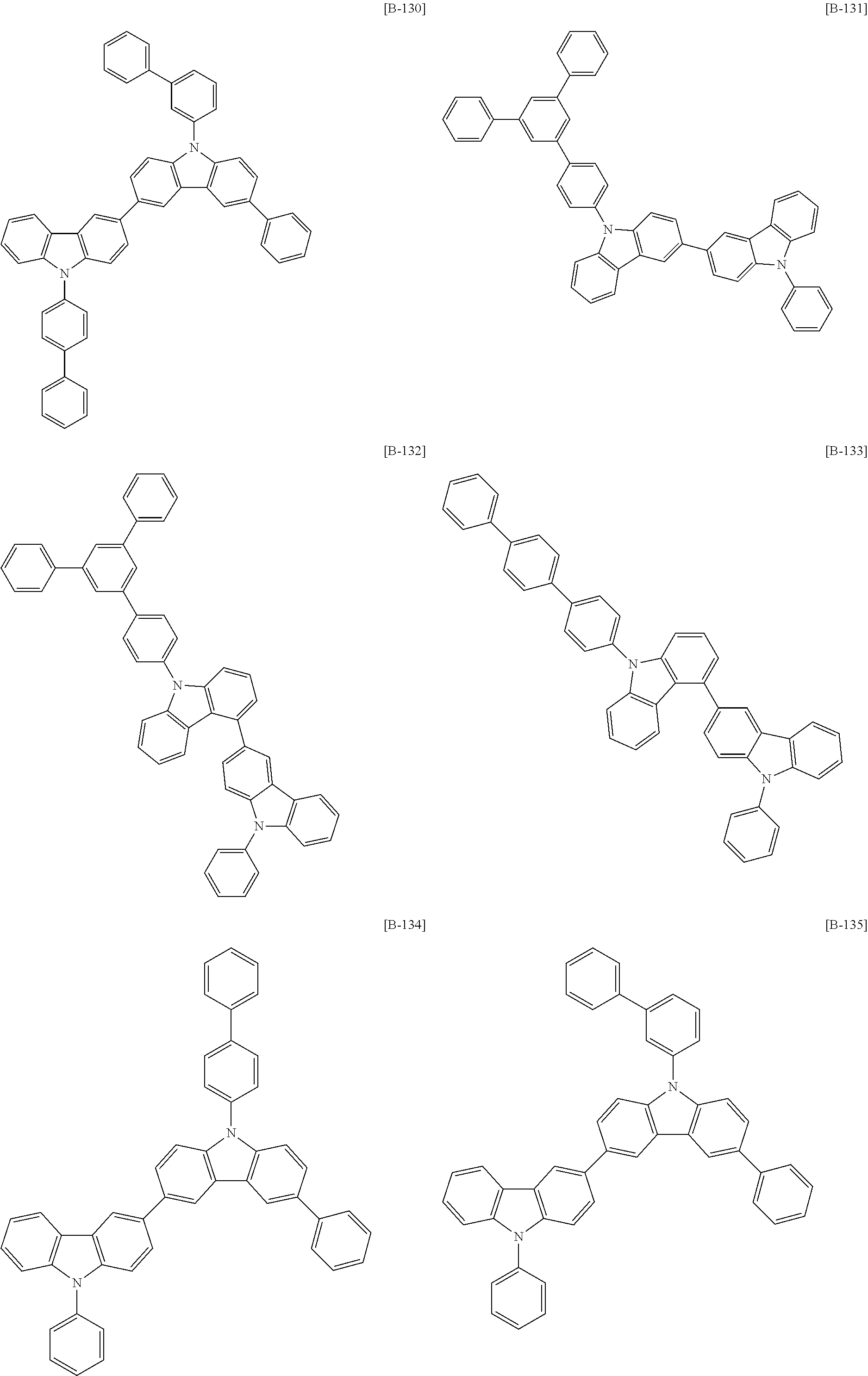

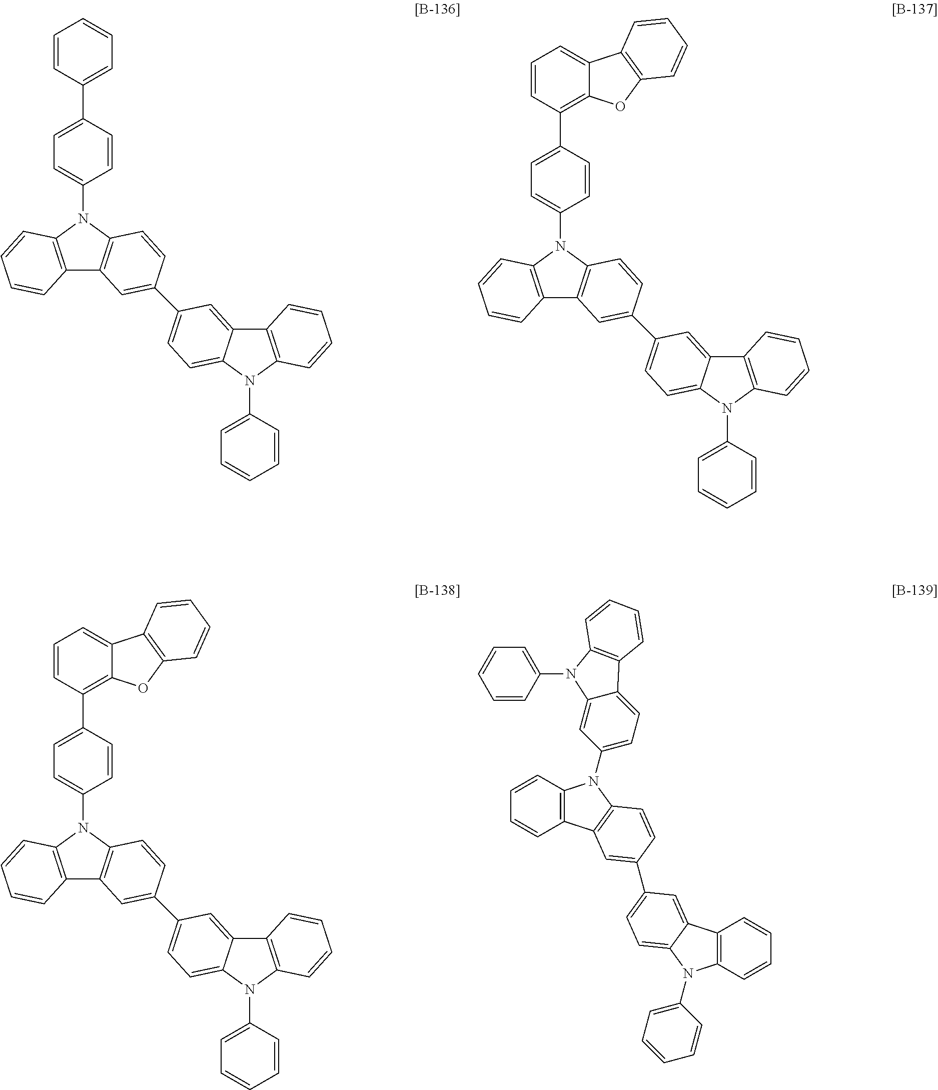 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1]

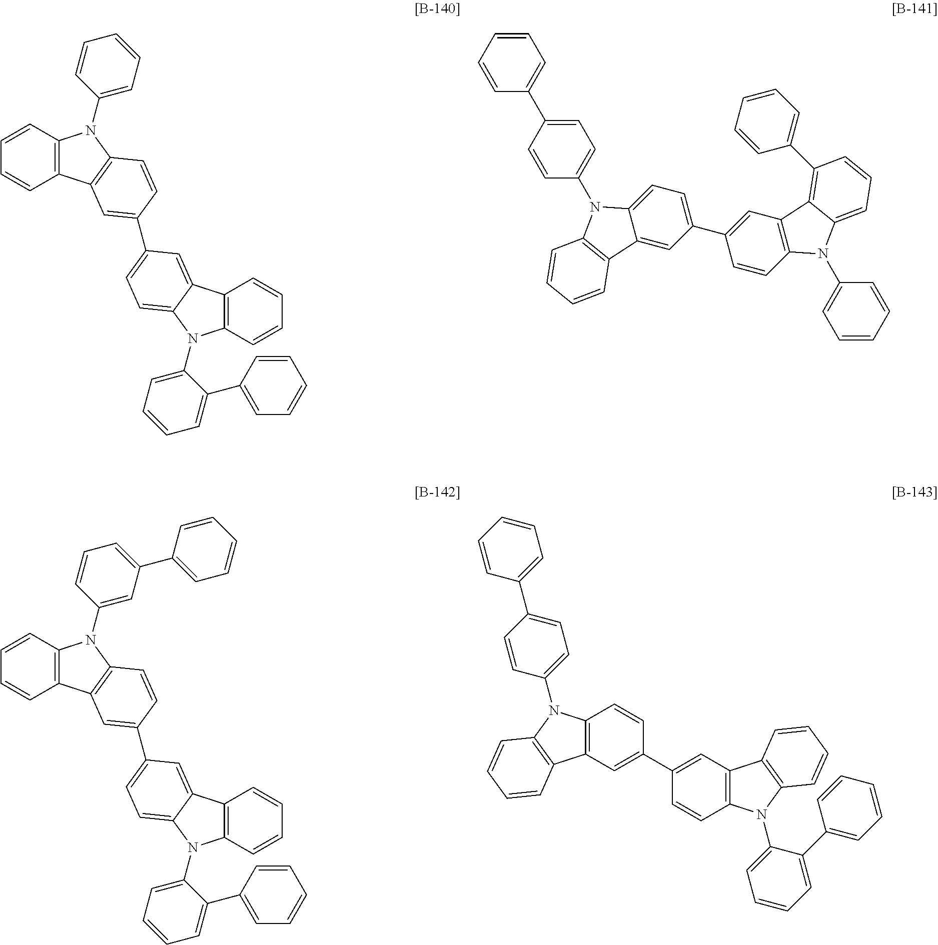 [SMILES: c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1]

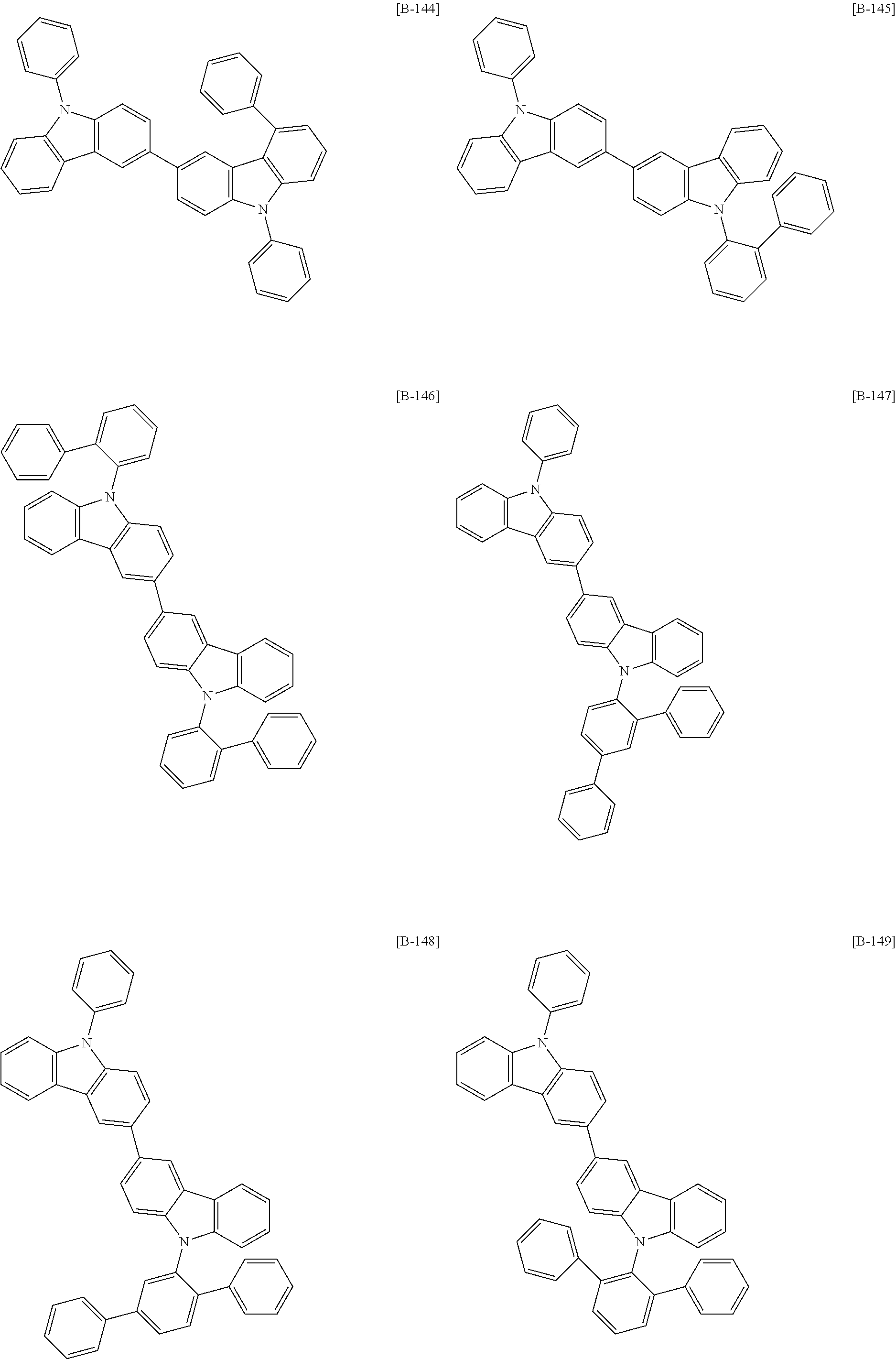 [SMILES: c1ccc(-c2cccc3c2c2cc(-c4ccc5c(c4)c4ccccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2ccc(-n3c4ccccc4c4cc(-c5ccc6c(c5)c5ccccc5n6-c5ccccc5)ccc43)c(-c3ccccc3)c2)cc1 ; c1ccc(-c2ccc(-c3ccccc3)c(-n3c4ccccc4c4cc(-c5ccc6c(c5)c5ccccc5n6-c5ccccc5)ccc43)c2)cc1 ; c1ccc(-c2cccc(-c3ccccc3)c2-n2c3ccccc3c3cc(-c4ccc5c(c4)c4ccccc4n5-c4ccccc4)ccc32)cc1]

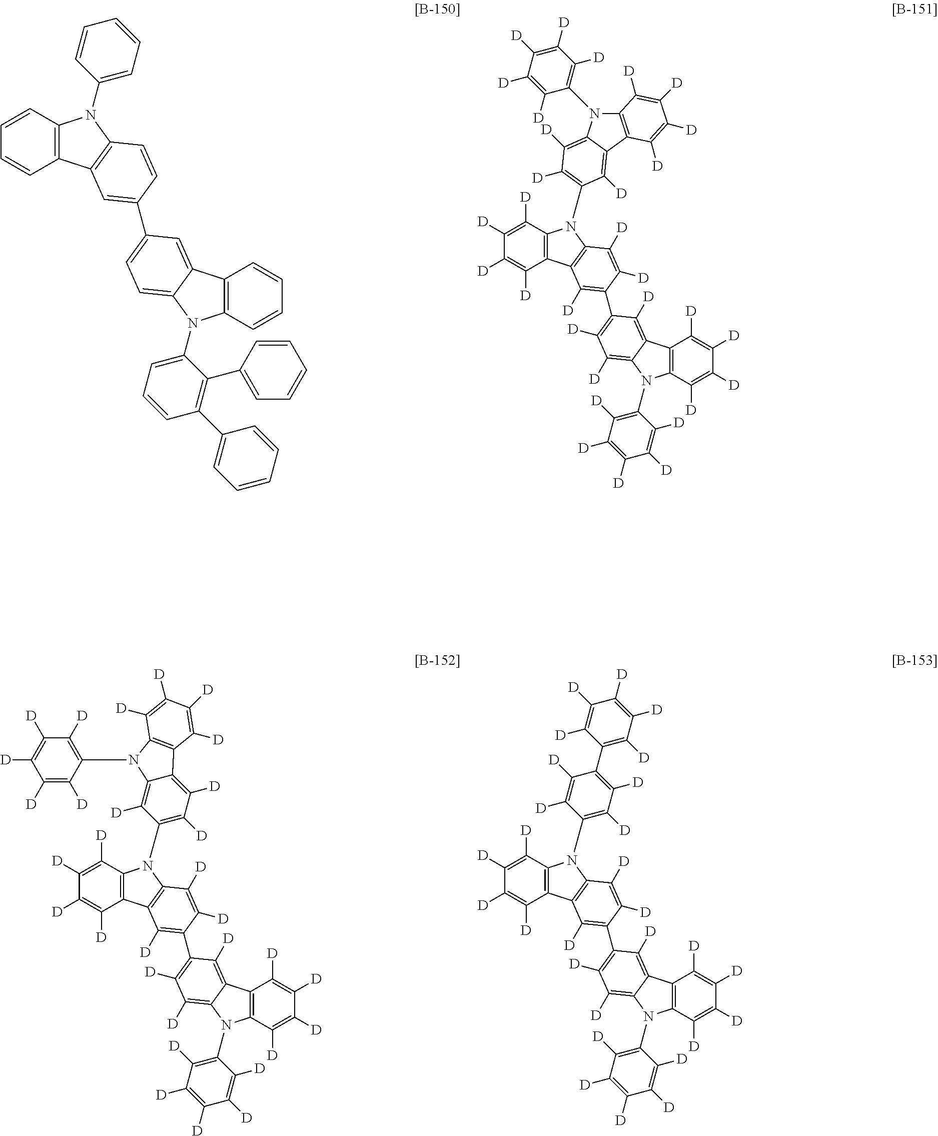

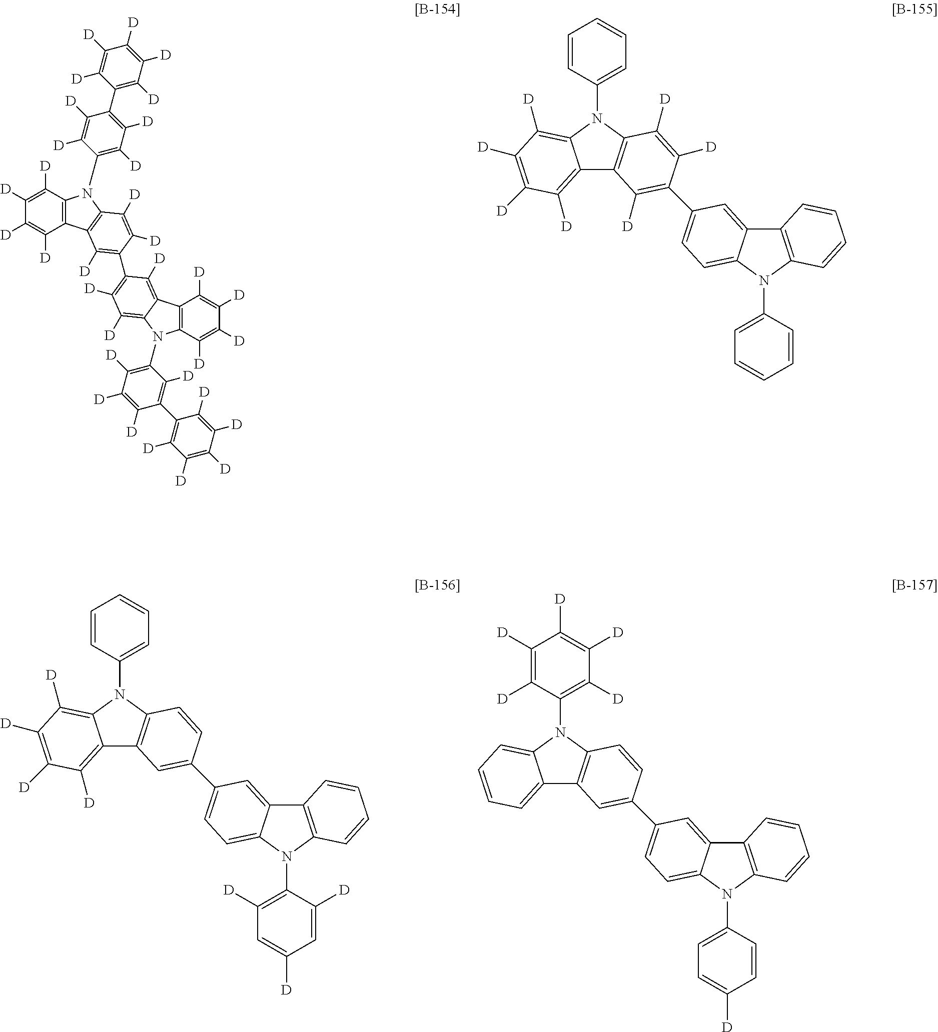

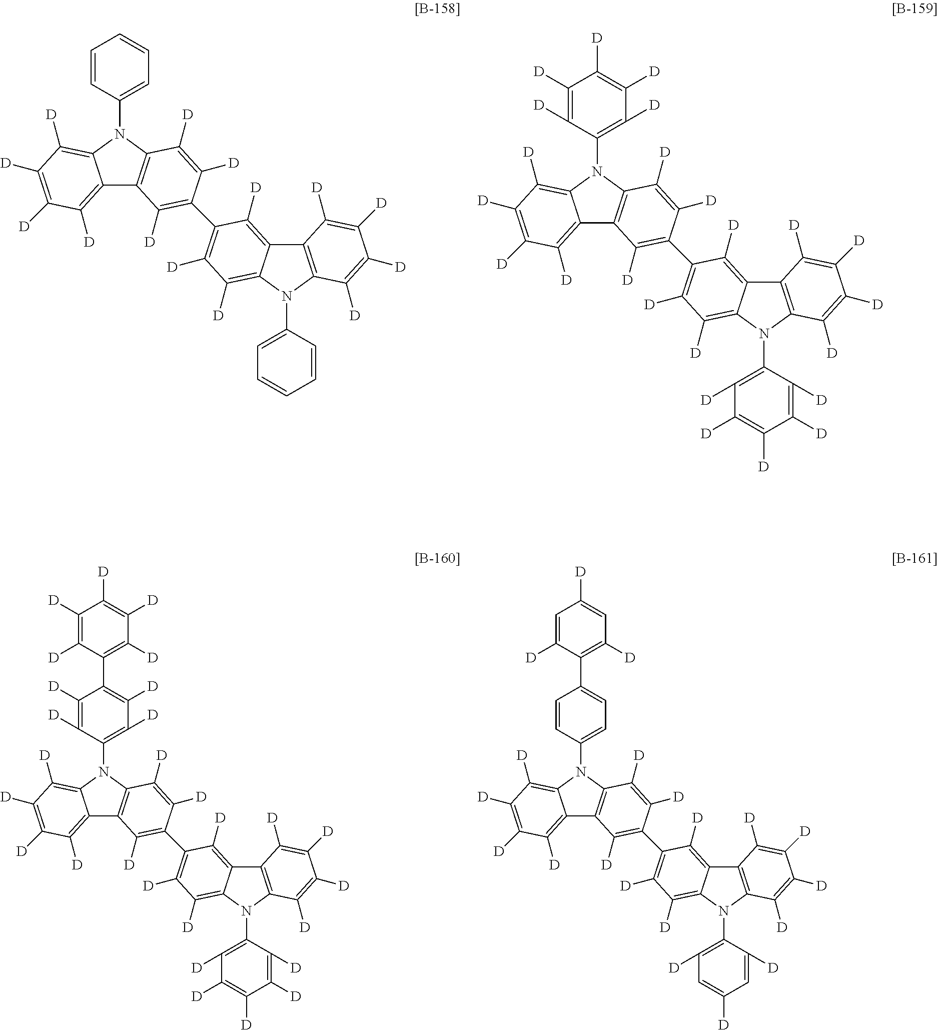

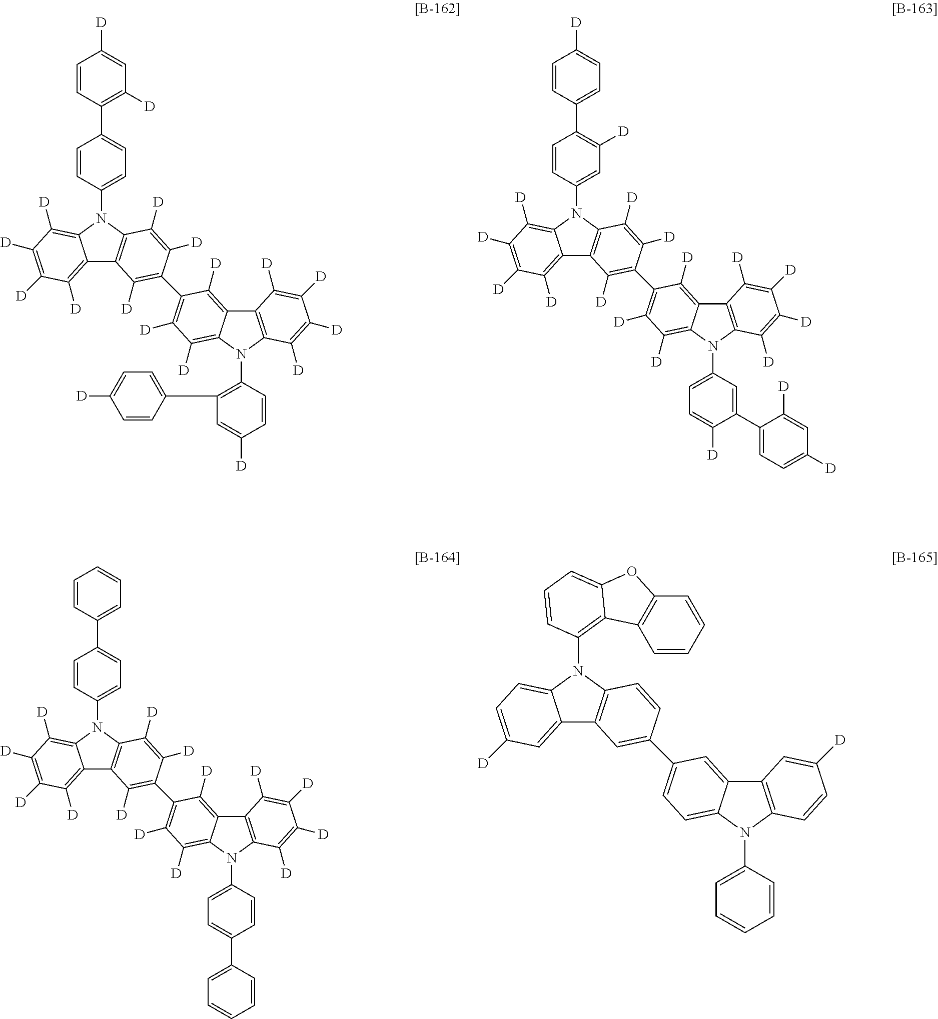

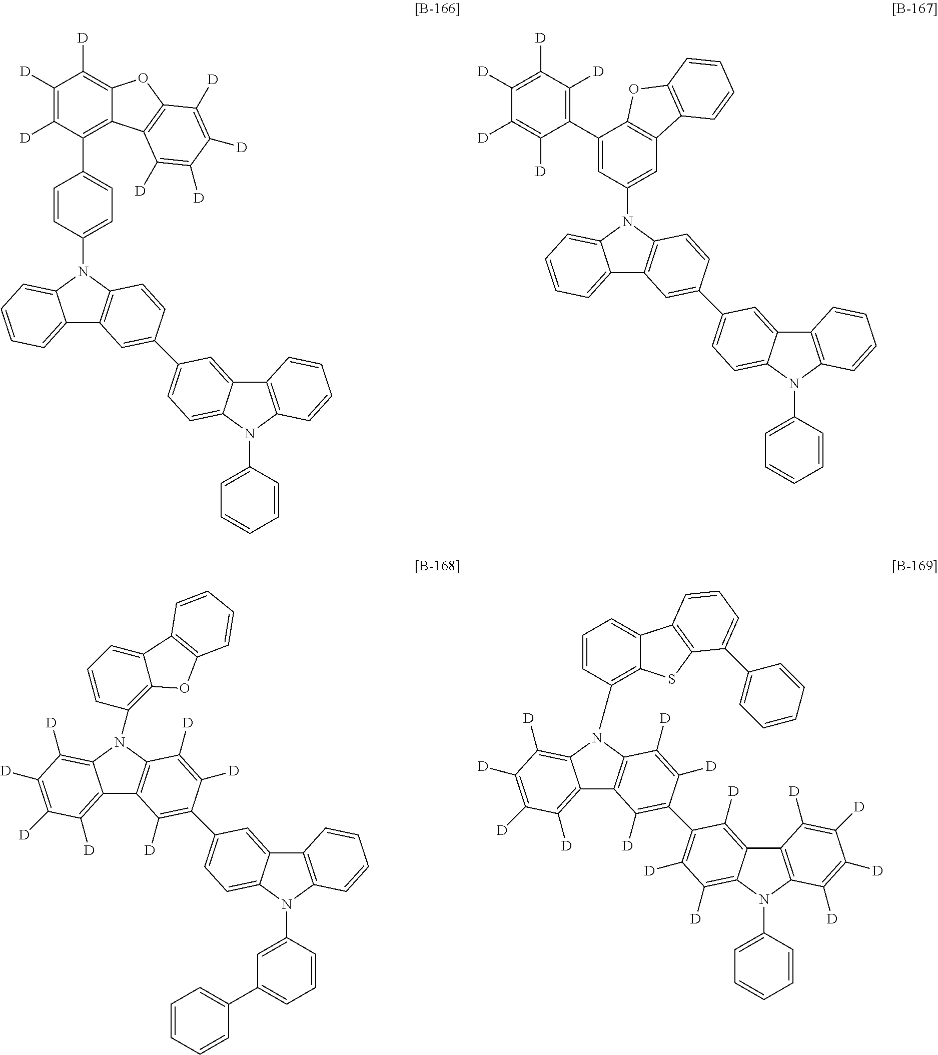

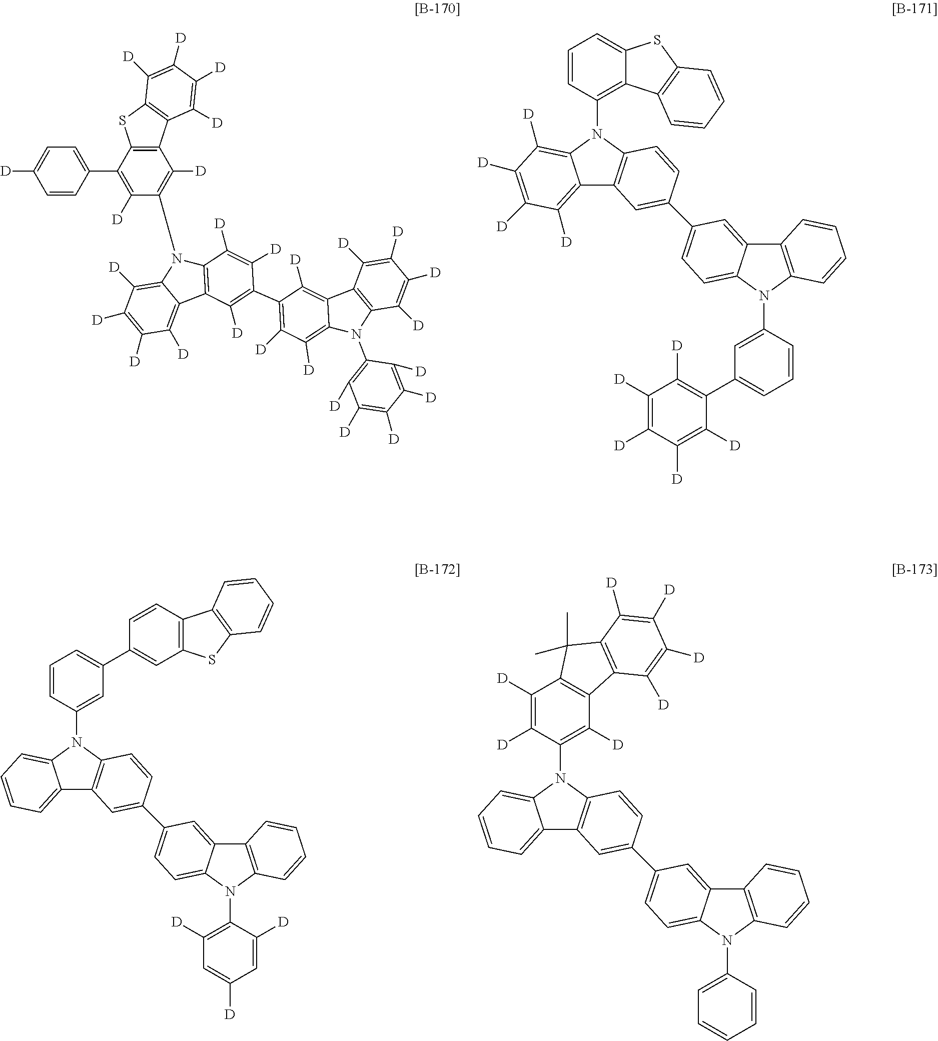

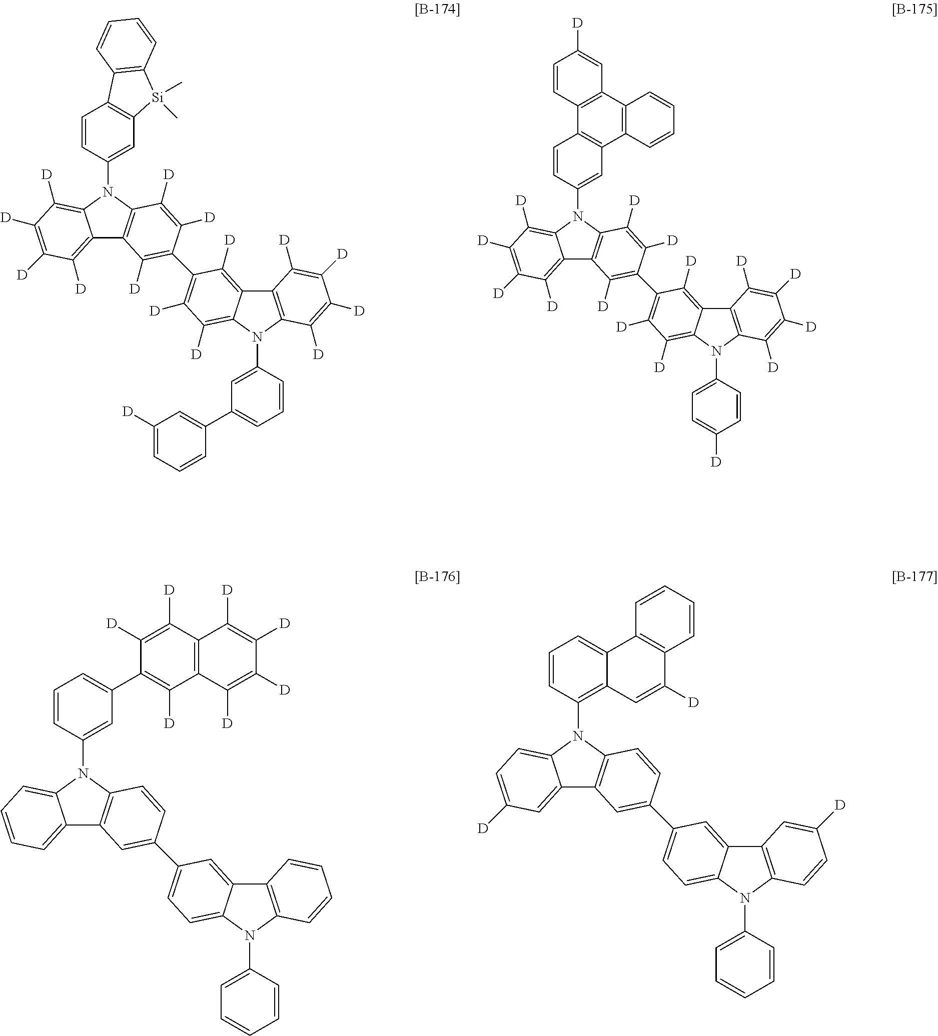

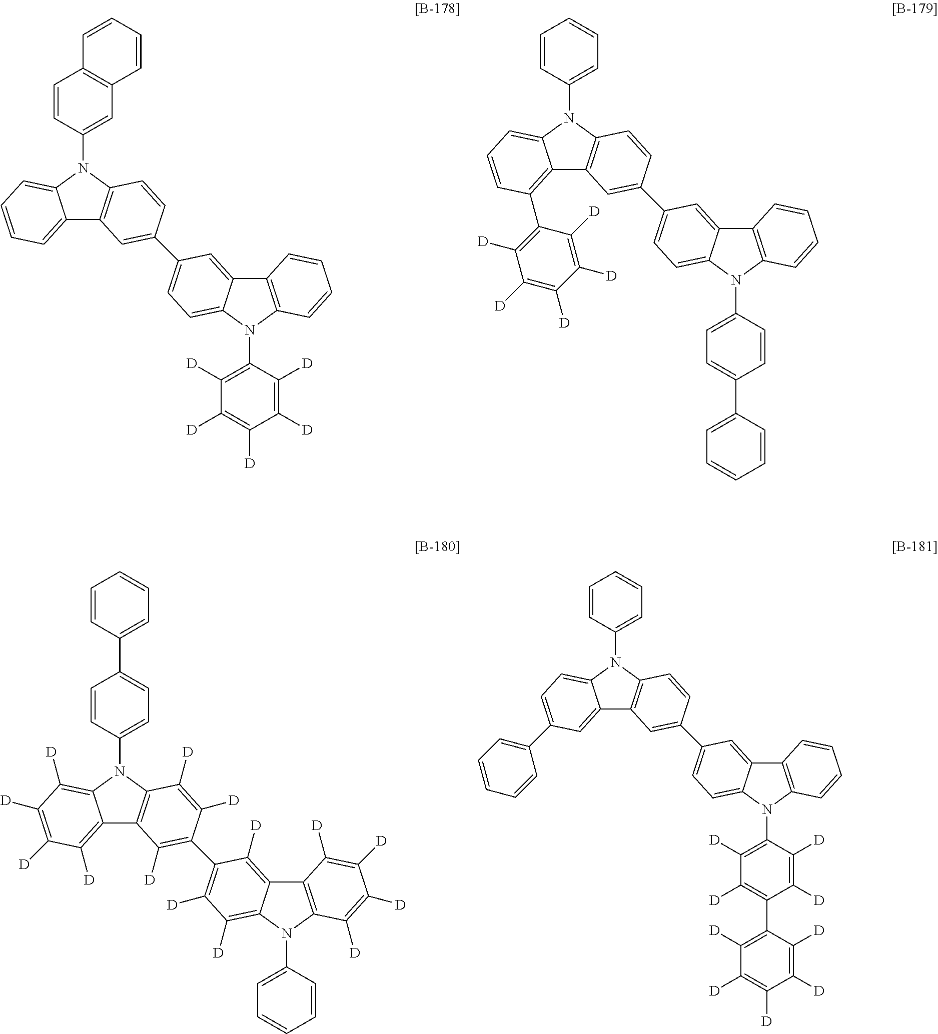 [SMILES: [2H]c1c([2H])c([2H])c(-n2c3ccccc3c3cc(-c4ccc5c(c4)c4ccccc4n5-c4ccc5ccccc5c4)ccc32)c([2H])c1[2H] ; [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccccc1 ; [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H]]

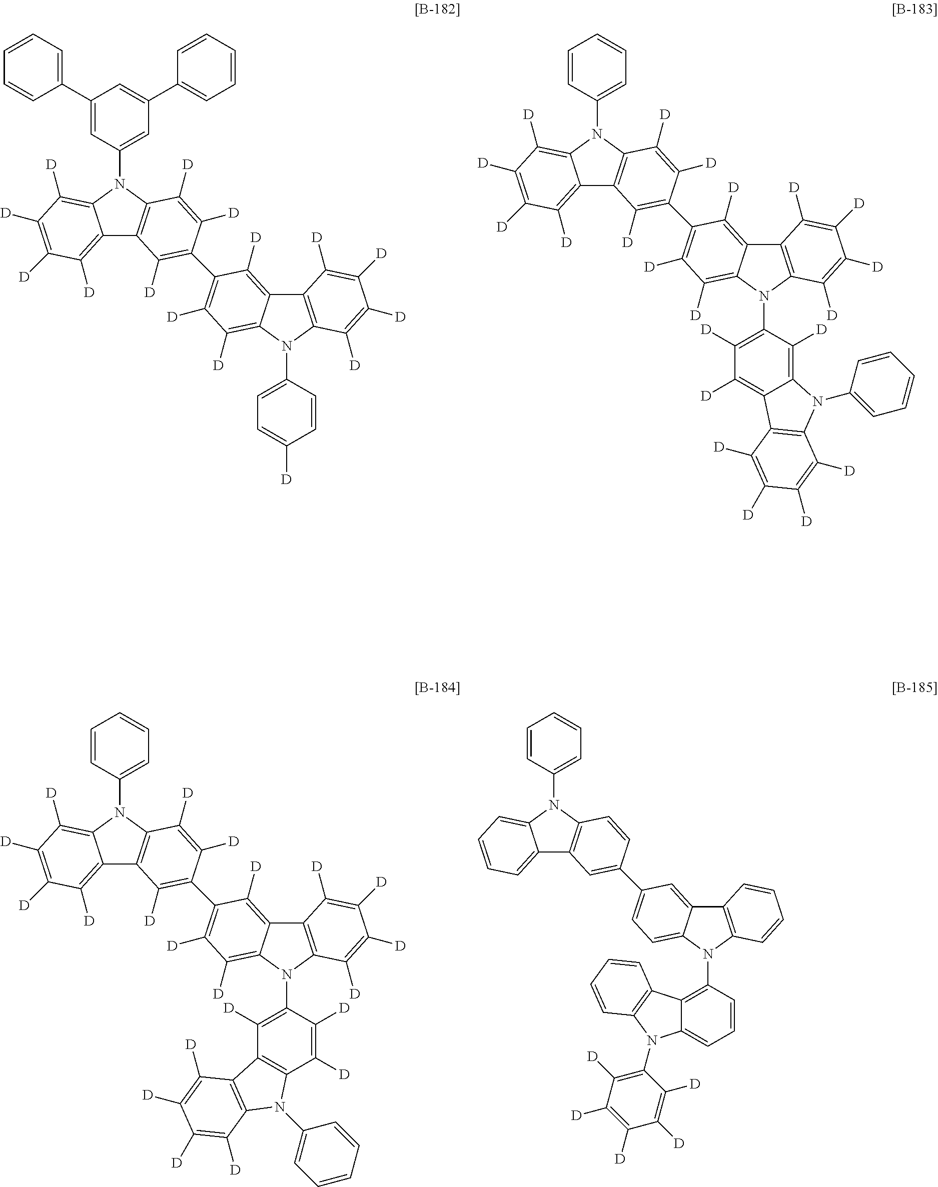

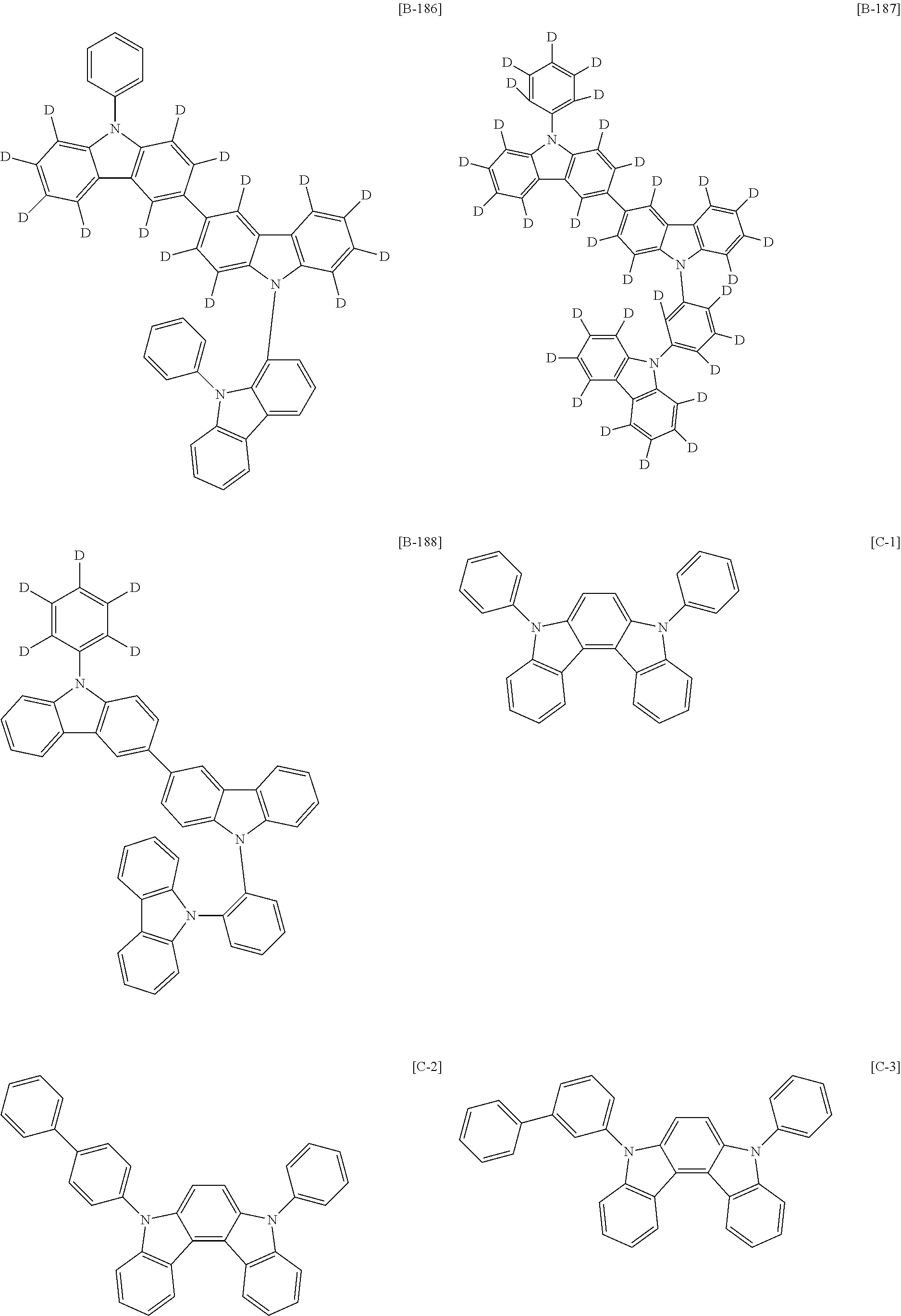

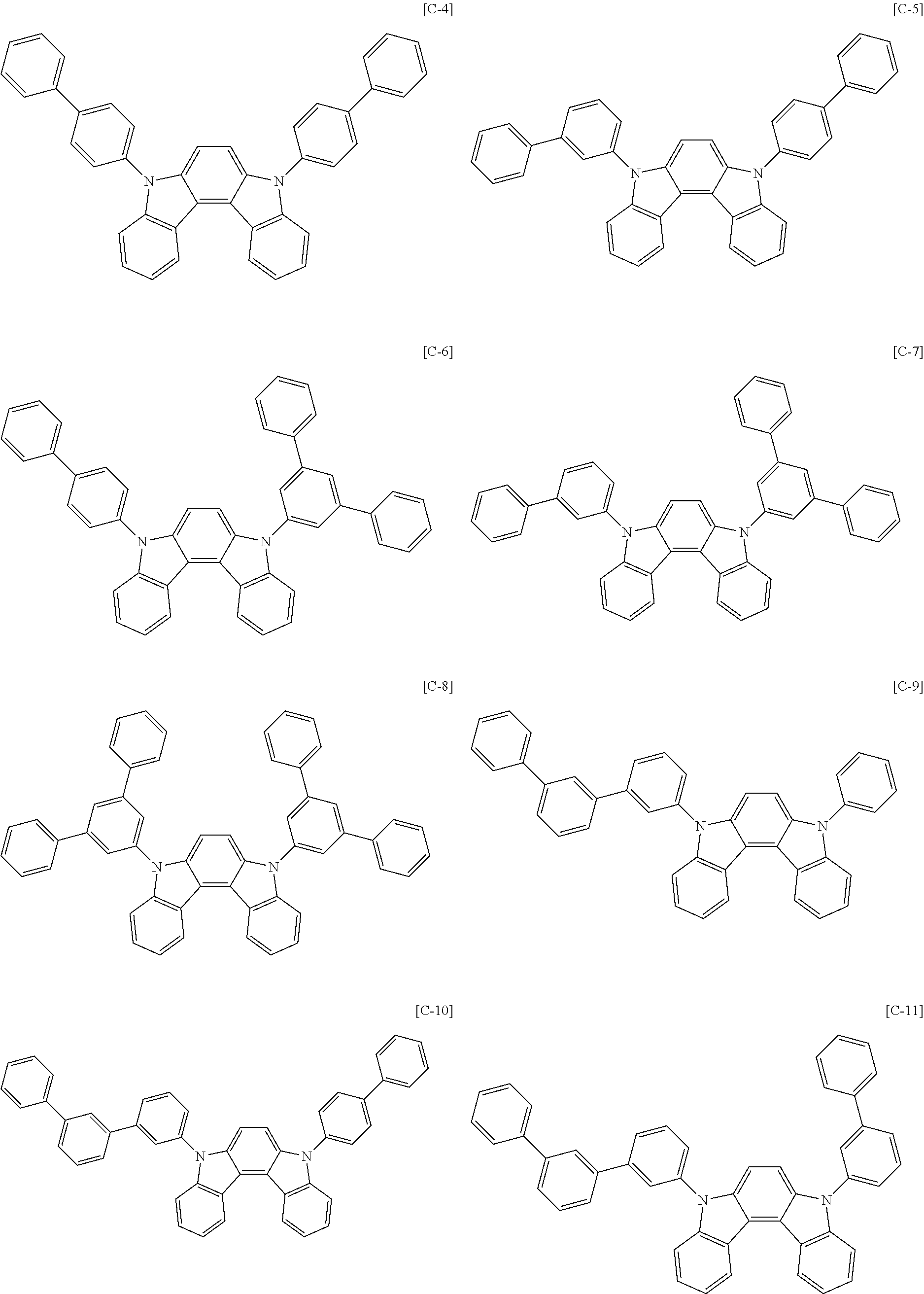

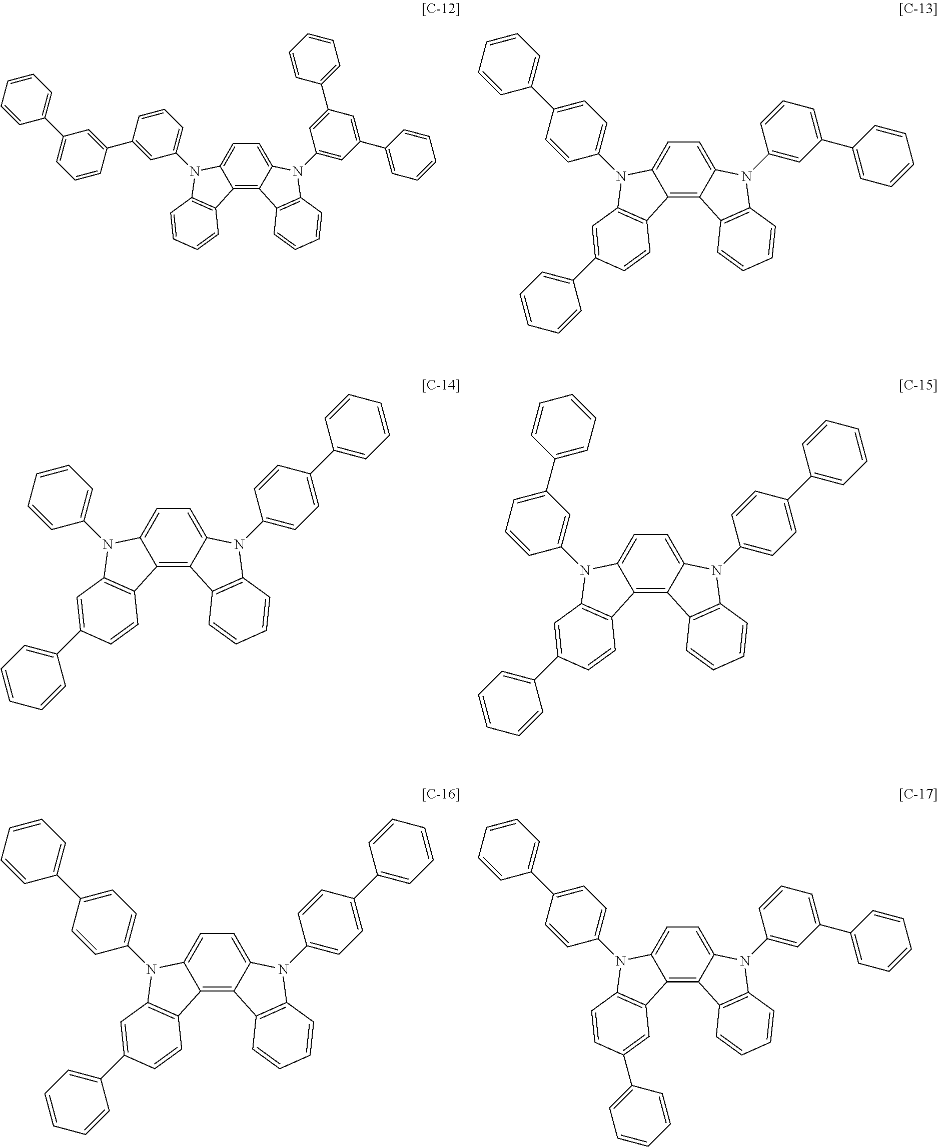

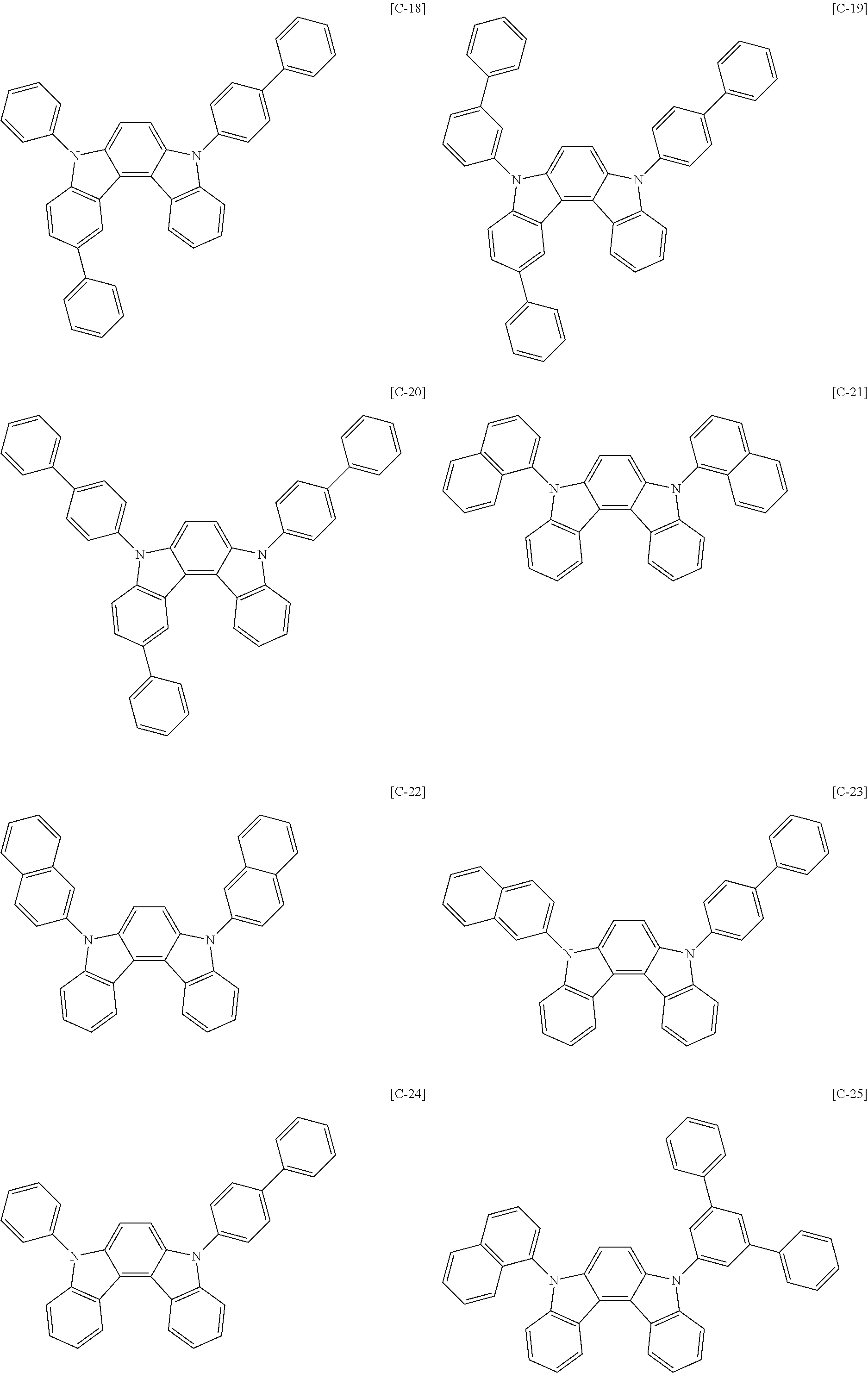

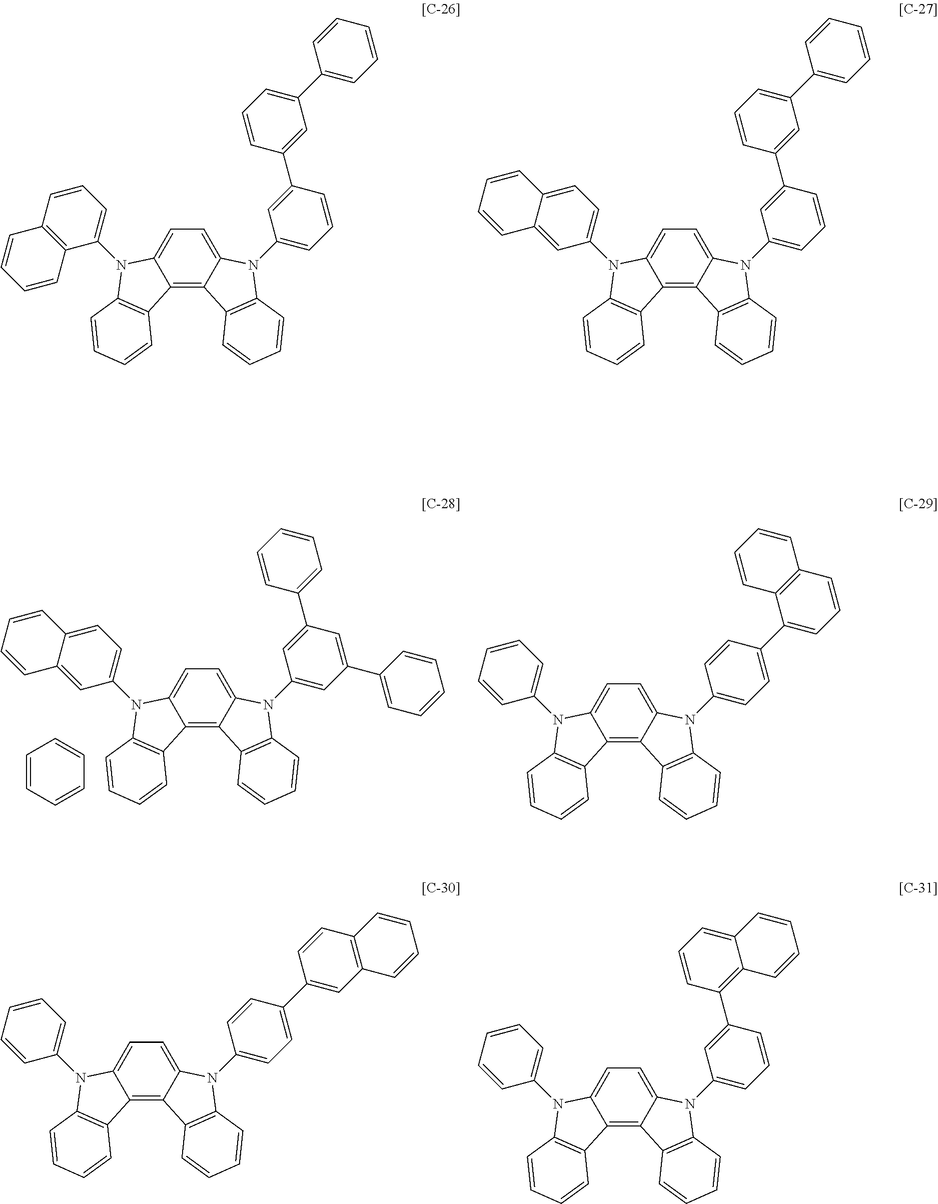

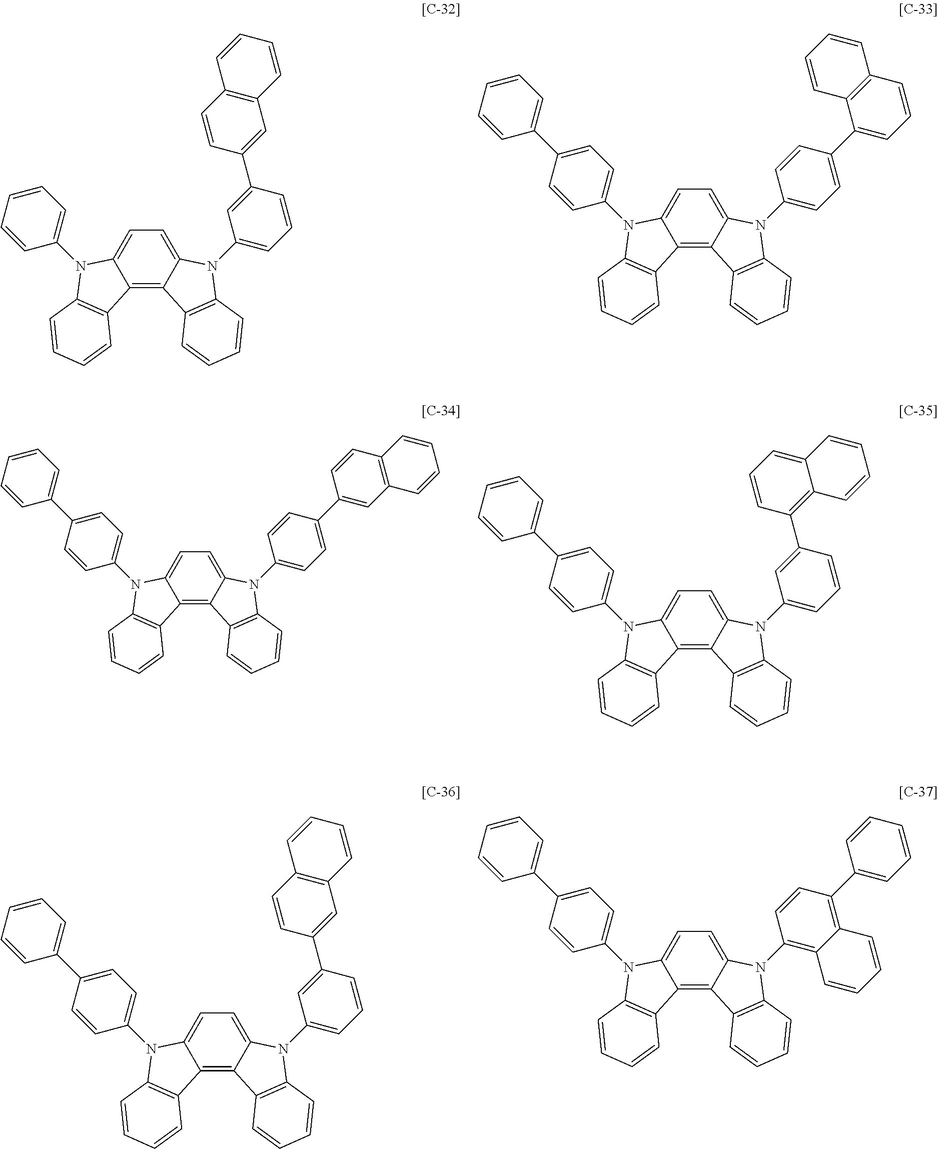 [SMILES: c1ccc(-n2c3ccccc3c3c4c5ccccc5n(-c5cccc(-c6ccc7ccccc7c6)c5)c4ccc32)cc1 ; c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1 ; c1ccc(-c2ccc(-n3c4ccccc4c4c5c6ccccc6n(-c6cccc(-c7ccc8ccccc8c7)c6)c5ccc43)cc2)cc1 ; c1ccc(-c2ccc(-n3c4ccccc4c4c5c6ccccc6n(-c6ccc(-c7ccccc7)c7ccccc67)c5ccc43)cc2)cc1]

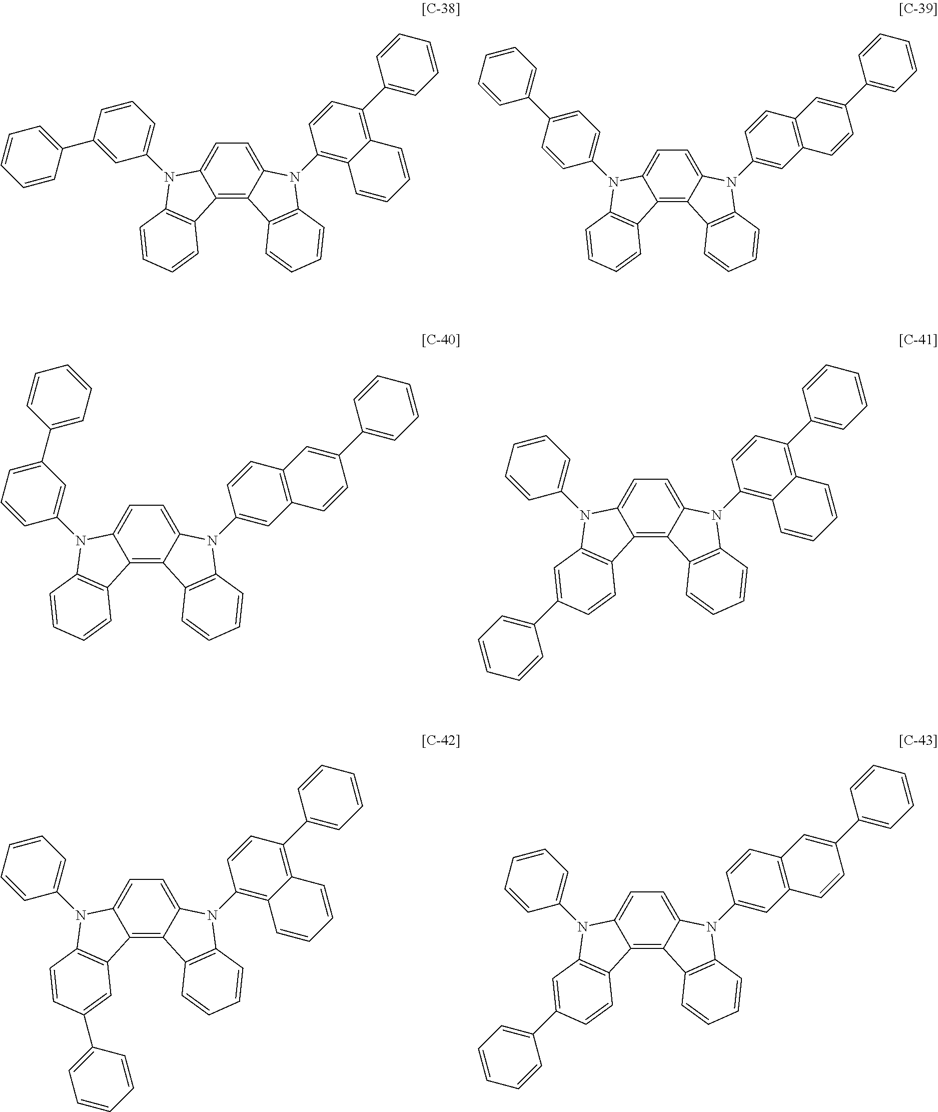

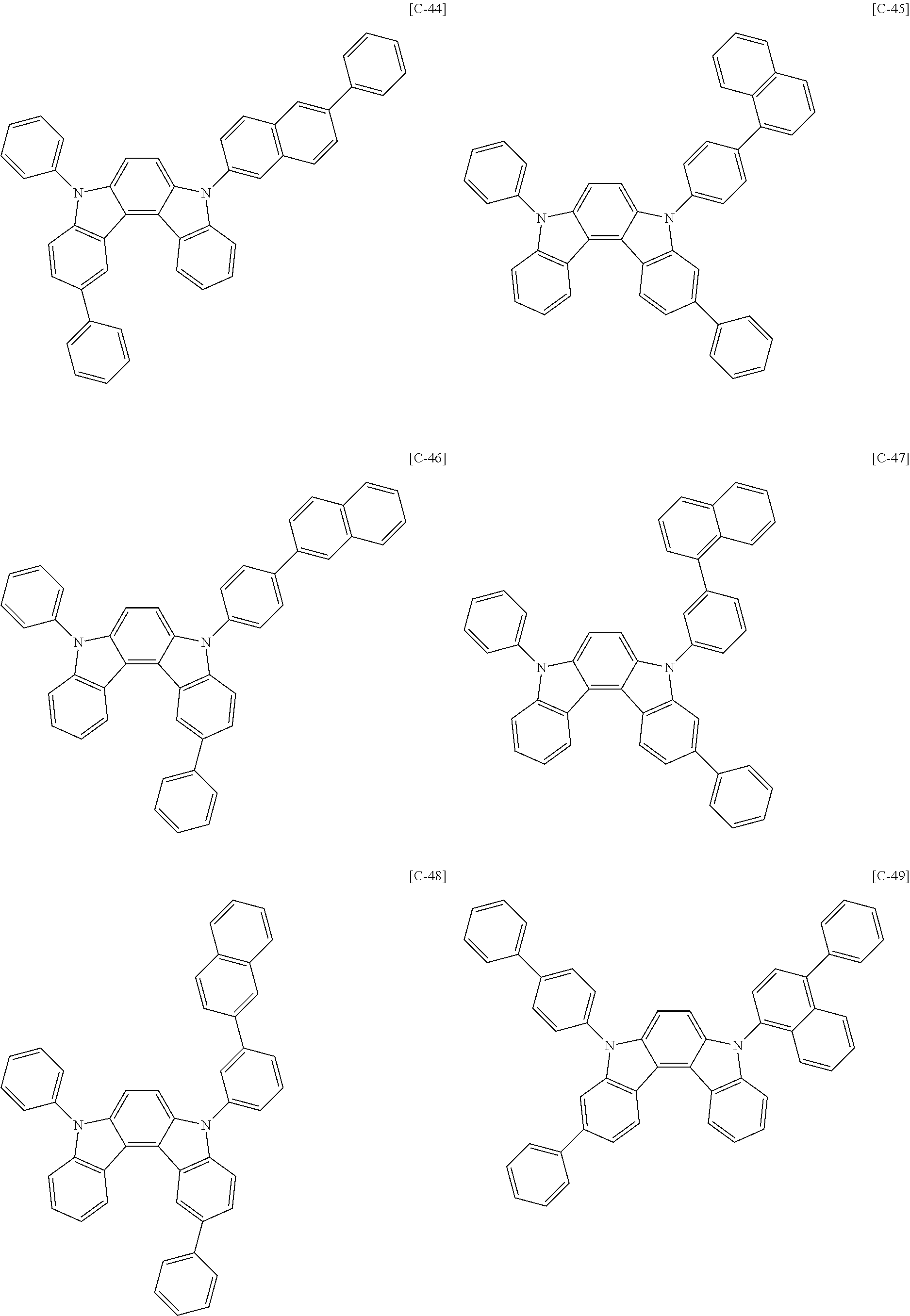

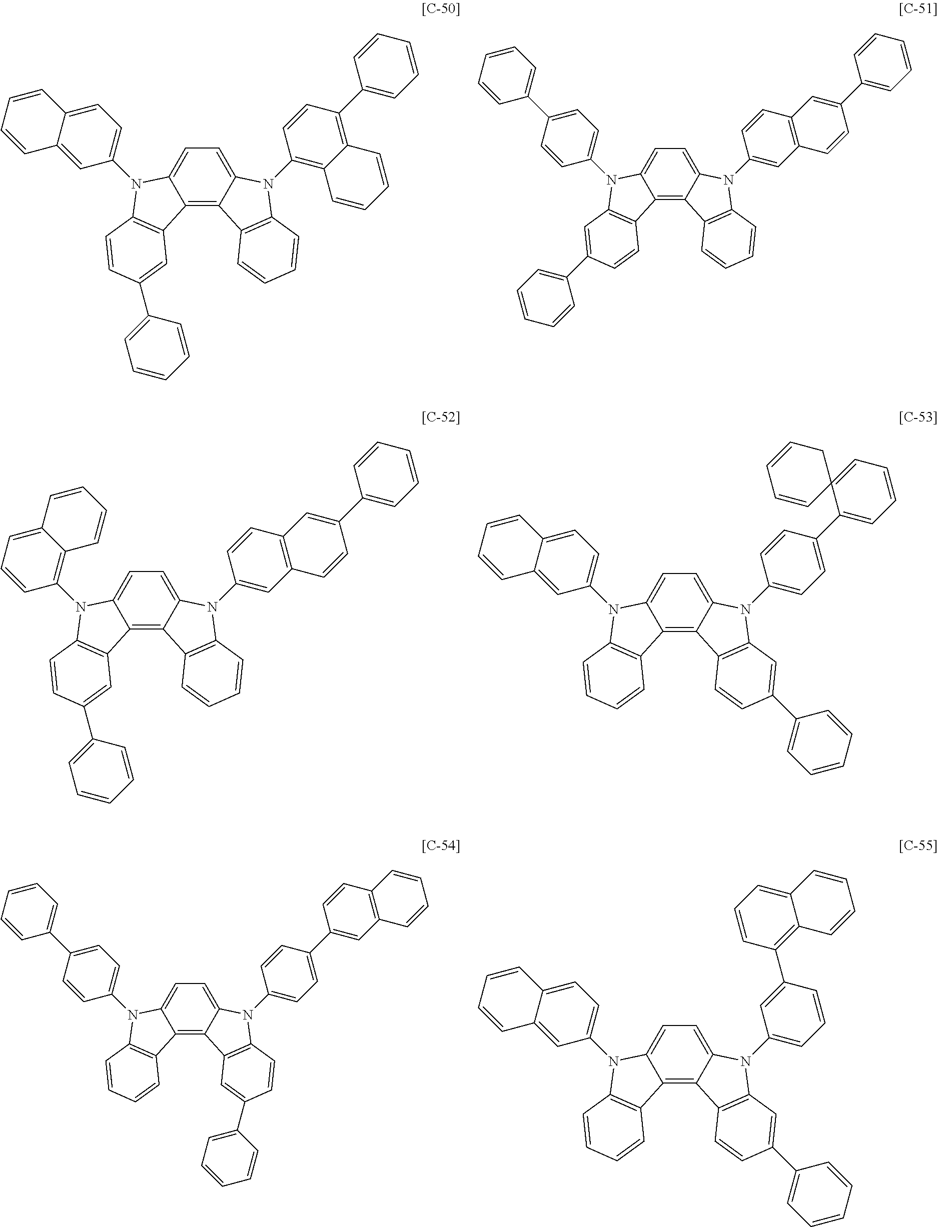

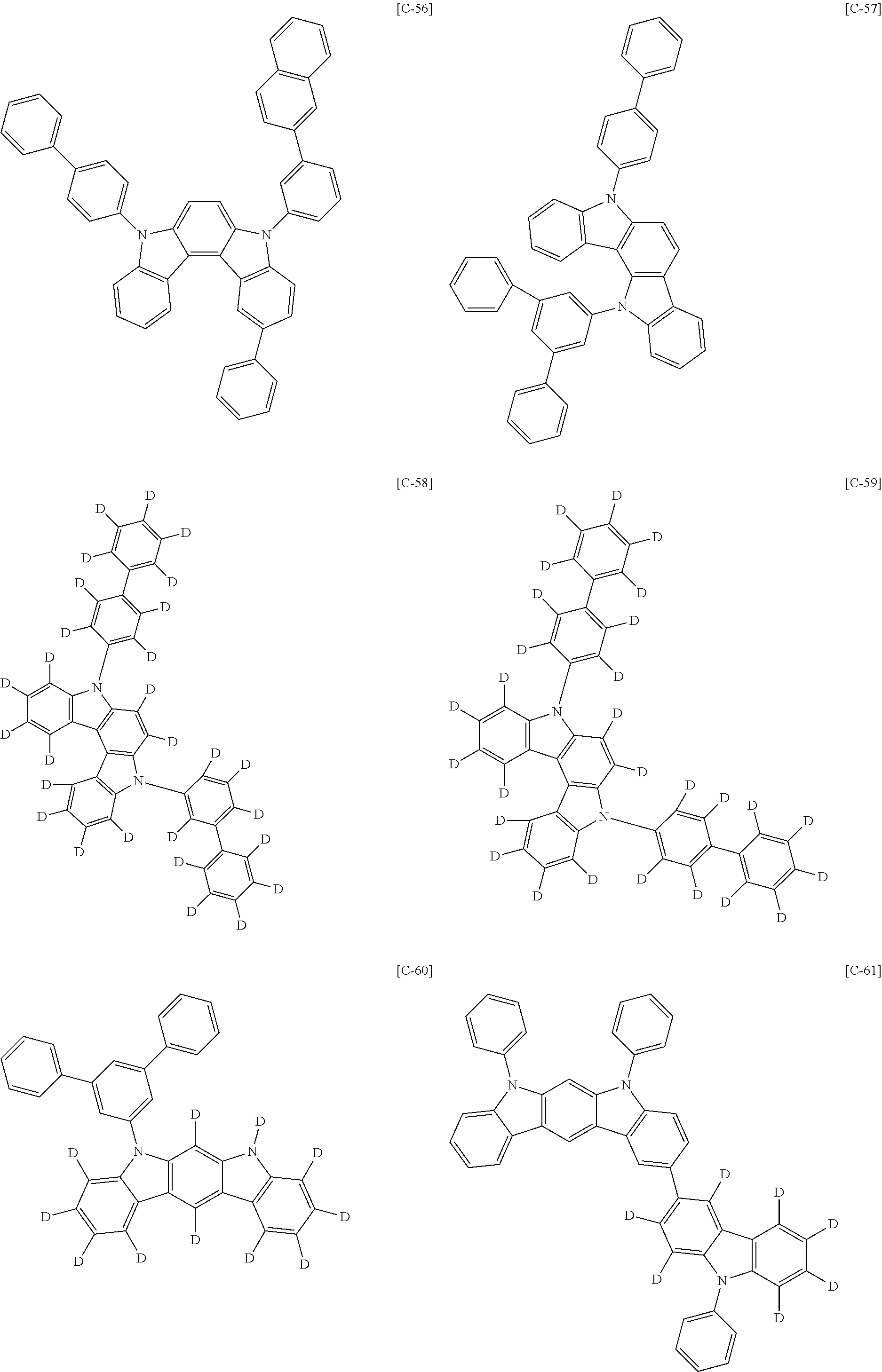

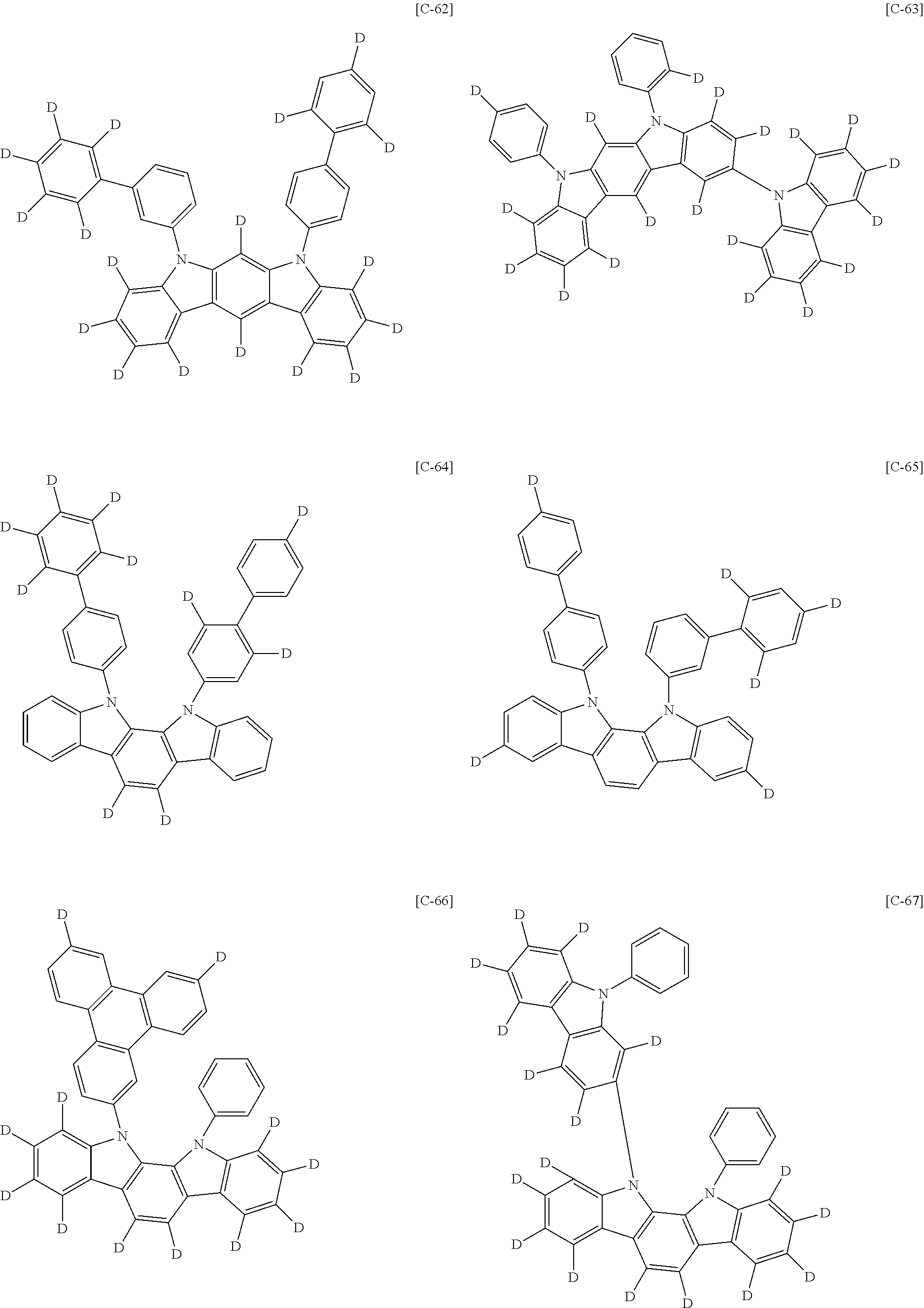

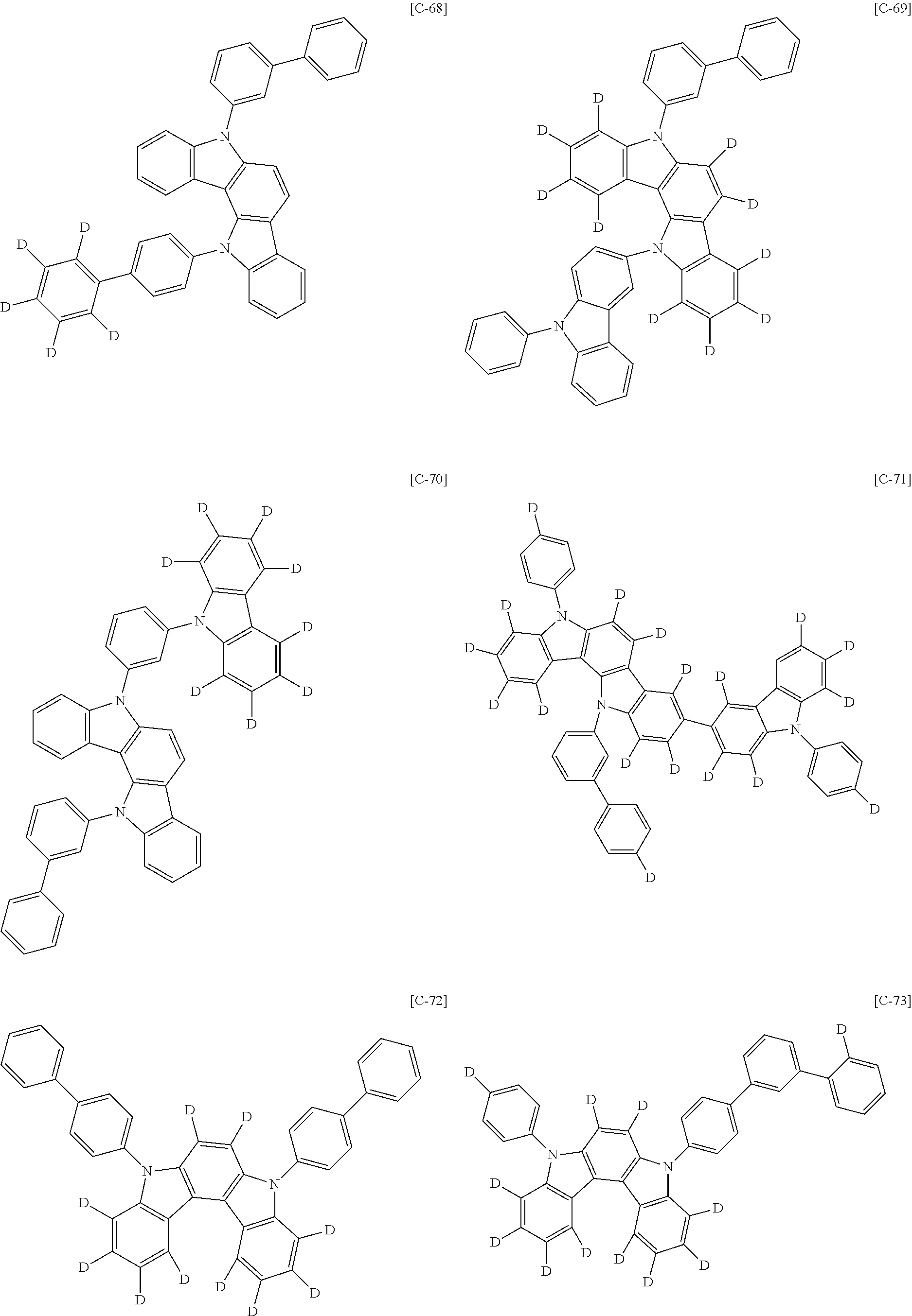

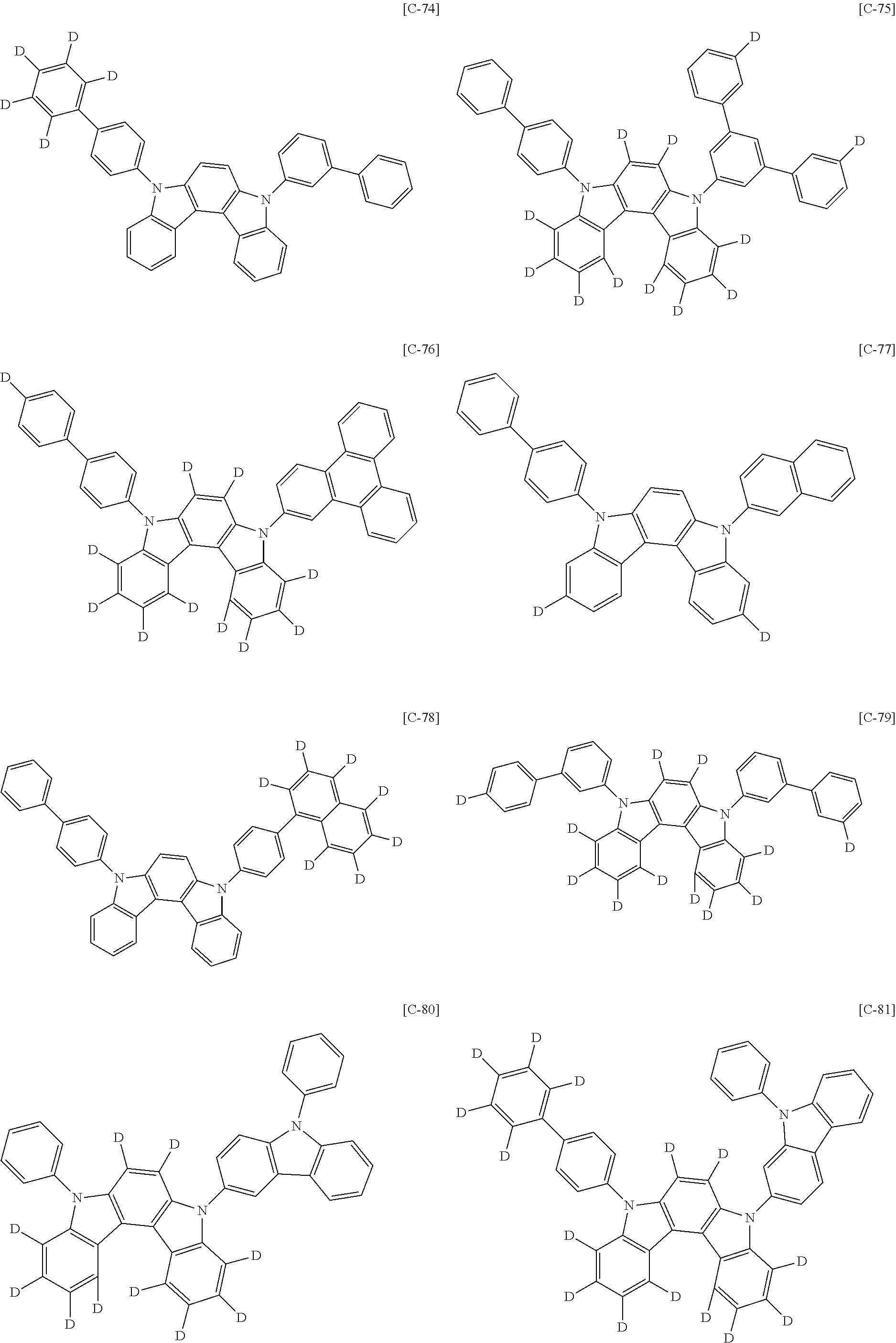

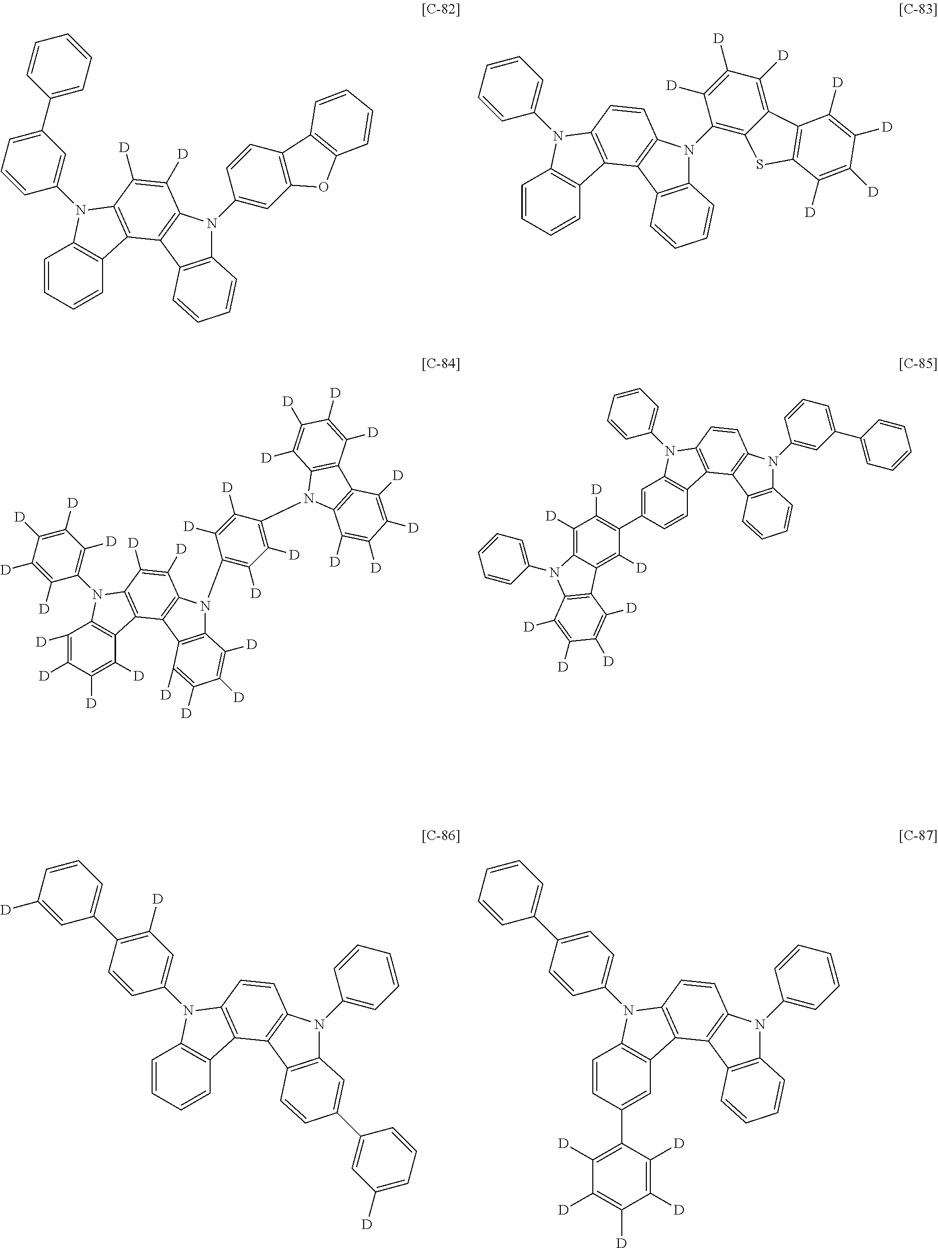

One type or two or more types of the second compound may be used.

In the light emitting layer 32, the first compound and the second compound may be included as a host, and may be included in a weight ratio of, e.g., about 1:99 to 99:1. Within the above range, bipolar properties may be implemented by matching an appropriate weight ratio using electron transport capability of the first compound and the hole transport capability of the second compound, to improve efficiency and life-span. Within this range, e.g., they may be included in a weight ratio of about 10:90 to 90:10, about 20:80 to 80:20, for example about 20:80 to about 70:30, about 20:80 to about 60:40, or about 20:80 to about 50:50. In an implementation, they may be included in a weight ratio of about 20:80, 30:70, or 40:60.

The light emitting layer 32 may further include one or more compounds other than the aforementioned first compound and second compound as a host.

The light emitting layer 32 may further include a dopant.

The dopant may be, e.g., a phosphorescent dopant, for example, a red, green, or blue phosphorescent dopant, and may be, for example, a red or green phosphorescent dopant.

The dopant is a material mixed with the compound for an organic optoelectronic device in a small amount to cause light emission, and may be generally a material such as a metal complex that emits light by multiple excitation into a triplet or more. The dopant may be, e.g., an inorganic, organic, or organic/inorganic compound, and one or more types thereof may be used.

Examples of the dopant may be a phosphorescent dopant and examples of the phosphorescent dopant may be an organometal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z.

L¹¹MX⁶ [Chemical Formula Z]

In Chemical Formula Z, M is a metal, L¹¹ and X⁶ may be the same or different and are a ligand to form a complex compound with M.

The M may be for example Ir, Pt, Os, Ti, Zr, Hf, Eu, Tb, Tm, Fe, Co, Ni, Ru, Rh, Pd, or a combination thereof and L¹¹ and X⁶ may be for example a bidentate ligand.

Examples of ligands represented by L¹¹ and X⁶ may be ligands of Group A.

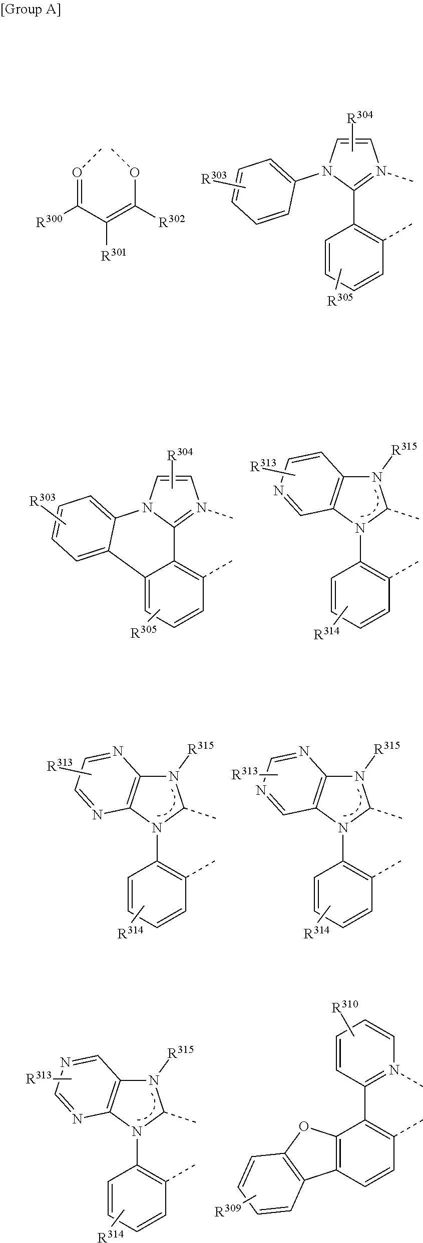

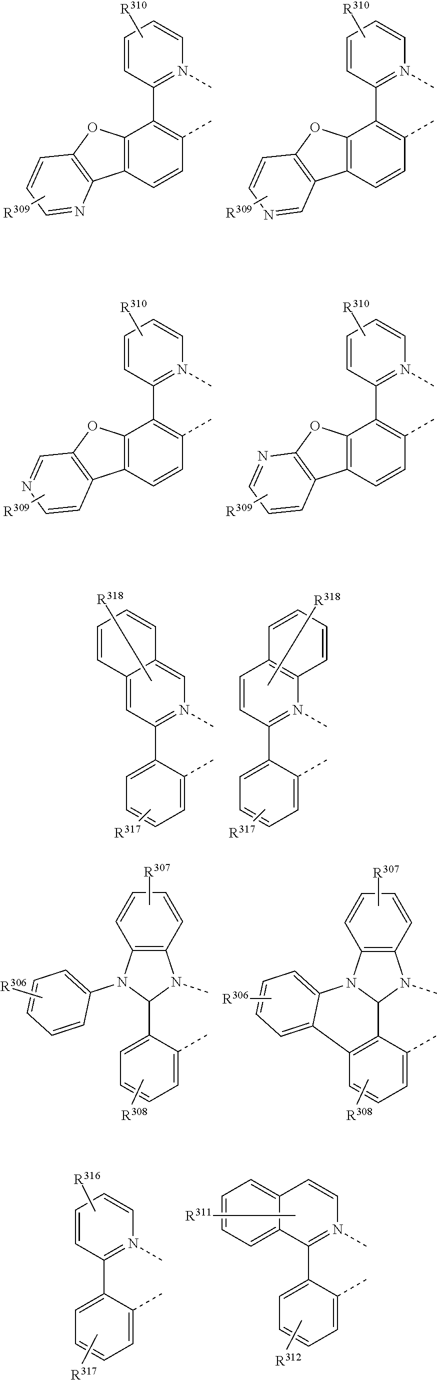

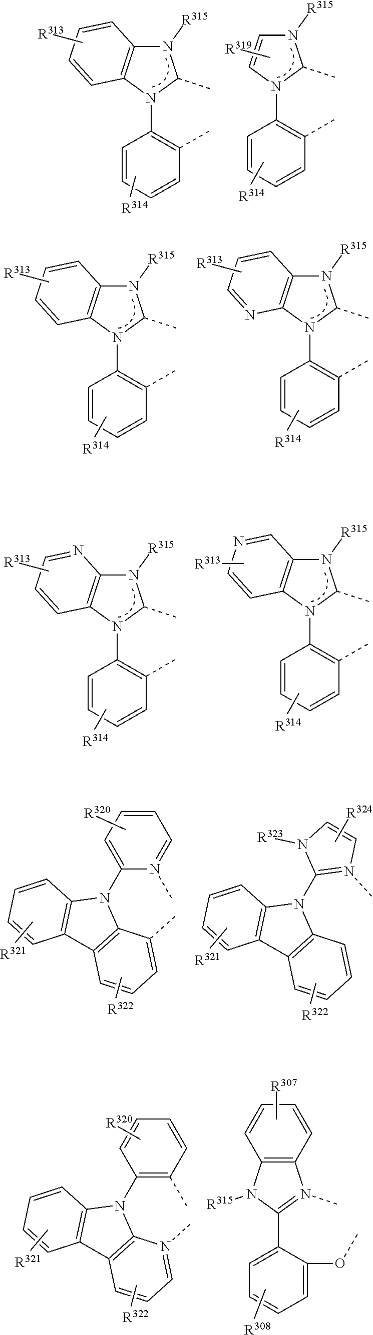

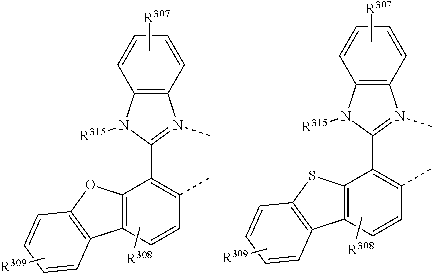

In Group A, R³⁰⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen.

R³⁰³ to R³²⁴ may each independently be, e.g., hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

In an implementation, it may include a dopant represented by Chemical Formula V.

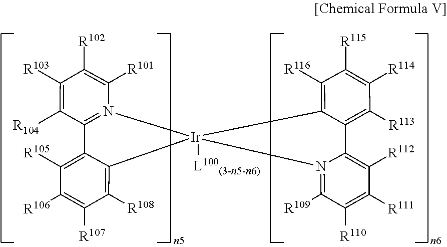

In Chemical Formula V, R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

In an implementation, at least one of R¹⁰¹ to R¹¹⁶ may be a functional group represented by Chemical Formula V-1.

L¹⁰⁰ may be, e.g., a bidentate ligand of a monovalent anion, and is a ligand that coordinates to iridium through a lone pair of carbons or heteroatoms.

n5 and n6 may each independently be an integer of 0 to 3, and n5+n6 is any one of integers of 1 to 3,

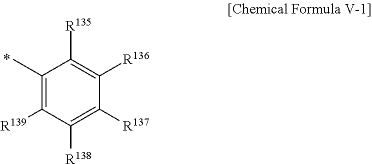

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

* is a linking point.

In an implementation, a dopant represented by Chemical Formula Z-1 may be included.

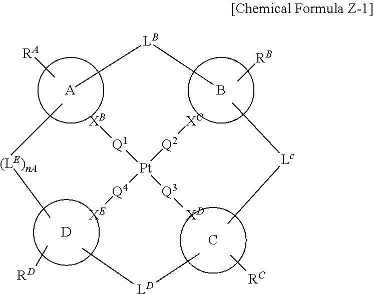

In Chemical Formula Z-1, rings A, B, C, and D may each independently be, e.g., a 5-membered or 6-membered carbocyclic or heterocyclic ring.

R^{A}, R^{B}, R^{C}, and R^{D} may each independently be, e.g., mono-, di-, tri-, or tetra-substitution, or unsubstitution.

L^{B}, L^{C}, and L^{D} may each independently be, e.g., a direct bond, BR, NR, PR, O, S, Se, C═O, S═O, SO₂, CRR′, SiRR′, GeRR′, or a combination thereof.

In an implementation, when nA is 1, L^{E} is selected from a direct bond, BR, NR, PR, O, S, Se, C═O, S═O, SO₂, CRR′, SiRR′, GeRR′, and a combination thereof; when nA is O, L^{E} does not exist.

R^{A}, R^{B}, R^{C}, R^{D}, R, and R′ may each independently be, e.g., hydrogen, deuterium, a halogen, alkyl group, a cycloalkyl group, a heteroalkyl group, arylalkyl group, an alkoxy group, aryloxy group, an amino group, a silyl group, an alkenyl group, a cycloalkenyl group, a heteroalkenyl group, an alkynyl group, aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The dopant according to an embodiment may be a platinum complex, and may be, e.g., represented by Chemical Formula VI.

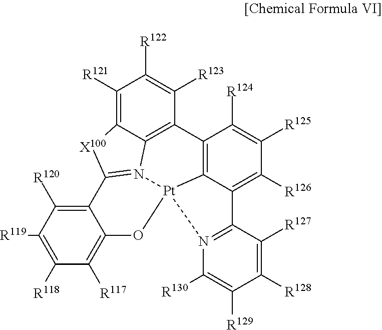

In Chemical Formula VI, X¹⁰⁰ may be, e.g., O, S, or NR¹³¹.

R¹¹⁷ to R¹³¹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

In an implementation, at least one of R¹¹⁷ to R¹³¹ may be —SiR¹³²R¹³³R¹³⁴ or a tert-butyl group.

R¹³² to R¹³⁴ may each independently be, e.g., a substituted or unsubstituted C1 to C6 alkyl group.

In an implementation, the hole transport auxiliary layer 33 may include a third compound having bipolar characteristics having relatively strong hole characteristics.

As described above, the light emitting layer 32 may include the first compound having bipolar characteristics in which electron characteristics are relatively strong and the second compound having relatively strong hole characteristics and thereby increases mobility of electrons and holes and remarkably improves luminous efficiency compared with the compounds alone.

When a material having biased electron or hole characteristics is used to form an light emitting layer, excitons in a device including the light emitting layer may be relatively more generated due to recombination of carriers on the interface between the light emitting layer and the electron or hole transport layer. As a result, the molecular excitons in the light emitting layer may interact with charges on the interface of the hole transport layer and thus, could cause a roll-off of sharply deteriorating efficiency and also, sharply deteriorate light emitting life-span characteristics.

In an implementation, the first and second compounds may be simultaneously included in the light emitting layer to make a light emitting region not be biased to either of the electron transport layer or the hole transport layer, and additionally, the hole transport auxiliary layer including the third compound may be between the hole transport layer and the light emitting layer, and charges may be prevented from being accumulated at the interface between the hole transport layer and the light emitting layer and a device capable of adjusting carrier balance in the light emitting layer may be provided. Accordingly, roll-off characteristics of an organic optoelectronic device may be improved and simultaneously life-span characteristics may be remarkably improved.

The third compound may be a compound represented by Chemical Formula 5.

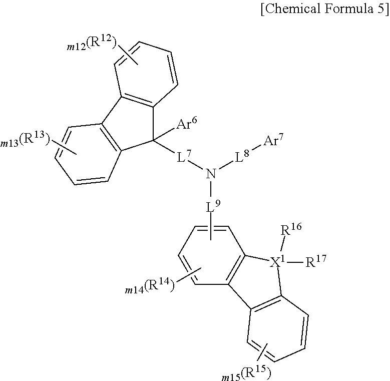

In Chemical Formula 5, X¹ may be, e.g., C or Si.

R¹² to R¹⁵ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

R¹⁶ and R¹⁷ may each independently be or include, e.g., a substituted or unsubstituted C1 to C30 alkyl group or a substituted or unsubstituted C6 to C30 aryl group.

Ar⁶ may be or include, e.g., a substituted or unsubstituted C6 to C30 aryl group.

Ar⁷ may be or include, e.g., a substituted or unsubstituted C6 to C30 aryl group or a substituted or unsubstituted dibenzosilolyl group.

L⁷ to L⁹ may each independently be or include, e.g., a single bond or a substituted or unsubstituted C6 to C20 arylene group.

m12, m13, and m15 may each independently be, e.g., an integer of 1 to 4.

m14 may be, e.g., an integer of 1 to 3.

The third compound may be an amine derivative simultaneously including a fluorene group substituted at the 9-position and a fluorene group substituted in the phenyl direction or a dibenzosilolyl group substituted in the phenyl direction.

Due to steric hindrance of the fluorene group substituted at the 9-position, a deposition temperature may be lowered, minimizing degradation decomposition and thereby, further improving life-span characteristics.

In addition, the fluorene group substituted in the phenyl direction or the dibenzosilolyl group substituted in the phenyl direction may be simultaneously included and thus may lower a HOMO energy level and thus facilitate hole injection.

In an implementation, the third compound may be represented by any one of Chemical Formula 5-1 to Chemical Formula 5-4.

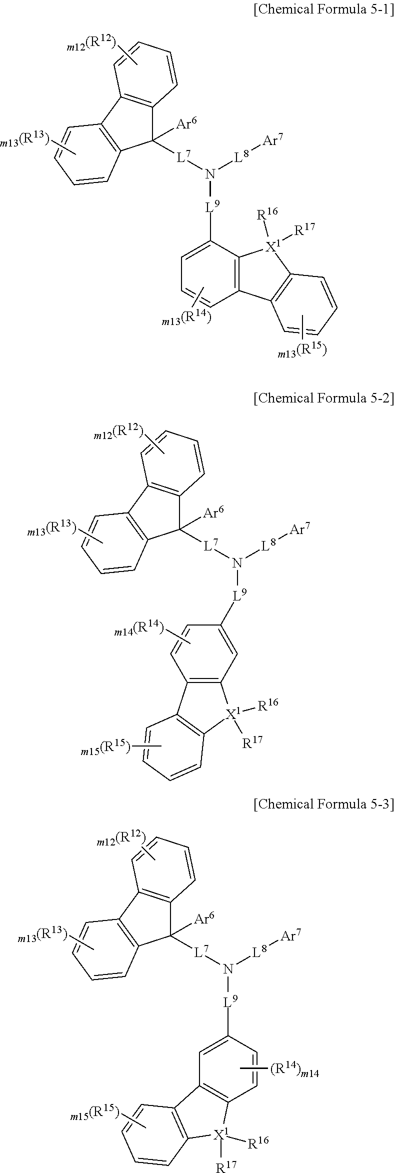

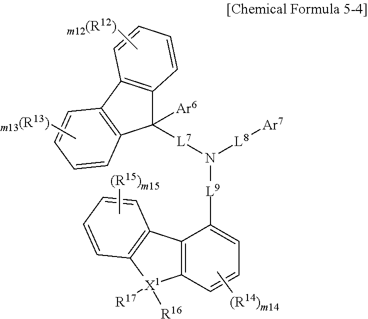

In Chemical Formula 5-1 to Chemical Formula 5-4, X¹, R¹² to R¹⁷, Ar⁶, Ar⁷, L⁷ to L⁹, and m12 to m15 may be defined the same as described above.

In an implementation, the third compound may be represented by Chemical Formula 5-2.

In an implementation, Ar⁶ in Chemical Formula 5 may be a substituted or unsubstituted phenyl group, a substituted or unsubstituted biphenyl group, or a substituted or unsubstituted terphenyl group.

In an implementation, Ar⁶ in Chemical Formula 5 may be a substituted or unsubstituted phenyl group or a substituted or unsubstituted biphenyl group.

In an implementation, Ar⁷ in Chemical Formula 5 may be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, or a substituted or unsubstituted dibenzosilolyl group.

In an implementation, Ar⁷ in Chemical Formula 5 may be a group of Group III.

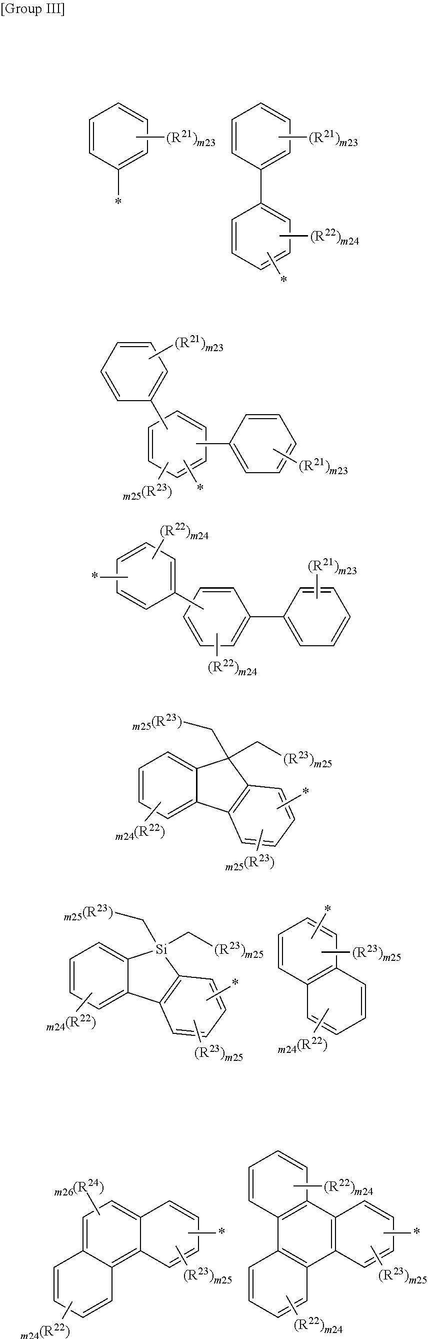

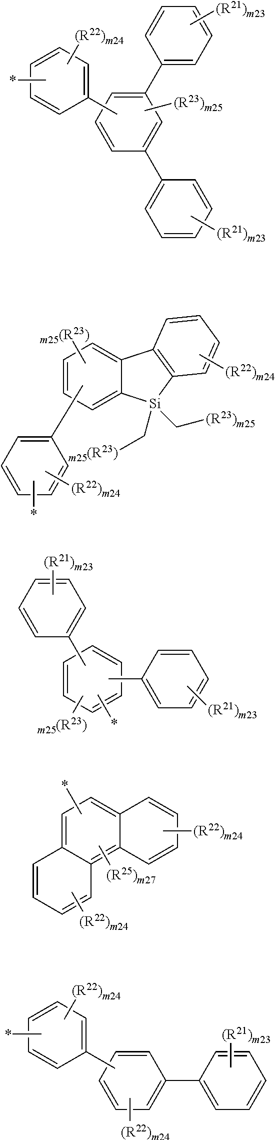

In Group III, R²¹ to R²⁵ may each independently be, e.g., hydrogen, deuterium, a cyano group, a substituted or unsubstituted C1 to C10 alkyl group, or a substituted or unsubstituted C6 to C12 aryl group.

m23 may be, e.g., an integer of 1 to 5.

m24 may be, e.g., an integer of 1 to 4.

m25 may be, e.g., an integer of 1 to 3.

m26 may be, e.g., 1 or 2.

m27 may be, e.g., 1.

* is a linking point.

In an implementation, the third compound may be a compound of Group 3.

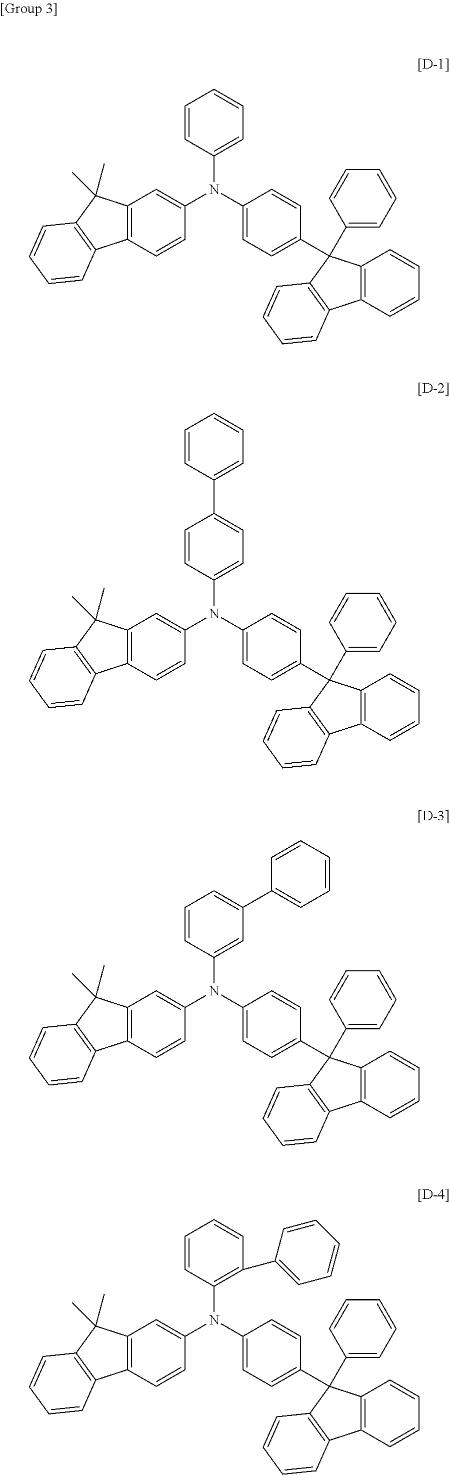 [SMILES: CC1(C)c2ccccc2-c2ccc(N(c3ccccc3)c3ccc(C4(c5ccccc5)c5ccccc5-c5ccccc54)cc3)cc21 ; CC1(C)c2ccccc2-c2ccc(N(c3ccc(-c4ccccc4)cc3)c3ccc(C4(c5ccccc5)c5ccccc5-c5ccccc54)cc3)cc21 ; CC1(C)c2ccccc2-c2ccc(N(c3ccc(C4(c5ccccc5)c5ccccc5-c5ccccc54)cc3)c3cccc(-c4ccccc4)c3)cc21 ; CC1(C)c2ccccc2-c2ccc(N(c3ccc(C4(c5ccccc5)c5ccccc5-c5ccccc54)cc3)c3ccccc3-c3ccccc3)cc21]

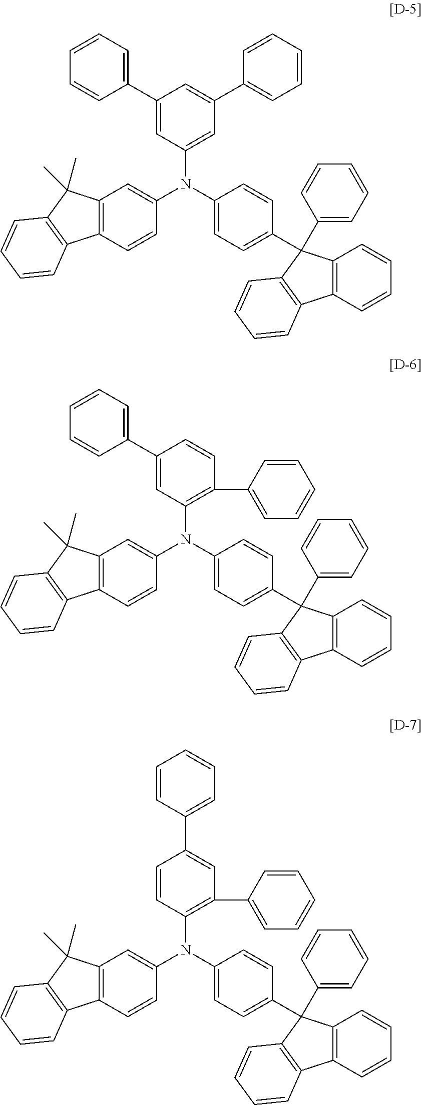 [SMILES: CC1(C)c2ccccc2-c2ccc(N(c3ccc(C4(c5ccccc5)c5ccccc5-c5ccccc54)cc3)c3cc(-c4ccccc4)cc(-c4ccccc4)c3)cc21 ; CC1(C)c2ccccc2-c2ccc(N(c3ccc(C4(c5ccccc5)c5ccccc5-c5ccccc54)cc3)c3cc(-c4ccccc4)ccc3-c3ccccc3)cc21 ; CC1(C)c2ccccc2-c2ccc(N(c3ccc(C4(c5ccccc5)c5ccccc5-c5ccccc54)cc3)c3ccc(-c4ccccc4)cc3-c3ccccc3)cc21]

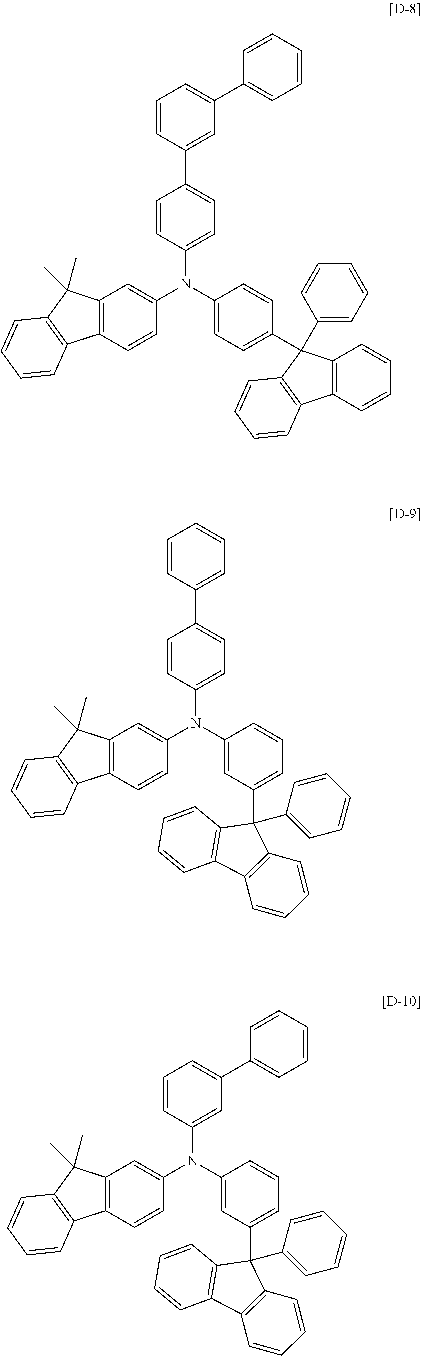 [SMILES: CC1(C)c2ccccc2-c2ccc(N(c3ccc(-c4cccc(-c5ccccc5)c4)cc3)c3ccc(C4(c5ccccc5)c5ccccc5-c5ccccc54)cc3)cc21 ; CC1(C)c2ccccc2-c2ccc(N(c3ccc(-c4ccccc4)cc3)c3cccc(C4(c5ccccc5)c5ccccc5-c5ccccc54)c3)cc21 ; CC1(C)c2ccccc2-c2ccc(N(c3cccc(-c4ccccc4)c3)c3cccc(C4(c5ccccc5)c5ccccc5-c5ccccc54)c3)cc21]

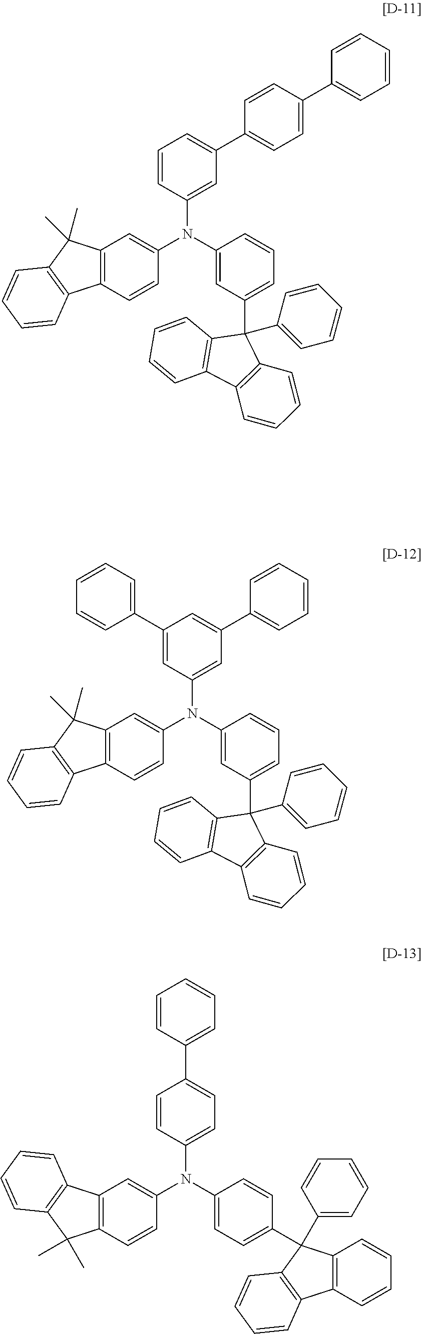 [SMILES: CC1(C)c2ccccc2-c2ccc(N(c3cccc(-c4ccc(-c5ccccc5)cc4)c3)c3cccc(C4(c5ccccc5)c5ccccc5-c5ccccc54)c3)cc21 ; CC1(C)c2ccccc2-c2ccc(N(c3cc(-c4ccccc4)cc(-c4ccccc4)c3)c3cccc(C4(c5ccccc5)c5ccccc5-c5ccccc54)c3)cc21 ; CC1(C)c2ccccc2-c2cc(N(c3ccc(-c4ccccc4)cc3)c3ccc(C4(c5ccccc5)c5ccccc5-c5ccccc54)cc3)ccc21]

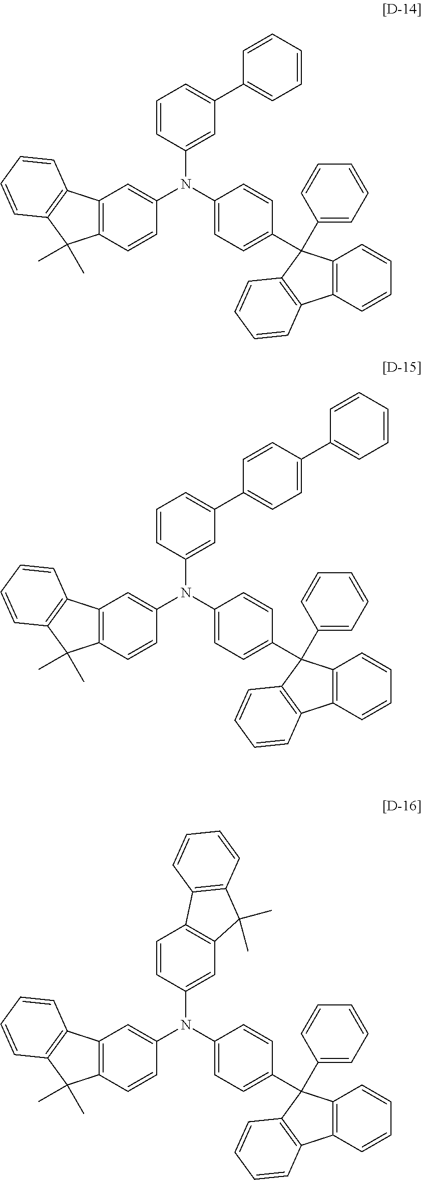 [SMILES: CC1(C)c2ccccc2-c2cc(N(c3ccc(C4(c5ccccc5)c5ccccc5-c5ccccc54)cc3)c3cccc(-c4ccccc4)c3)ccc21 ; CC1(C)c2ccccc2-c2cc(N(c3ccc(C4(c5ccccc5)c5ccccc5-c5ccccc54)cc3)c3cccc(-c4ccc(-c5ccccc5)cc4)c3)ccc21 ; CC1(C)c2ccccc2-c2cc(N(c3ccc(C4(c5ccccc5)c5ccccc5-c5ccccc54)cc3)c3ccc4c(c3)C(C)(C)c3ccccc3-4)ccc21]

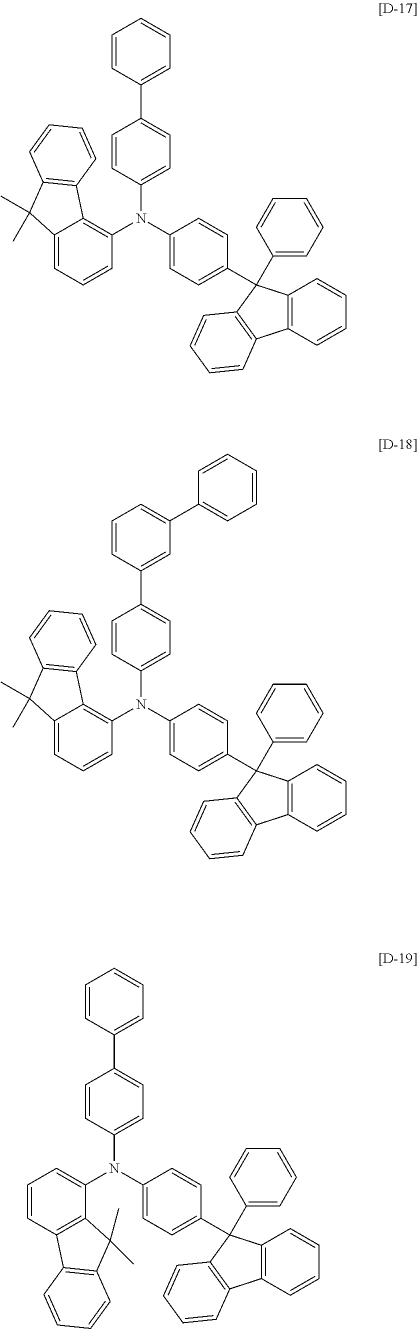 [SMILES: CC1(C)c2ccccc2-c2c(N(c3ccc(-c4ccccc4)cc3)c3ccc(C4(c5ccccc5)c5ccccc5-c5ccccc54)cc3)cccc21 ; CC1(C)c2ccccc2-c2c(N(c3ccc(-c4cccc(-c5ccccc5)c4)cc3)c3ccc(C4(c5ccccc5)c5ccccc5-c5ccccc54)cc3)cccc21 ; CC1(C)c2ccccc2-c2cccc(N(c3ccc(-c4ccccc4)cc3)c3ccc(C4(c5ccccc5)c5ccccc5-c5ccccc54)cc3)c21]

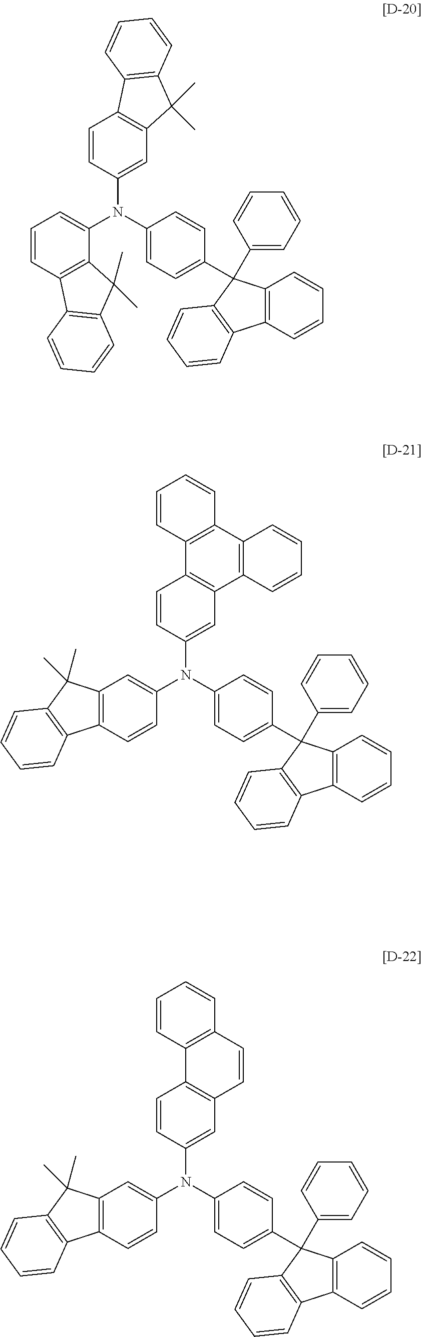 [SMILES: CC1(C)c2ccccc2-c2ccc(N(c3ccc(C4(c5ccccc5)c5ccccc5-c5ccccc54)cc3)c3cccc4c3C(C)(C)c3ccccc3-4)cc21 ; CC1(C)c2ccccc2-c2ccc(N(c3ccc(C4(c5ccccc5)c5ccccc5-c5ccccc54)cc3)c3ccc4c5ccccc5c5ccccc5c4c3)cc21 ; CC1(C)c2ccccc2-c2ccc(N(c3ccc(C4(c5ccccc5)c5ccccc5-c5ccccc54)cc3)c3ccc4c(ccc5ccccc54)c3)cc21]

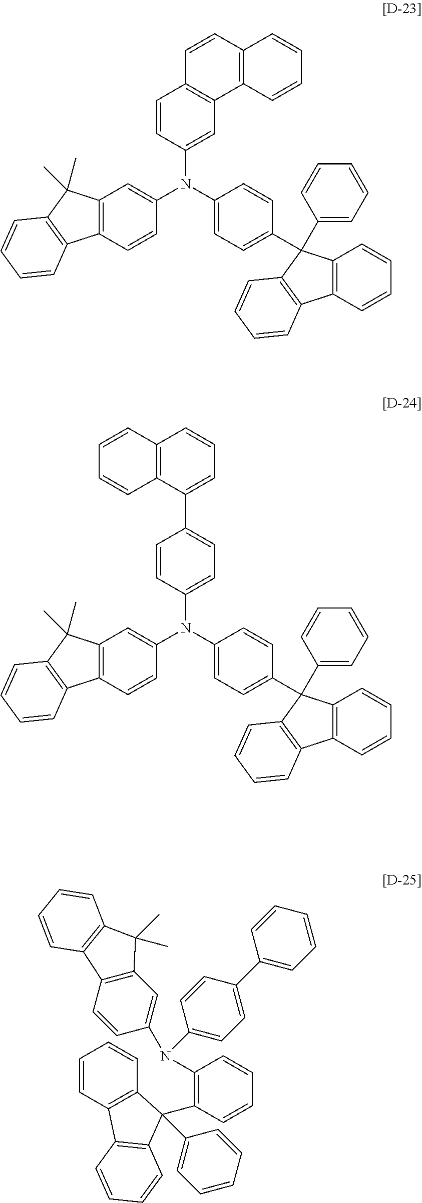 [SMILES: CC1(C)c2ccccc2-c2ccc(N(c3ccc(C4(c5ccccc5)c5ccccc5-c5ccccc54)cc3)c3ccc4ccc5ccccc5c4c3)cc21 ; CC1(C)c2ccccc2-c2ccc(N(c3ccc(-c4cccc5ccccc45)cc3)c3ccc(C4(c5ccccc5)c5ccccc5-c5ccccc54)cc3)cc21 ; CC1(C)c2ccccc2-c2ccc(N(c3ccc(-c4ccccc4)cc3)c3ccccc3C3(c4ccccc4)c4ccccc4-c4ccccc43)cc21]

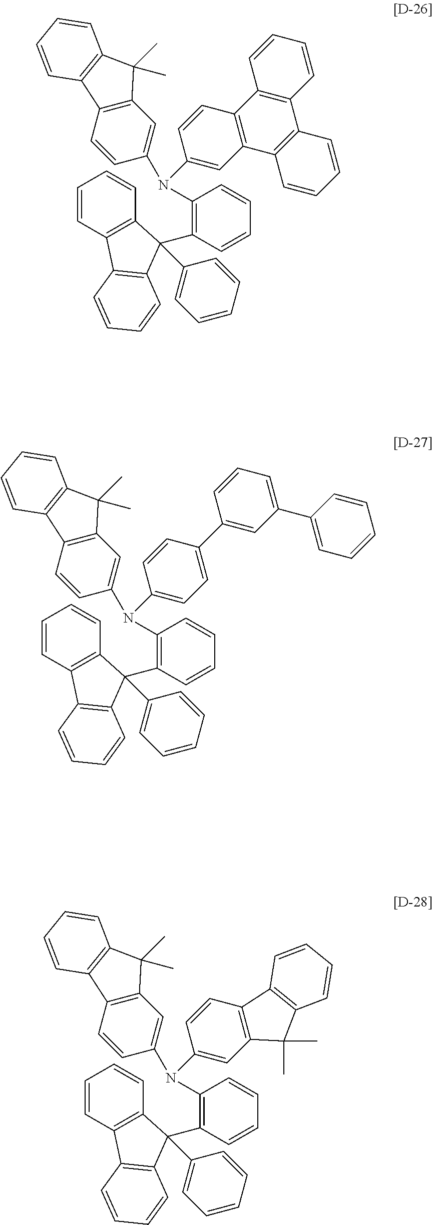 [SMILES: CC1(C)c2ccccc2-c2ccc(N(c3ccc4c5ccccc5c5ccccc5c4c3)c3ccccc3C3(c4ccccc4)c4ccccc4-c4ccccc43)cc21 ; CC1(C)c2ccccc2-c2ccc(N(c3ccc(-c4cccc(-c5ccccc5)c4)cc3)c3ccccc3C3(c4ccccc4)c4ccccc4-c4ccccc43)cc21 ; CC1(C)c2ccccc2-c2ccc(N(c3ccc4c(c3)C(C)(C)c3ccccc3-4)c3ccccc3C3(c4ccccc4)c4ccccc4-c4ccccc43)cc21]

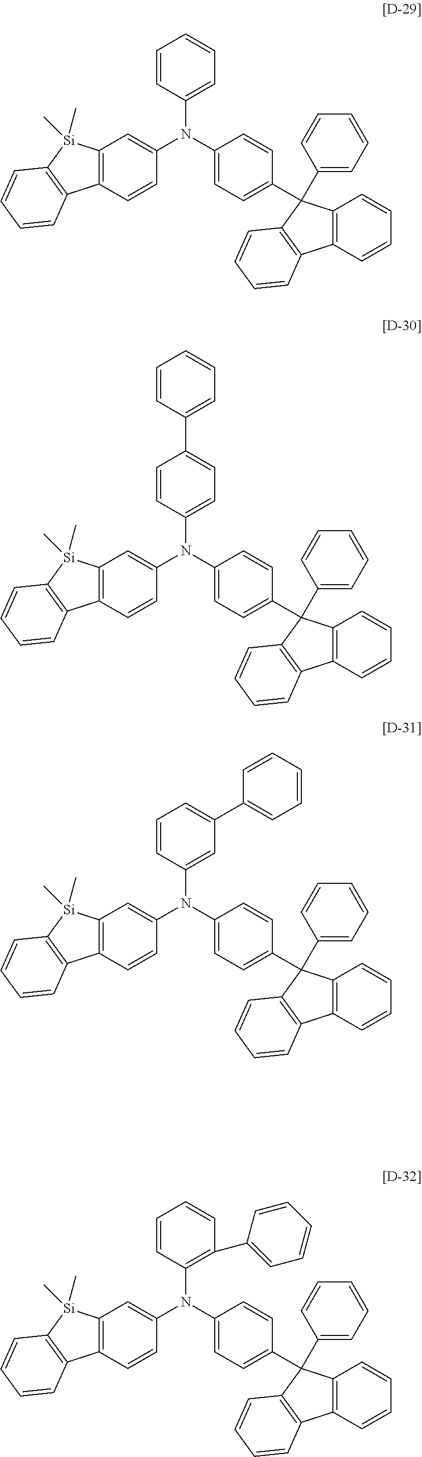 [SMILES: C[Si]1(C)c2ccccc2-c2ccc(N(c3ccccc3)c3ccc(C4(c5ccccc5)c5ccccc5-c5ccccc54)cc3)cc21 ; C[Si]1(C)c2ccccc2-c2ccc(N(c3ccc(-c4ccccc4)cc3)c3ccc(C4(c5ccccc5)c5ccccc5-c5ccccc54)cc3)cc21 ; C[Si]1(C)c2ccccc2-c2ccc(N(c3ccc(C4(c5ccccc5)c5ccccc5-c5ccccc54)cc3)c3cccc(-c4ccccc4)c3)cc21 ; C[Si]1(C)c2ccccc2-c2ccc(N(c3ccc(C4(c5ccccc5)c5ccccc5-c5ccccc54)cc3)c3ccccc3-c3ccccc3)cc21]

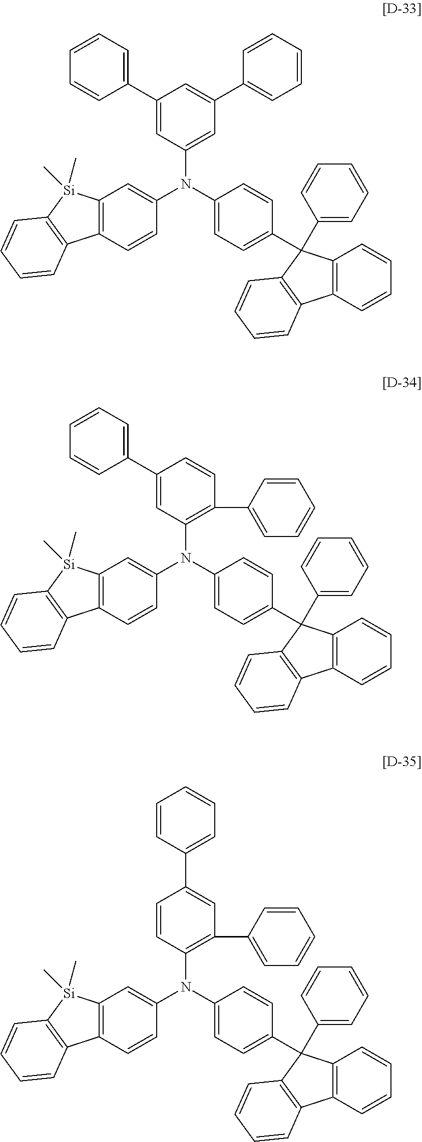 [SMILES: C[Si]1(C)c2ccccc2-c2ccc(N(c3ccc(C4(c5ccccc5)c5ccccc5-c5ccccc54)cc3)c3cc(-c4ccccc4)cc(-c4ccccc4)c3)cc21 ; C[Si]1(C)c2ccccc2-c2ccc(N(c3ccc(C4(c5ccccc5)c5ccccc5-c5ccccc54)cc3)c3cc(-c4ccccc4)ccc3-c3ccccc3)cc21 ; C[Si]1(C)c2ccccc2-c2ccc(N(c3ccc(C4(c5ccccc5)c5ccccc5-c5ccccc54)cc3)c3ccc(-c4ccccc4)cc3-c3ccccc3)cc21]

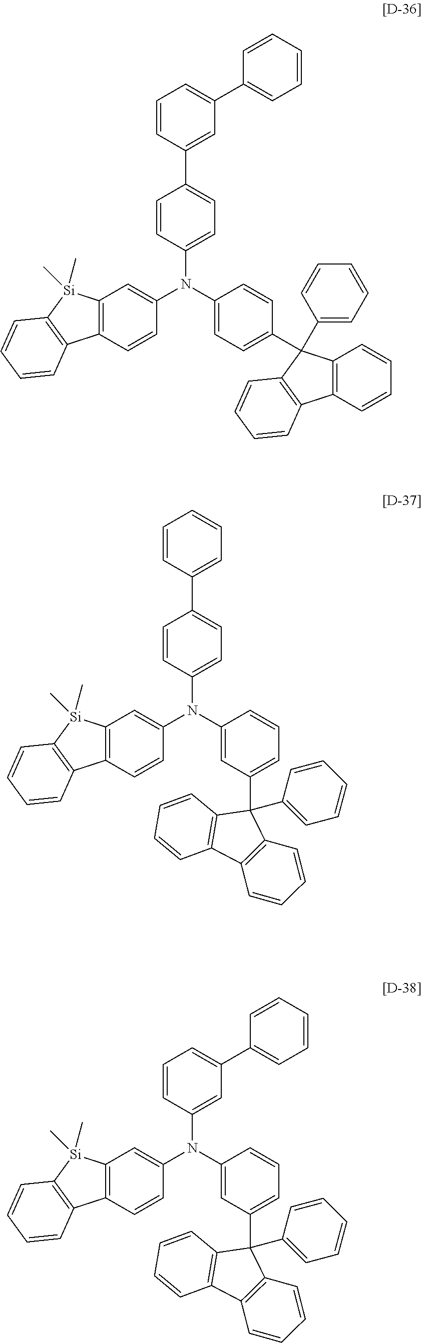 [SMILES: C[Si]1(C)c2ccccc2-c2ccc(N(c3ccc(-c4cccc(-c5ccccc5)c4)cc3)c3ccc(C4(c5ccccc5)c5ccccc5-c5ccccc54)cc3)cc21 ; C[Si]1(C)c2ccccc2-c2ccc(N(c3ccc(-c4ccccc4)cc3)c3cccc(C4(c5ccccc5)c5ccccc5-c5ccccc54)c3)cc21 ; C[Si]1(C)c2ccccc2-c2ccc(N(c3cccc(-c4ccccc4)c3)c3cccc(C4(c5ccccc5)c5ccccc5-c5ccccc54)c3)cc21]

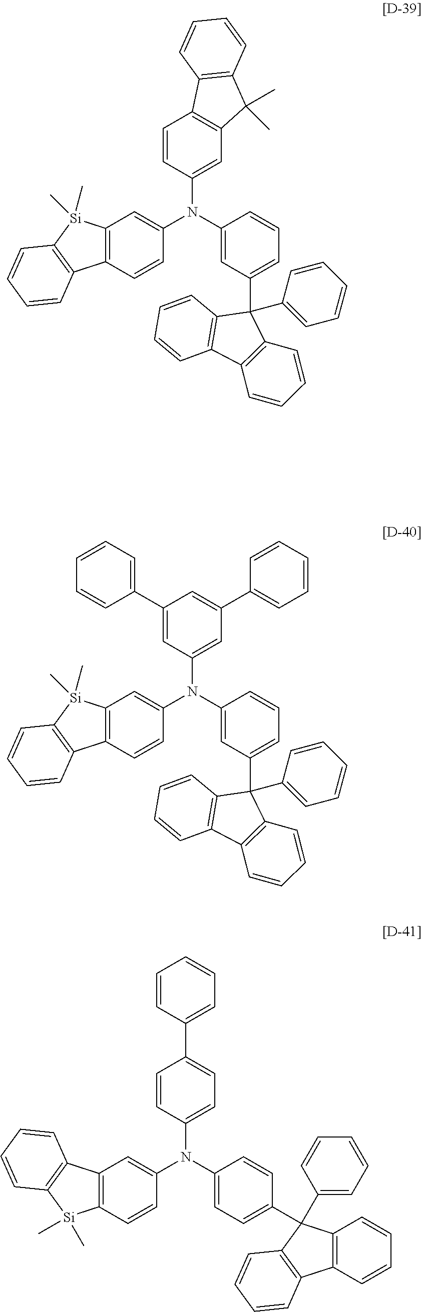 [SMILES: CC1(C)c2ccccc2-c2ccc(N(c3cccc(C4(c5ccccc5)c5ccccc5-c5ccccc54)c3)c3ccc4c(c3)[Si](C)(C)c3ccccc3-4)cc21 ; C[Si]1(C)c2ccccc2-c2ccc(N(c3cc(-c4ccccc4)cc(-c4ccccc4)c3)c3cccc(C4(c5ccccc5)c5ccccc5-c5ccccc54)c3)cc21 ; C[Si]1(C)c2ccccc2-c2cc(N(c3ccc(-c4ccccc4)cc3)c3ccc(C4(c5ccccc5)c5ccccc5-c5ccccc54)cc3)ccc21]

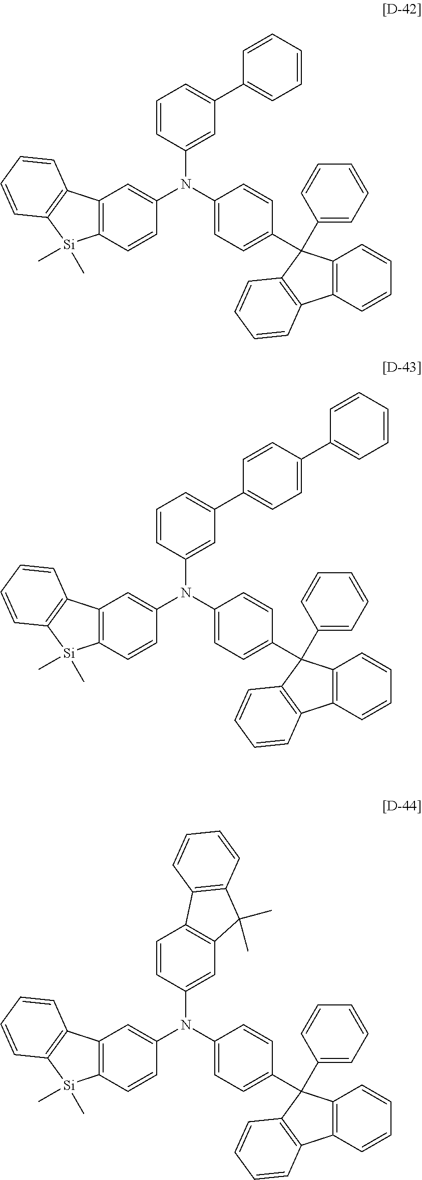 [SMILES: C[Si]1(C)c2ccccc2-c2cc(N(c3ccc(C4(c5ccccc5)c5ccccc5-c5ccccc54)cc3)c3cccc(-c4ccccc4)c3)ccc21 ; C[Si]1(C)c2ccccc2-c2cc(N(c3ccc(C4(c5ccccc5)c5ccccc5-c5ccccc54)cc3)c3cccc(-c4ccc(-c5ccccc5)cc4)c3)ccc21 ; CC1(C)c2ccccc2-c2ccc(N(c3ccc(C4(c5ccccc5)c5ccccc5-c5ccccc54)cc3)c3ccc4c(c3)-c3ccccc3[Si]4(C)C)cc21]

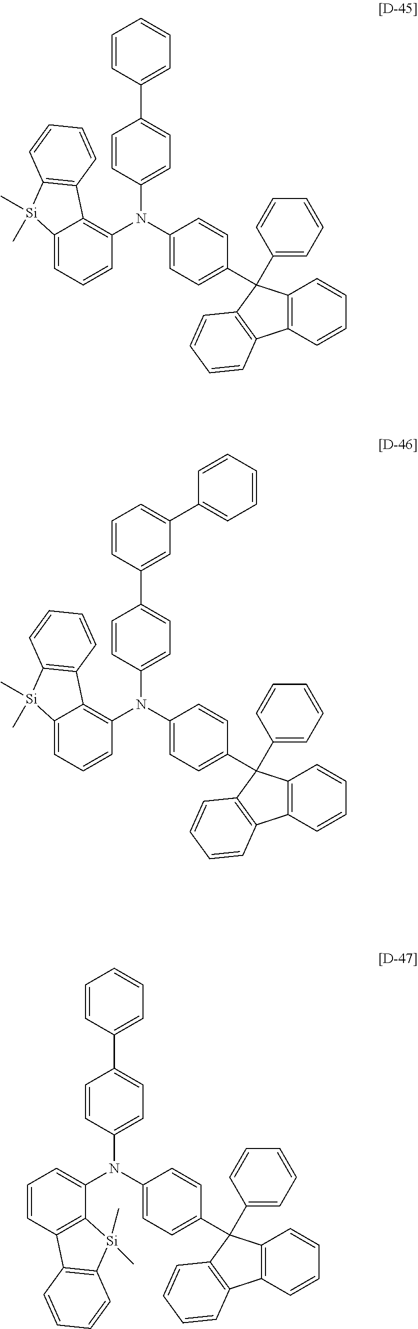 [SMILES: C[Si]1(C)c2ccccc2-c2c(N(c3ccc(-c4ccccc4)cc3)c3ccc(C4(c5ccccc5)c5ccccc5-c5ccccc54)cc3)cccc21 ; C[Si]1(C)c2ccccc2-c2c(N(c3ccc(-c4cccc(-c5ccccc5)c4)cc3)c3ccc(C4(c5ccccc5)c5ccccc5-c5ccccc54)cc3)cccc21 ; C[Si]1(C)c2ccccc2-c2cccc(N(c3ccc(-c4ccccc4)cc3)c3ccc(C4(c5ccccc5)c5ccccc5-c5ccccc54)cc3)c21]

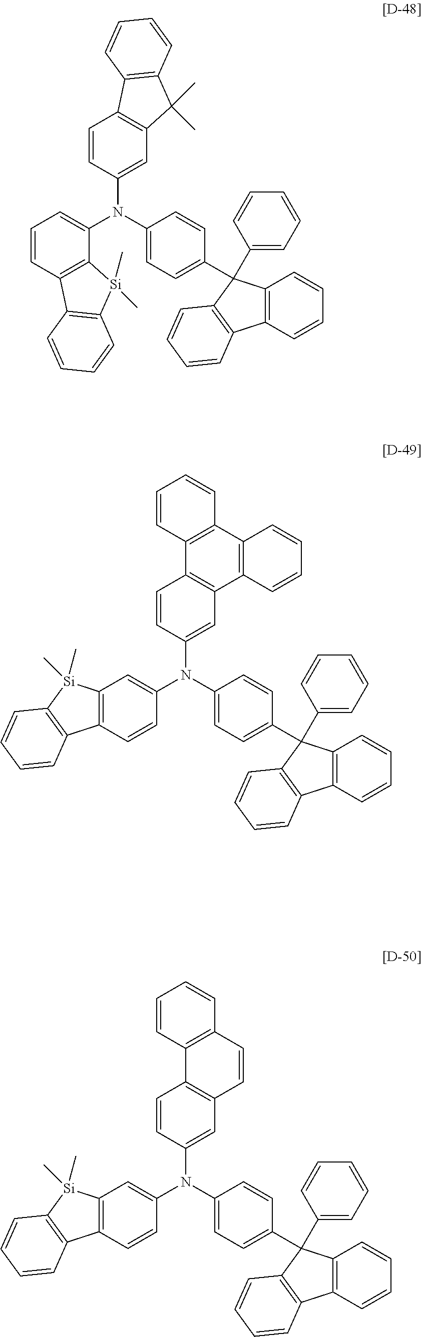 [SMILES: CC1(C)c2ccccc2-c2ccc(N(c3ccc(C4(c5ccccc5)c5ccccc5-c5ccccc54)cc3)c3cccc4c3[Si](C)(C)c3ccccc3-4)cc21 ; C[Si]1(C)c2ccccc2-c2ccc(N(c3ccc(C4(c5ccccc5)c5ccccc5-c5ccccc54)cc3)c3ccc4c5ccccc5c5ccccc5c4c3)cc21 ; C[Si]1(C)c2ccccc2-c2ccc(N(c3ccc(C4(c5ccccc5)c5ccccc5-c5ccccc54)cc3)c3ccc4c(ccc5ccccc54)c3)cc21]

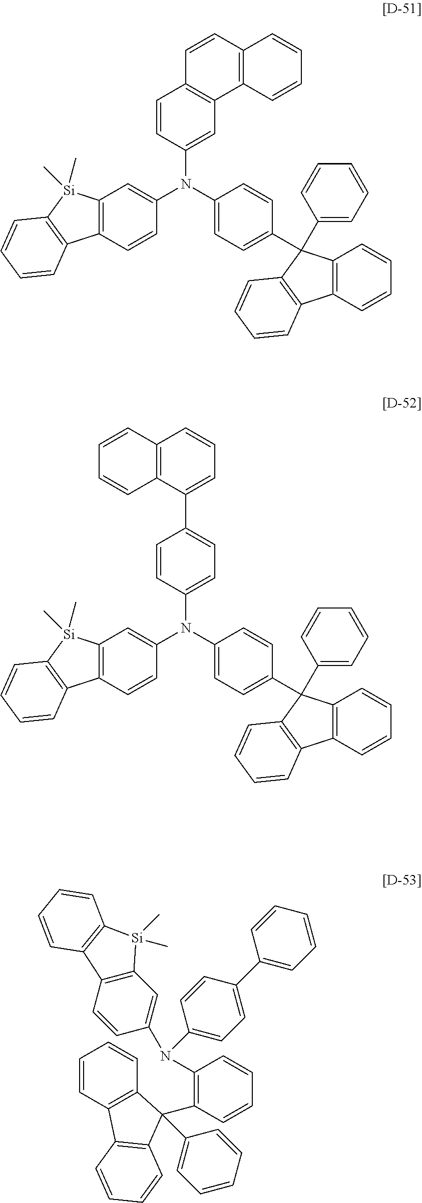 [SMILES: C[Si]1(C)c2ccccc2-c2ccc(N(c3ccc(C4(c5ccccc5)c5ccccc5-c5ccccc54)cc3)c3ccc4ccc5ccccc5c4c3)cc21 ; C[Si]1(C)c2ccccc2-c2ccc(N(c3ccc(-c4cccc5ccccc45)cc3)c3ccc(C4(c5ccccc5)c5ccccc5-c5ccccc54)cc3)cc21 ; C[Si]1(C)c2ccccc2-c2ccc(N(c3ccc(-c4ccccc4)cc3)c3ccccc3C3(c4ccccc4)c4ccccc4-c4ccccc43)cc21]

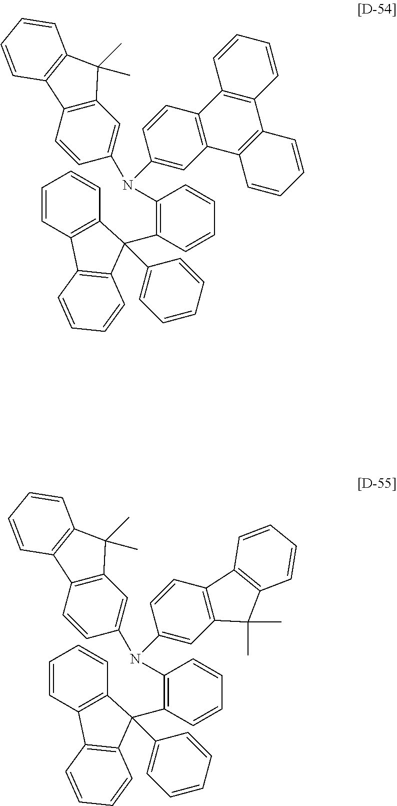 [SMILES: CC1(C)c2ccccc2-c2ccc(N(c3ccc4c5ccccc5c5ccccc5c4c3)c3ccccc3C3(c4ccccc4)c4ccccc4-c4ccccc43)cc21 ; CC1(C)c2ccccc2-c2ccc(N(c3ccc4c(c3)C(C)(C)c3ccccc3-4)c3ccccc3C3(c4ccccc4)c4ccccc4-c4ccccc43)cc21]

In an implementation, the first compound may be the same as described above, the second compound may be represented by Chemical Formula 2-8, and the third compound may be represented by Chemical Formula 5-2.

In an implementation, the organic layer 30 may further include an electron transport region.

The electron transport region may help further increase electron injection or electron mobility and may block holes between the cathode 20 and the light emitting layer 32.

In an implementation, the electron transport region may include an electron transport layer 34 between the cathode 20 and the light emitting layer 32, and an electron transport auxiliary layer between the light emitting layer 32 and the electron transport layer 34. In an implementation, a compound of Group B may be included in the electron transport layer or the electron transport auxiliary layer.

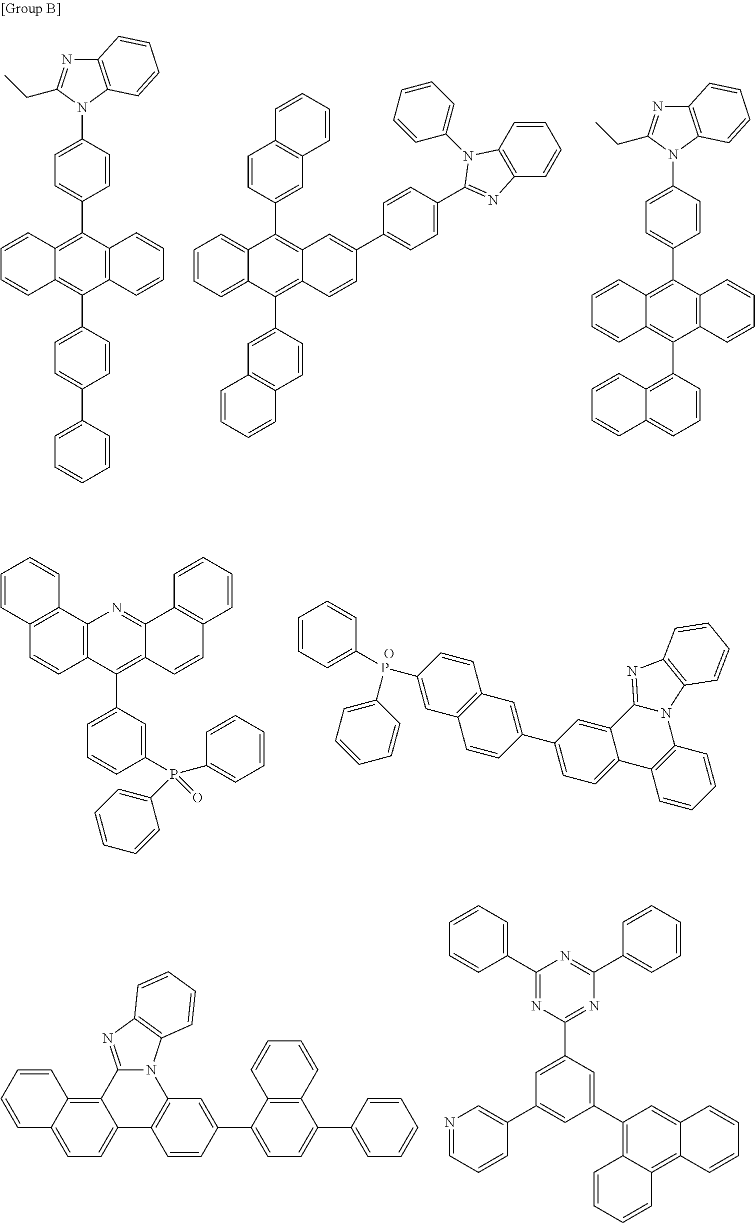

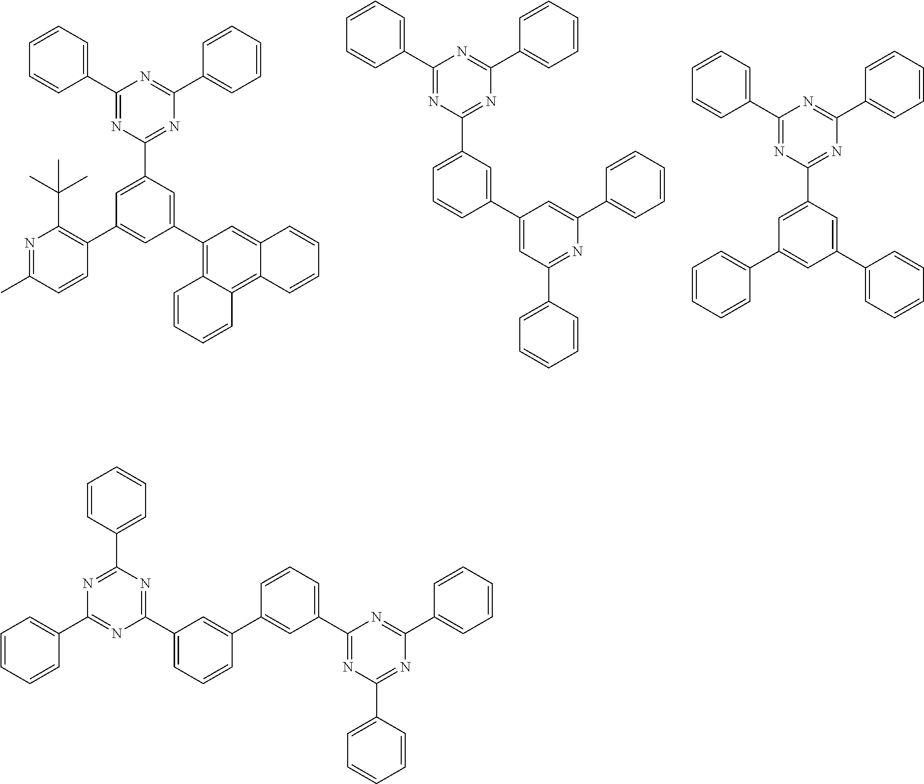 [SMILES: Cc1ccc(-c2cc(-c3nc(-c4ccccc4)nc(-c4ccccc4)n3)cc(-c3cc4ccccc4c4ccccc34)c2)c(C(C)(C)C)n1 ; c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccccc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1]

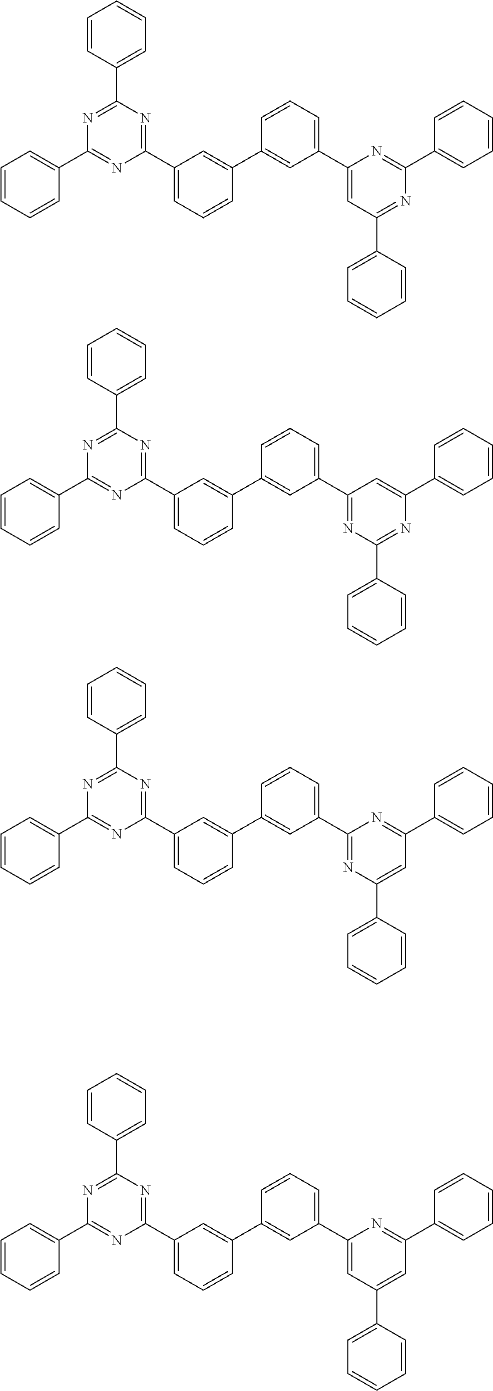 [SMILES: c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1]

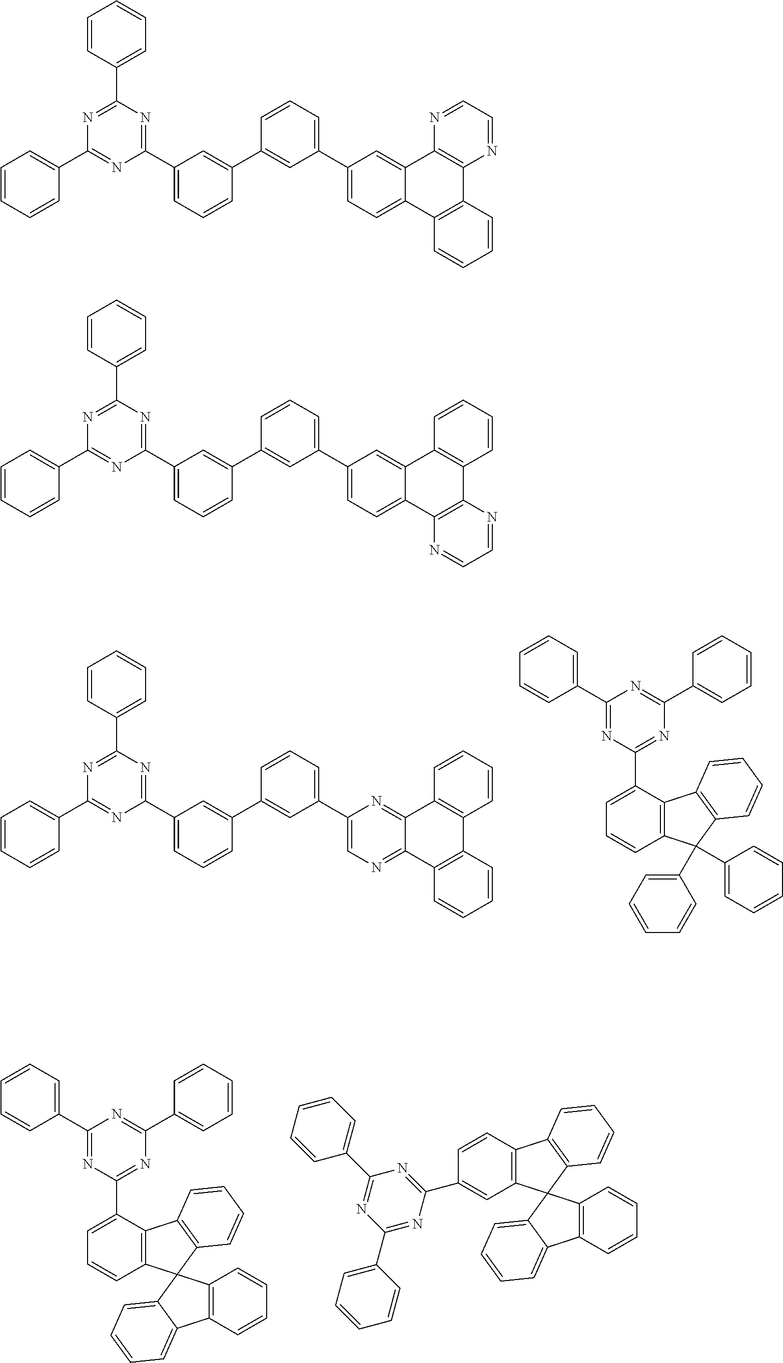 [SMILES: c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1 ; c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1]

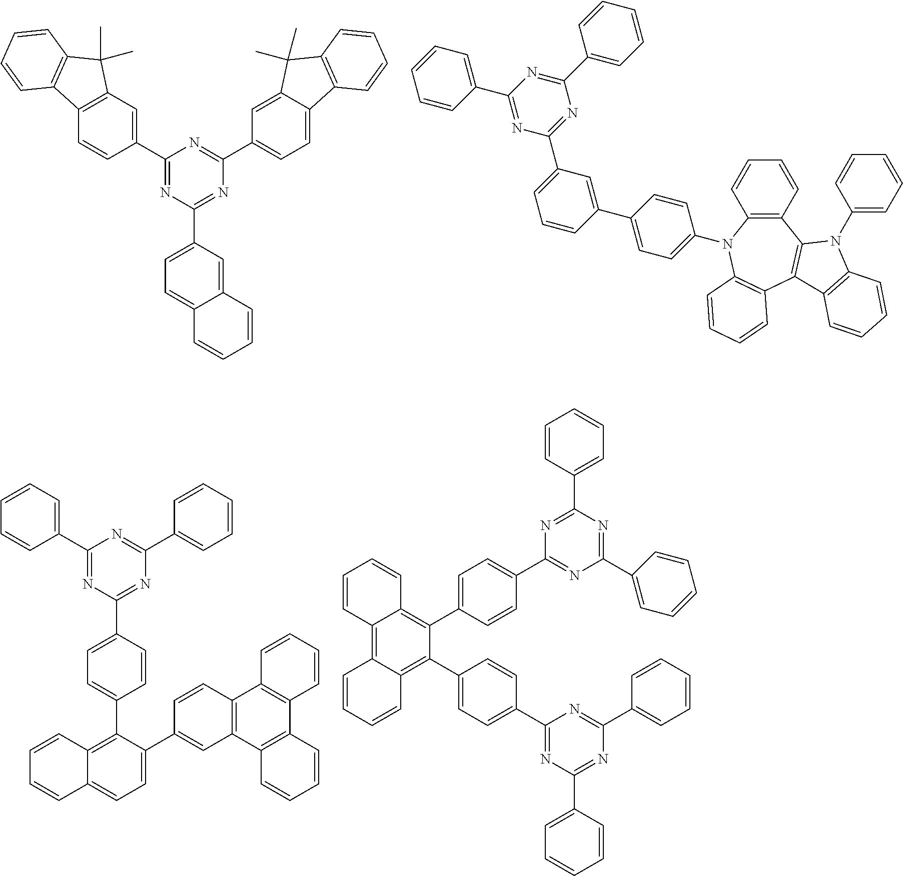

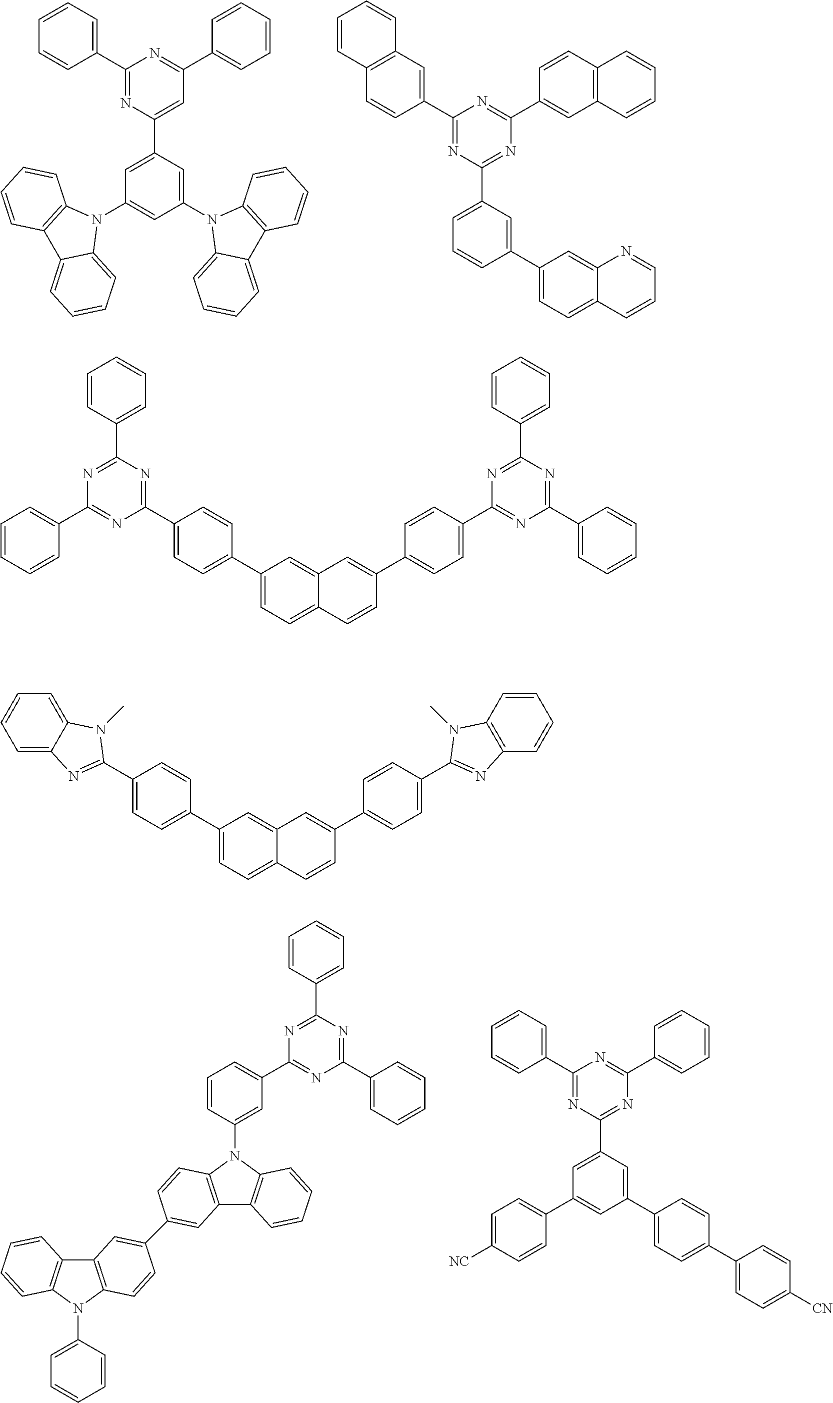

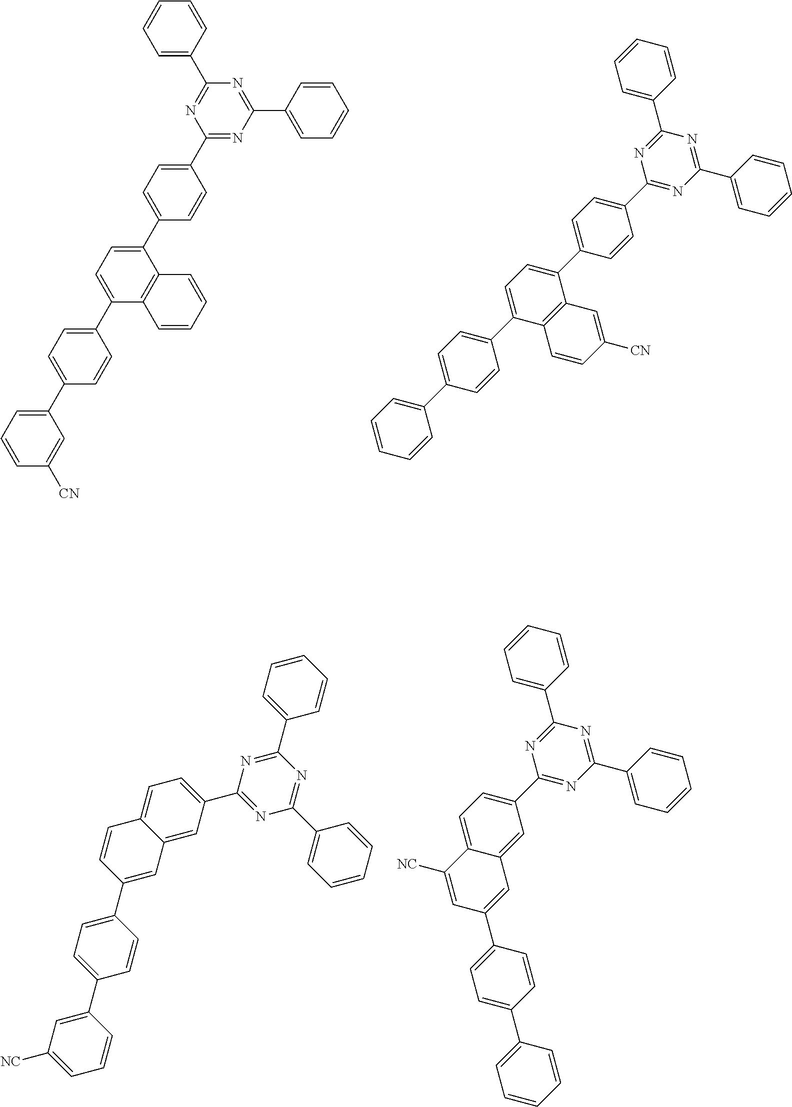

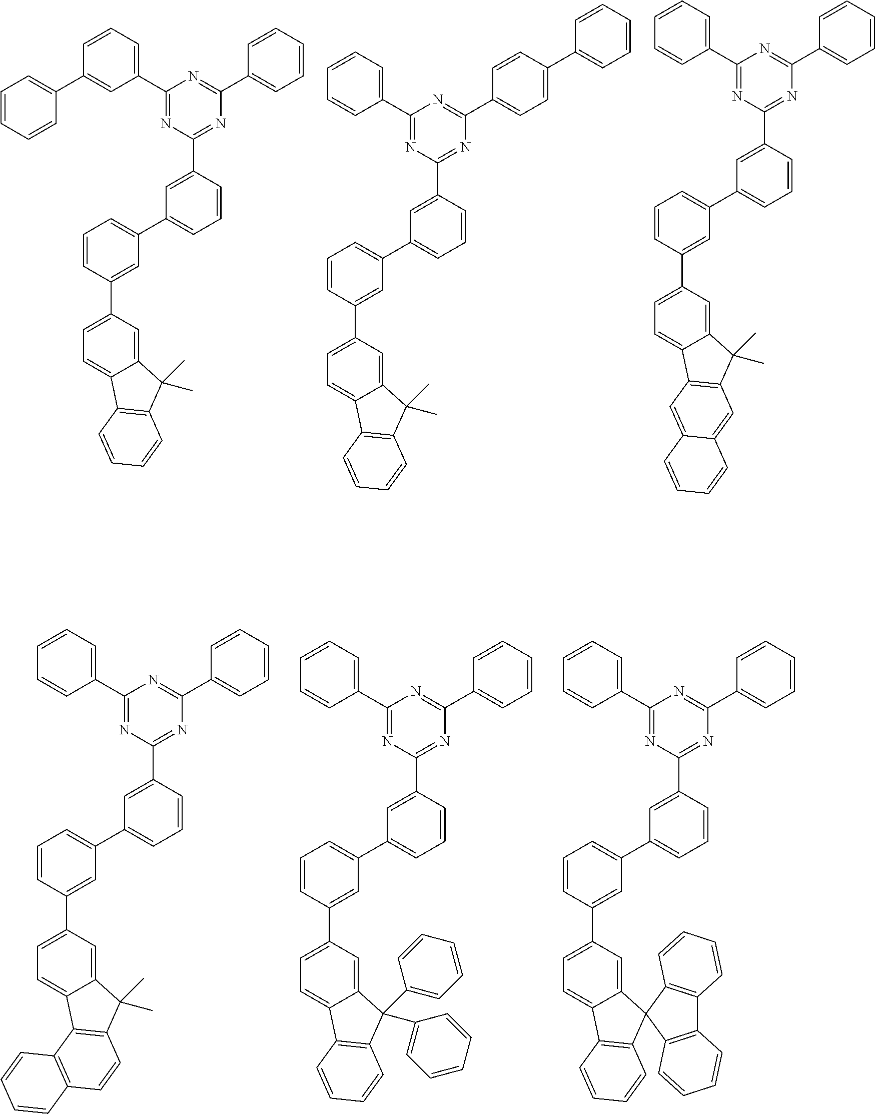

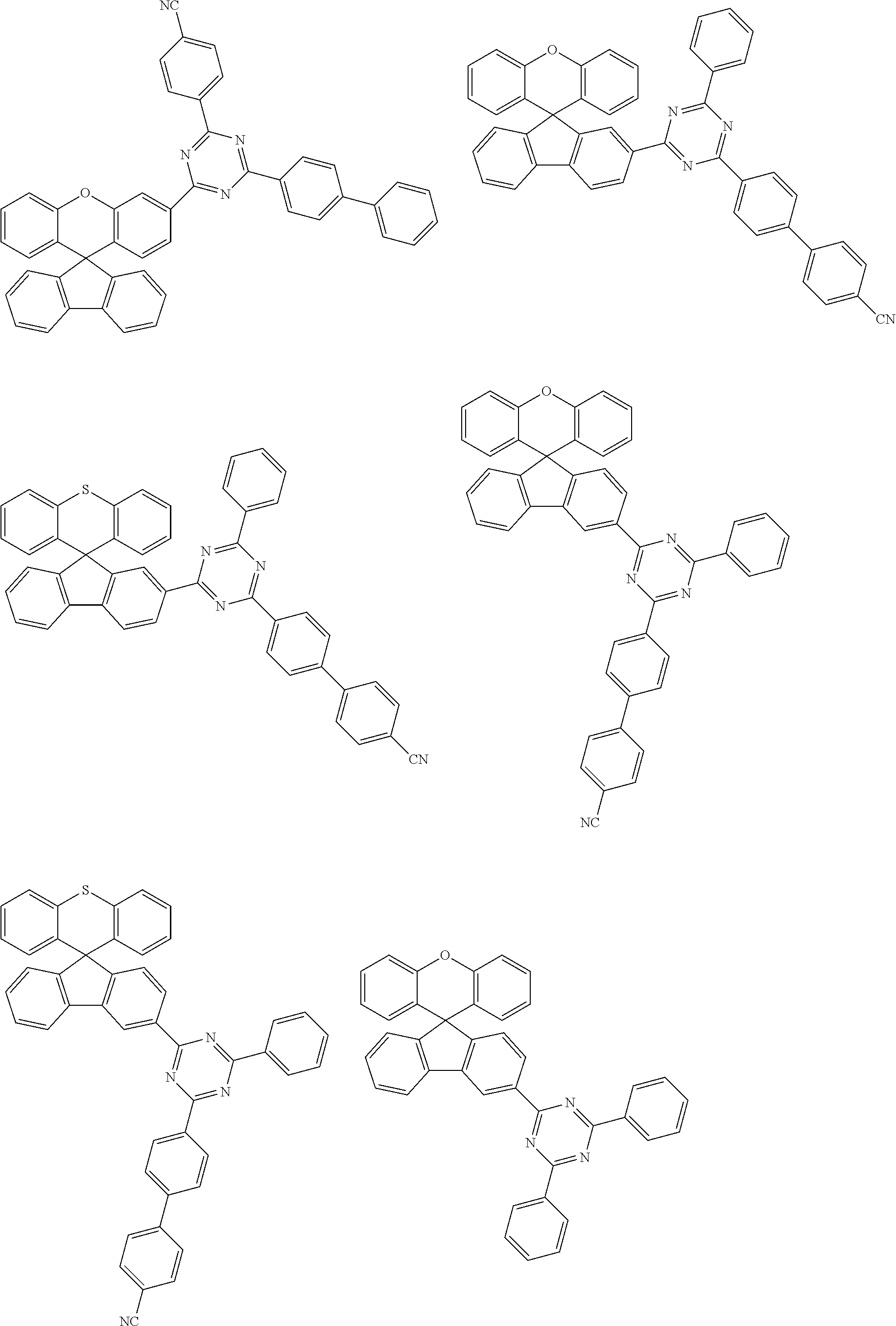

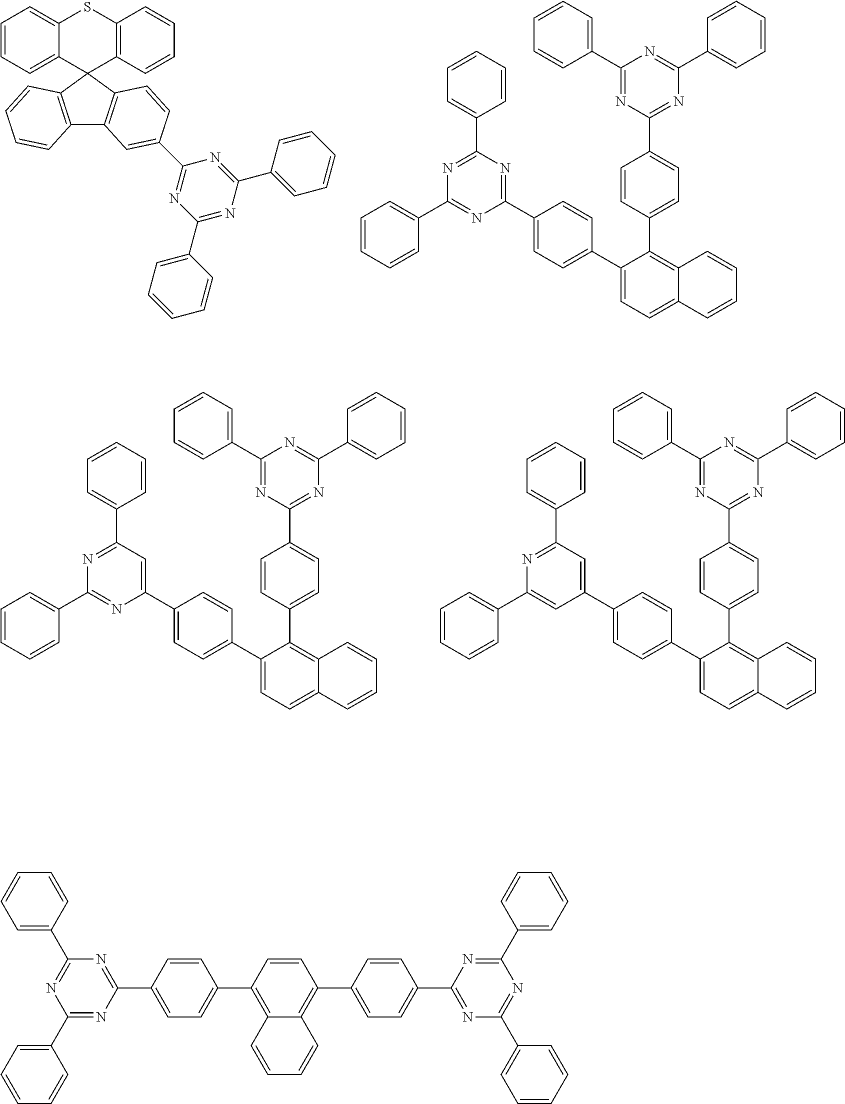

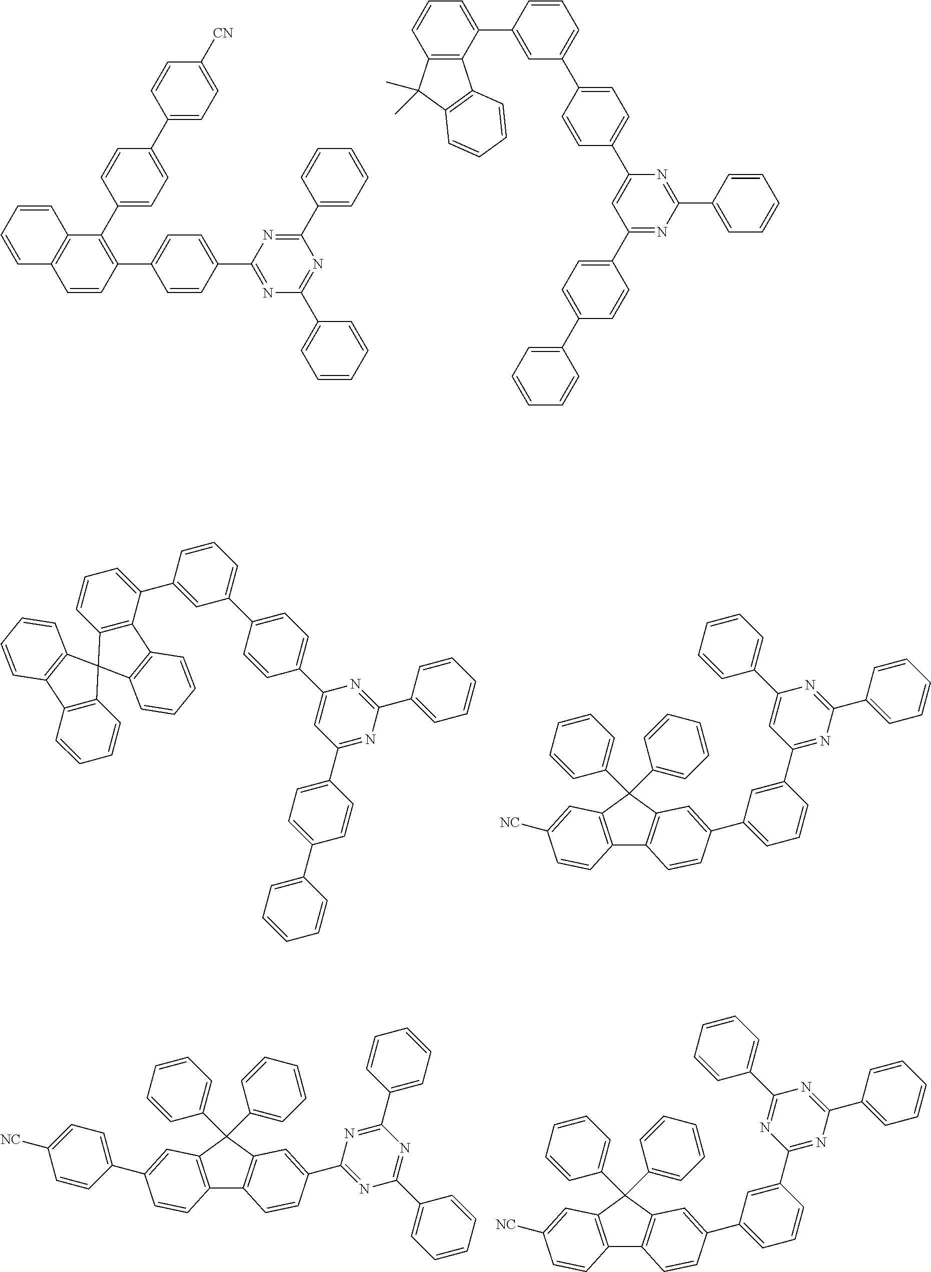

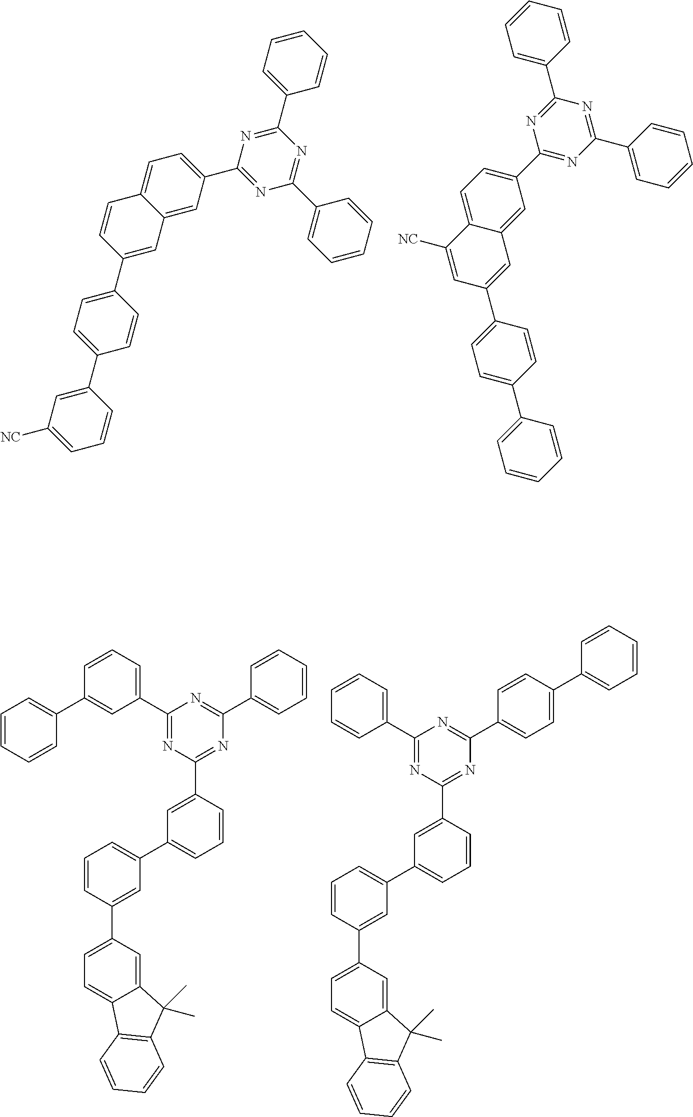

In an implementation, an organic light emitting diode may further include an electron injection layer, a hole injection layer, or the like, in addition to the light emitting layer.

The organic light emitting diodes may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo Chemical Industry, or P&H Tech as far as there in no particular comment or were synthesized by known methods.

(Preparation of Compound for Organic Optoelectronic Device)

Synthesis of First Compound

### Synthesis Example 1: Synthesis of Compound A-52

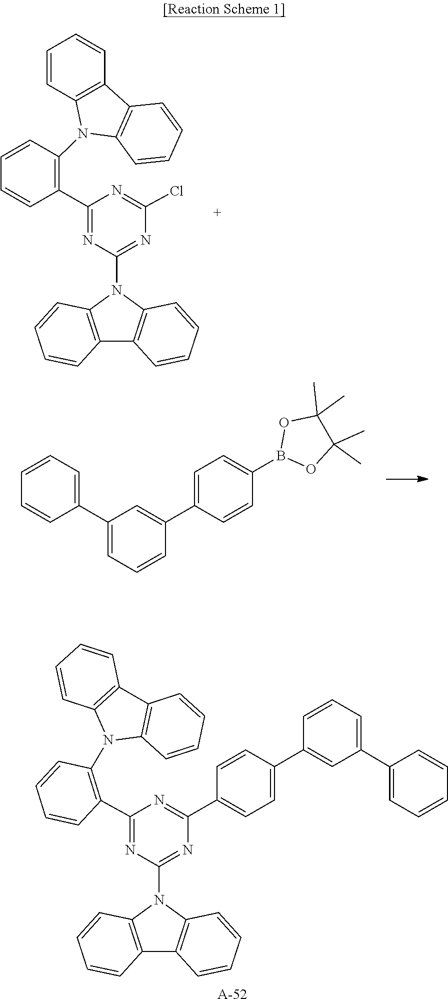

Compound A-52 was synthesized by referring to a synthesis method described in published patent application KR 10-2023-0036973.

### Synthesis Example 2: Synthesis of Compound A-79

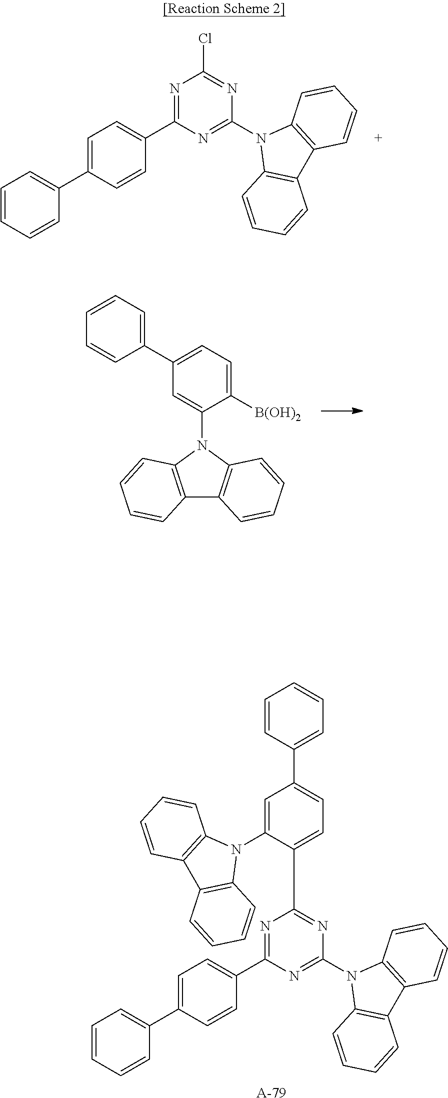 [SMILES: Clc1nc(-c2ccc(-c3ccccc3)cc2)nc(-n2c3ccccc3c3ccccc32)n1 ; OB(O)c1ccc(-c2ccccc2)cc1-n1c2ccccc2c2ccccc21 ; c1ccc(-c2ccc(-c3nc(-c4ccc(-c5ccccc5)cc4-n4c5ccccc5c5ccccc54)nc(-n4c5ccccc5c5ccccc54)n3)cc2)cc1 ; "A-79"]

Compound A-79 was synthesized by referring to the synthesis method described in the patent application KR 10-2021-0126582.

### Comparative Synthesis Example 1: Synthesis of Compound ET-1

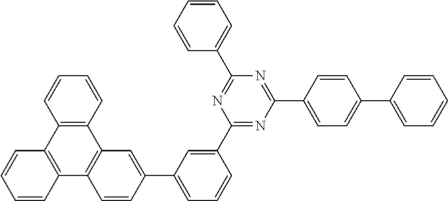 [SMILES: c1ccc(-c2ccc(-c3nc(-c4ccccc4)nc(-c4cccc(-c5ccc6c7ccccc7c7ccccc7c6c5)c4)n3)cc2)cc1]

Compound ET-1 was synthesized by referring to the synthesis method described in the registered patent of KR 10-2275343.

Synthesis of Second Compound

### Synthesis Example 3: Synthesis of Compound B-183

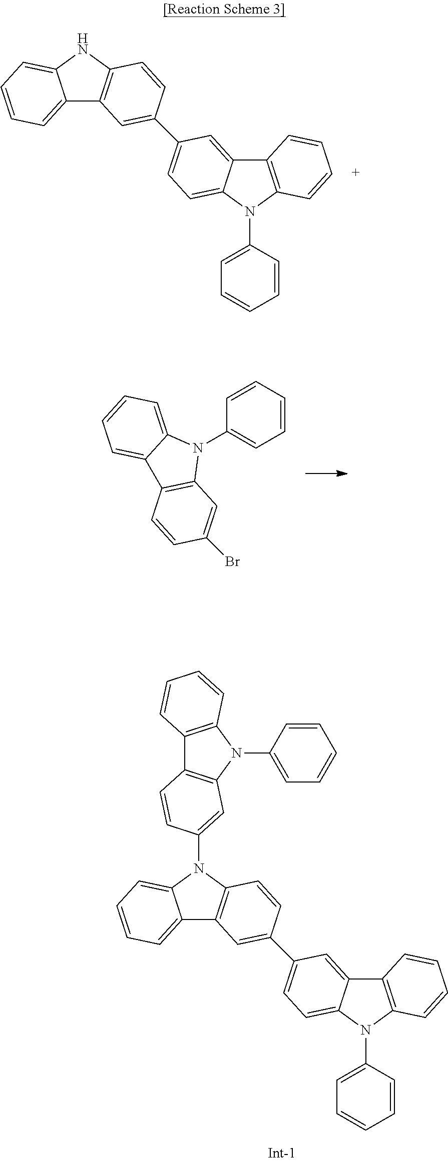 [SMILES: c1ccc(-n2c3ccccc3c3cc(-c4ccc5[nH]c6ccccc6c5c4)ccc32)cc1 ; Brc1ccc2c3ccccc3n(-c3ccccc3)c2c1 ; c1ccc(-n2c3ccccc3c3cc(-c4ccc5c(c4)c4ccccc4n5-c4ccc5c6ccccc6n(-c6ccccc6)c5c4)ccc32)cc1]

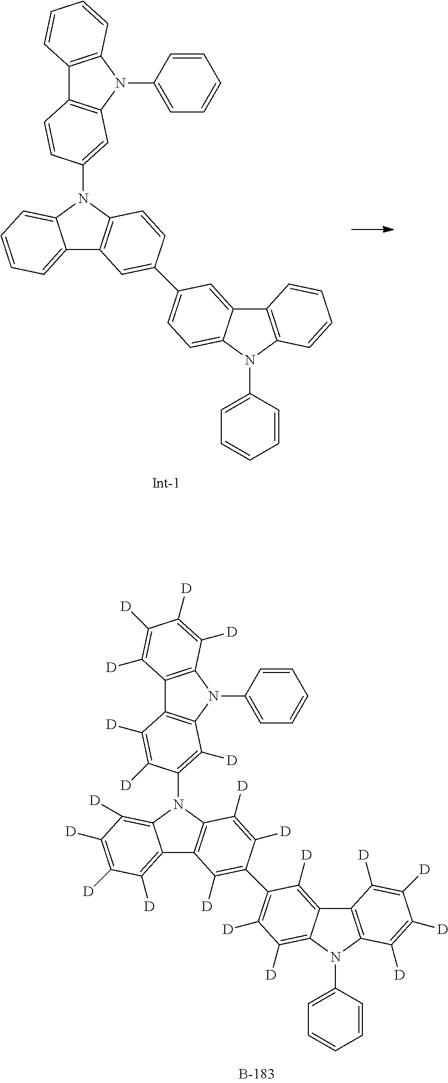 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1]

1st step: Synthesis of Intermediate Int-1

9-phenyl-3,3′-bi-9H-carbazole (20 g, 49.0 mmol), 2-bromo-9-phenylcarbazole (15.8 g, 49.0 mmol), NaOtBu (7.1 g, 73.5 mmol), Pd₂(dba)₃ (2.2 g, 2.5 mmol), and P(t-Bu)₃ (1.5 g, 7.4 mmol) were put in a round-bottomed flask under a nitrogen atmosphere and dissolved in 245 ml of xylene and then, stirred under reflux at 120° C. for 12 hours. When a reaction was completed, an excess of distilled water was poured thereinto and then, stirred for 1 hour. Subsequently, a solid was filtered therefrom and dissolved in toluene at a high temperature. The solution was treated with MgSO₄ to remove moisture, and after filtering an organic solvent with a silica gel pad, a filtrate therefrom was stirred. A solid, which was formed there, was filtered and vacuum-dried, obtaining 21.6 g (68%) of Intermediate Int-1.

2nd step: Synthesis of Compound B-183

Intermediate Int-1 (21.6 g, 33.27 mmol), trifilc acid (24.96 g, 166.35 mmol), and benzene-D₆ (174.97 g, 2079.32 mmol) were put in a round-bottomed flask under a nitrogen condition and stirred under reflux at 50° C. for 20 hours. When a reaction was completed, D₂₀ (124.82 ml) was slowly poured thereinto for quenching and then, sufficiently stirred. The resultant was titrated and neutralized with a K₃PO₄(aq) saturated solution. When a reaction was completed, after removing an aqueous layer with a separatory funnel, a solid was obtained therefrom by removing an organic solvent under a reduced pressure.

The obtained solid was dissolved in toluene at a high temperature. After removing moisture with MgSO₄ and filtering an organic solvent therefrom, a filtrate therefrom was stirred. A solid, which was formed there, was filtered and vacuum-dried, obtaining 16.73 g (75%) of Compound B-183.

Synthesis of Third Compound

### Synthesis Example 4: Synthesis of Compound D-9

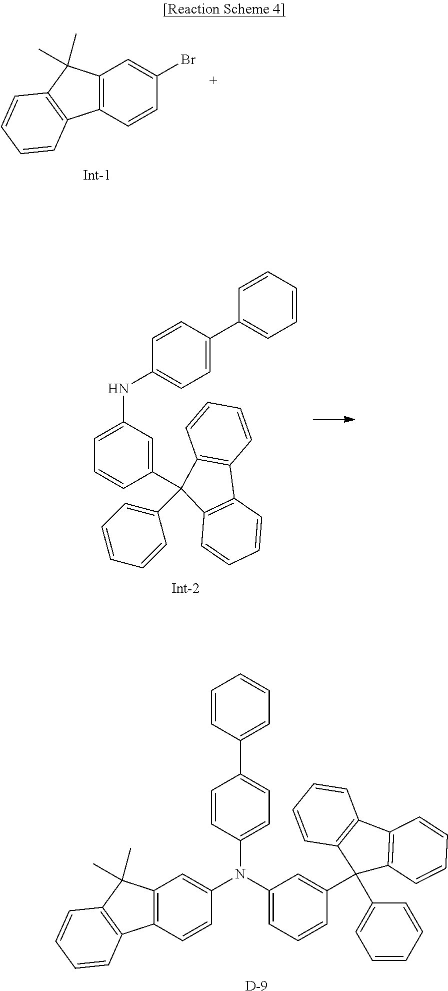

7.12 g (26.1 mmol) of Int-1, 11 g (22.7 mmol) of Int-2 (CAS No. 2305719-96-2), and 3.48 g (36.2 mmol) of sodium t-butoxide were put in a round-bottomed flask and dissolved in 230 ml of toluene. Subsequently, 1.04 g (1.13 mmol) of Pd₂(dba)₃ and 0.92 g (2.27 mmol) of tri-tertiary-butylphosphine were sequentially added thereto and then, stirred under reflux for 6 hours under a nitrogen atmosphere. When a reaction was completed, after removing the toluene solvent, an organic layer was extracted with dichloromethane and distilled water, dried with magnesium sulfate, and filtered, and a filtrate therefrom was concentrated under a reduced pressure. A product therefrom was recrystallized and purified with n-hexane/dichloromethane, obtaining 13.4 g (Yield: 87%) of Compound D-9.

(Manufacture of Organic Light Emitting Diode)

### Example 1

A glass substrate coated with ITO/Ag/ITO as a thin film was washed with a distilled water ultrasonic wave. When the washing with distilled water was completed, the substrate was ultrasonically washed with acetone or isopropyl alcohol, dried, transferred to a plasma cleaner, cleaned with by using oxygen plasma for 10 minutes, and transferred to a vacuum evaporator. This prepared ITO/Ag/ITO (reflecting electrode) was used as an anode, and Compound A doped with 3% NDP-9 (commercially available from Novaled) was vacuum-deposited thereon to form a 100 Å-thick hole injection layer, and a 1,350 Å-thick hole transport layer was formed thereon by depositing Compound A. On the hole transport layer, Compound D-9 according to Synthesis Example 4 was deposited to form a 335 Å-thick hole transport auxiliary layer, on the hole transport auxiliary layer, both Compound A-52 of Synthesis Example 1 and Compound B-183 of Synthesis Example 3 as a host, which were doped with 10 wt % of GD as a dopant, were vacuum-deposited to form a 330 Å-thick light emitting layer. Subsequently, on the light emitting layer, Compound B was deposited to form a 50 Å-thick electron transport auxiliary layer, and a 310 Å-thick electron transport layer was formed thereon by vacuum-depositing Compound C and Liq in a weight ratio of 1:1, simultaneously. On the electron transport layer (ETL), Yb and AgMg were sequentially vacuum-deposited to form a cathode, manufacturing an organic light emitting diode.

ITO/Ag/ITO/Compound A (3% NDP-9 doping, 100 Å)/Compound A (1,350 Å)/hole transport auxiliary layer (Compound D-9, 335 Å)/light emitting layer [host (Compound A-52: Compound B-183=40:60):GD=90 wt %: 10 wt %] (330 Å)/Compound B (50 Å)/Compound C: Liq (310 Å)/Yb/AgMg.

Compound A: N-(9,9-diphenyl-9H-fluoren-2-yl)-N,9-diphenyl-9H-carbazol-2-amine

Compound B: 4-{4-[4-(9,9-dimethyl-9H-fluoren-4-yl)phenyl]phenyl}-2-phenyl-6-(4-phenylphenyl)pyrimidine

Compound C: 2-(4-{1-[4-(diphenyl-1,3,5-triazin-2-yl)phenyl]naphthalene-2-yl}-4,6-diphenyl-1,3,5-triazine

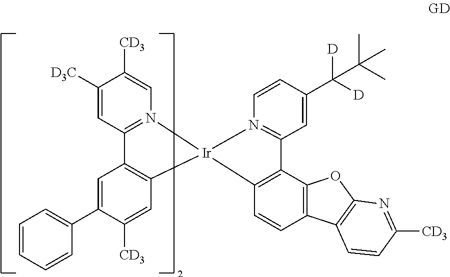

### Example 2 and Comparative Example 1

Organic light emitting diodes were manufactured in the same method as in Example 1, except for changing the compositions shown in Table 1.

Evaluation

Luminous efficiency and life-span characteristics of the organic light emitting diodes according to Example 1 to 2 and Comparative Example 1 were evaluated.

Specific measurement methods are as follows, and the results are shown in Table 1.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

Current efficiency (cd/A) at the same current density (10 mA/cm 2) were calculated by using the luminance and current density from the items (1) and (2), and a voltage.

(4) Measurement of Life-span

The results were obtained by measuring a time when current efficiency (cd/A) was decreased down to 97%, while luminance (cd/m²) was maintained to be 45,000 cd/m².

Life-span measurements of Examples 1 to 2 and Comparative Example 1 were calculated as relative values based on that of Comparative Example 1, and the results are shown in Table 1.

TABLE 1

| | Host | | Hole transport | |
|---|---|---|---|---|
| | First | Second | auxiliary layer | |
| | compound | compound | Third | Life-span |
| Nos. | (wt %) | (wt %) | compound | (%) |
| Example 1 | A-52 | B-183 | D-9 | 188 |
| Example 2 | A-79 | B-183 | D-9 | 170 |
| Comparative | ET-1 | B-183 | D-9 | 100 |
| Example 1 | | | | |

Referring to Table 1, the organic light emitting diodes including the compositions according to the Examples exhibited significantly improved life-span characteristics compared to the organic light emitting diode according to the Comparative Example.

One or more embodiments may provide an organic optoelectronic device capable of implementing high efficiency and long life-span characteristics.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. An organic optoelectronic device, comprising:
an anode and a cathode facing each other,
a light emitting layer between the anode and the cathode,
a hole transport layer between the anode and the light emitting layer, and
a hole transport auxiliary layer between the light emitting layer and the hole transport layer,
wherein:
the light emitting layer includes:
a first compound represented by Chemical Formula 1, and
a second compound represented by Chemical Formula 2 or a combination of Chemical Formula 3 and Chemical Formula 4,
the hole transport auxiliary layer includes a third compound represented by Chemical Formula 5: 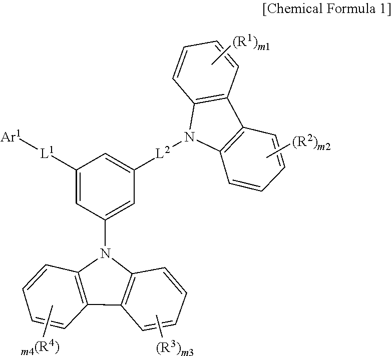
in Chemical Formula 1,
Ar¹ is a substituted or unsubstituted C6 to C30 aryl group, a substituted or unsubstituted dibenzofuranyl group, or a substituted or unsubstituted dibenzothiophenyl group,
L¹ is a single bond or a substituted or unsubstituted C6 to C20 arylene group,
L² is a substituted or unsubstituted C6 to C20 arylene group,
R¹ to R⁴ are each independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
m1 to m4 are each independently an integer of 1 to 4; 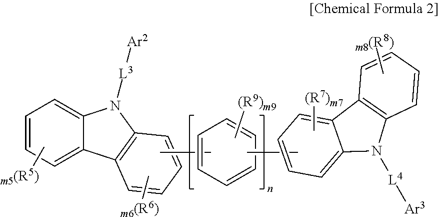
in Chemical Formula 2,
Ar² and Ar³ are each independently a substituted or unsubstituted C6 to C20 aryl group or a substituted or unsubstituted C2 to C30 heterocyclic group,
L³ and L⁴ are each independently a single bond or a substituted or unsubstituted C6 to C20 arylene group,
R⁵ to R⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m5, m8, and m9 are each independently an integer of 1 to 4,
m6 and m7 are each independently an integer of 1 to 3, and
n is an integer of 0 to 2; 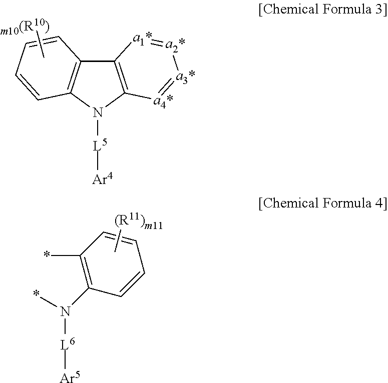
in Chemical Formula 3 and Chemical Formula 4,
Ar⁴ and Ar⁵ are each independently a substituted or unsubstituted C6 to C20 aryl group or a substituted or unsubstituted C2 to C30 heterocyclic group,
two adjacent ones of a₁* to a₄* of Chemical Formula 3 are linking carbons linked at * of Chemical Formula 4, the remaining two of a1* to a4* of Chemical Formula 3, not linked at * of Chemical Formula 4, are C-L^{a}-R^{a},
L^{a}, L⁵, and L⁶ are each independently a single bond or a substituted or unsubstituted C6 to C20 arylene group,
R^{a}, R¹⁰, and R¹¹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and
m10 and m11 are each independently an integer of 1 to 4; 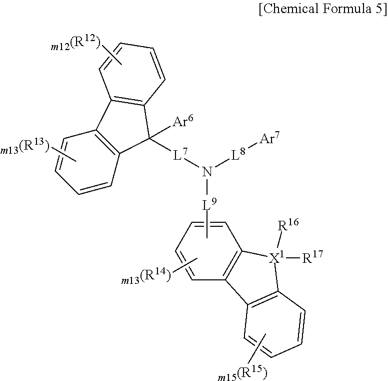
in Chemical Formula 5,
X¹ is C or Si,
R¹² to R¹⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
R¹⁶ and R¹⁷ are each independently a substituted or unsubstituted C1 to C30 alkyl group or a substituted or unsubstituted C6 to C30 aryl group,
Ar⁶ is a substituted or unsubstituted C6 to C30 aryl group,
Ar⁷ is a substituted or unsubstituted C6 to C30 aryl group or a substituted or unsubstituted dibenzosilolyl group,
L⁷ to L⁹ are each independently a single bond or a substituted or unsubstituted C6 to C20 arylene group,
m12, m13, and m15 are each independently an integer of 1 to 4, and
m14 is an integer of 1 to 3.

2. The organic optoelectronic device as claimed in claim 1, wherein:
L² in Chemical Formula 1 is a linking group of Group I: 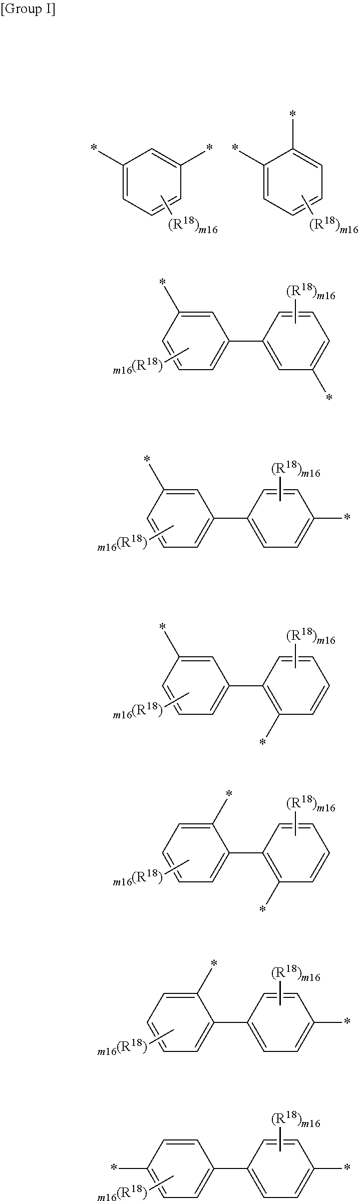 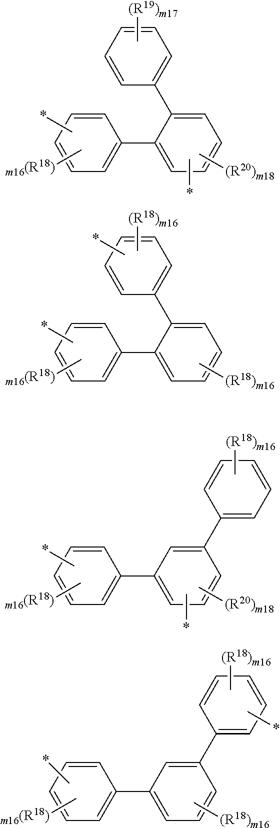
in Group I,
R¹⁸ to R²⁰ are each independently hydrogen, deuterium, a halogen, a substituted or unsubstituted C1 to C20 alkyl group, a substituted or unsubstituted C3 to C20 cycloalkyl group, a substituted or unsubstituted C1 to C20 alkoxy group, a substituted or unsubstituted C3 to C20 cycloalkoxy group, a substituted or unsubstituted C1 to C20 alkylthio group, a substituted or unsubstituted C6 to C30 aralkyl group, a substituted or unsubstituted C6 to C30 aryl group, a substituted or unsubstituted C6 to C30 aryloxy group, a substituted or unsubstituted C6 to C30 arylthio group, a substituted or unsubstituted C2 to C30 heteroaryl group, a substituted or unsubstituted C2 to C30 amino group, a substituted or unsubstituted C3 to C30 silyl group, a cyano group, a nitro group, a hydroxyl group, a carboxyl group, or a combination thereof,
m16 is an integer of 1 to 4,
m17 is an integer of 1 to 5,
m18 is an integer of 1 to 3, and
* is a linking point.

3. The organic optoelectronic device as claimed in claim 1, wherein Ar¹ in Chemical Formula 1 is a substituted or unsubstituted phenyl group, a substituted or unsubstituted biphenyl group, a substituted or unsubstituted terphenyl group, a substituted or unsubstituted quaterphenyl group, a substituted or unsubstituted fluorenyl group, a substituted or unsubstituted dibenzofuranyl group, or a substituted or unsubstituted dibenzothiophenyl group.

4. The organic optoelectronic device as claimed in claim 1, wherein:
the second compound is represented by Chemical Formula 2-8 or Chemical Formula 3C: 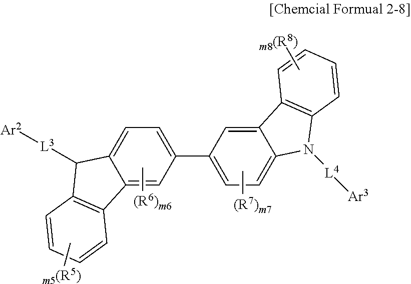
in Chemical Formula 2-8,
R⁵ to R⁸ are each independently hydrogen, deuterium, or a substituted or unsubstituted C6 to C12 aryl group,
m5 and m8 are each independently one of integers of 1 to 4,
m6 and m7 are each independently one of integers of 1 to 3, and
moieties *-L³-Ar² and *-L⁴-Ar³ are each independently a moiety of Group II, 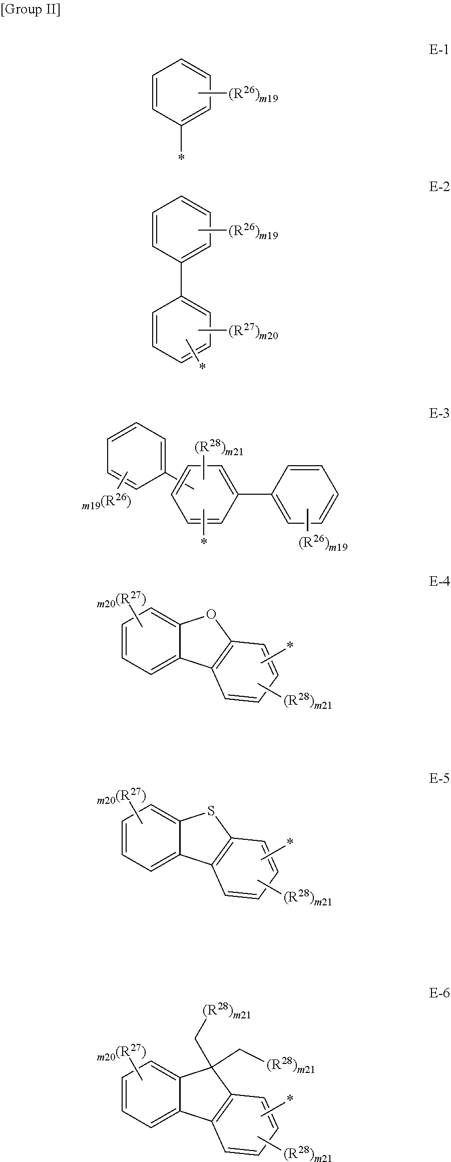 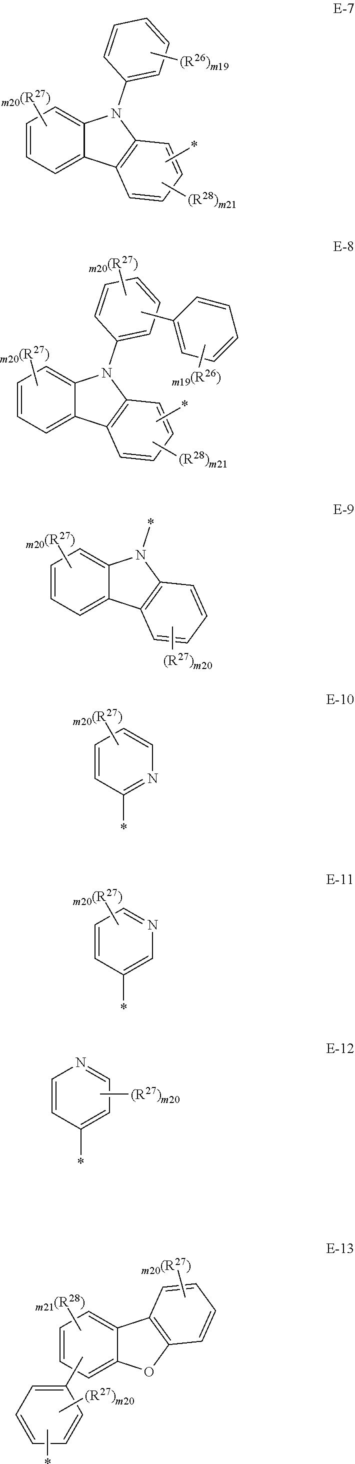 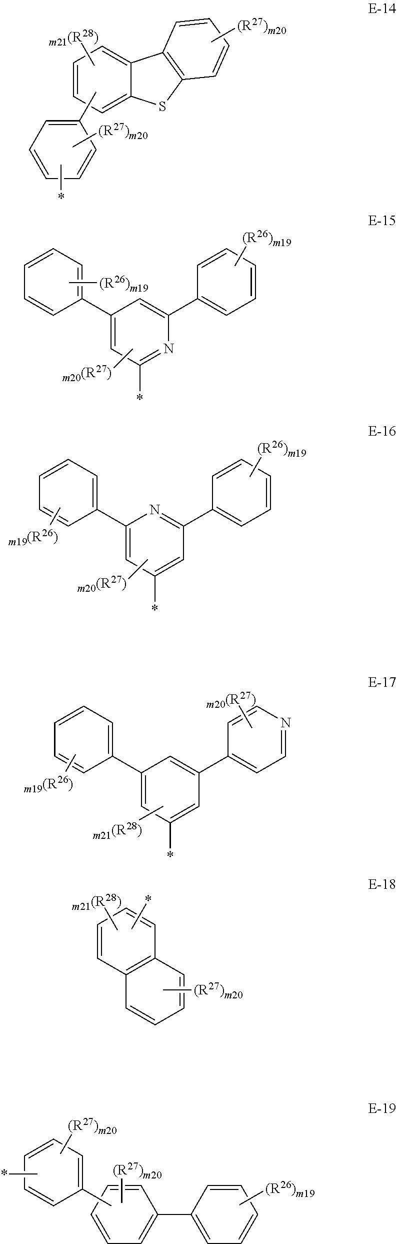 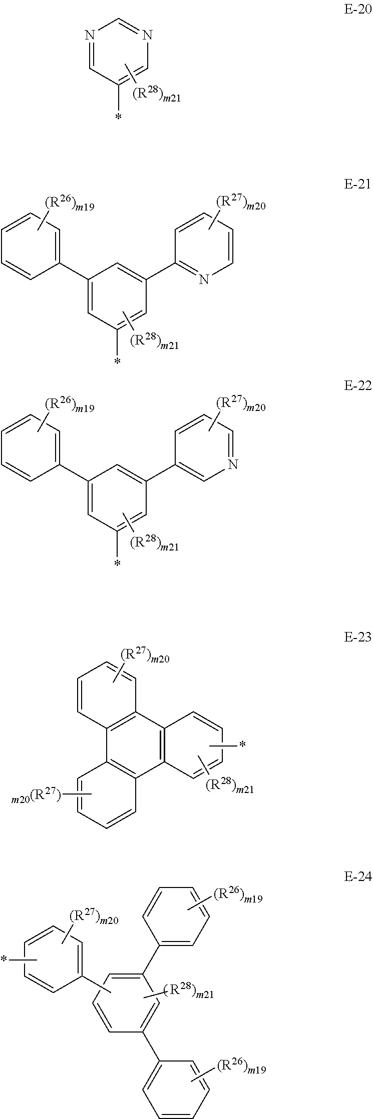 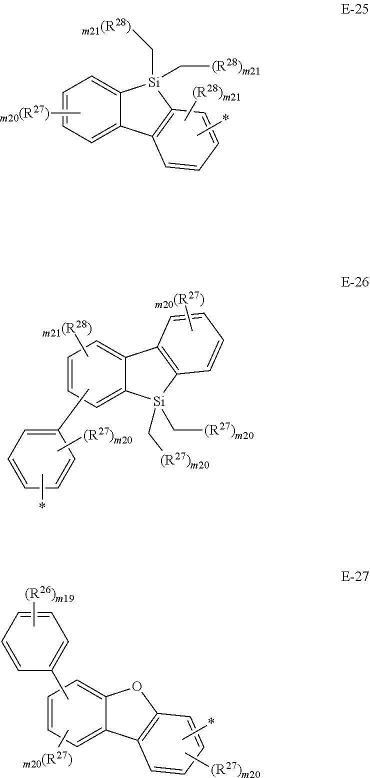 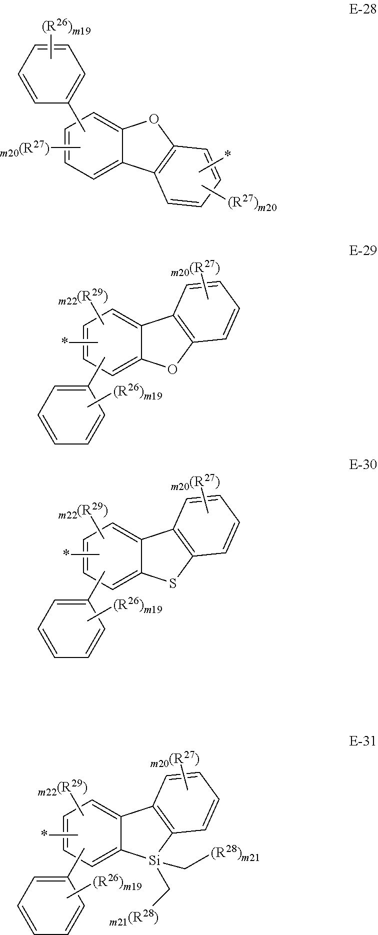
in Group II,
R²⁶ to R²⁹ are each independently hydrogen, deuterium, a cyano group, a substituted or unsubstituted C1 to C10 alkyl group, or a substituted or unsubstituted C6 to C12 aryl group,
m19 is an integer of 1 to 5,
m20 is an integer of 1 to 4,
m21 is an integer of 1 to 3,
m22 is 1 or 2, and
* is a linking point; 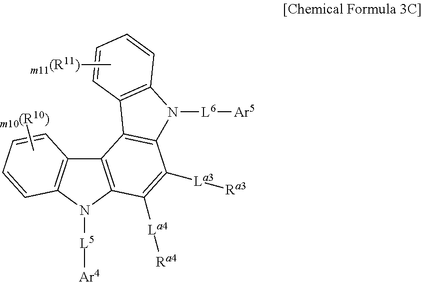
in Chemical Formula 3C,
L^{a3} and L^{a4} are a single bond,
L⁵ and L⁶ are each independently a single bond or a substituted or unsubstituted C6 to C12 arylene group,
R¹⁰, R¹¹, R^{a3}, and R^{a4} are each independently hydrogen, deuterium, or a substituted or unsubstituted C6 to C12 aryl group,
Ar⁴ and Ar⁵ are each independently a substituted or unsubstituted phenyl group, a substituted or unsubstituted naphthyl group, or a substituted or unsubstituted biphenyl group, and
m10 and m11 are each independently an integer of 1 to 4.

5. The organic optoelectronic device as claimed in claim 1, wherein:
the third compound is represented by one of Chemical Formula 5-1 to Chemical Formula 5-4: 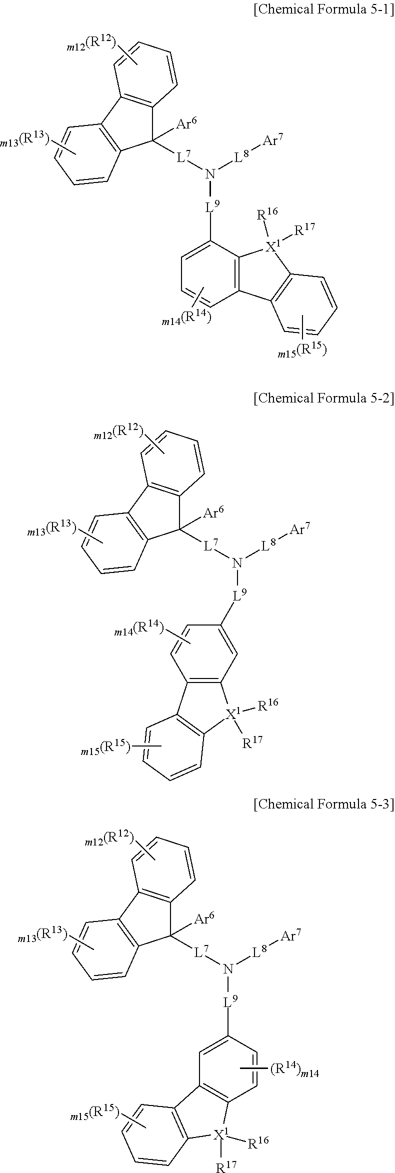 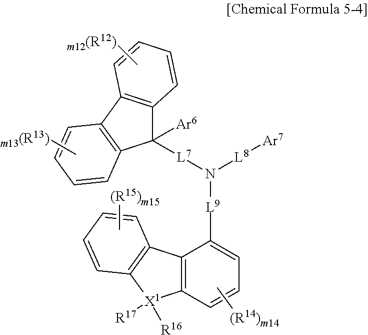
in Chemical Formula 5-1 to Chemical Formula 5-4, X¹, R¹² to R¹⁷, Ar⁶, Ar⁷, L⁷ to L⁹, and m12 to m15 are defined the same as those of Chemical Formula 5.

6. The organic optoelectronic device as claimed in claim 1, wherein Ar⁶ in Chemical Formula 5 is a substituted or unsubstituted phenyl group, a substituted or unsubstituted biphenyl group, or a substituted or unsubstituted terphenyl group.

7. The organic optoelectronic device as claimed in claim 1, wherein Ar⁷ in Chemical Formula 5 is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, or a substituted or unsubstituted dibenzosilolyl group.

8. The organic optoelectronic device as claimed in claim 1, wherein:
Ar⁷ in Chemical Formula 5 is a group of Group III: 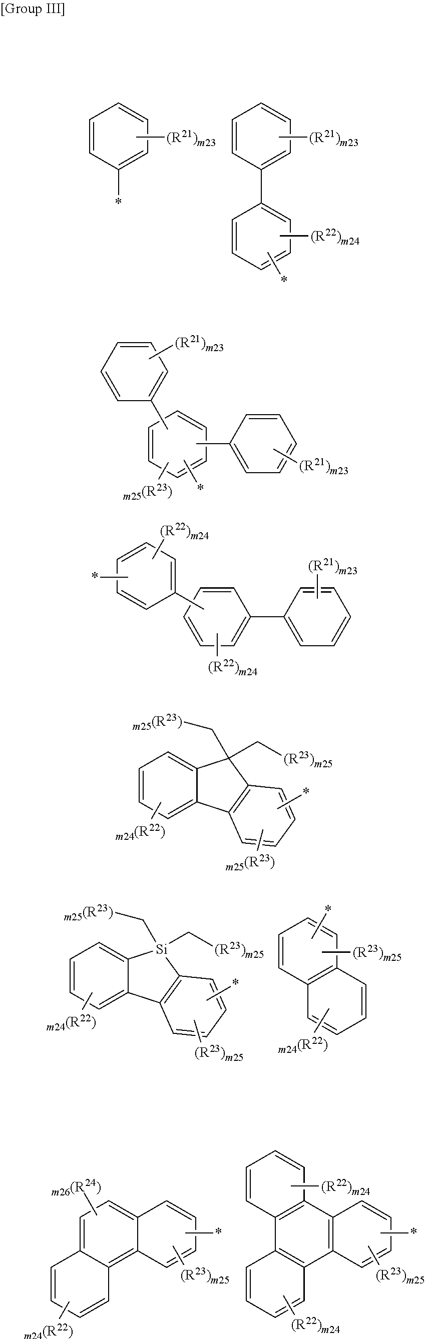 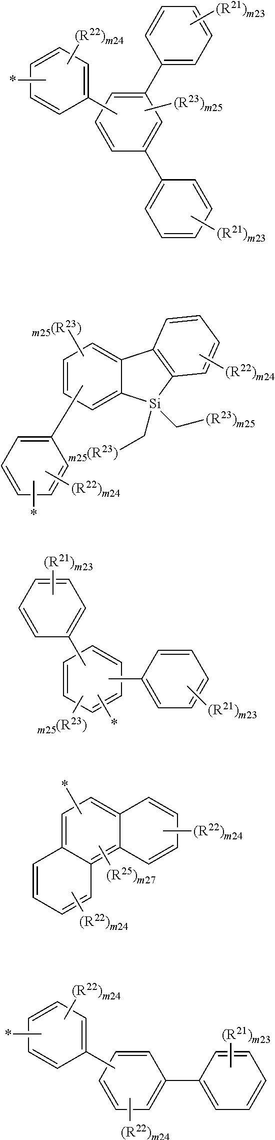
in Group III,
R²¹ to R²⁵ are each independently hydrogen, deuterium, a cyano group, a substituted or unsubstituted C1 to C10 alkyl group, or a substituted or unsubstituted C6 to C12 aryl group,
m23 is an integer of 1 to 5,
m24 is an integer of 1 to 4,
m25 is an integer of 1 to 3,
m26 is 1 or 2,
m27 is 1, and
* is a linking point.

9. The organic optoelectronic device as claimed in claim 1, wherein the third compound is a compound of Group 3:

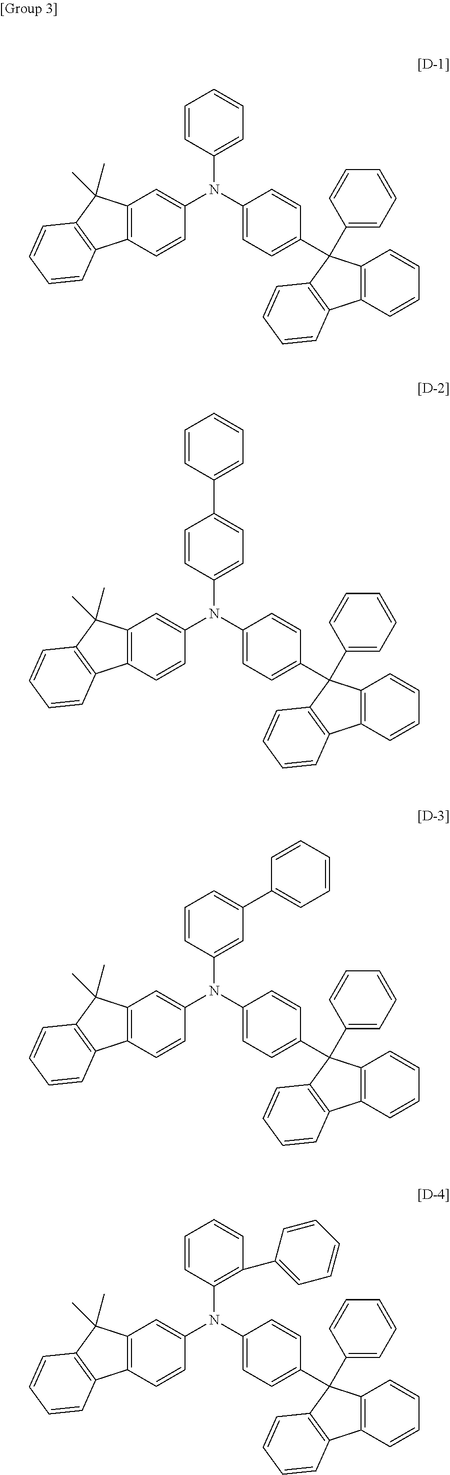 [SMILES: CC1(C)c2ccccc2-c2ccc(N(c3ccccc3)c3ccc(C4(c5ccccc5)c5ccccc5-c5ccccc54)cc3)cc21 ; CC1(C)c2ccccc2-c2ccc(N(c3ccc(-c4ccccc4)cc3)c3ccc(C4(c5ccccc5)c5ccccc5-c5ccccc54)cc3)cc21 ; CC1(C)c2ccccc2-c2ccc(N(c3ccc(C4(c5ccccc5)c5ccccc5-c5ccccc54)cc3)c3cccc(-c4ccccc4)c3)cc21 ; CC1(C)c2ccccc2-c2ccc(N(c3ccc(C4(c5ccccc5)c5ccccc5-c5ccccc54)cc3)c3ccccc3-c3ccccc3)cc21]

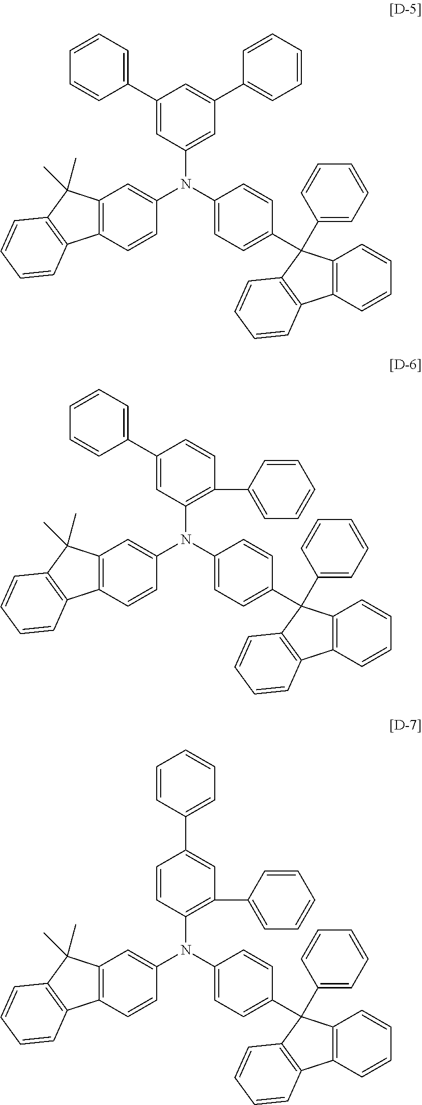 [SMILES: CC1(C)c2ccccc2-c2ccc(N(c3ccc(C4(c5ccccc5)c5ccccc5-c5ccccc54)cc3)c3cc(-c4ccccc4)cc(-c4ccccc4)c3)cc21 ; CC1(C)c2ccccc2-c2ccc(N(c3ccc(C4(c5ccccc5)c5ccccc5-c5ccccc54)cc3)c3cc(-c4ccccc4)ccc3-c3ccccc3)cc21 ; CC1(C)c2ccccc2-c2ccc(N(c3ccc(C4(c5ccccc5)c5ccccc5-c5ccccc54)cc3)c3ccc(-c4ccccc4)cc3-c3ccccc3)cc21]

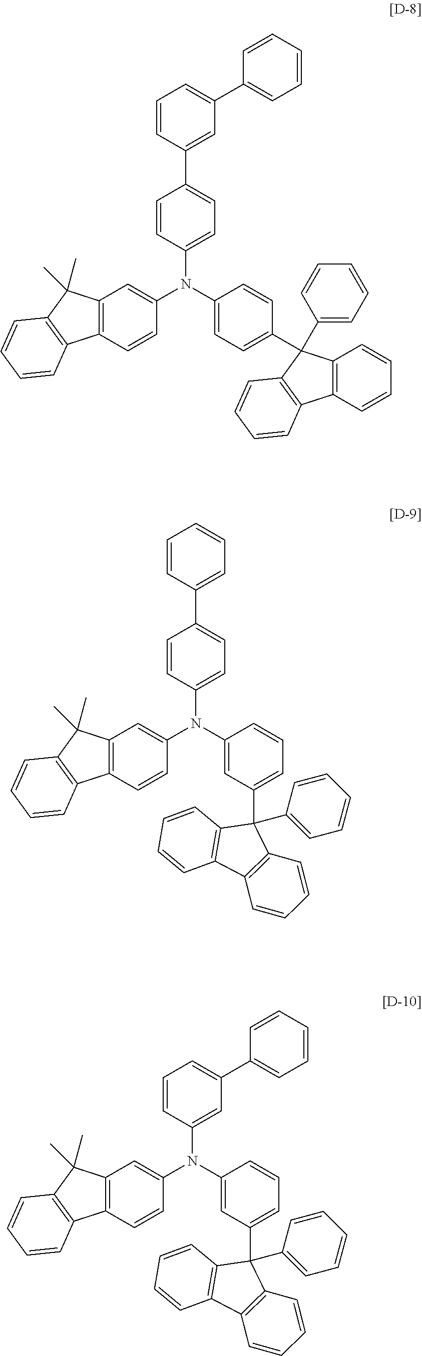 [SMILES: CC1(C)c2ccccc2-c2ccc(N(c3ccc(-c4cccc(-c5ccccc5)c4)cc3)c3ccc(C4(c5ccccc5)c5ccccc5-c5ccccc54)cc3)cc21 ; CC1(C)c2ccccc2-c2ccc(N(c3ccc(-c4ccccc4)cc3)c3cccc(C4(c5ccccc5)c5ccccc5-c5ccccc54)c3)cc21 ; CC1(C)c2ccccc2-c2ccc(N(c3cccc(-c4ccccc4)c3)c3cccc(C4(c5ccccc5)c5ccccc5-c5ccccc54)c3)cc21]

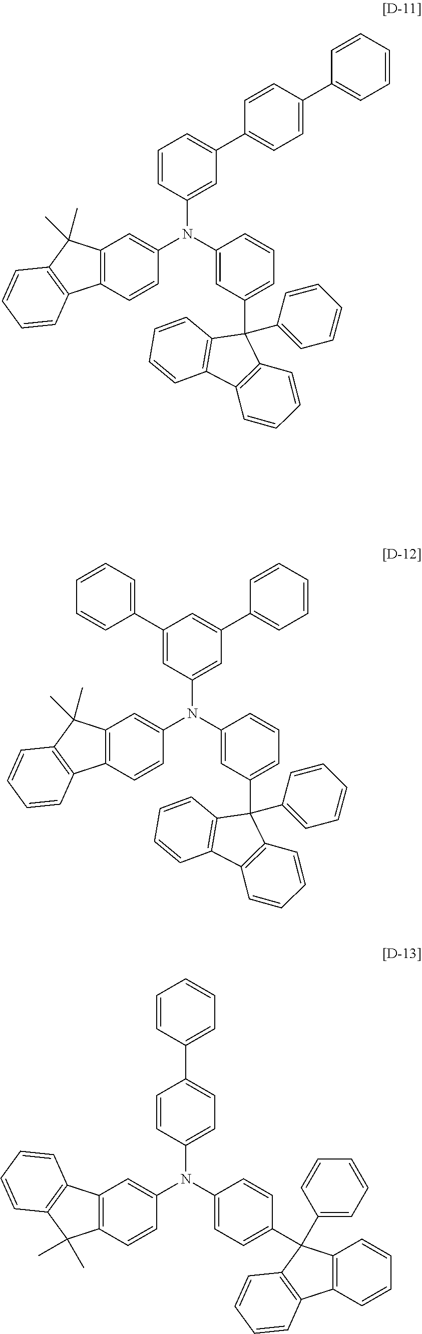 [SMILES: CC1(C)c2ccccc2-c2ccc(N(c3cccc(-c4ccc(-c5ccccc5)cc4)c3)c3cccc(C4(c5ccccc5)c5ccccc5-c5ccccc54)c3)cc21 ; CC1(C)c2ccccc2-c2ccc(N(c3cc(-c4ccccc4)cc(-c4ccccc4)c3)c3cccc(C4(c5ccccc5)c5ccccc5-c5ccccc54)c3)cc21 ; CC1(C)c2ccccc2-c2cc(N(c3ccc(-c4ccccc4)cc3)c3ccc(C4(c5ccccc5)c5ccccc5-c5ccccc54)cc3)ccc21]

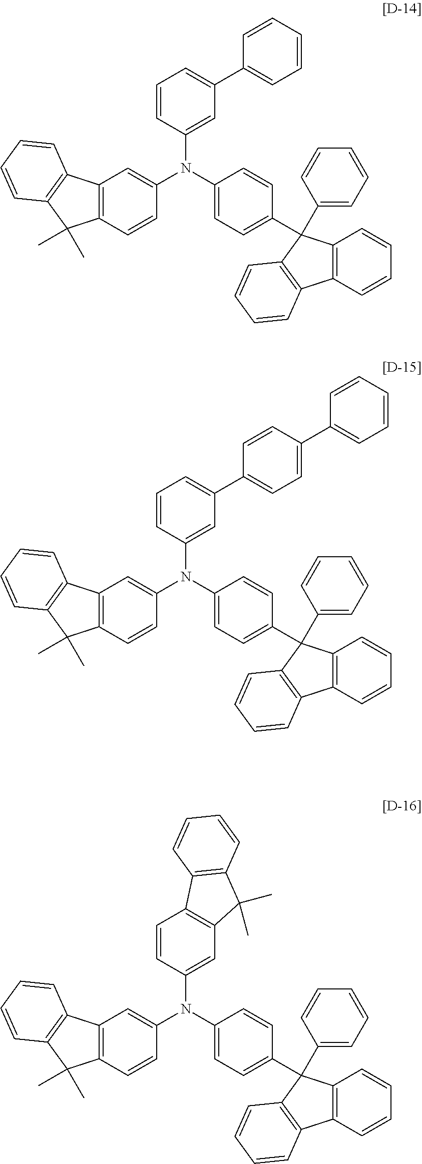 [SMILES: CC1(C)c2ccccc2-c2cc(N(c3ccc(C4(c5ccccc5)c5ccccc5-c5ccccc54)cc3)c3cccc(-c4ccccc4)c3)ccc21 ; CC1(C)c2ccccc2-c2cc(N(c3ccc(C4(c5ccccc5)c5ccccc5-c5ccccc54)cc3)c3cccc(-c4ccc(-c5ccccc5)cc4)c3)ccc21 ; CC1(C)c2ccccc2-c2cc(N(c3ccc(C4(c5ccccc5)c5ccccc5-c5ccccc54)cc3)c3ccc4c(c3)C(C)(C)c3ccccc3-4)ccc21]

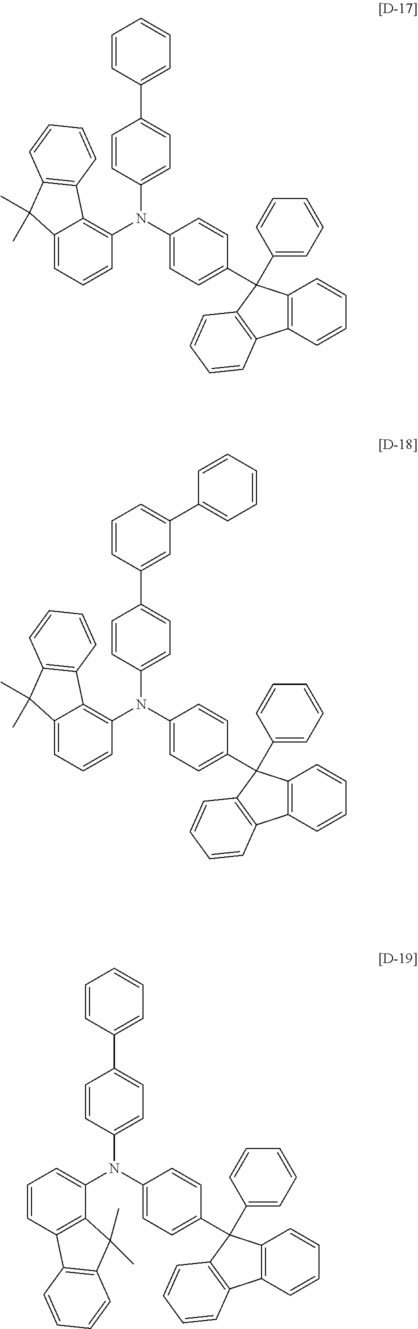 [SMILES: CC1(C)c2ccccc2-c2c(N(c3ccc(-c4ccccc4)cc3)c3ccc(C4(c5ccccc5)c5ccccc5-c5ccccc54)cc3)cccc21 ; CC1(C)c2ccccc2-c2c(N(c3ccc(-c4cccc(-c5ccccc5)c4)cc3)c3ccc(C4(c5ccccc5)c5ccccc5-c5ccccc54)cc3)cccc21 ; CC1(C)c2ccccc2-c2cccc(N(c3ccc(-c4ccccc4)cc3)c3ccc(C4(c5ccccc5)c5ccccc5-c5ccccc54)cc3)c21]

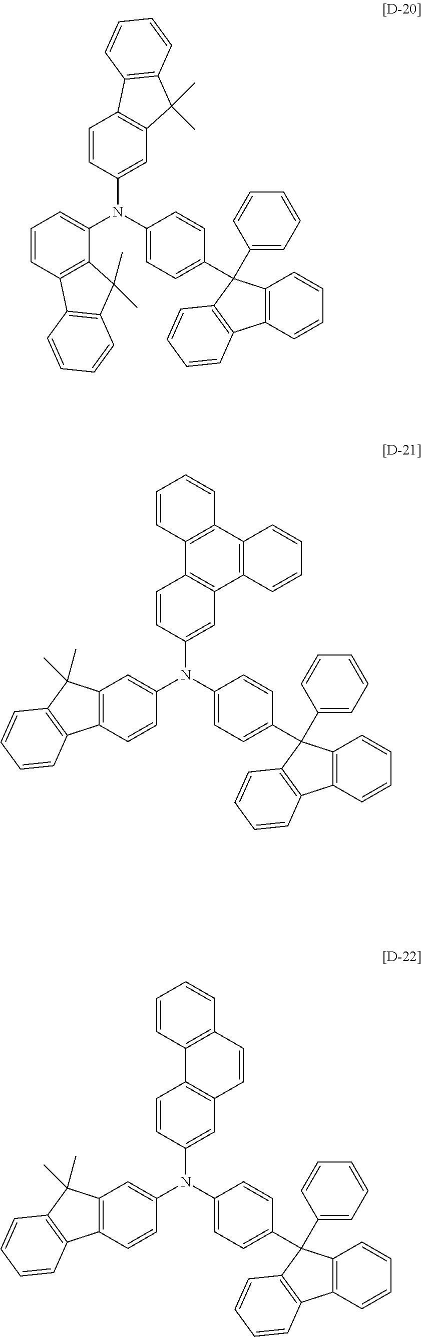 [SMILES: CC1(C)c2ccccc2-c2ccc(N(c3ccc(C4(c5ccccc5)c5ccccc5-c5ccccc54)cc3)c3cccc4c3C(C)(C)c3ccccc3-4)cc21 ; CC1(C)c2ccccc2-c2ccc(N(c3ccc(C4(c5ccccc5)c5ccccc5-c5ccccc54)cc3)c3ccc4c5ccccc5c5ccccc5c4c3)cc21 ; CC1(C)c2ccccc2-c2ccc(N(c3ccc(C4(c5ccccc5)c5ccccc5-c5ccccc54)cc3)c3ccc4c(ccc5ccccc54)c3)cc21]

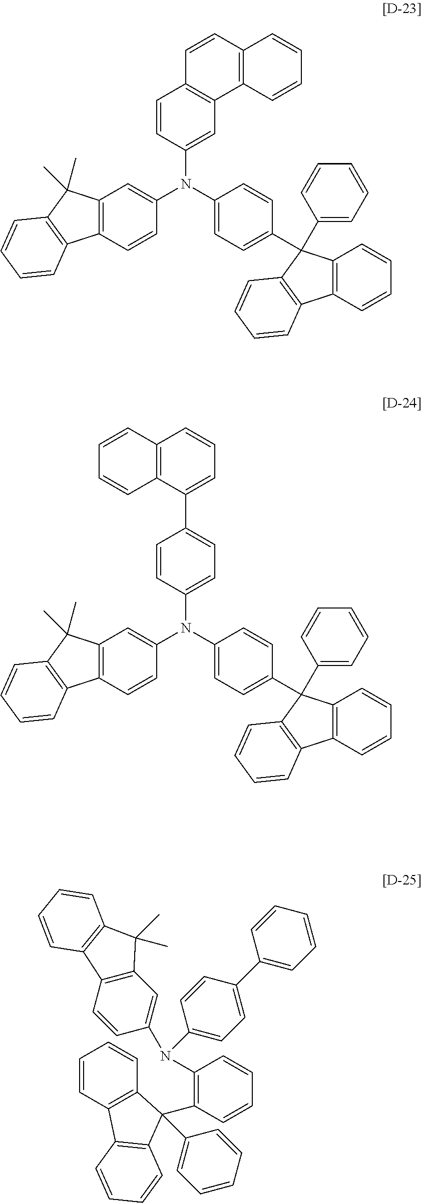 [SMILES: CC1(C)c2ccccc2-c2ccc(N(c3ccc(C4(c5ccccc5)c5ccccc5-c5ccccc54)cc3)c3ccc4ccc5ccccc5c4c3)cc21 ; CC1(C)c2ccccc2-c2ccc(N(c3ccc(-c4cccc5ccccc45)cc3)c3ccc(C4(c5ccccc5)c5ccccc5-c5ccccc54)cc3)cc21 ; CC1(C)c2ccccc2-c2ccc(N(c3ccc(-c4ccccc4)cc3)c3ccccc3C3(c4ccccc4)c4ccccc4-c4ccccc43)cc21]

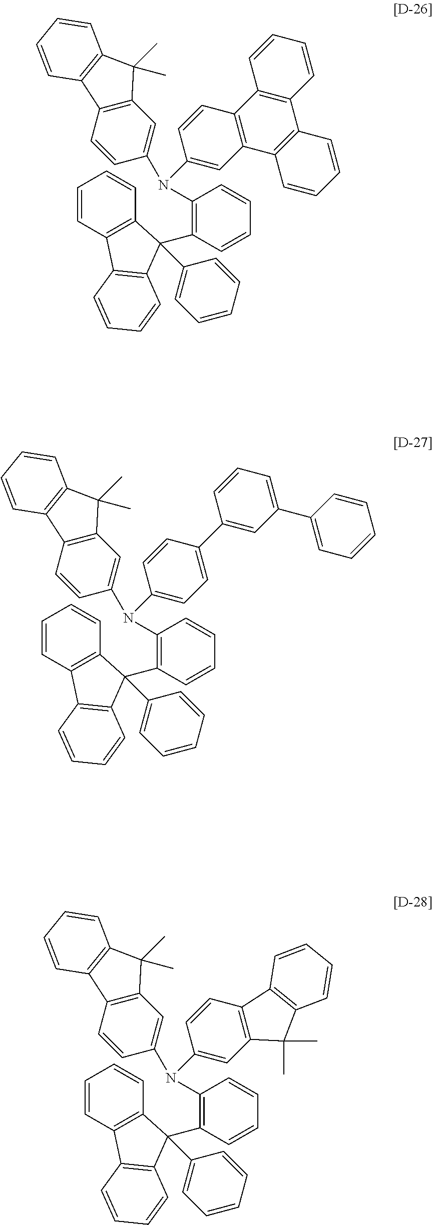 [SMILES: CC1(C)c2ccccc2-c2ccc(N(c3ccc4c5ccccc5c5ccccc5c4c3)c3ccccc3C3(c4ccccc4)c4ccccc4-c4ccccc43)cc21 ; CC1(C)c2ccccc2-c2ccc(N(c3ccc(-c4cccc(-c5ccccc5)c4)cc3)c3ccccc3C3(c4ccccc4)c4ccccc4-c4ccccc43)cc21 ; CC1(C)c2ccccc2-c2ccc(N(c3ccc4c(c3)C(C)(C)c3ccccc3-4)c3ccccc3C3(c4ccccc4)c4ccccc4-c4ccccc43)cc21]

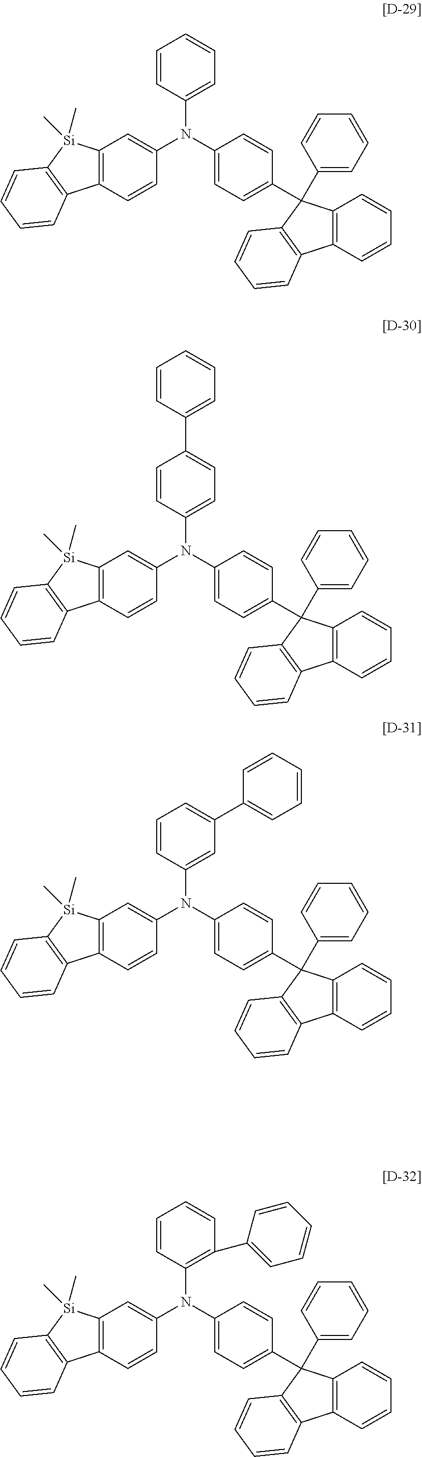 [SMILES: C[Si]1(C)c2ccccc2-c2ccc(N(c3ccccc3)c3ccc(C4(c5ccccc5)c5ccccc5-c5ccccc54)cc3)cc21 ; C[Si]1(C)c2ccccc2-c2ccc(N(c3ccc(-c4ccccc4)cc3)c3ccc(C4(c5ccccc5)c5ccccc5-c5ccccc54)cc3)cc21 ; C[Si]1(C)c2ccccc2-c2ccc(N(c3ccc(C4(c5ccccc5)c5ccccc5-c5ccccc54)cc3)c3cccc(-c4ccccc4)c3)cc21 ; C[Si]1(C)c2ccccc2-c2ccc(N(c3ccc(C4(c5ccccc5)c5ccccc5-c5ccccc54)cc3)c3ccccc3-c3ccccc3)cc21]

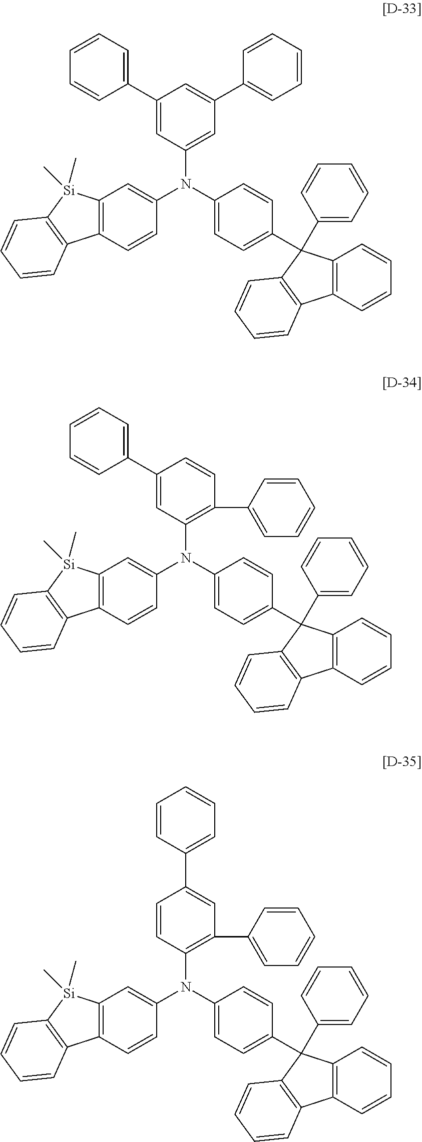 [SMILES: C[Si]1(C)c2ccccc2-c2ccc(N(c3ccc(C4(c5ccccc5)c5ccccc5-c5ccccc54)cc3)c3cc(-c4ccccc4)cc(-c4ccccc4)c3)cc21 ; C[Si]1(C)c2ccccc2-c2ccc(N(c3ccc(C4(c5ccccc5)c5ccccc5-c5ccccc54)cc3)c3cc(-c4ccccc4)ccc3-c3ccccc3)cc21 ; C[Si]1(C)c2ccccc2-c2ccc(N(c3ccc(C4(c5ccccc5)c5ccccc5-c5ccccc54)cc3)c3ccc(-c4ccccc4)cc3-c3ccccc3)cc21]

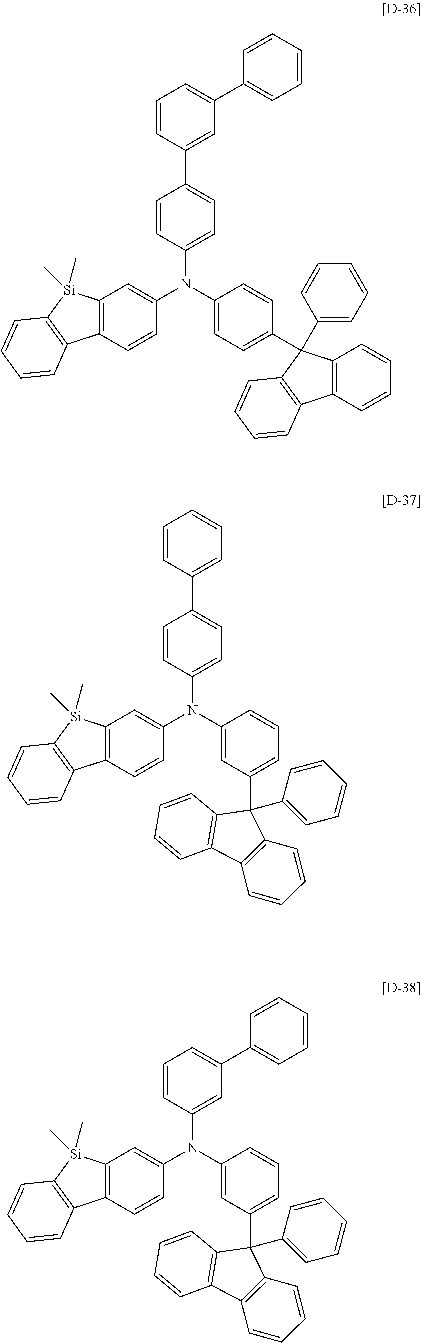 [SMILES: C[Si]1(C)c2ccccc2-c2ccc(N(c3ccc(-c4cccc(-c5ccccc5)c4)cc3)c3ccc(C4(c5ccccc5)c5ccccc5-c5ccccc54)cc3)cc21 ; C[Si]1(C)c2ccccc2-c2ccc(N(c3ccc(-c4ccccc4)cc3)c3cccc(C4(c5ccccc5)c5ccccc5-c5ccccc54)c3)cc21 ; C[Si]1(C)c2ccccc2-c2ccc(N(c3cccc(-c4ccccc4)c3)c3cccc(C4(c5ccccc5)c5ccccc5-c5ccccc54)c3)cc21]

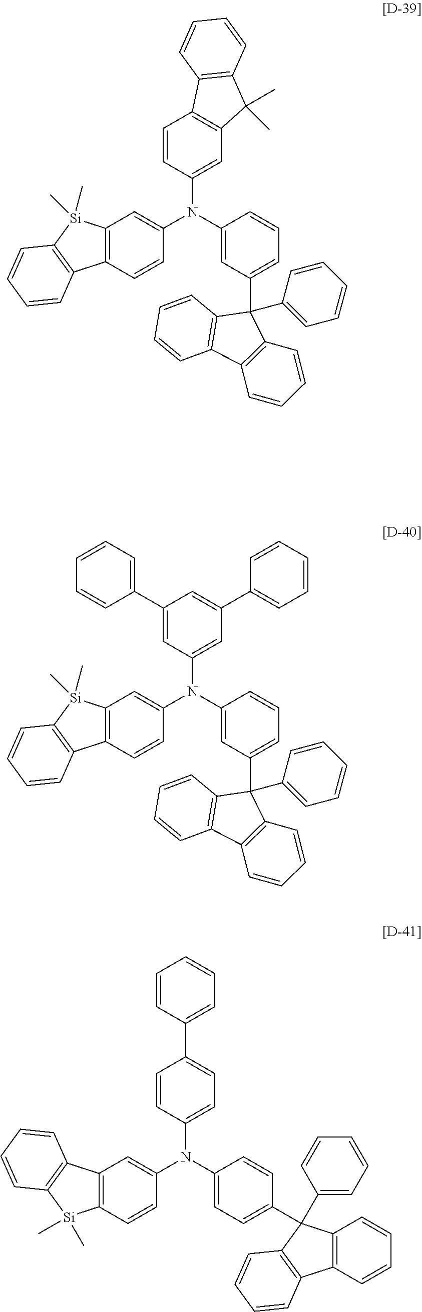 [SMILES: CC1(C)c2ccccc2-c2ccc(N(c3cccc(C4(c5ccccc5)c5ccccc5-c5ccccc54)c3)c3ccc4c(c3)[Si](C)(C)c3ccccc3-4)cc21 ; C[Si]1(C)c2ccccc2-c2ccc(N(c3cc(-c4ccccc4)cc(-c4ccccc4)c3)c3cccc(C4(c5ccccc5)c5ccccc5-c5ccccc54)c3)cc21 ; C[Si]1(C)c2ccccc2-c2cc(N(c3ccc(-c4ccccc4)cc3)c3ccc(C4(c5ccccc5)c5ccccc5-c5ccccc54)cc3)ccc21]

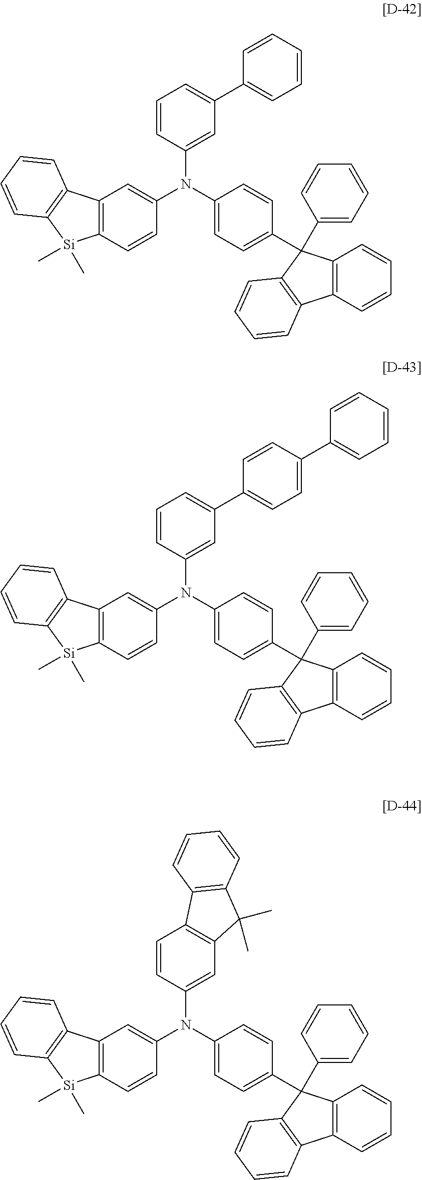 [SMILES: C[Si]1(C)c2ccccc2-c2cc(N(c3ccc(C4(c5ccccc5)c5ccccc5-c5ccccc54)cc3)c3cccc(-c4ccccc4)c3)ccc21 ; C[Si]1(C)c2ccccc2-c2cc(N(c3ccc(C4(c5ccccc5)c5ccccc5-c5ccccc54)cc3)c3cccc(-c4ccc(-c5ccccc5)cc4)c3)ccc21 ; CC1(C)c2ccccc2-c2ccc(N(c3ccc(C4(c5ccccc5)c5ccccc5-c5ccccc54)cc3)c3ccc4c(c3)-c3ccccc3[Si]4(C)C)cc21]

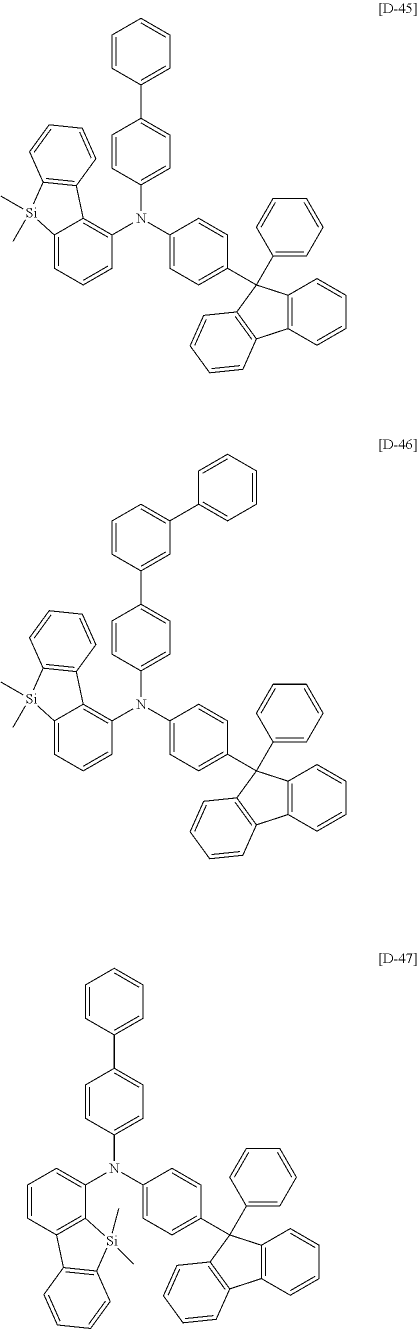 [SMILES: C[Si]1(C)c2ccccc2-c2c(N(c3ccc(-c4ccccc4)cc3)c3ccc(C4(c5ccccc5)c5ccccc5-c5ccccc54)cc3)cccc21 ; C[Si]1(C)c2ccccc2-c2c(N(c3ccc(-c4cccc(-c5ccccc5)c4)cc3)c3ccc(C4(c5ccccc5)c5ccccc5-c5ccccc54)cc3)cccc21 ; C[Si]1(C)c2ccccc2-c2cccc(N(c3ccc(-c4ccccc4)cc3)c3ccc(C4(c5ccccc5)c5ccccc5-c5ccccc54)cc3)c21]

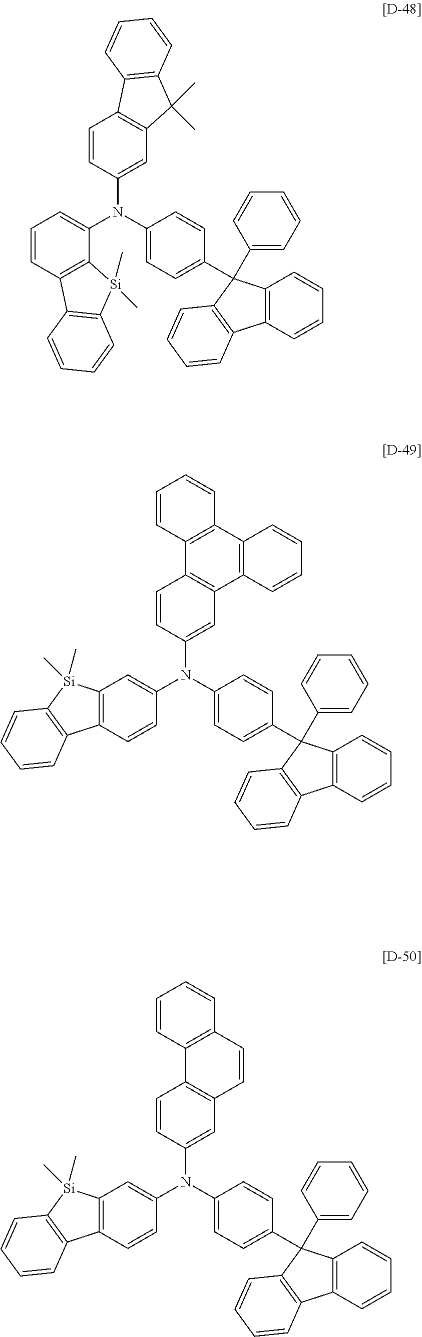 [SMILES: CC1(C)c2ccccc2-c2ccc(N(c3ccc(C4(c5ccccc5)c5ccccc5-c5ccccc54)cc3)c3cccc4c3[Si](C)(C)c3ccccc3-4)cc21 ; C[Si]1(C)c2ccccc2-c2ccc(N(c3ccc(C4(c5ccccc5)c5ccccc5-c5ccccc54)cc3)c3ccc4c5ccccc5c5ccccc5c4c3)cc21 ; C[Si]1(C)c2ccccc2-c2ccc(N(c3ccc(C4(c5ccccc5)c5ccccc5-c5ccccc54)cc3)c3ccc4c(ccc5ccccc54)c3)cc21]

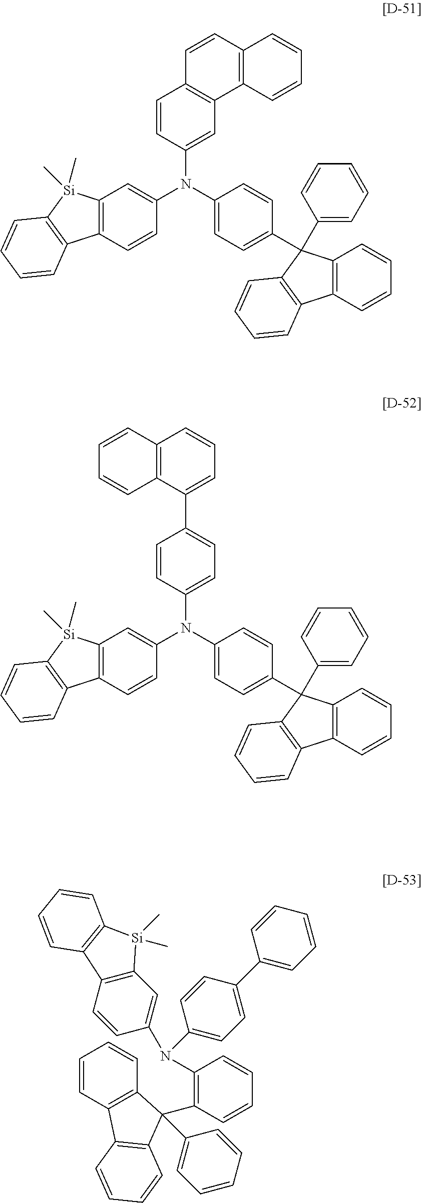 [SMILES: C[Si]1(C)c2ccccc2-c2ccc(N(c3ccc(C4(c5ccccc5)c5ccccc5-c5ccccc54)cc3)c3ccc4ccc5ccccc5c4c3)cc21 ; C[Si]1(C)c2ccccc2-c2ccc(N(c3ccc(-c4cccc5ccccc45)cc3)c3ccc(C4(c5ccccc5)c5ccccc5-c5ccccc54)cc3)cc21 ; C[Si]1(C)c2ccccc2-c2ccc(N(c3ccc(-c4ccccc4)cc3)c3ccccc3C3(c4ccccc4)c4ccccc4-c4ccccc43)cc21]

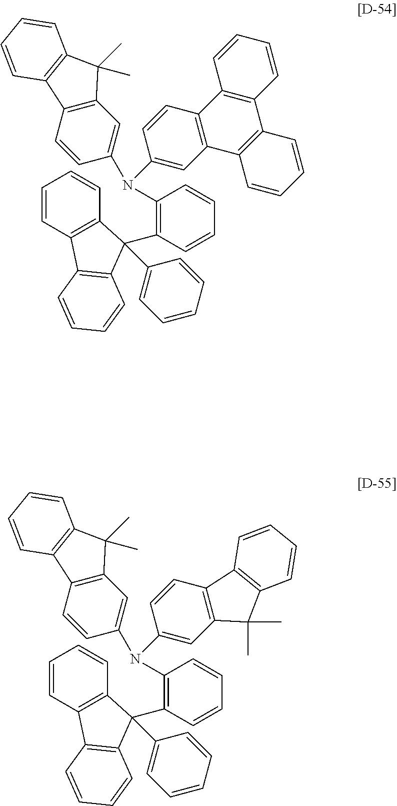 [SMILES: CC1(C)c2ccccc2-c2ccc(N(c3ccc4c5ccccc5c5ccccc5c4c3)c3ccccc3C3(c4ccccc4)c4ccccc4-c4ccccc43)cc21 ; CC1(C)c2ccccc2-c2ccc(N(c3ccc4c(c3)C(C)(C)c3ccccc3-4)c3ccccc3C3(c4ccccc4)c4ccccc4-c4ccccc43)cc21]

10. The organic optoelectronic device as claimed in claim 1, wherein:
the second compound is represented by Chemical Formula 2-8, and
the third compound is represented by Chemical Formula 5-2: 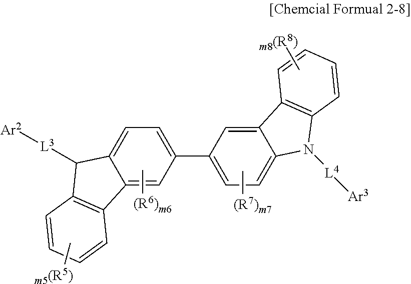
in Chemical Formula 2-8,
R⁵ to R⁸ are each independently hydrogen, deuterium, or a substituted or unsubstituted C6 to C12 aryl group,
m5 and m8 are each independently an integer of 1 to 4,
m6 and m7 are each independently an integer of 1 to 3, and
moieties *-L³-Ar² and *-L⁴-Ar³ are each independently a moiety of Group II, 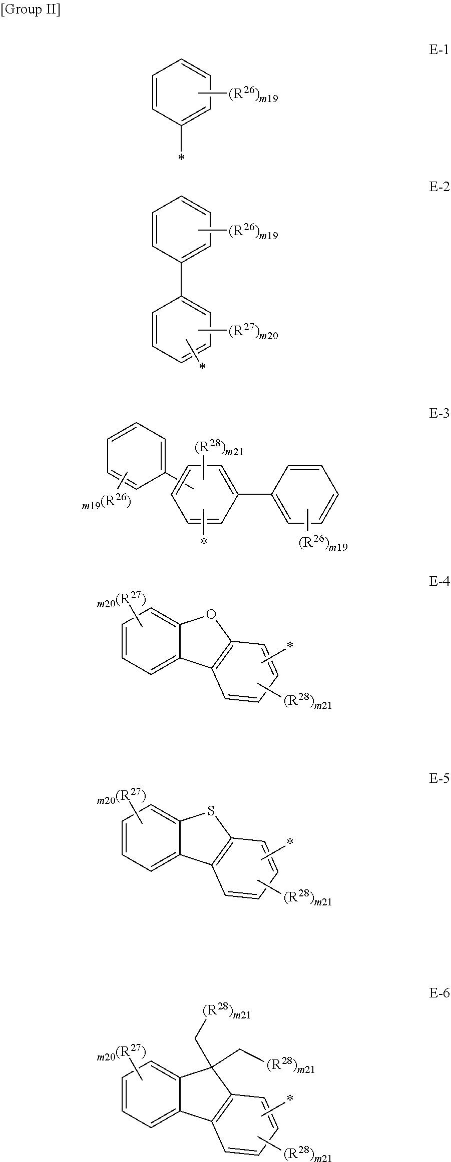 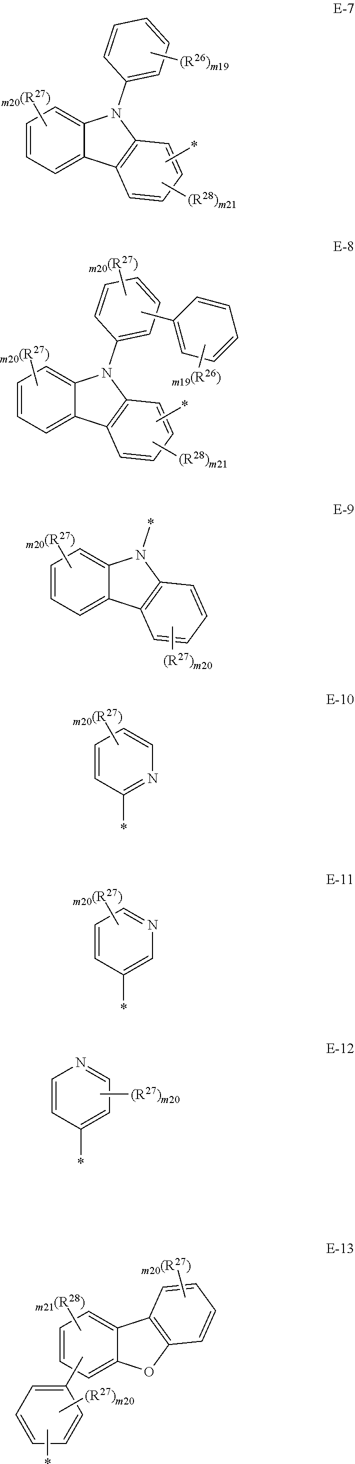 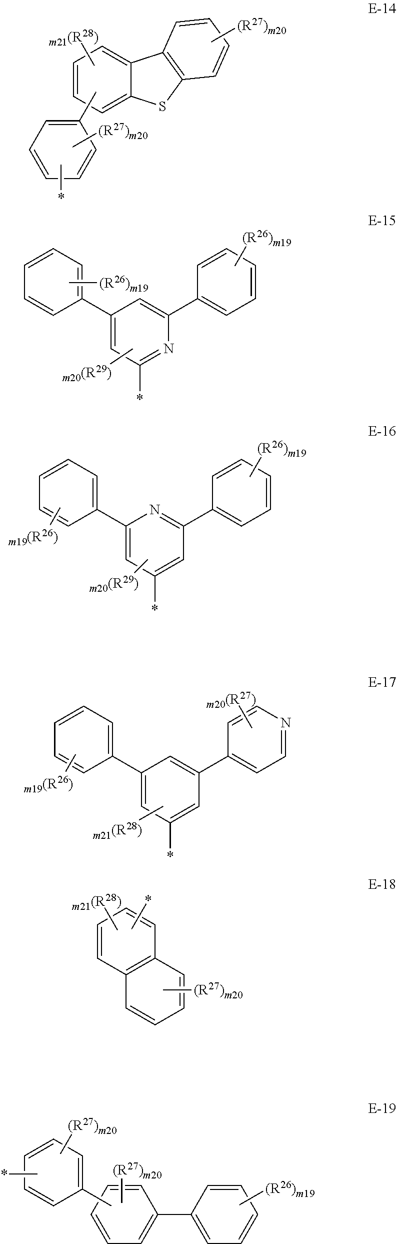 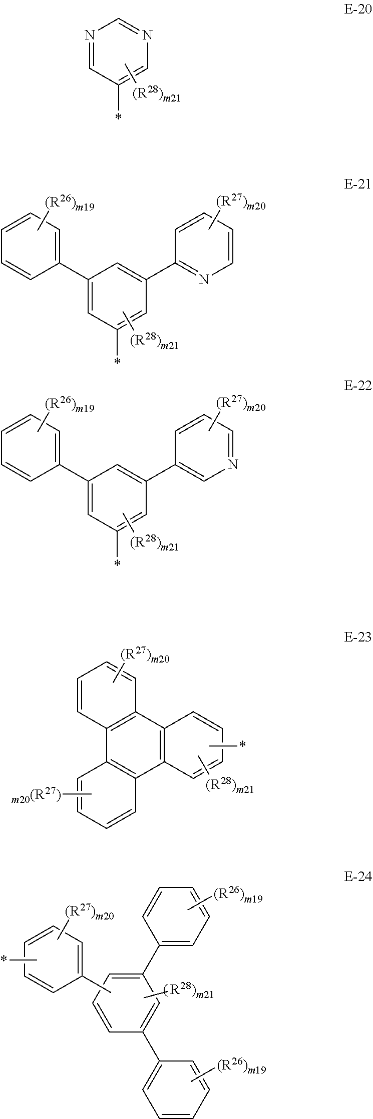 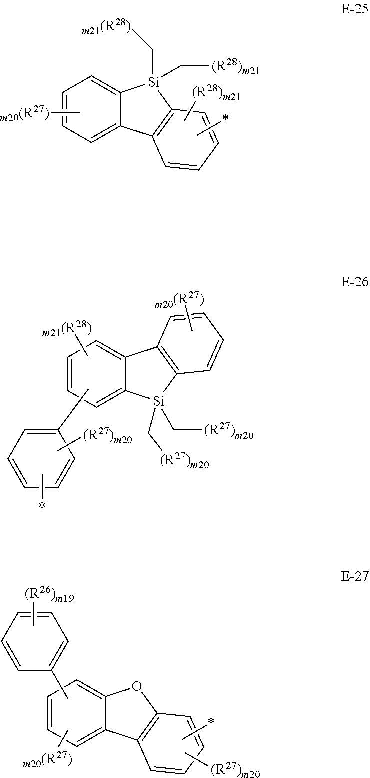 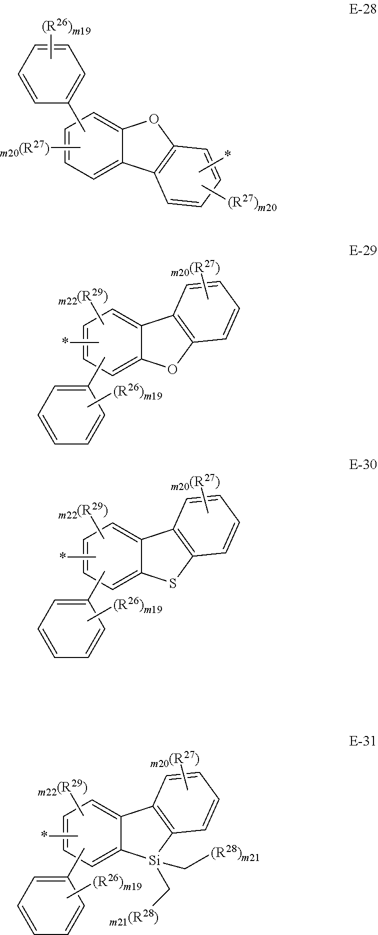
in Group II,
R²⁶ to R²⁹ are each independently hydrogen, deuterium, a cyano group, a substituted or unsubstituted C1 to C10 alkyl group, or a substituted or unsubstituted C6 to C12 aryl group,
m19 is an integer of 1 to 5,
m20 is an integer of 1 to 4,
m21 is an integer of 1 to 3,
m22 is 1 or 2, and
* is a linking point; 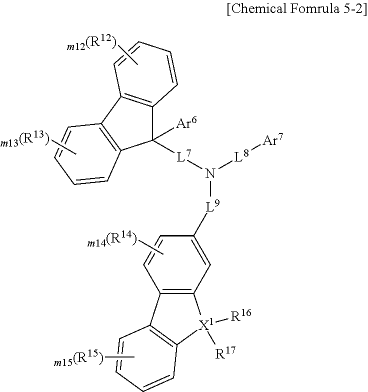
in Chemical Formula 5-2,
X¹ is C or Si,
R¹² to R¹⁵ are each independently hydrogen, deuterium, a cyano group, a substituted or unsubstituted C1 to C10 alkyl group, or a substituted or unsubstituted C6 to C30 aryl group,
R¹⁶ and R¹⁷ are each independently a substituted or unsubstituted C1 to C10 alkyl group, or a substituted or unsubstituted C6 to C20 aryl group,
Ar⁶ is a substituted or unsubstituted phenyl group, a substituted or unsubstituted biphenyl group, or a substituted or unsubstituted terphenyl group,
Ar⁷ is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, or a substituted or unsubstituted dibenzosilolyl group,
L⁷ to L⁹ are each independently a single bond or a substituted or unsubstituted C6 to C12 arylene group,
m12, m13, and m15 are each independently an integer of 1 to 4, and
m14 is an integer of 1 to 3.

11. A display device comprising the organic optoelectronic device as claimed in claim 1.
